(19) United States
(12) Patent Application Publication
(10) Pub. No.: US 2025/0311619 A1
(43) Pub. Date: Oct. 2, 2025

(54) ORGANIC COMPOUND, LIGHT-EMITTING DEVICE INCLUDING THE SAME, AND ELECTRONIC APPARATUS AND ELECTRONIC EQUIPMENT INCLUDING THE LIGHT-EMITTING DEVICE

(71) Applicant: Samsung Display Co., Ltd., Yongin-si (KR)

(72) Inventors: Minji Kim, Yongin-si (KR); Chaeyeong Kim, Yongin-si (KR); Bora Lee, Yongin-si (KR); Hanna Cha, Yongin-si (KR)

(21) Appl. No.: 19/088,497

(22) Filed: Mar. 24, 2025

(30) Foreign Application Priority Data
Apr. 2, 2024 (KR) 10-2024-0044789

(51) Int. Cl. H10K 85/60; C07C 211/61; C07D 307/91; C09K 11/06; H10K 50/12; H10K 50/15; H10K 85/30

(52) U.S. Cl. CPC H10K 85/633; C07C 211/61; C07D 307/91; C09K 11/06; H10K 85/346; H10K 85/658; C07C 2601/14; C07C 2603/18; C09K 2211/1018; H10K 50/121; H10K 50/156; H10K 85/615; H10K 85/626

(57) ABSTRACT

A light-emitting device includes a first electrode, a second electrode facing the first electrode, an interlayer between the first electrode and the second electrode and including an emission layer, and at least one organic compound represented by Formula 1 and satisfying at least one selected from among Condition 1 and Condition 2:

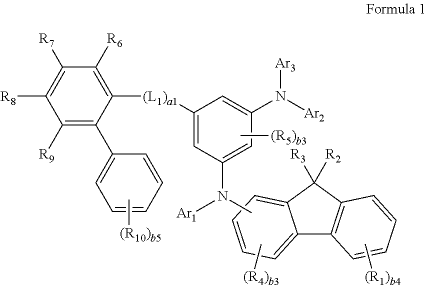

Condition 1
at least one selected from among R₆ to R₉ is a C₆-C₆₀ aryl group unsubstituted or substituted with at least one R₁₀ₐ or a C₁-C₆₀ heteroaryl group unsubstituted or substituted with at least one R₁₀ₐ
Condition 2
R₈ and R₉ are bonded to each other to form a C₃-C₆₀ carbocyclic group or a C₁-C₆₀ heterocyclic group.

### CROSS-REFERENCE TO RELATED APPLICATION

The present application claims priority to and the benefit of Korean Patent Application No. 10-2024-0044789, filed on Apr. 2, 2024, in the Korean Intellectual Property Office, the entire disclosure of which is incorporated herein by reference.

### BACKGROUND

### 1. Field

One or more embodiments of the present disclosure relate to an organic compound, a light-emitting device including the organic compound, and an electronic apparatus and electronic equipment each of which includes the light-emitting device.

### 2. Description of the Related Art

Among (various types or kinds of) light-emitting devices, self-emissive devices (for example, organic light-emitting devices) have relatively wide viewing angles, high contrast ratios, short (fast) response times, and excellent or suitable (desirable) characteristics in terms of luminance, driving voltage, and response speed.

A light-emitting device may include a first electrode, a hole transport region, an emission layer, an electron transport region, and a second electrode that are sequentially arranged in the stated order. Holes injected from the first electrode may move toward the emission layer through the hole transport region. Electrons injected from the second electrode may move toward the emission layer through the electron transport region. Carriers, such as the holes and the electrons, may recombine in the emission layer to produce excitons. As the excitons transition and decay from an excited state to a ground state, light may be generated.

### SUMMARY

One or more aspects of embodiments of the present disclosure are directed toward an organic compound that is a hole-transporting material for improving the driving voltage, luminescence efficiency, and lifespan of a light-emitting device emitting blue light, toward a light-emitting device having a low driving voltage, a high luminescence efficiency, and a long lifespan by including the organic compound, and toward an electronic apparatus and electronic equipment that have improved display quality by each including the light-emitting device. For example, one or more aspects of embodiments of the present disclosure are directed toward an organic compound that improves the driving voltage, brightness, and longevity of blue-light-emitting devices. Incorporating this compound results in devices with low power requirements, high efficiency, and extended lifespans, thereby improving the display quality of electronic devices.

Additional aspects will be set forth in part in the description which follows and, in part, will be apparent from the description, or may be learned by practice of the presented embodiments of the disclosure.

According to one or more embodiments of the present disclosure, a light-emitting device includes
a first electrode,
a second electrode opposite to (e.g., facing) the first electrode,
an interlayer between the first electrode and the second electrode and including an emission layer, and
at least one organic compound represented by Formula 1 and satisfying at least one selected from among Condition 1 and Condition 2:

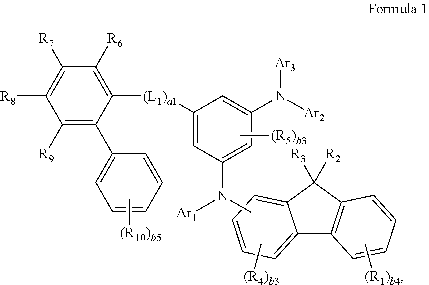

wherein, in Formula 1,
L₁ may be a C₃-C₆₀ carbocyclic group unsubstituted or substituted with at least one R₁₀ₐ or a C₁-C₆₀ heterocyclic group unsubstituted or substituted with at least one R₁₀ₐ,
a1 may be an integer from 0 to 2, wherein, if (e.g., when) a1 is 0, (L₁)ₐ₁ may be a single bond,
Ar₁ to Ar₃ and R₁ to R₁₀ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
b3 may be an integer from 0 to 3,
b4 may be an integer from 0 to 4,
b5 may be an integer from 0 to 5,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, -Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, -Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, a hydroxyl group, a cyano group, or a nitro group; or
a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof;

### Condition 1

at least one selected from among R₆ to R₉ is a C₆-C₆₀ aryl group unsubstituted or substituted with at least one R₁₀ₐ or a C₁-C₆₀ heteroaryl group unsubstituted or substituted with at least one R₁₀ₐ

### Condition 2

R₈ and R₉ are bonded to each other to form a C₃-C₆₀ carbocyclic group or a C₁-C₆₀ heterocyclic group.

According to one or more embodiments of the present disclosure, an electronic apparatus includes the light-emitting device and a thin-film transistor electrically connected to the light-emitting device.

According to one or more embodiments of the present disclosure, electronic equipment includes the light-emitting device,
wherein the electronic equipment may be at least one of a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality display, an augmented reality display, a vehicle, a video wall with multiple displays tiled together, a theater screen, a stadium screen, a phototherapy device, or a signboard.

According to one or more embodiments of the present disclosure, provided is an organic compound represented by Formula 1 and satisfying at least one selected from among Condition 1 and Condition 2.

### BRIEF DESCRIPTION OF DRAWINGS

The accompanying drawings are included to provide a further understanding of the present disclosure and are incorporated in and constitute a part of the present disclosure. The drawings illustrate embodiments of the present disclosure and, together with the description, serve to explain principles of the present disclosure. The above and other aspects, features, and advantages of certain embodiments of the disclosure will be more apparent from the following description taken in conjunction with the accompanying drawings, in which:

FIG. 1 is a schematic view of a light-emitting device according to one or more embodiments of the present disclosure;

FIG. 2 is a schematic view of an electronic apparatus according to one or more embodiments of the present disclosure;

FIG. 3 is a schematic view of an electronic apparatus according to one or more embodiments of the present disclosure;

FIG. 4 is a schematic perspective view of electronic equipment including a light-emitting device according to one or more embodiments of the present disclosure;

FIG. 5 is a schematic view of an exterior of a vehicle as electronic equipment including a light-emitting device according to one or more embodiments of the present disclosure; and

FIGS. 6A-6C are each a schematic view of an interior of the vehicle of FIG. 5.

### DETAILED DESCRIPTION

Reference will now be made in more detail to embodiments, examples of which are illustrated in the accompanying drawings, wherein like reference numerals refer to like elements throughout the present disclosure, and duplicative descriptions thereof may not be provided for conciseness. In this regard, the embodiments of the present disclosure may have different forms and should not be construed as being limited to the descriptions set forth herein. Accordingly, the embodiments of the present disclosure are merely described, by referring to the drawings, to explain aspects of the present disclosure. As used herein, the term “and/or” or “or” may include any and all combinations of one or more of the associated listed items. Throughout the disclosure, the expressions such as “at least one of,” “one of,” and “selected from,” when preceding a list of elements, modify the entire list of elements and do not modify the individual elements of the list. For example, “at least one of a, b, or c”, “at least one selected from a, b, and c”, “at least one selected from among a to c”, etc., may indicates only a, only b, only c, both (e.g., simultaneously) a and b, both (e.g., simultaneously) a and c, both (e.g., simultaneously) b and c, all of a, b, and c, or variations thereof. The “/” utilized herein may be interpreted as “and” or as “or” depending on the situation.

According to one or more embodiments of the present disclosure, a light-emitting device may include: a first electrode; a second electrode opposite to (e.g., facing) the first electrode; an interlayer between the first electrode and the second electrode and including an emission layer; and at least one organic compound represented by Formula 1 and satisfying at least one selected from among Condition 1 and Condition 2:

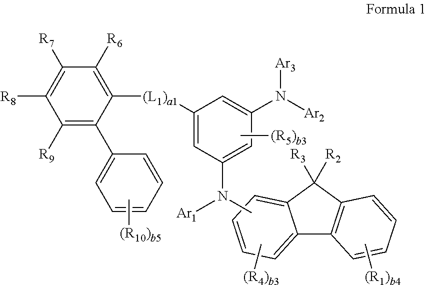

wherein, in Formula 1,
L₁ may be a C₃-C₆₀ carbocyclic group unsubstituted or substituted with at least one R₁₀ₐ or a C₁-C₆₀ heterocyclic group unsubstituted or substituted with at least one R₁₀ₐ,
a1 may be an integer from 0 to 2, wherein, if (e.g., when) a1 is 0, (L₁)ₐ₁ may be a single bond,
Ar₁ to Ar₃ and R₁ to R₁₀ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
b3 may be an integer from 0 to 3,
b4 may be an integer from 0 to 4,
b5 may be an integer from 0 to 5,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, a hydroxyl group, a cyano group, or a nitro group; or
a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof,

### Condition 1

at least one selected from among R₆ to R₉ is a C₆-C₆₀ aryl group unsubstituted or substituted with at least one R₁₀ₐ or a C₁-C₆₀ heteroaryl group unsubstituted or substituted with at least one R₁₀ₐ

### Condition 2

R₈ and R₉ are bonded to each other to form a C₃-C₆₀ carbocyclic group or a C₁-C₆₀ heterocyclic group.

The term “interlayer” as used herein refers to a single layer and/or all of multiple layers arranged between the first electrode and the second electrode of the light-emitting device.

In one or more embodiments, the interlayer may further include a hole transport region between the first electrode and the emission layer and an electron transport region between the emission layer and the second electrode. The hole transport region may include a hole injection layer on the first electrode, a hole transport layer on the hole injection layer, and an emission auxiliary layer on the hole transport layer.

In one or more embodiments, the interlayer may include the organic compound. For example, in one or more embodiments, the hole transport region may include the organic compound, for example, the hole transport layer may include the organic compound.

In one or more embodiments, the light-emitting device may further include a capping layer. For example, in one or more embodiments, the light-emitting device may further include at least one of a first capping layer arranged outside (e.g., on) the first electrode or a second capping layer arranged outside (e.g., on) the second electrode. The capping layer may include the organic compound, for example, at least one of the first capping layer and/or the second capping layer may include the organic compound.

In one or more embodiments, the light-emitting device may further include a first capping layer arranged outside (e.g., on) the first electrode, wherein the first capping layer may include the organic compound represented by Formula 1.

In one or more embodiments, the light-emitting device may further include a second capping layer arranged outside (e.g., on) the second electrode, wherein the second capping layer may include the organic compound represented by Formula 1.

In one or more embodiments, the light-emitting device may further include a first capping layer arranged outside (e.g., on) the first electrode and a second capping layer arranged outside (e.g., on) the second electrode, wherein at least one of the first capping layer and/or the second capping layer may include the organic compound represented by Formula 1.

The wording “(interlayer and/or capping layer) includes at least one organic compound represented by Formula 1” as used herein may be understood as “(interlayer and/or capping layer) may include one kind of organic compound represented by Formula 1 or two or more different kinds of organic compounds, each represented by Formula 1.”

In one or more embodiments, the interlayer and/or the capping layer may include only Compound 1, which will be described in more detail later, as the organic compound. Compound 1 may be present in the hole transport layer of the light-emitting device.

In one or more embodiments, the interlayer and/or the capping layer may include Compound 1 and Compound 2, which will be described in more detail later, as the organic compound. Compound 1 and Compound 2 may be present in substantially the same layer (for example, both (e.g., simultaneously) Compound 1 and Compound 2 may be present in the hole transport layer), or may be present in different layers (for example, Compound 1 may be present in the hole transport layer, and Compound 2 may be present in the capping layer, or for example, Compound 1 may be present in the hole transport layer, and Compound 2 may be present in the hole injection layer).

In one or more embodiments, the emission layer may be to emit blue light. The blue light may be deep blue light. For example, the blue light may have a maximum emission wavelength (e.g., wavelength of maximum emission peak) in a range of about 430 nanometers (nm) to about 475 nm, about 440 nm to about 475 nm, about 450 nm to about 475 nm, about 430 nm to about 470 nm, about 440 nm to about 470 nm, about 450 nm to about 470 nm, about 430 nm to about 465 nm, about 440 nm to about 465 nm, about 450 nm to about 465 nm, about 430 nm to about 460 nm, about 440 nm to about 460 nm, or about 450 nm to about 460 nm. The blue light may have a CIEx coordinate in a range of about 0.125 to about 0.140 or about 0.130 to about 0.140. The blue light may have a CIEy coordinate in a range of about 0.120 to about 0.210.

In one or more embodiments, the emission layer may include: an electron-transporting host including at least one π electron-deficient nitrogen-containing C₁-C₆₀ heterocyclic group; a hole-transporting host including a group represented by Formula 3; a sensitizer including a transition metal; a dopant including at least one cyclic group including both (e.g., simultaneously) boron and nitrogen as ring-forming atoms; or any combination thereof, wherein the organic compound, the electron-transporting host, the hole-transporting host, the sensitizer, and the dopant may be different from each other:

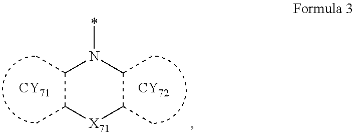

wherein, in Formula 3,
ring CY₇₁ and ring CY₇₂ may each independently be a π electron-rich C₃-C₆₀ cyclic group or a pyridine group,
X₇₁ may be a single bond, or a linking group including O, S, N, B, C, Si, or any combination thereof, and
* indicates a binding site to any atom included in the remaining portion of the hole-transporting host other than the group represented by Formula 3.

The electron-transporting host may be represented by Formula 2:

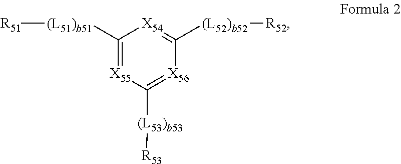

wherein, in Formula 2,
L₅₁ to L₅₃ may each independently be a single bond, a C₃-C₆₀ carbocyclic group unsubstituted or substituted with at least one R₁₀ₐ, or a C₁-C₆₀ heterocyclic group unsubstituted or substituted with at least one R₁₀ₐ,
b51 to b53 may each independently be an integer from 1 to 5,
X₅₄ may be N or C(R₅₄), X₅₅ may be N or C(R₅₅), X₅₆ may be N or C(R₅₆), and at least one selected from among X₅₄ to X₅₆ may be N,
R₅₁ to R₅₆ may each independently be the same as described with respect to R₁, and
R₁₀ₐ may be the same as described herein.

In one or more embodiments, the hole-transporting host may be represented by any one selected from among Formulae 3-1 to 3-5:

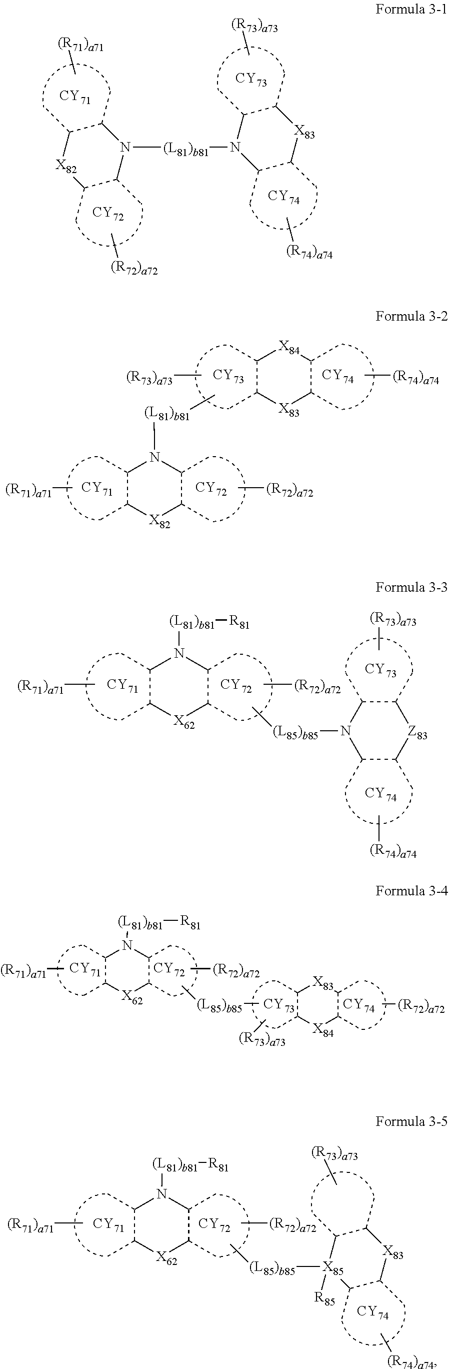

wherein, in Formulae 3-1 to 3-5,
ring CY₁ to ring CY₇₄ may each independently be a π electron-rich C₃-C₆₀ cyclic group or a pyridine group,
X₈₂ may be a single bond, O, S, N[(L₈₂)_{b82}-R₈₂], B(R₈₂), C(R₈₂ₐ)(R_{82b}), or Si(R₈₂ₐ)(R_{82b}),
X₈₃ may be a single bond, O, S, N[(L₈₃)_{b83}-R₈₃], B(R₈₃), C(R₈₃ₐ)(R_{83b}), or Si(R₈₃ₐ)(R_{83b}),
X₈₄ may be O, S, N[(L₈₄)_{b84}-R₈₄], B(R₈₄), C(R₈₄ₐ)(R_{84b}), or Si(R₈₄ₐ)(R_{84b}),
X₈₅ may be C or Si,
L₈₁ to L₈₅ may each independently be a single bond, *—C(Q₄)(Q₅)-*′, *—Si(Q₄)(Q₅)-*′, a π electron-rich C₃-C₆₀ cyclic group unsubstituted or substituted with at least one R₁₀ₐ, or a pyridine group unsubstituted or substituted with at least one R₁₀ₐ, wherein Q₄ and Q₅ may each independently be the same as described with respect to Q₁,
b81 to b85 may each independently be an integer from 1 to 5,
R₇₁ to R₇₄, R₈₁ to R₈₅, R₈₂ₐ, R_{82b}, R₈₃ₐ, R_{83b}, R₈₄ₐ, and R_{84b} may each independently be the same as described with respect to R₁,
a71 to a74 may each independently be an integer from 0 to 20, and
R₁₀ₐ may be the same as described herein.

In one or more embodiments, the sensitizer may be a compound including a transition metal and a ligand. The transition metal may be platinum (Pt), palladium (Pd), gold (Au), silver (Ag), nickel (Ni), or copper (Cu), and the ligand may include a monodentate ligand, a bidentate ligand, a tridentate ligand, a tetradentate ligand, or any combination thereof. For example, in one or more embodiments, the sensitizer may be represented by Formula 4:

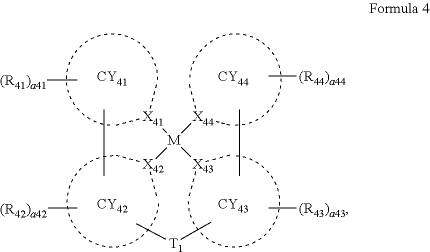

wherein, in Formula 4,
M may be platinum (Pt), palladium (Pd), gold (Au), silver (Ag), nickel (Ni), or copper(Cu),
X₄₁ to X₄₄ may each be carbon (C) or nitrogen (N),
T₁ may be a single bond, O, N(Z₁), or C(Z₁)(Z₂),
ring CY₄₁ to ring CY₄₄ may each independently be a C₃-C₆₀ carbocyclic group or a C₁-C₆₀ heterocyclic group,
Z₁, Z₂, and R₄₁ to R₄₄ may each independently be hydrogen, deuterium, —F, -Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a41 to a44 may each independently be an integer from 0 to 20, and
R₁₀ₐ and Q₁ to Q₃ may each be the same as described herein.

In one or more embodiments, the dopant may be a C₈-C₆₀ polycyclic group-containing compound including two or more cyclic groups that are condensed while sharing boron (B). In one or more embodiments, the dopant may include a condensed ring in which at least one third ring is condensed with at least one fourth ring, for example, to form the condensed ring including four or more rings,
wherein the third ring of the dopant may be a cyclopentane group, a cyclohexane group, a cycloheptane group, a cyclooctane group, a cyclopentene group, a cyclohexene group, a cycloheptene group, a cyclooctene group, an adamantane group, a norbornene group, a norbornane group, a bicyclo[1.1.1]pentane group, a bicyclo[2.1.1]hexane group, a bicyclo[2.2.2]octane group, a benzene group, a pyridine group, a pyrimidine group, a pyridazine group, a pyrazine group, or a triazine group, and
the fourth ring of the dopant may be a 1,2-azaborinine group, a 1,3-azaborinine group, a 1,4-azaborinine group, a 1,2-dihydro-1,2-azaborinine group, a 1,4-oxaborinine group, a 1,4-thiaborinine group, or a 1,4-dihydroborinine group.

In one or more embodiments, the dopant may include a compound represented by Formula 5-1, a compound represented by Formula 5-2, or any combination thereof:

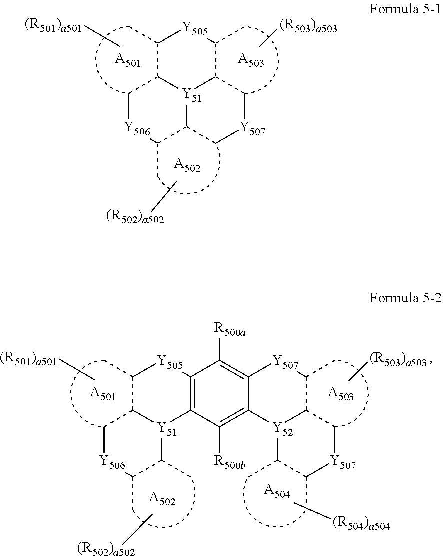

wherein, in Formulae 5-1 and 5-2,
ring A₅₀₁ to ring A₅₀₄ may each independently be a C₃-C₆₀ carbocyclic group or a C₁-C₆₀ heterocyclic group,
Y₅₀₅ may be O, S, N(R₅₀₅), B(R₅₀₅), C(R₅₀₅ₐ)(R_{505b}), or Si(R₅₀₅ₐ)(R_{505b}),
Y₅₀₆ may be O, S, N(R₅₀₆), B(R₅₀₆), C(R₅₀₆ₐ)(R_{506b}), or Si(R₅₀₆ₐ)(R_{506b}),
Y₅₀₇ may be O, S, N(R₅₀₇), B(R₅₀₇), C(R₅₀₇ₐ)(R_{507b}), or Si(R₅₀₇ₐ)(R_{507b}),
Y₅₀₈ may be O, S, N(R₅₀₈), B(R₅₀₈), C(R₅₀₈ₐ)(R_{508b}), or Si(R₅₀₈ₐ)(R_{508b}),
Y₅₁ and Y₅₂ may each independently be B, P(═O), or S(═O),
R₅₀₀ₐ, R_{500b}, R₅₀₁ to R₅₀₈, R₅₀₅ₐ, R_{505b}, R₅₀₆ₐ, R_{506b}, R₅₀₇ₐ, R_{507b}, R₅₀₈ₐ, and R_{508b} may each independently be the same as described with respect to R₁, and
a501 to a504 may each independently be an integer from 0 to 20.

According to one or more embodiments of the present disclosure, an electronic apparatus may include: the light-emitting device; and a thin-film transistor electrically connected to the light-emitting device. For example, the thin-film transistor may include a source electrode and a drain electrode, and the first electrode of the light-emitting device may be electrically connected to the source electrode or the drain electrode. In one or more embodiments, the electronic apparatus may further include a color filter, a color conversion layer, a touch screen layer, a polarizing layer, or any combination thereof. More details on the electronic apparatus may be the same as described herein.

According to one or more embodiments of the present disclosure, electronic equipment may include the light-emitting device, wherein the electronic equipment may be at least one of a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality display, an augmented reality display, a vehicle, a video wall with multiple displays tiled together, a theater screen, a stadium screen, a phototherapy device, or a signboard.

According to one or more embodiments of the present disclosure, an organic compound is represented by Formula 1 and satisfies at least one selected from among Condition 1 and Condition 2.

### Description of Formula 1

The organic compound may be represented by Formula 1 and may satisfy at least one selected from among Condition 1 and Condition 2:

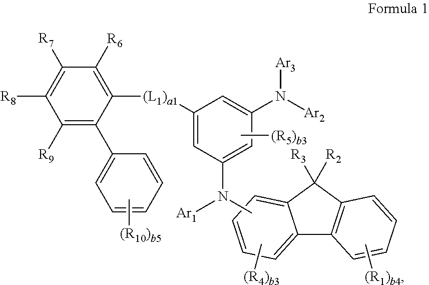

wherein, in Formula 1,
L₁ may be a C₃-C₆₀ carbocyclic group unsubstituted or substituted with at least one R₁₀ₐ or a C₁-C₆₀ heterocyclic group unsubstituted or substituted with at least one R₁₀ₐ,
a1 may be an integer from 0 to 2, wherein, if (e.g., when) a1 is 0, (L₁)ₐ₁ may be a single bond,
Ar₁ to Ar₃ and R₁ to R₁₀ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
b3 may be an integer from 0 to 3,
b4 may be an integer from 0 to 4,
b5 may be an integer from 0 to 5,
R₁₀ₐ maybe:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, a hydroxyl group, a cyano group, or a nitro group; or
a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof,

### Condition 1

at least one selected from among R₆ to R₉ is a C₆-C₆₀ aryl group unsubstituted or substituted with at least one R₁₀ₐ or a C₁-C₆₀ heteroaryl group unsubstituted or substituted with at least one R₁₀ₐ

### Condition 2

R₈ and R₉ are bonded to each other to form a C₃-C₆₀ carbocyclic group or a C₁-C₆₀ heterocyclic group.

In one or more embodiments, the organic compound may be represented by Formula 1-1:

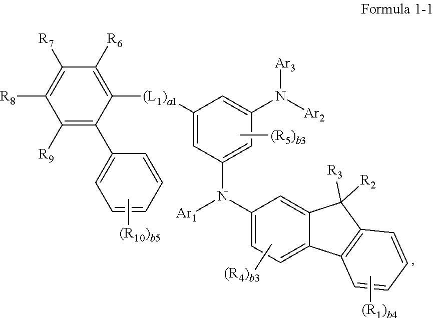

wherein, in Formula 1-1, L₁, a1, Ar₁ to Ar₃, R₁ to R₁₀, and b3 to b5 may each be the same as described in Formula 1.

In one or more embodiments, in Formulae 1 and 1-1, L₁ may be a phenylene group; or a1 may be 0. For example, L₁ may be an ortho-phenylene group, a meta-phenylene group, or a para-phenylene group, or (L₁)ₐ₁ may be a single bond.

In one or more embodiments, Ar₁ to Ar₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ. In one or more embodiments, Ar₁ to Ar₃ may each independently be a C₆-C₆₀ aryl group unsubstituted or substituted with at least one R₁₀ₐ, a C₁-C₆₀ heteroaryl group unsubstituted or substituted with at least one R₁₀ₐ, a monovalent non-aromatic condensed polycyclic group unsubstituted or substituted with at least one R₁₀ₐ, or a monovalent non-aromatic condensed heteropolycyclic group unsubstituted or substituted with at least one R₁₀ₐ. For example, in one or more embodiments, Ar₁ to Ar₃ may each independently be a phenyl group, a naphthyl group, a fluorenyl group, a dibenzofuranyl group, or a dibenzothiophenyl group, each substituted with a C₁-C₁₀ alkyl group, a C₁-C₁₀ cycloalkyl group, a phenyl group, a naphthyl group, or any combination thereof.

In one or more embodiments, R₁ to R₅ and R₁₀ may each independently be hydrogen, deuterium, a C₁-C₆₀ alkyl group unsubstituted or substituted with at least one R₁₀ₐ, a C₆-C₆₀ aryl group unsubstituted or substituted with at least one R₁₀ₐ, or a C₁-C₆₀ heteroaryl group unsubstituted or substituted with at least one R₁₀ₐ. For example, in one or more embodiments, R₁ to R₅ and R₁₀ may each independently be hydrogen, deuterium, a phenyl group, or a naphthyl group.

In one or more embodiments, the organic compound i) may satisfy Condition and may not satisfy Condition 2, ii) may not satisfy Condition 1 and may satisfy Condition 2, or iii) may satisfy both (e.g., simultaneously) Condition 1 and Condition 2.

According to Condition 1, in Formulae 1 and 1-1, a moiety represented by

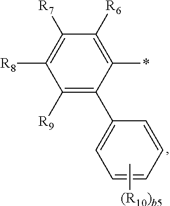

to which a core represented by

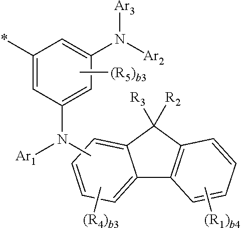

is linked via a linking group (L₁)ₐ₁, may be a moiety in which at least three rings are bonded to each other, and may not be a biphenyl group unsubstituted or substituted with any substituent. In the moiety, with respect to the linking group (L₁)ₐ₁ and *, which indicates a binding site, i) a phenyl group may be located at an ortho position, and ii) at least one of R₆ (at an ortho position with respect to *), R₇ (at a meta position with respect to *), R₈ (at a para position with respect to *), or R₉ (at a meta position with respect to *) may be a C₆-C₆₀ aryl group unsubstituted or substituted with at least one R₁₀ₐ or a C₁-C₆₀ heteroaryl group unsubstituted or substituted with at least one R₁₀ₐ.

In one or more embodiments, in Condition 1, R₆ may be a C₆-C₆₀ aryl group unsubstituted or substituted with at least one R₁₀ₐ or a C₁-C₆₀ heteroaryl group unsubstituted or substituted with at least one R₁₀ₐ, and R₇ to R₉ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

In one or more embodiments, in Condition 1, R₇ may be a C₆-C₆₀ aryl group unsubstituted or substituted with at least one R₁₀ₐ or a C₁-C₆₀ heteroaryl group unsubstituted or substituted with at least one R₁₀ₐ, and R₆, R₈, and R₉ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

In one or more embodiments, in Condition 1, at least one selected from among R₆ to R₉ may be a phenyl group unsubstituted or substituted with at least one R₁₀ₐ or a naphthyl group unsubstituted or substituted with at least one R₁₀ₐ.

In one or more embodiments, in Condition 1, R₆ may be a phenyl group unsubstituted or substituted with at least one R₁₀ₐ or a naphthyl group unsubstituted or substituted with at least one R₁₀ₐ, and R₇ to R₉ may each independently be hydrogen or deuterium.

In one or more embodiments, in Condition 1, R₇ may be a phenyl group unsubstituted or substituted with at least one R₁₀ₐ or a naphthyl group unsubstituted or substituted with at least one R₁₀ₐ, and R₆, R₈, and R₉ may each independently be hydrogen or deuterium.

According to Condition 2, in Formulae 1 and 1-1, in the moiety represented by

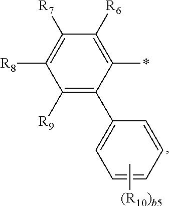

to which the core represented by

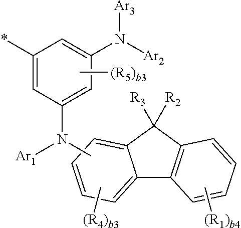

is linked via the linking group (L₁)ₐ₁, R₈ and R₉ may be bonded to each other to form a condensed ring.

In one or more embodiments, in Condition 2, R₈ and R₉ may be bonded to each other to form a benzene group or a naphthalene group.

In one or more embodiments, the moiety may be represented by

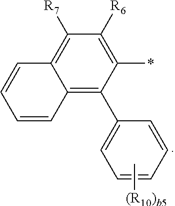

The moiety may be bonded to a phenyl group unsubstituted or substituted with R₁₀ at a number 1 carbon atom of naphthalene, and may be bonded to the linking group (L₁)ₐ₁ at a number 2 carbon atom of naphthalene (see *). Accordingly, it is clear that compounds wherein, in Formulae 1 and 1-1, R₆ and R₇ are bonded to each other to form a C₃-C₆₀ carbocyclic group or a C₁-C₆₀ heterocyclic group (for example, a moiety that is represented by

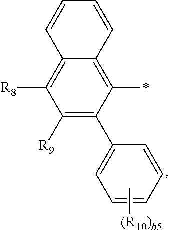

bonded to the linking group (L₁)ₐ₁ at the number 1 carbon atom of naphthalene, and bonded to a phenyl group unsubstituted or substituted with R₁₀ at the number 2 carbon atom of naphthalene), or R₇ and R₈ are bonded to each other to form a C₃-C₆₀ carbocyclic group or a C₁-C₆₀ heterocyclic group (for example, a moiety that is represented by

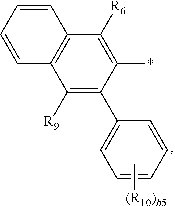

bonded to the linking group (L₁)ₐ₁ at the number 2 carbon atom of naphthalene, and bonded to a phenyl group unsubstituted or substituted with R₁₀ at a number 3 carbon atom of naphthalene) do not satisfy Condition 2.

In the organic compound satisfying Condition 1, in Formulae 1 and 1-1, the moiety represented by

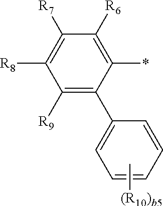

may be a moiety represented by

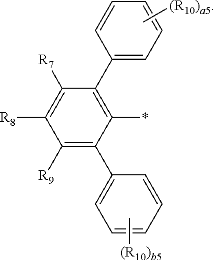

In the organic compound satisfying Condition 2, in Formulae 1 and 1-1, the moiety represented by

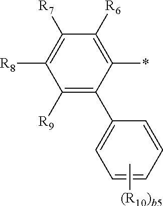

may be a moiety represented by

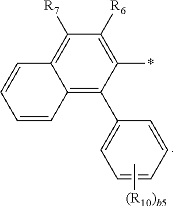

In the organic compound satisfying both (e.g., simultaneously) Condition 1 and Condition 2, in Formulae 1 and 1-1, the moiety represented by

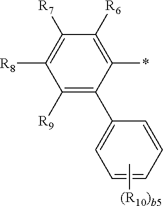

may be a moiety represented by

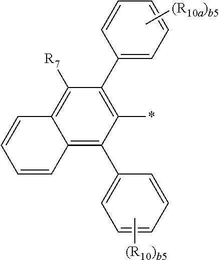

or a moiety represented by

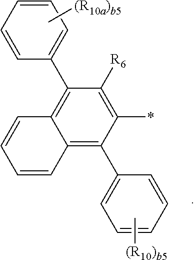

### Examples of Organic Compounds

In one or more embodiments, the organic compound may be any one of (e.g., selected from among) Compounds 1 to 208:

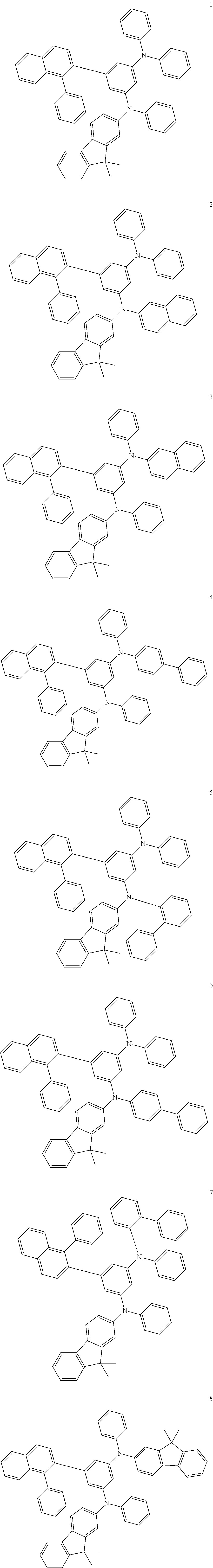

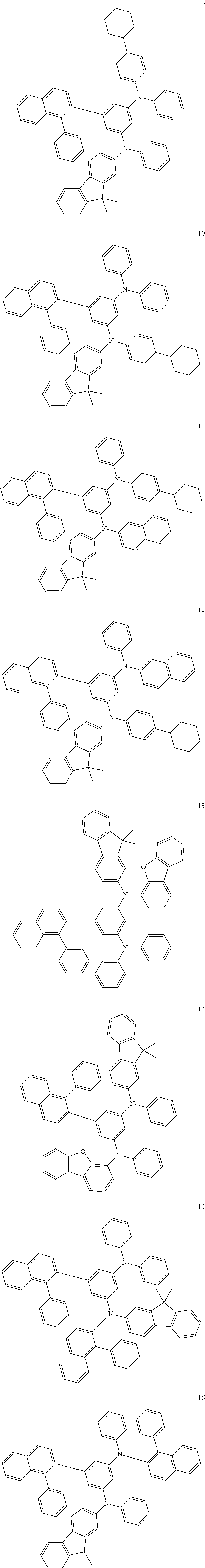

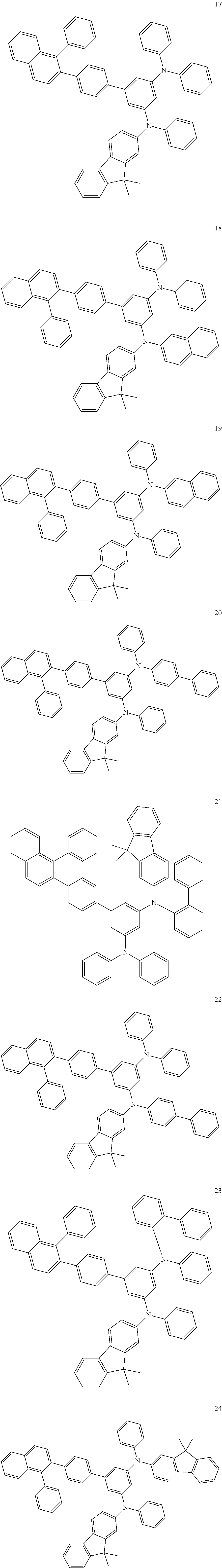

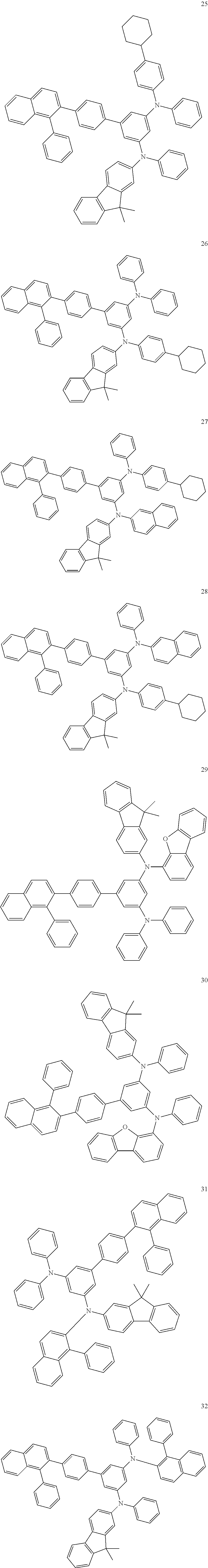

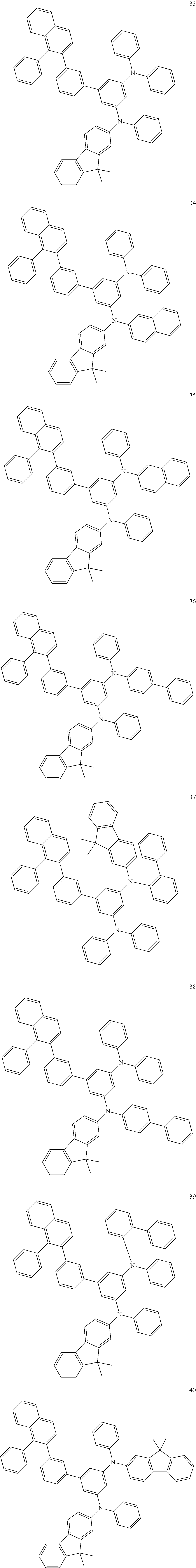

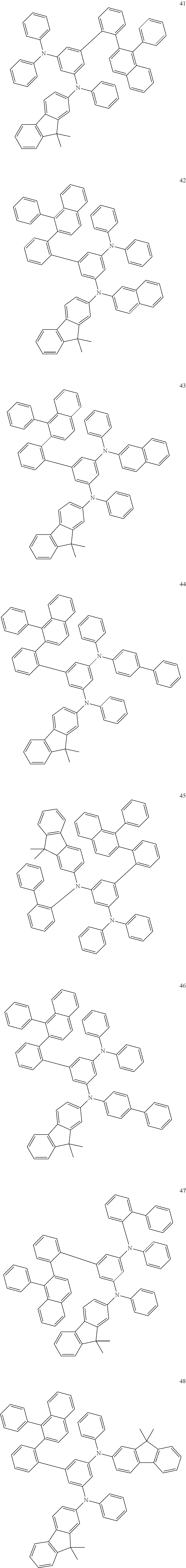

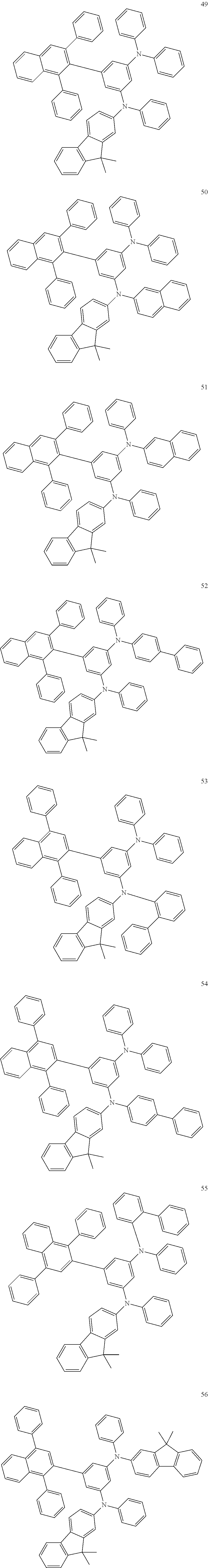

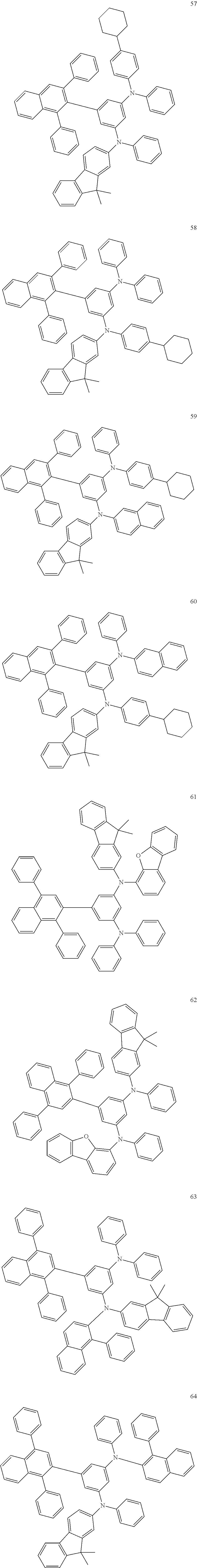

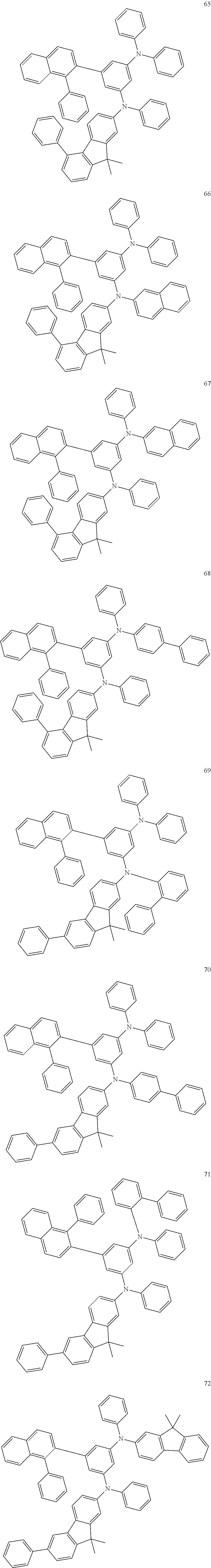

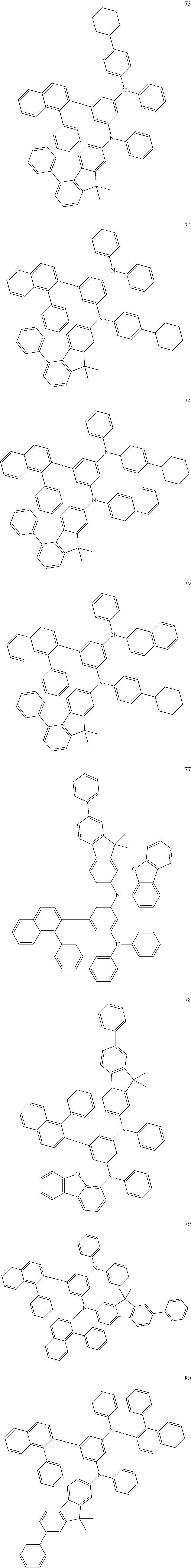

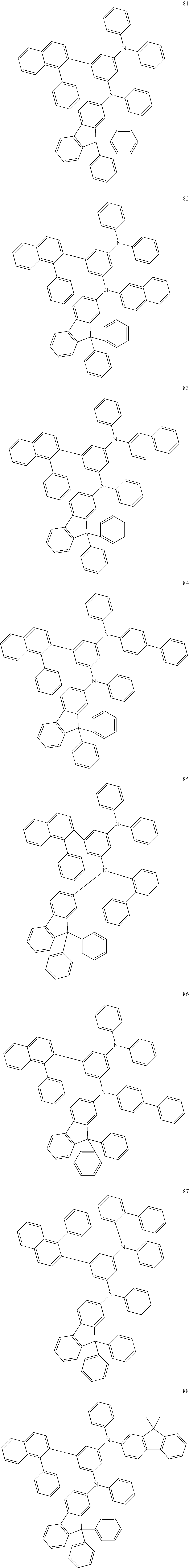

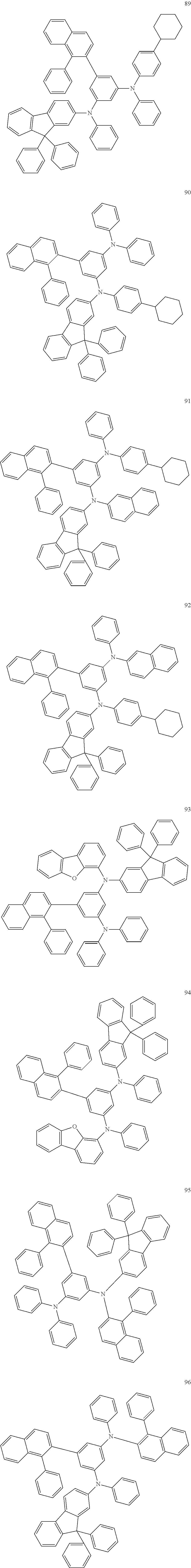

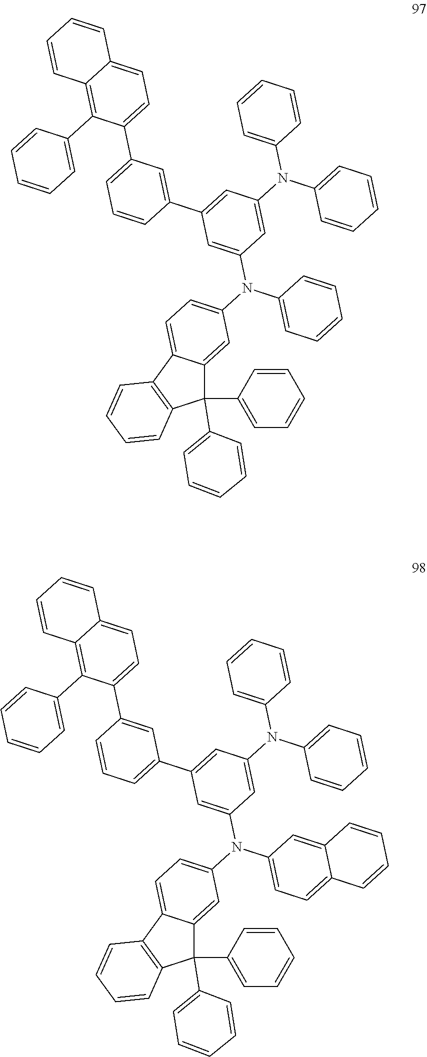 [SMILES: c1ccc(-c2c(-c3cccc(-c4cc(N(c5ccccc5)c5ccccc5)cc(N(c5ccccc5)c5ccc6c(c5)C(c5ccccc5)(c5ccccc5)c5ccccc5-6)c4)c3)ccc3ccccc23)cc1 ; c1ccc(-c2c(-c3cccc(-c4cc(N(c5ccccc5)c5ccccc5)cc(N(c5ccc6c(c5)C(c5ccccc5)(c5ccccc5)c5ccccc5-6)c5ccc6ccccc6c5)c4)c3)ccc3ccccc23)cc1]

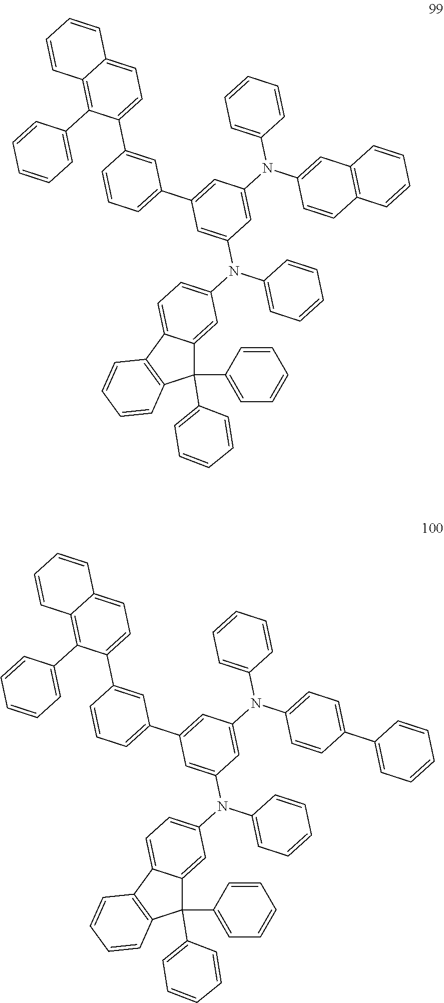 [SMILES: c1ccc(-c2c(-c3cccc(-c4cc(N(c5ccccc5)c5ccc6c(c5)C(c5ccccc5)(c5ccccc5)c5ccccc5-6)cc(N(c5ccccc5)c5ccc6ccccc6c5)c4)c3)ccc3ccccc23)cc1 ; c1ccc(-c2ccc(N(c3ccccc3)c3cc(-c4cccc(-c5ccc6ccccc6c5-c5ccccc5)c4)cc(N(c4ccccc4)c4ccc5c(c4)C(c4ccccc4)(c4ccccc4)c4ccccc4-5)c3)cc2)cc1]

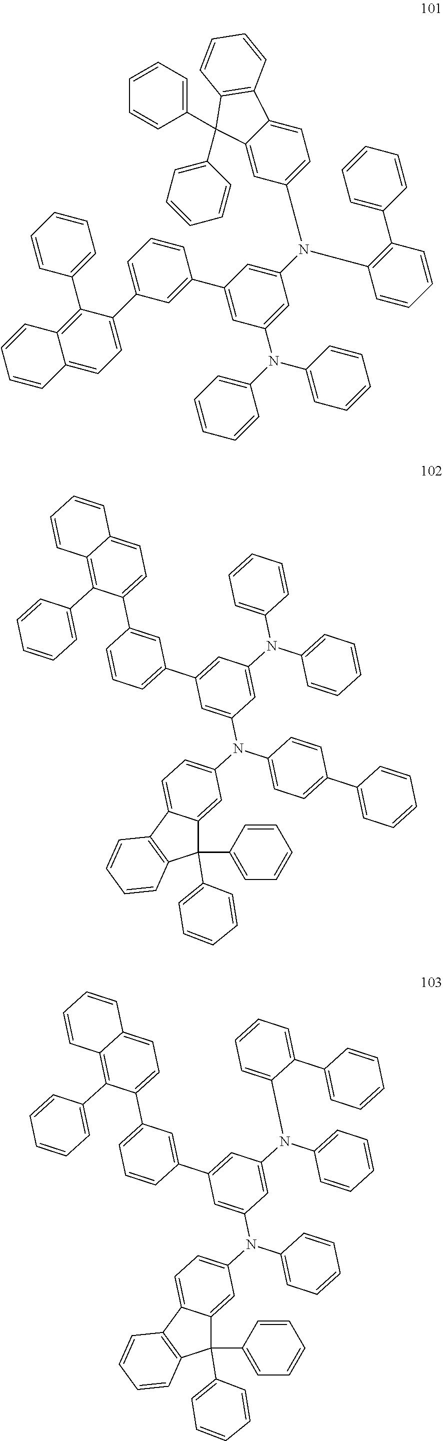 [SMILES: c1ccc(-c2ccccc2N(c2cc(-c3cccc(-c4ccc5ccccc5c4-c4ccccc4)c3)cc(N(c3ccccc3)c3ccccc3)c2)c2ccc3c(c2)C(c2ccccc2)(c2ccccc2)c2ccccc2-3)cc1 ; c1ccc(-c2ccc(N(c3cc(-c4cccc(-c5ccc6ccccc6c5-c5ccccc5)c4)cc(N(c4ccccc4)c4ccccc4)c3)c3ccc4c(c3)C(c3ccccc3)(c3ccccc3)c3ccccc3-4)cc2)cc1 ; c1ccc(-c2ccccc2N(c2ccccc2)c2cc(-c3cccc(-c4ccc5ccccc5c4-c4ccccc4)c3)cc(N(c3ccccc3)c3ccc4c(c3)C(c3ccccc3)(c3ccccc3)c3ccccc3-4)c2)cc1]

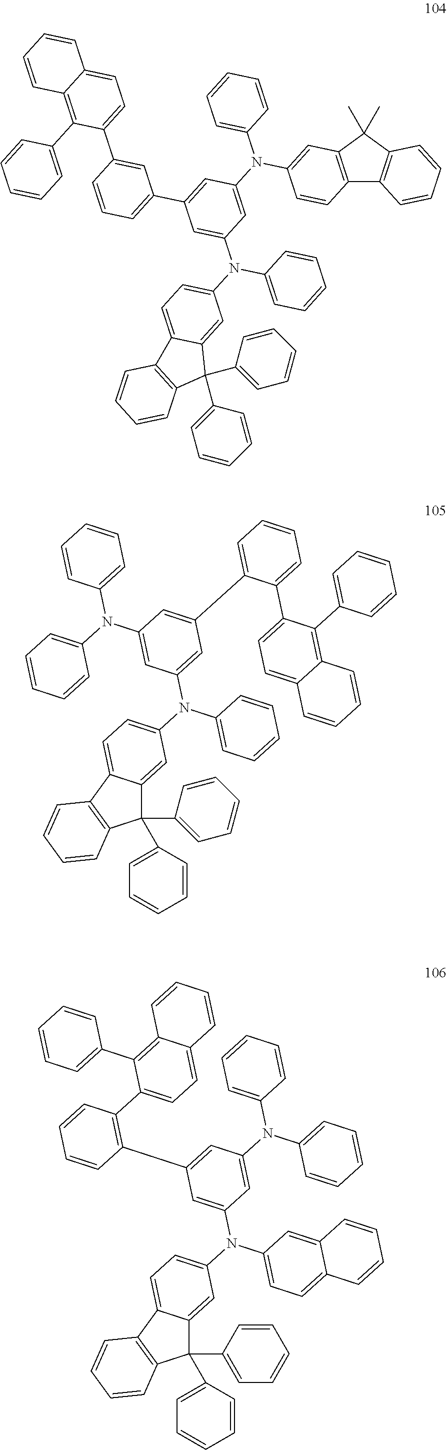 [SMILES: CC1(C)c2ccccc2-c2ccc(N(c3ccccc3)c3cc(-c4cccc(-c5ccc6ccccc6c5-c5ccccc5)c4)cc(N(c4ccccc4)c4ccc5c(c4)C(c4ccccc4)(c4ccccc4)c4ccccc4-5)c3)cc21 ; c1ccc(-c2c(-c3ccccc3-c3cc(N(c4ccccc4)c4ccccc4)cc(N(c4ccccc4)c4ccc5c(c4)C(c4ccccc4)(c4ccccc4)c4ccccc4-5)c3)ccc3ccccc23)cc1 ; c1ccc(-c2c(-c3ccccc3-c3cc(N(c4ccccc4)c4ccccc4)cc(N(c4ccc5c(c4)C(c4ccccc4)(c4ccccc4)c4ccccc4-5)c4ccc5ccccc5c4)c3)ccc3ccccc23)cc1]

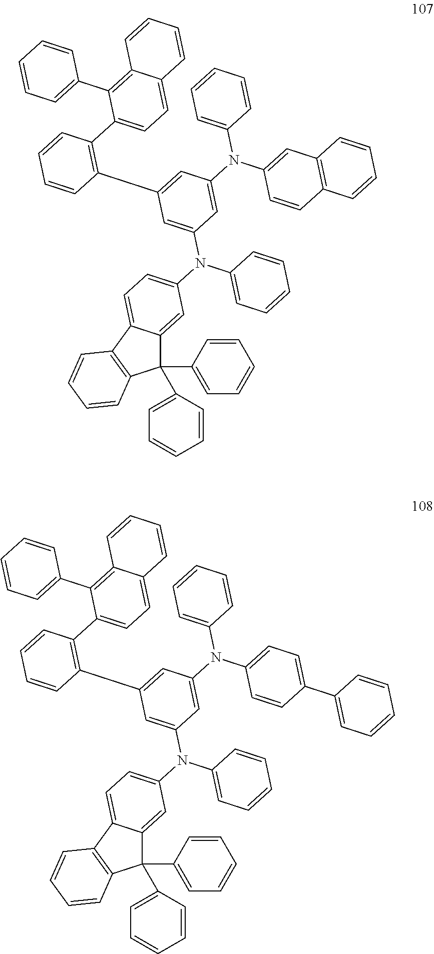 [SMILES: c1ccc(-c2c(-c3ccccc3-c3cc(N(c4ccccc4)c4ccc5c(c4)C(c4ccccc4)(c4ccccc4)c4ccccc4-5)cc(N(c4ccccc4)c4ccc5ccccc5c4)c3)ccc3ccccc23)cc1 ; c1ccc(-c2ccc(N(c3ccccc3)c3cc(-c4ccccc4-c4ccc5ccccc5c4-c4ccccc4)cc(N(c4ccccc4)c4ccc5c(c4)C(c4ccccc4)(c4ccccc4)c4ccccc4-5)c3)cc2)cc1]

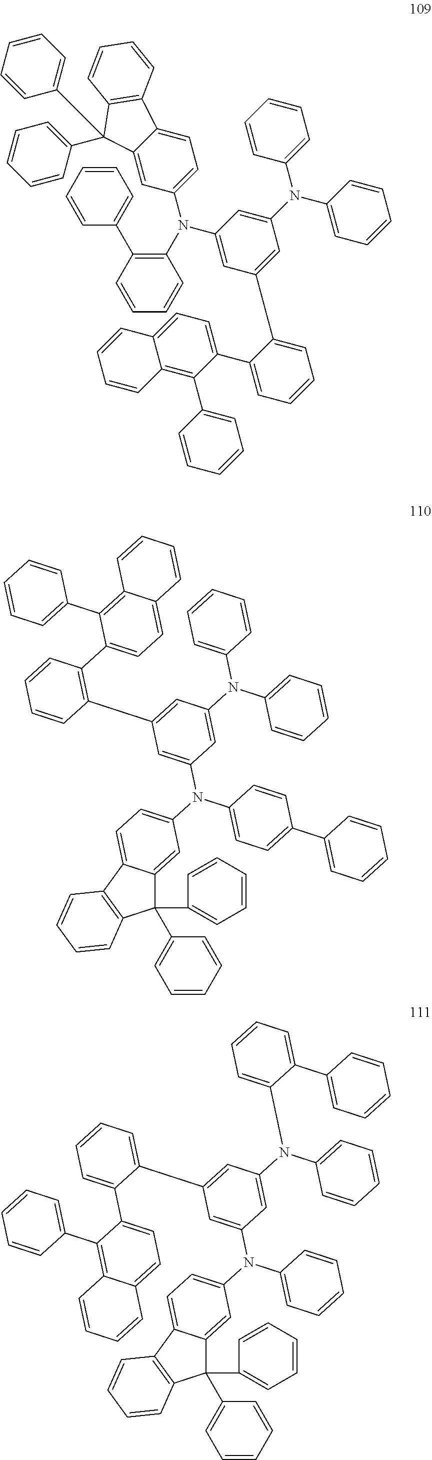 [SMILES: c1ccc(-c2ccccc2N(c2cc(-c3ccccc3-c3ccc4ccccc4c3-c3ccccc3)cc(N(c3ccccc3)c3ccccc3)c2)c2ccc3c(c2)C(c2ccccc2)(c2ccccc2)c2ccccc2-3)cc1 ; c1ccc(-c2ccc(N(c3cc(-c4ccccc4-c4ccc5ccccc5c4-c4ccccc4)cc(N(c4ccccc4)c4ccccc4)c3)c3ccc4c(c3)C(c3ccccc3)(c3ccccc3)c3ccccc3-4)cc2)cc1 ; c1ccc(-c2ccccc2N(c2ccccc2)c2cc(-c3ccccc3-c3ccc4ccccc4c3-c3ccccc3)cc(N(c3ccccc3)c3ccc4c(c3)C(c3ccccc3)(c3ccccc3)c3ccccc3-4)c2)cc1]

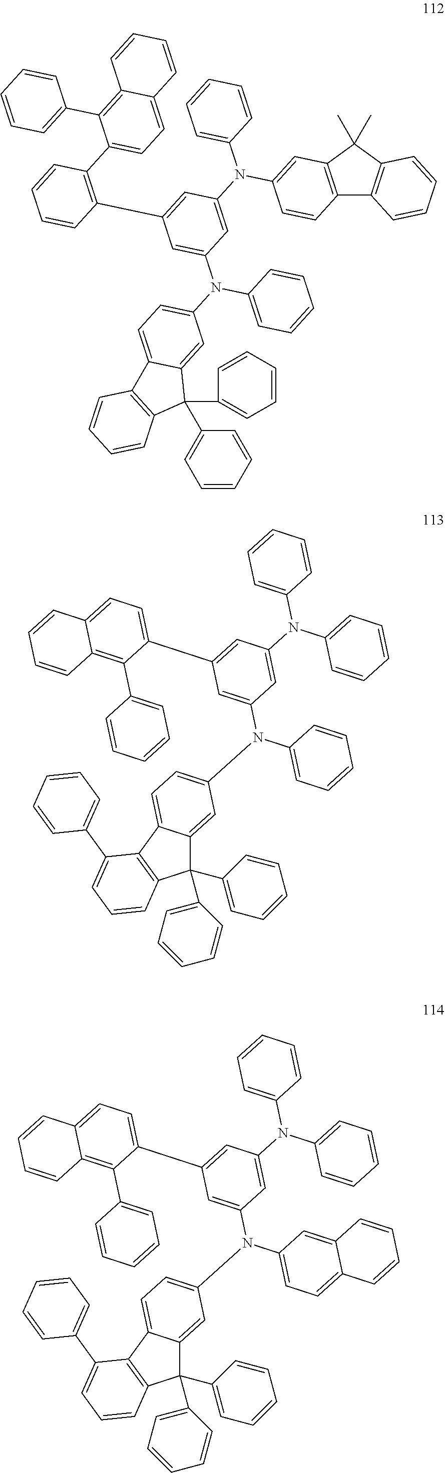 [SMILES: CC1(C)c2ccccc2-c2ccc(N(c3ccccc3)c3cc(-c4ccccc4-c4ccc5ccccc5c4-c4ccccc4)cc(N(c4ccccc4)c4ccc5c(c4)C(c4ccccc4)(c4ccccc4)c4ccccc4-5)c3)cc21 ; c1ccc(-c2cccc3c2-c2ccc(N(c4ccccc4)c4cc(-c5ccc6ccccc6c5-c5ccccc5)cc(N(c5ccccc5)c5ccccc5)c4)cc2C3(c2ccccc2)c2ccccc2)cc1 ; c1ccc(-c2cccc3c2-c2ccc(N(c4cc(-c5ccc6ccccc6c5-c5ccccc5)cc(N(c5ccccc5)c5ccccc5)c4)c4ccc5ccccc5c4)cc2C3(c2ccccc2)c2ccccc2)cc1]

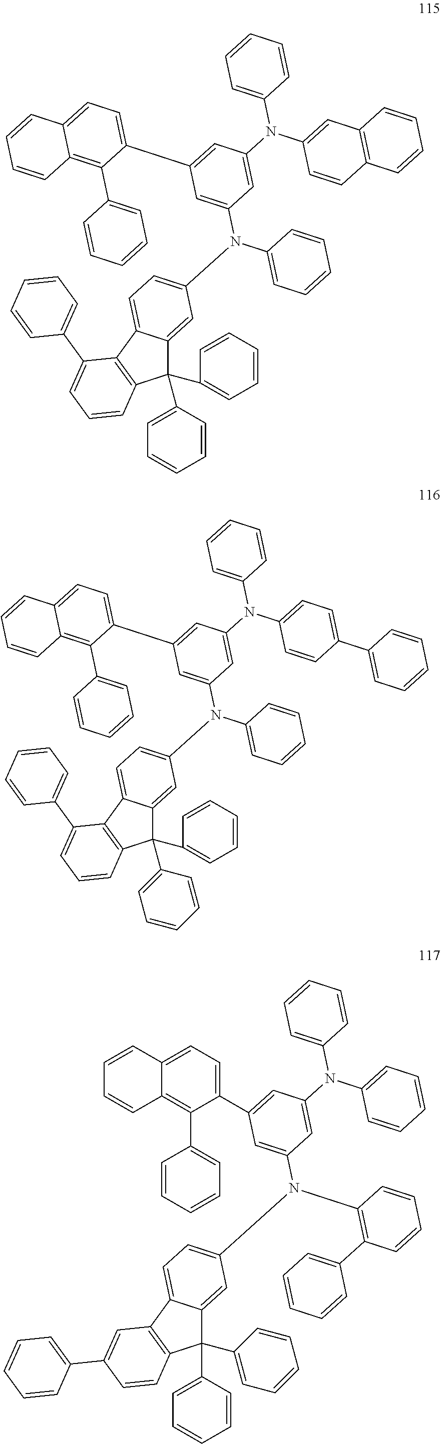 [SMILES: c1ccc(-c2cccc3c2-c2ccc(N(c4ccccc4)c4cc(-c5ccc6ccccc6c5-c5ccccc5)cc(N(c5ccccc5)c5ccc6ccccc6c5)c4)cc2C3(c2ccccc2)c2ccccc2)cc1 ; c1ccc(-c2ccc(N(c3ccccc3)c3cc(-c4ccc5ccccc5c4-c4ccccc4)cc(N(c4ccccc4)c4ccc5c(c4)C(c4ccccc4)(c4ccccc4)c4cccc(-c6ccccc6)c4-5)c3)cc2)cc1 ; c1ccc(-c2ccc3c(c2)-c2ccc(N(c4cc(-c5ccc6ccccc6c5-c5ccccc5)cc(N(c5ccccc5)c5ccccc5)c4)c4ccccc4-c4ccccc4)cc2C3(c2ccccc2)c2ccccc2)cc1]

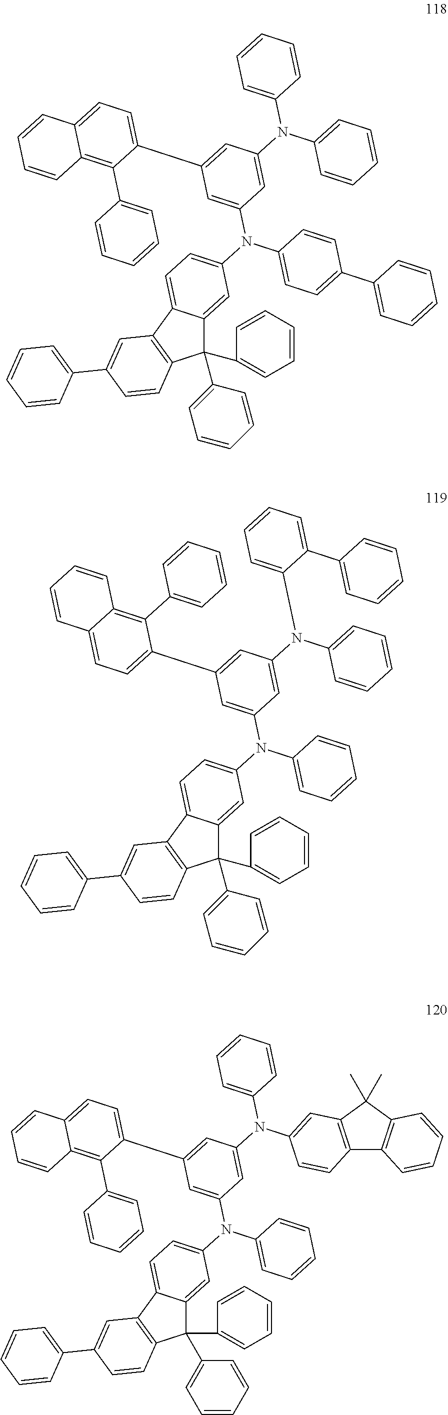 [SMILES: c1ccc(-c2ccc(N(c3cc(-c4ccc5ccccc5c4-c4ccccc4)cc(N(c4ccccc4)c4ccccc4)c3)c3ccc4c(c3)C(c3ccccc3)(c3ccccc3)c3ccc(-c5ccccc5)cc3-4)cc2)cc1 ; c1ccc(-c2ccc3c(c2)-c2ccc(N(c4ccccc4)c4cc(-c5ccc6ccccc6c5-c5ccccc5)cc(N(c5ccccc5)c5ccccc5-c5ccccc5)c4)cc2C3(c2ccccc2)c2ccccc2)cc1 ; CC1(C)c2ccccc2-c2ccc(N(c3ccccc3)c3cc(-c4ccc5ccccc5c4-c4ccccc4)cc(N(c4ccccc4)c4ccc5c(c4)C(c4ccccc4)(c4ccccc4)c4ccc(-c6ccccc6)cc4-5)c3)cc21]

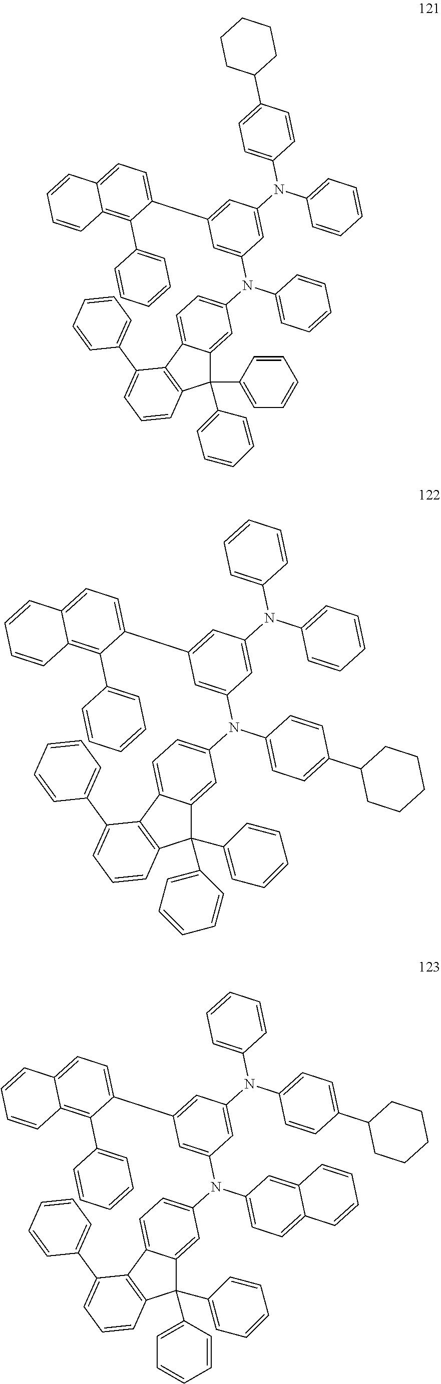 [SMILES: c1ccc(-c2cccc3c2-c2ccc(N(c4ccccc4)c4cc(-c5ccc6ccccc6c5-c5ccccc5)cc(N(c5ccccc5)c5ccc(C6CCCCC6)cc5)c4)cc2C3(c2ccccc2)c2ccccc2)cc1 ; c1ccc(-c2cccc3c2-c2ccc(N(c4ccc(C5CCCCC5)cc4)c4cc(-c5ccc6ccccc6c5-c5ccccc5)cc(N(c5ccccc5)c5ccccc5)c4)cc2C3(c2ccccc2)c2ccccc2)cc1 ; c1ccc(-c2cccc3c2-c2ccc(N(c4cc(-c5ccc6ccccc6c5-c5ccccc5)cc(N(c5ccccc5)c5ccc(C6CCCCC6)cc5)c4)c4ccc5ccccc5c4)cc2C3(c2ccccc2)c2ccccc2)cc1]

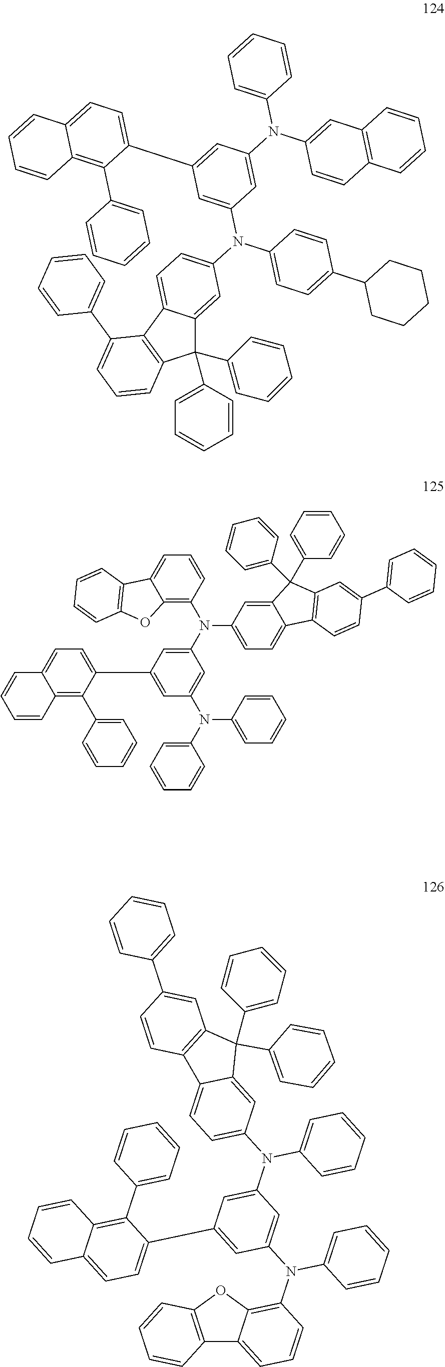 [SMILES: c1ccc(-c2cccc3c2-c2ccc(N(c4ccc(C5CCCCC5)cc4)c4cc(-c5ccc6ccccc6c5-c5ccccc5)cc(N(c5ccccc5)c5ccc6ccccc6c5)c4)cc2C3(c2ccccc2)c2ccccc2)cc1 ; c1ccc(-c2ccc3c(c2)C(c2ccccc2)(c2ccccc2)c2cc(N(c4cc(-c5ccc6ccccc6c5-c5ccccc5)cc(N(c5ccccc5)c5ccccc5)c4)c4cccc5c4oc4ccccc45)ccc2-3)cc1 ; c1ccc(-c2ccc3c(c2)C(c2ccccc2)(c2ccccc2)c2cc(N(c4ccccc4)c4cc(-c5ccc6ccccc6c5-c5ccccc5)cc(N(c5ccccc5)c5cccc6c5oc5ccccc56)c4)ccc2-3)cc1]

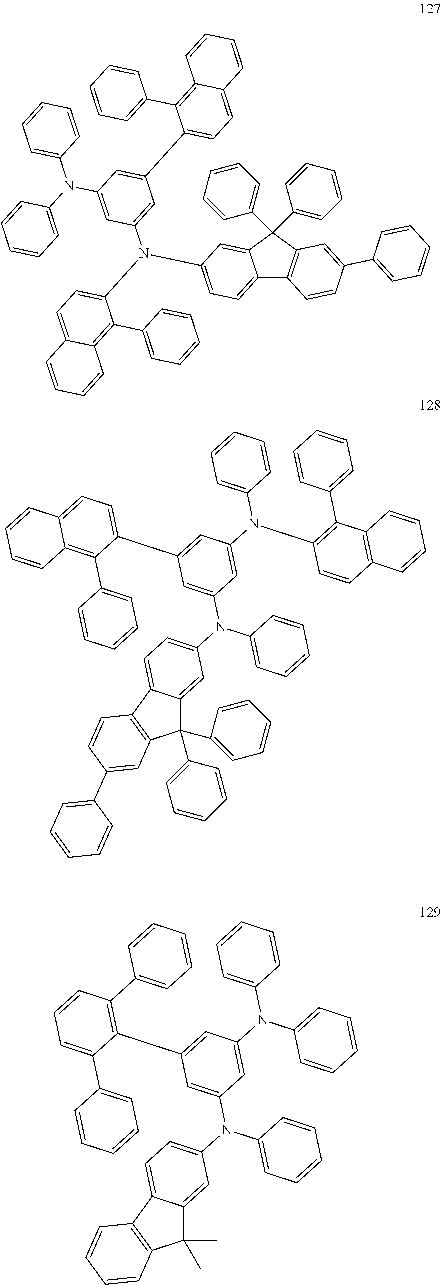 [SMILES: c1ccc(-c2ccc3c(c2)C(c2ccccc2)(c2ccccc2)c2cc(N(c4cc(-c5ccc6ccccc6c5-c5ccccc5)cc(N(c5ccccc5)c5ccccc5)c4)c4ccc5ccccc5c4-c4ccccc4)ccc2-3)cc1 ; c1ccc(-c2ccc3c(c2)C(c2ccccc2)(c2ccccc2)c2cc(N(c4ccccc4)c4cc(-c5ccc6ccccc6c5-c5ccccc5)cc(N(c5ccccc5)c5ccc6ccccc6c5-c5ccccc5)c4)ccc2-3)cc1 ; CC1(C)c2ccccc2-c2ccc(N(c3ccccc3)c3cc(-c4c(-c5ccccc5)cccc4-c4ccccc4)cc(N(c4ccccc4)c4ccccc4)c3)cc21]

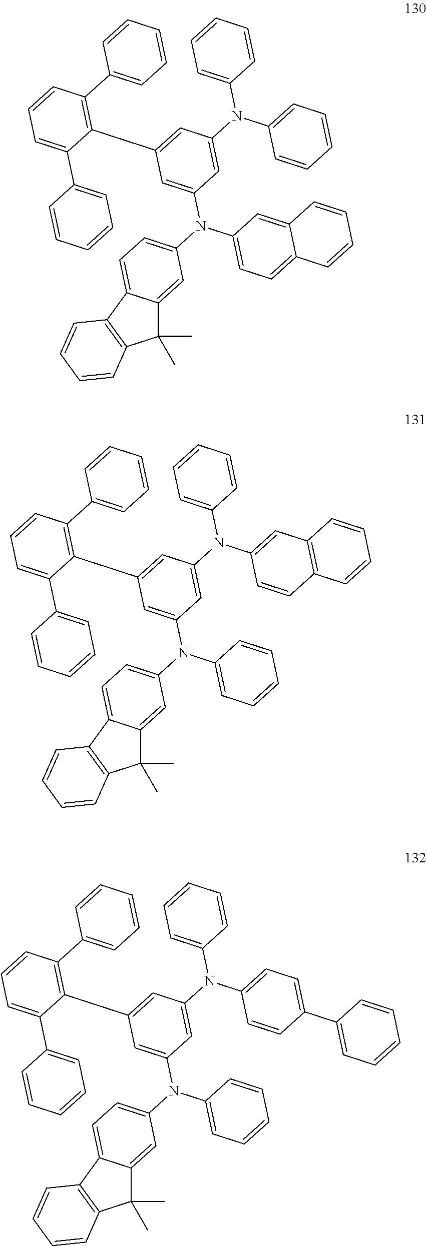 [SMILES: CC1(C)c2ccccc2-c2ccc(N(c3cc(-c4c(-c5ccccc5)cccc4-c4ccccc4)cc(N(c4ccccc4)c4ccccc4)c3)c3ccc4ccccc4c3)cc21 ; CC1(C)c2ccccc2-c2ccc(N(c3ccccc3)c3cc(-c4c(-c5ccccc5)cccc4-c4ccccc4)cc(N(c4ccccc4)c4ccc5ccccc5c4)c3)cc21 ; CC1(C)c2ccccc2-c2ccc(N(c3ccccc3)c3cc(-c4c(-c5ccccc5)cccc4-c4ccccc4)cc(N(c4ccccc4)c4ccc(-c5ccccc5)cc4)c3)cc21]

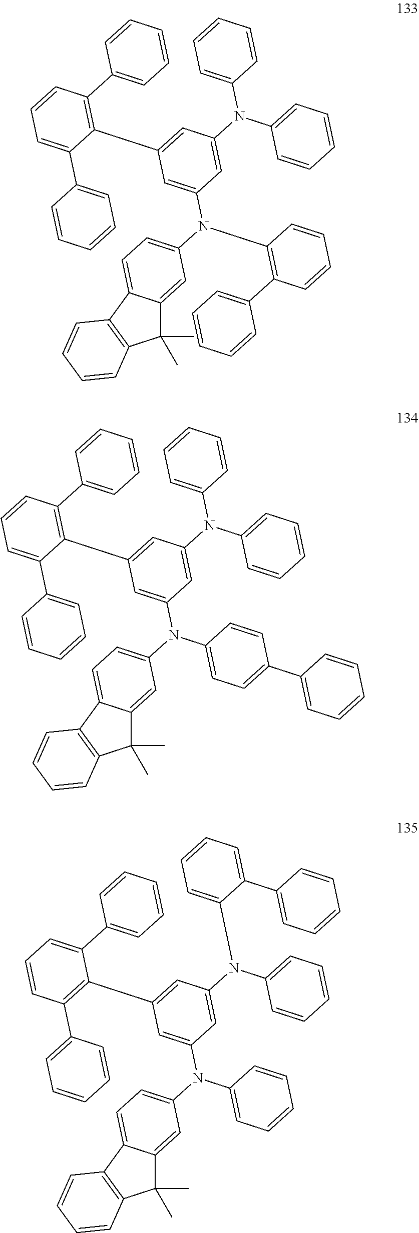 [SMILES: CC1(C)c2ccccc2-c2ccc(N(c3cc(-c4c(-c5ccccc5)cccc4-c4ccccc4)cc(N(c4ccccc4)c4ccccc4)c3)c3ccccc3-c3ccccc3)cc21 ; CC1(C)c2ccccc2-c2ccc(N(c3ccc(-c4ccccc4)cc3)c3cc(-c4c(-c5ccccc5)cccc4-c4ccccc4)cc(N(c4ccccc4)c4ccccc4)c3)cc21 ; CC1(C)c2ccccc2-c2ccc(N(c3ccccc3)c3cc(-c4c(-c5ccccc5)cccc4-c4ccccc4)cc(N(c4ccccc4)c4ccccc4-c4ccccc4)c3)cc21]

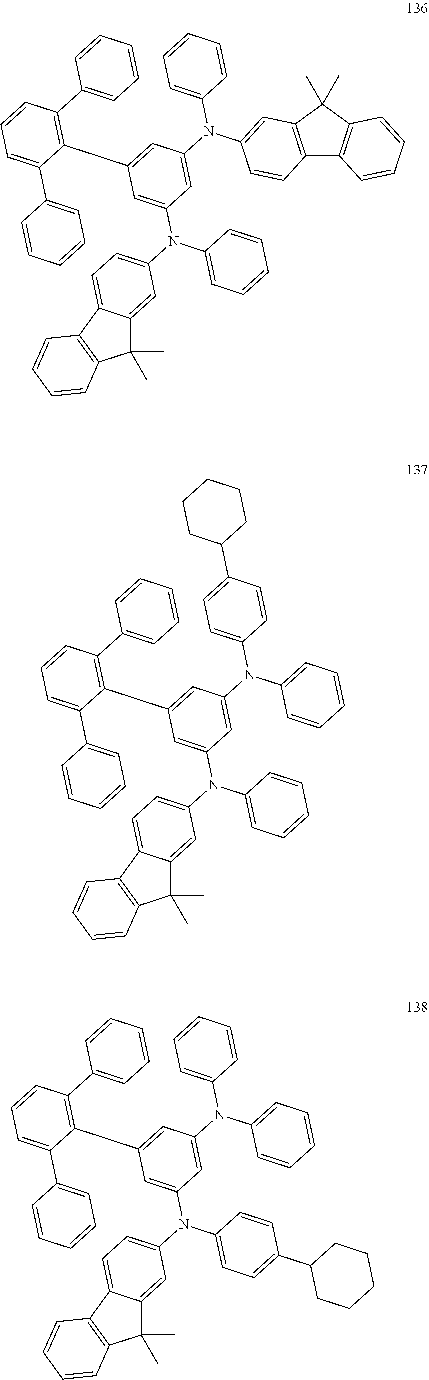 [SMILES: CC1(C)c2ccccc2-c2ccc(N(c3ccccc3)c3cc(-c4c(-c5ccccc5)cccc4-c4ccccc4)cc(N(c4ccccc4)c4ccc5c(c4)C(C)(C)c4ccccc4-5)c3)cc21 ; CC1(C)c2ccccc2-c2ccc(N(c3ccccc3)c3cc(-c4c(-c5ccccc5)cccc4-c4ccccc4)cc(N(c4ccccc4)c4ccc(C5CCCCC5)cc4)c3)cc21 ; CC1(C)c2ccccc2-c2ccc(N(c3ccc(C4CCCCC4)cc3)c3cc(-c4c(-c5ccccc5)cccc4-c4ccccc4)cc(N(c4ccccc4)c4ccccc4)c3)cc21]

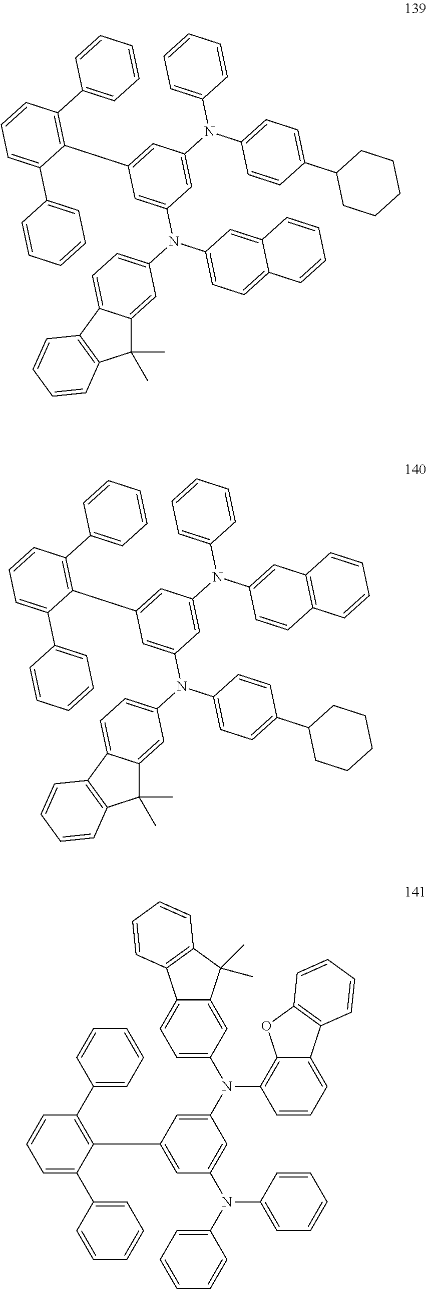 [SMILES: CC1(C)c2ccccc2-c2ccc(N(c3cc(-c4c(-c5ccccc5)cccc4-c4ccccc4)cc(N(c4ccccc4)c4ccc(C5CCCCC5)cc4)c3)c3ccc4ccccc4c3)cc21 ; CC1(C)c2ccccc2-c2ccc(N(c3ccc(C4CCCCC4)cc3)c3cc(-c4c(-c5ccccc5)cccc4-c4ccccc4)cc(N(c4ccccc4)c4ccc5ccccc5c4)c3)cc21 ; CC1(C)c2ccccc2-c2ccc(N(c3cc(-c4c(-c5ccccc5)cccc4-c4ccccc4)cc(N(c4ccccc4)c4ccccc4)c3)c3cccc4c3oc3ccccc34)cc21]

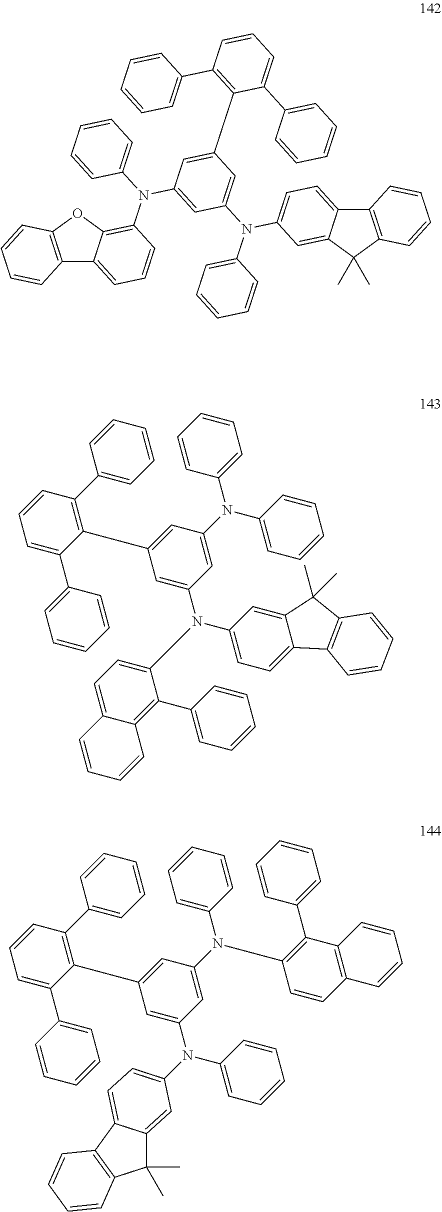 [SMILES: CC1(C)c2ccccc2-c2ccc(N(c3ccccc3)c3cc(-c4c(-c5ccccc5)cccc4-c4ccccc4)cc(N(c4ccccc4)c4cccc5c4oc4ccccc45)c3)cc21 ; CC1(C)c2ccccc2-c2ccc(N(c3cc(-c4c(-c5ccccc5)cccc4-c4ccccc4)cc(N(c4ccccc4)c4ccccc4)c3)c3ccc4ccccc4c3-c3ccccc3)cc21 ; CC1(C)c2ccccc2-c2ccc(N(c3ccccc3)c3cc(-c4c(-c5ccccc5)cccc4-c4ccccc4)cc(N(c4ccccc4)c4ccc5ccccc5c4-c4ccccc4)c3)cc21]

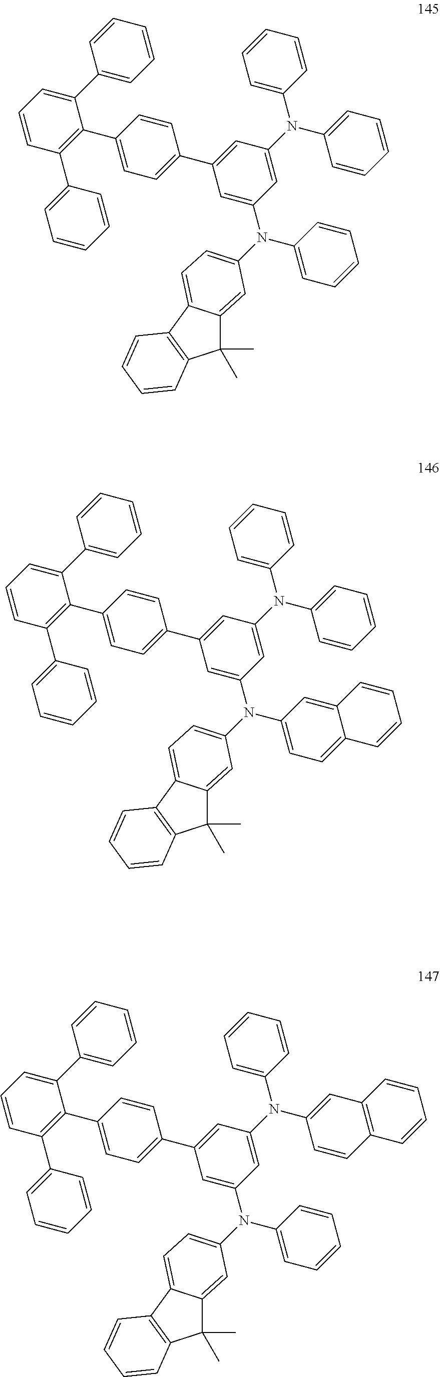 [SMILES: CC1(C)c2ccccc2-c2ccc(N(c3ccccc3)c3cc(-c4ccc(-c5c(-c6ccccc6)cccc5-c5ccccc5)cc4)cc(N(c4ccccc4)c4ccccc4)c3)cc21 ; CC1(C)c2ccccc2-c2ccc(N(c3cc(-c4ccc(-c5c(-c6ccccc6)cccc5-c5ccccc5)cc4)cc(N(c4ccccc4)c4ccccc4)c3)c3ccc4ccccc4c3)cc21 ; CC1(C)c2ccccc2-c2ccc(N(c3ccccc3)c3cc(-c4ccc(-c5c(-c6ccccc6)cccc5-c5ccccc5)cc4)cc(N(c4ccccc4)c4ccc5ccccc5c4)c3)cc21]

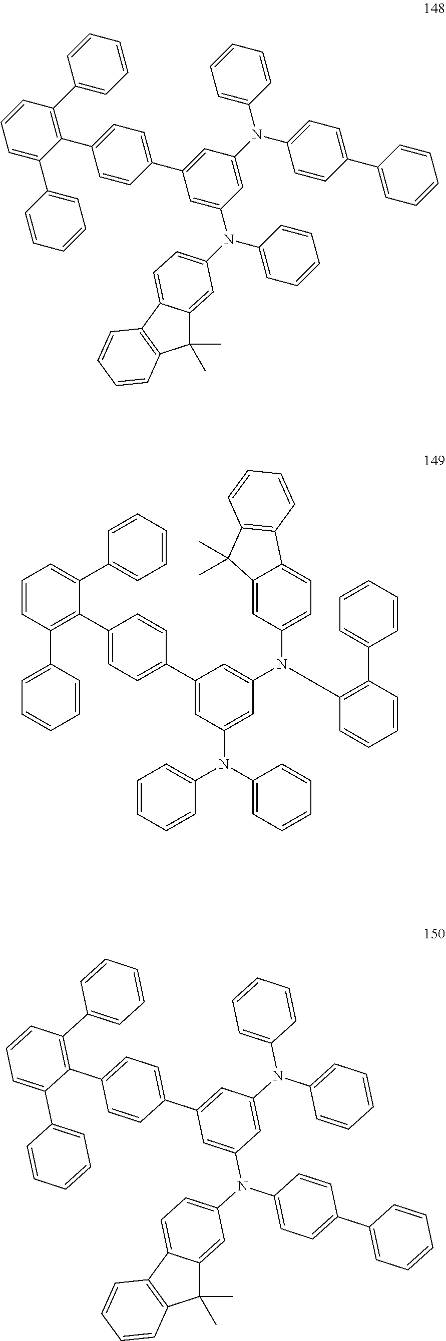 [SMILES: CC1(C)c2ccccc2-c2ccc(N(c3ccccc3)c3cc(-c4ccc(-c5c(-c6ccccc6)cccc5-c5ccccc5)cc4)cc(N(c4ccccc4)c4ccc(-c5ccccc5)cc4)c3)cc21 ; CC1(C)c2ccccc2-c2ccc(N(c3cc(-c4ccc(-c5c(-c6ccccc6)cccc5-c5ccccc5)cc4)cc(N(c4ccccc4)c4ccccc4)c3)c3ccccc3-c3ccccc3)cc21 ; CC1(C)c2ccccc2-c2ccc(N(c3ccc(-c4ccccc4)cc3)c3cc(-c4ccc(-c5c(-c6ccccc6)cccc5-c5ccccc5)cc4)cc(N(c4ccccc4)c4ccccc4)c3)cc21]

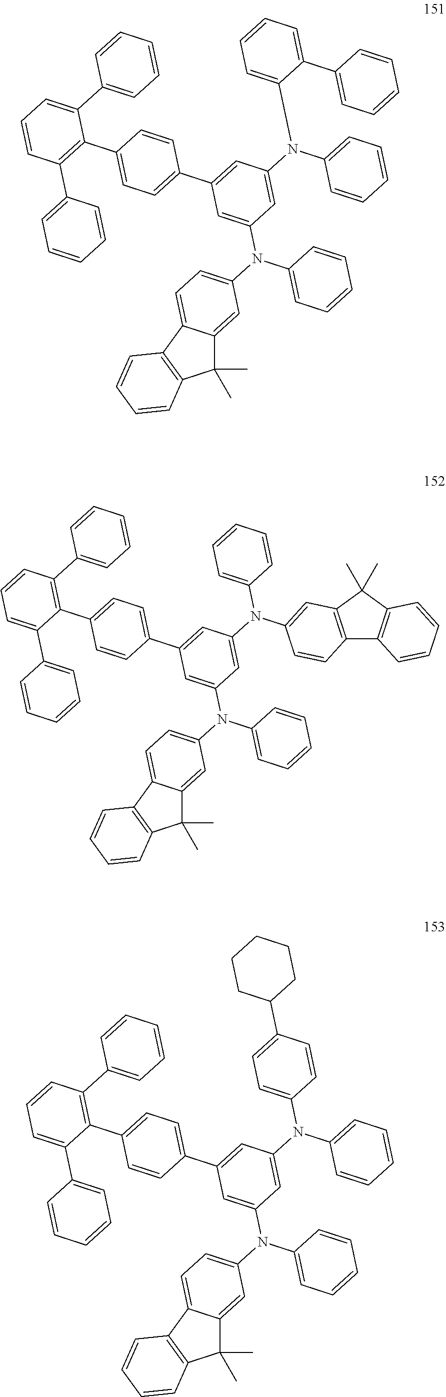 [SMILES: CC1(C)c2ccccc2-c2ccc(N(c3ccccc3)c3cc(-c4ccc(-c5c(-c6ccccc6)cccc5-c5ccccc5)cc4)cc(N(c4ccccc4)c4ccccc4-c4ccccc4)c3)cc21 ; CC1(C)c2ccccc2-c2ccc(N(c3ccccc3)c3cc(-c4ccc(-c5c(-c6ccccc6)cccc5-c5ccccc5)cc4)cc(N(c4ccccc4)c4ccc5c(c4)C(C)(C)c4ccccc4-5)c3)cc21 ; CC1(C)c2ccccc2-c2ccc(N(c3ccccc3)c3cc(-c4ccc(-c5c(-c6ccccc6)cccc5-c5ccccc5)cc4)cc(N(c4ccccc4)c4ccc(C5CCCCC5)cc4)c3)cc21]

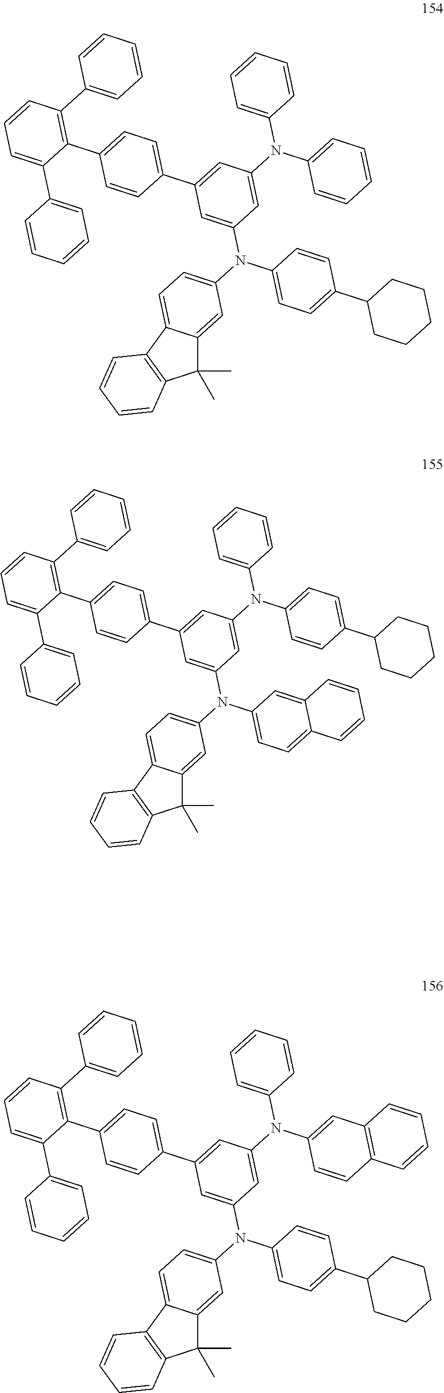 [SMILES: CC1(C)c2ccccc2-c2ccc(N(c3ccc(C4CCCCC4)cc3)c3cc(-c4ccc(-c5c(-c6ccccc6)cccc5-c5ccccc5)cc4)cc(N(c4ccccc4)c4ccccc4)c3)cc21 ; CC1(C)c2ccccc2-c2ccc(N(c3cc(-c4ccc(-c5c(-c6ccccc6)cccc5-c5ccccc5)cc4)cc(N(c4ccccc4)c4ccc(C5CCCCC5)cc4)c3)c3ccc4ccccc4c3)cc21 ; CC1(C)c2ccccc2-c2ccc(N(c3ccc(C4CCCCC4)cc3)c3cc(-c4ccc(-c5c(-c6ccccc6)cccc5-c5ccccc5)cc4)cc(N(c4ccccc4)c4ccc5ccccc5c4)c3)cc21]

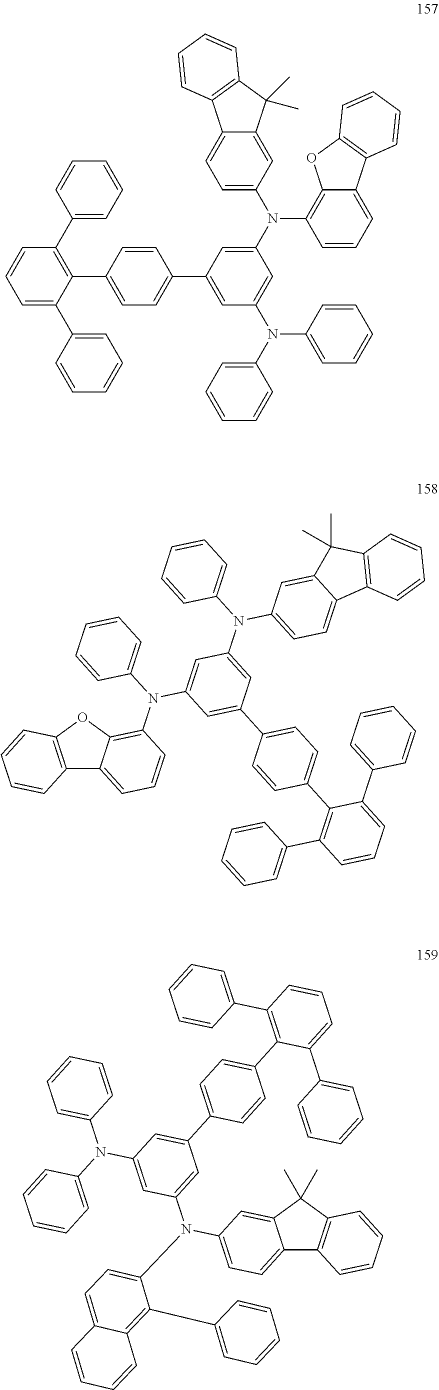 [SMILES: CC1(C)c2ccccc2-c2ccc(N(c3cc(-c4ccc(-c5c(-c6ccccc6)cccc5-c5ccccc5)cc4)cc(N(c4ccccc4)c4ccccc4)c3)c3cccc4c3oc3ccccc34)cc21 ; CC1(C)c2ccccc2-c2ccc(N(c3ccccc3)c3cc(-c4ccc(-c5c(-c6ccccc6)cccc5-c5ccccc5)cc4)cc(N(c4ccccc4)c4cccc5c4oc4ccccc45)c3)cc21 ; CC1(C)c2ccccc2-c2ccc(N(c3cc(-c4ccc(-c5c(-c6ccccc6)cccc5-c5ccccc5)cc4)cc(N(c4ccccc4)c4ccccc4)c3)c3ccc4ccccc4c3-c3ccccc3)cc21]

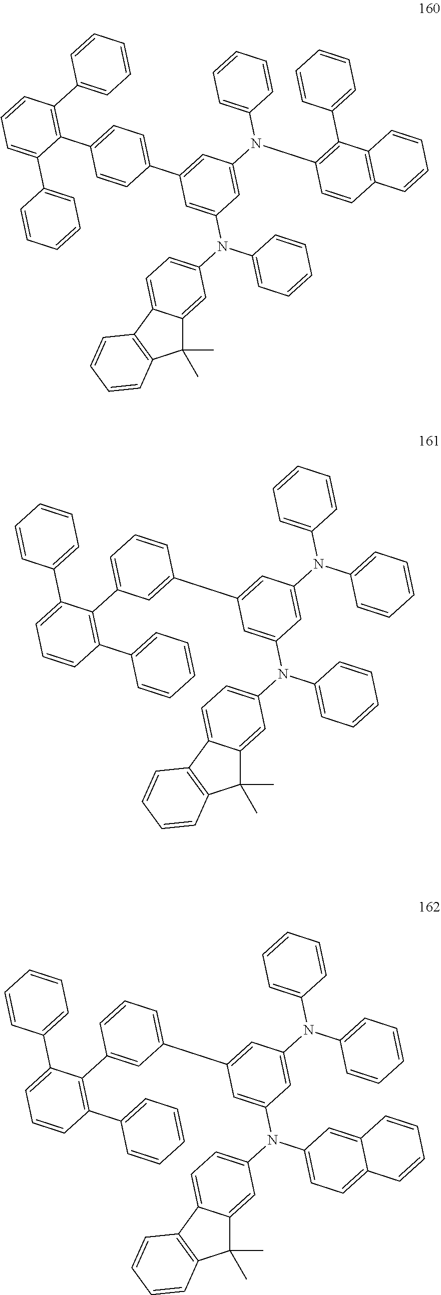 [SMILES: CC1(C)c2ccccc2-c2ccc(N(c3ccccc3)c3cc(-c4ccc(-c5c(-c6ccccc6)cccc5-c5ccccc5)cc4)cc(N(c4ccccc4)c4ccc5ccccc5c4-c4ccccc4)c3)cc21 ; CC1(C)c2ccccc2-c2ccc(N(c3ccccc3)c3cc(-c4cccc(-c5c(-c6ccccc6)cccc5-c5ccccc5)c4)cc(N(c4ccccc4)c4ccccc4)c3)cc21 ; CC1(C)c2ccccc2-c2ccc(N(c3cc(-c4cccc(-c5c(-c6ccccc6)cccc5-c5ccccc5)c4)cc(N(c4ccccc4)c4ccccc4)c3)c3ccc4ccccc4c3)cc21]

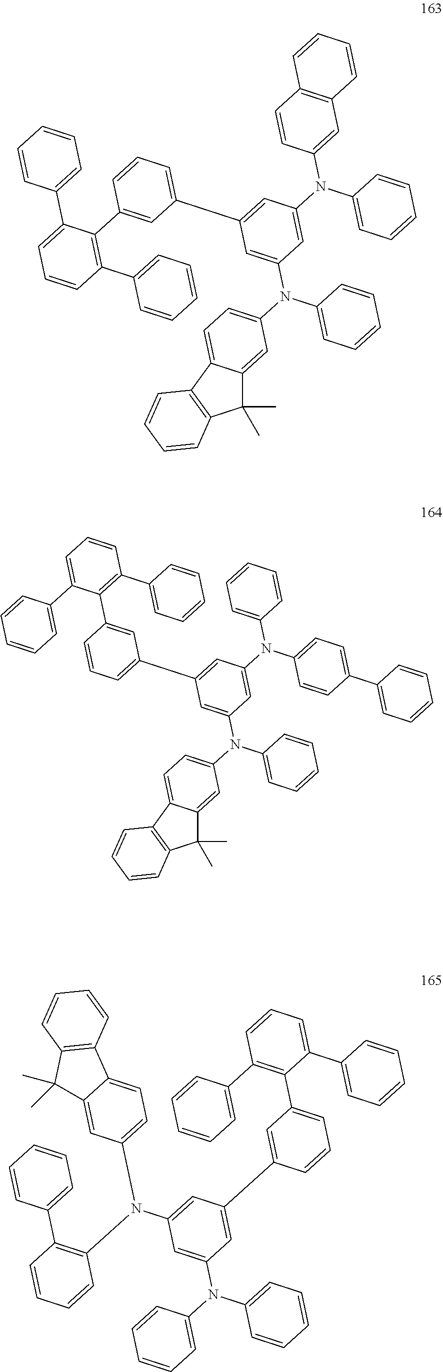 [SMILES: CC1(C)c2ccccc2-c2ccc(N(c3ccccc3)c3cc(-c4cccc(-c5c(-c6ccccc6)cccc5-c5ccccc5)c4)cc(N(c4ccccc4)c4ccc5ccccc5c4)c3)cc21 ; CC1(C)c2ccccc2-c2ccc(N(c3ccccc3)c3cc(-c4cccc(-c5c(-c6ccccc6)cccc5-c5ccccc5)c4)cc(N(c4ccccc4)c4ccc(-c5ccccc5)cc4)c3)cc21 ; CC1(C)c2ccccc2-c2ccc(N(c3cc(-c4cccc(-c5c(-c6ccccc6)cccc5-c5ccccc5)c4)cc(N(c4ccccc4)c4ccccc4)c3)c3ccccc3-c3ccccc3)cc21]

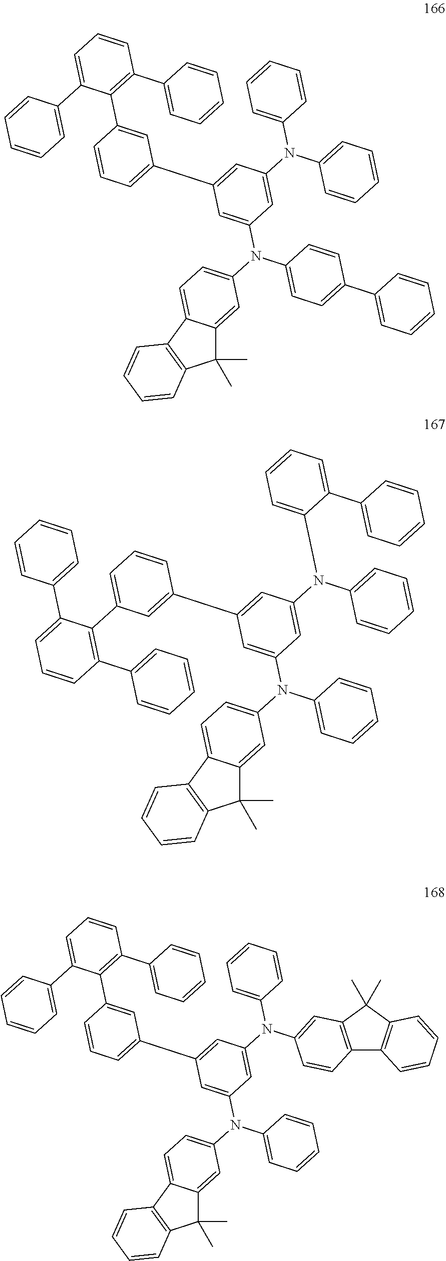 [SMILES: CC1(C)c2ccccc2-c2ccc(N(c3ccc(-c4ccccc4)cc3)c3cc(-c4cccc(-c5c(-c6ccccc6)cccc5-c5ccccc5)c4)cc(N(c4ccccc4)c4ccccc4)c3)cc21 ; CC1(C)c2ccccc2-c2ccc(N(c3ccccc3)c3cc(-c4cccc(-c5c(-c6ccccc6)cccc5-c5ccccc5)c4)cc(N(c4ccccc4)c4ccccc4-c4ccccc4)c3)cc21 ; CC1(C)c2ccccc2-c2ccc(N(c3ccccc3)c3cc(-c4cccc(-c5c(-c6ccccc6)cccc5-c5ccccc5)c4)cc(N(c4ccccc4)c4ccc5c(c4)C(C)(C)c4ccccc4-5)c3)cc21]

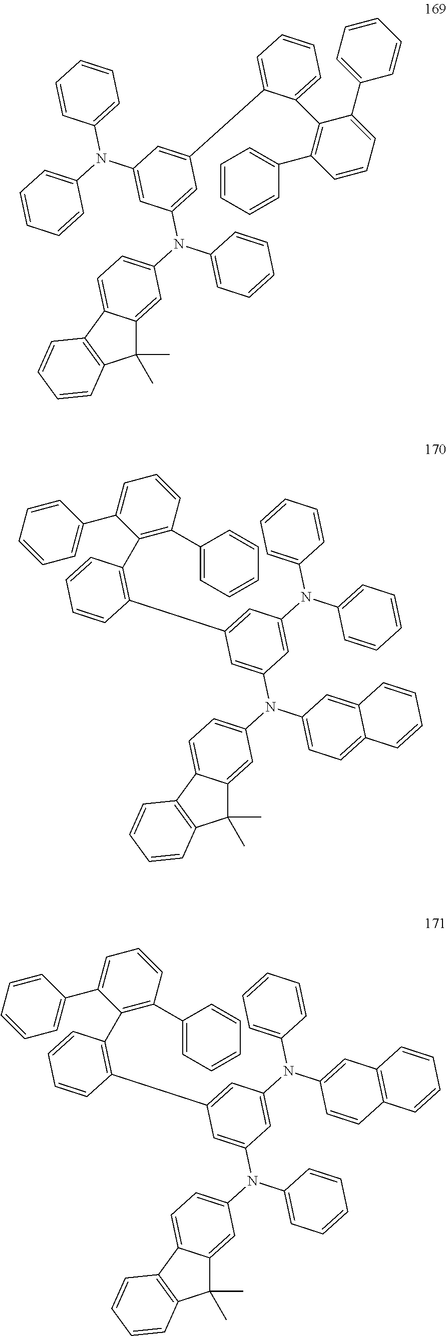 [SMILES: CC1(C)c2ccccc2-c2ccc(N(c3ccccc3)c3cc(-c4ccccc4-c4c(-c5ccccc5)cccc4-c4ccccc4)cc(N(c4ccccc4)c4ccccc4)c3)cc21 ; CC1(C)c2ccccc2-c2ccc(N(c3cc(-c4ccccc4-c4c(-c5ccccc5)cccc4-c4ccccc4)cc(N(c4ccccc4)c4ccccc4)c3)c3ccc4ccccc4c3)cc21 ; CC1(C)c2ccccc2-c2ccc(N(c3ccccc3)c3cc(-c4ccccc4-c4c(-c5ccccc5)cccc4-c4ccccc4)cc(N(c4ccccc4)c4ccc5ccccc5c4)c3)cc21]

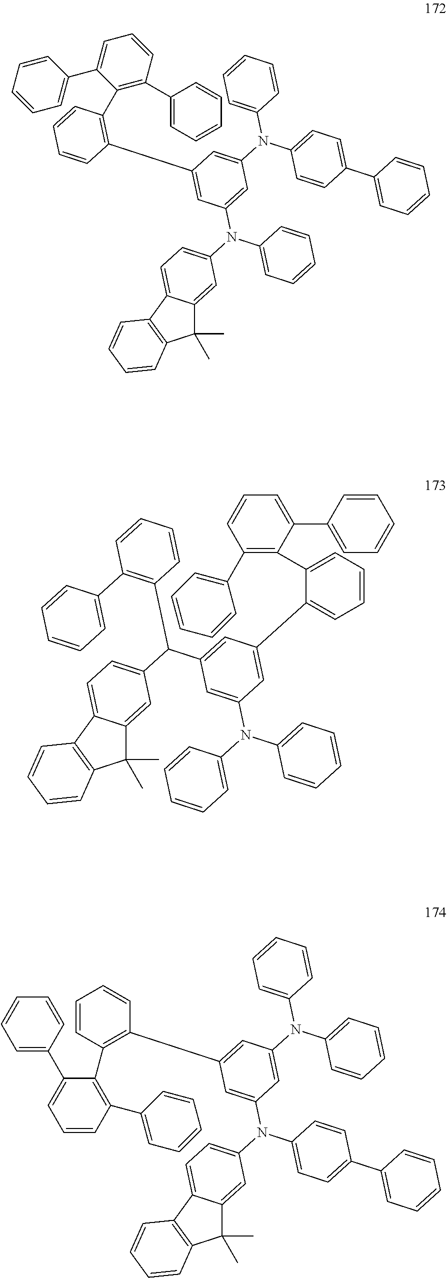 [SMILES: CC1(C)c2ccccc2-c2ccc(N(c3ccccc3)c3cc(-c4ccccc4-c4c(-c5ccccc5)cccc4-c4ccccc4)cc(N(c4ccccc4)c4ccc(-c5ccccc5)cc4)c3)cc21 ; CC1(C)c2ccccc2-c2ccc(C(c3cc(-c4ccccc4-c4c(-c5ccccc5)cccc4-c4ccccc4)cc(N(c4ccccc4)c4ccccc4)c3)c3ccccc3-c3ccccc3)cc21 ; CC1(C)c2ccccc2-c2ccc(N(c3ccc(-c4ccccc4)cc3)c3cc(-c4ccccc4-c4c(-c5ccccc5)cccc4-c4ccccc4)cc(N(c4ccccc4)c4ccccc4)c3)cc21]

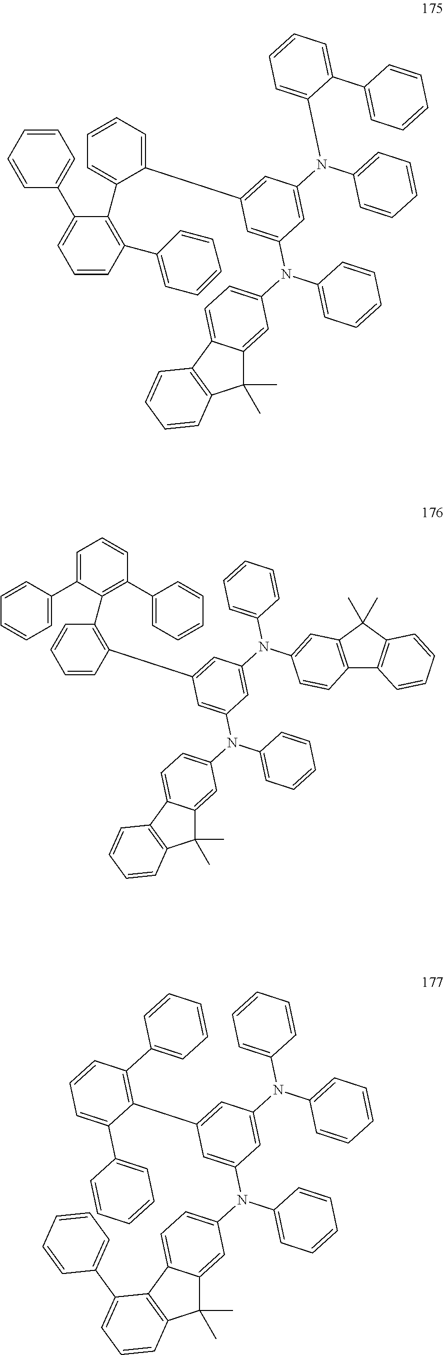 [SMILES: CC1(C)c2ccccc2-c2ccc(N(c3ccccc3)c3cc(-c4ccccc4-c4c(-c5ccccc5)cccc4-c4ccccc4)cc(N(c4ccccc4)c4ccccc4-c4ccccc4)c3)cc21 ; CC1(C)c2ccccc2-c2ccc(N(c3ccccc3)c3cc(-c4ccccc4-c4c(-c5ccccc5)cccc4-c4ccccc4)cc(N(c4ccccc4)c4ccc5c(c4)C(C)(C)c4ccccc4-5)c3)cc21 ; CC1(C)c2cc(N(c3ccccc3)c3cc(-c4c(-c5ccccc5)cccc4-c4ccccc4)cc(N(c4ccccc4)c4ccccc4)c3)ccc2-c2c(-c3ccccc3)cccc21]

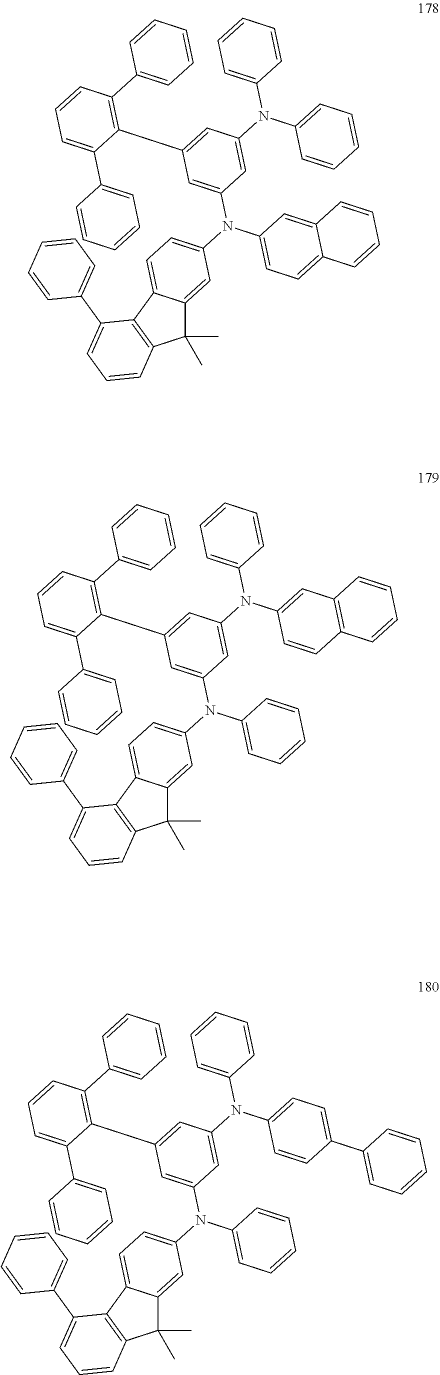 [SMILES: CC1(C)c2cc(N(c3cc(-c4c(-c5ccccc5)cccc4-c4ccccc4)cc(N(c4ccccc4)c4ccccc4)c3)c3ccc4ccccc4c3)ccc2-c2c(-c3ccccc3)cccc21 ; CC1(C)c2cc(N(c3ccccc3)c3cc(-c4c(-c5ccccc5)cccc4-c4ccccc4)cc(N(c4ccccc4)c4ccc5ccccc5c4)c3)ccc2-c2c(-c3ccccc3)cccc21 ; CC1(C)c2cc(N(c3ccccc3)c3cc(-c4c(-c5ccccc5)cccc4-c4ccccc4)cc(N(c4ccccc4)c4ccc(-c5ccccc5)cc4)c3)ccc2-c2c(-c3ccccc3)cccc21]

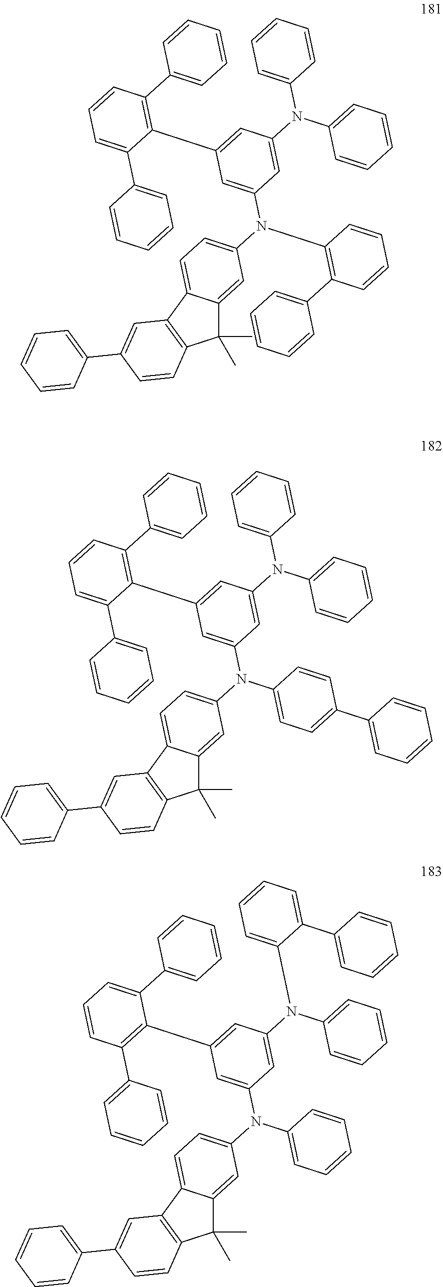 [SMILES: CC1(C)c2ccc(-c3ccccc3)cc2-c2ccc(N(c3cc(-c4c(-c5ccccc5)cccc4-c4ccccc4)cc(N(c4ccccc4)c4ccccc4)c3)c3ccccc3-c3ccccc3)cc21 ; CC1(C)c2ccc(-c3ccccc3)cc2-c2ccc(N(c3ccc(-c4ccccc4)cc3)c3cc(-c4c(-c5ccccc5)cccc4-c4ccccc4)cc(N(c4ccccc4)c4ccccc4)c3)cc21 ; CC1(C)c2ccc(-c3ccccc3)cc2-c2ccc(N(c3ccccc3)c3cc(-c4c(-c5ccccc5)cccc4-c4ccccc4)cc(N(c4ccccc4)c4ccccc4-c4ccccc4)c3)cc21]

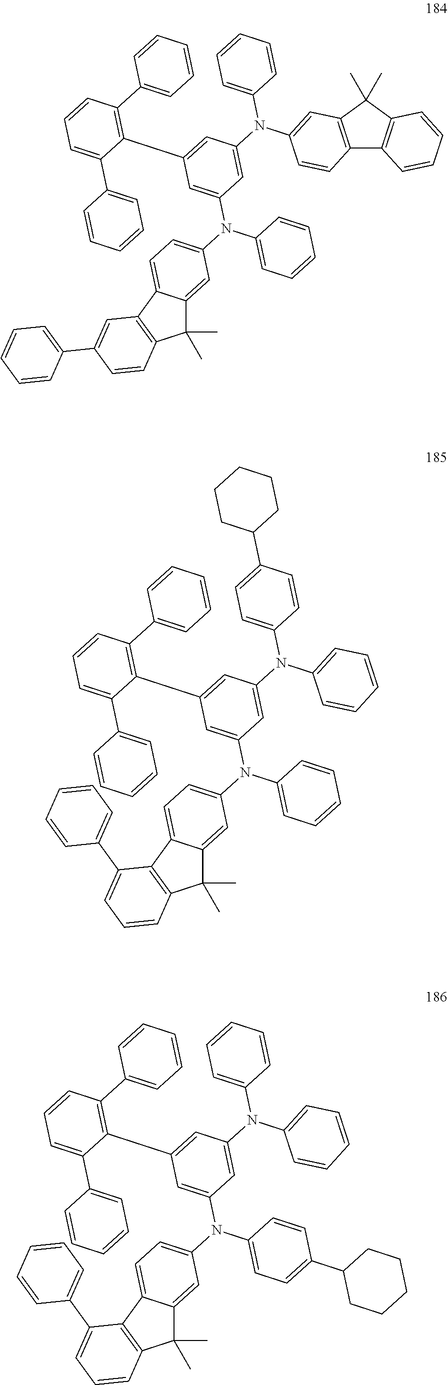 [SMILES: CC1(C)c2ccccc2-c2ccc(N(c3ccccc3)c3cc(-c4c(-c5ccccc5)cccc4-c4ccccc4)cc(N(c4ccccc4)c4ccc5c(c4)C(C)(C)c4ccc(-c6ccccc6)cc4-5)c3)cc21 ; CC1(C)c2cc(N(c3ccccc3)c3cc(-c4c(-c5ccccc5)cccc4-c4ccccc4)cc(N(c4ccccc4)c4ccc(C5CCCCC5)cc4)c3)ccc2-c2c(-c3ccccc3)cccc21 ; CC1(C)c2cc(N(c3ccc(C4CCCCC4)cc3)c3cc(-c4c(-c5ccccc5)cccc4-c4ccccc4)cc(N(c4ccccc4)c4ccccc4)c3)ccc2-c2c(-c3ccccc3)cccc21]

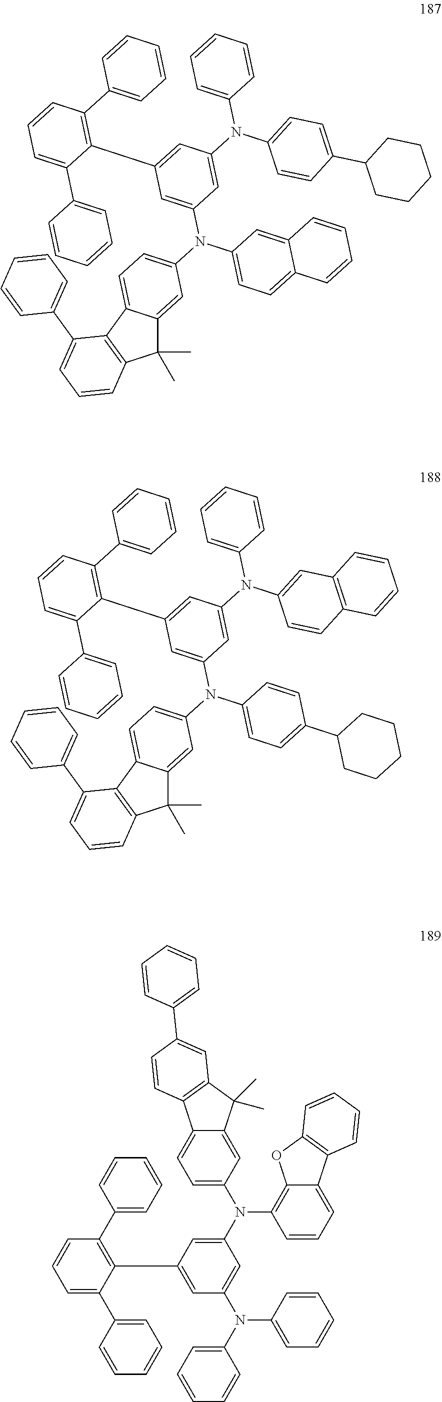 [SMILES: CC1(C)c2cc(N(c3cc(-c4c(-c5ccccc5)cccc4-c4ccccc4)cc(N(c4ccccc4)c4ccc(C5CCCCC5)cc4)c3)c3ccc4ccccc4c3)ccc2-c2c(-c3ccccc3)cccc21 ; CC1(C)c2cc(N(c3ccc(C4CCCCC4)cc3)c3cc(-c4c(-c5ccccc5)cccc4-c4ccccc4)cc(N(c4ccccc4)c4ccc5ccccc5c4)c3)ccc2-c2c(-c3ccccc3)cccc21 ; CC1(C)c2cc(-c3ccccc3)ccc2-c2ccc(N(c3cc(-c4c(-c5ccccc5)cccc4-c4ccccc4)cc(N(c4ccccc4)c4ccccc4)c3)c3cccc4c3oc3ccccc34)cc21]

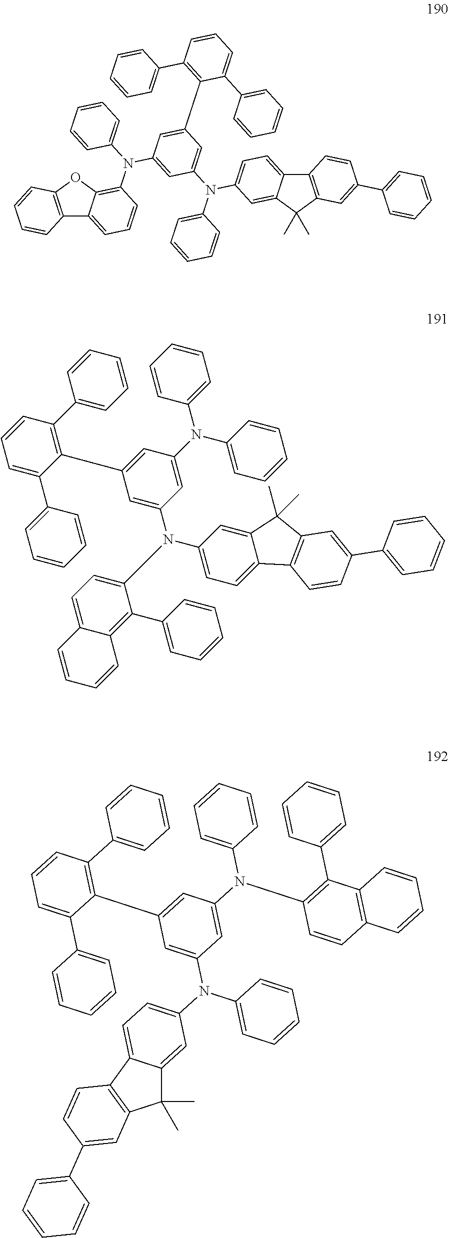 [SMILES: CC1(C)c2cc(-c3ccccc3)ccc2-c2ccc(N(c3ccccc3)c3cc(-c4c(-c5ccccc5)cccc4-c4ccccc4)cc(N(c4ccccc4)c4cccc5c4oc4ccccc45)c3)cc21 ; CC1(C)c2cc(-c3ccccc3)ccc2-c2ccc(N(c3cc(-c4c(-c5ccccc5)cccc4-c4ccccc4)cc(N(c4ccccc4)c4ccccc4)c3)c3ccc4ccccc4c3-c3ccccc3)cc21 ; CC1(C)c2cc(-c3ccccc3)ccc2-c2ccc(N(c3ccccc3)c3cc(-c4c(-c5ccccc5)cccc4-c4ccccc4)cc(N(c4ccccc4)c4ccc5ccccc5c4-c4ccccc4)c3)cc21]

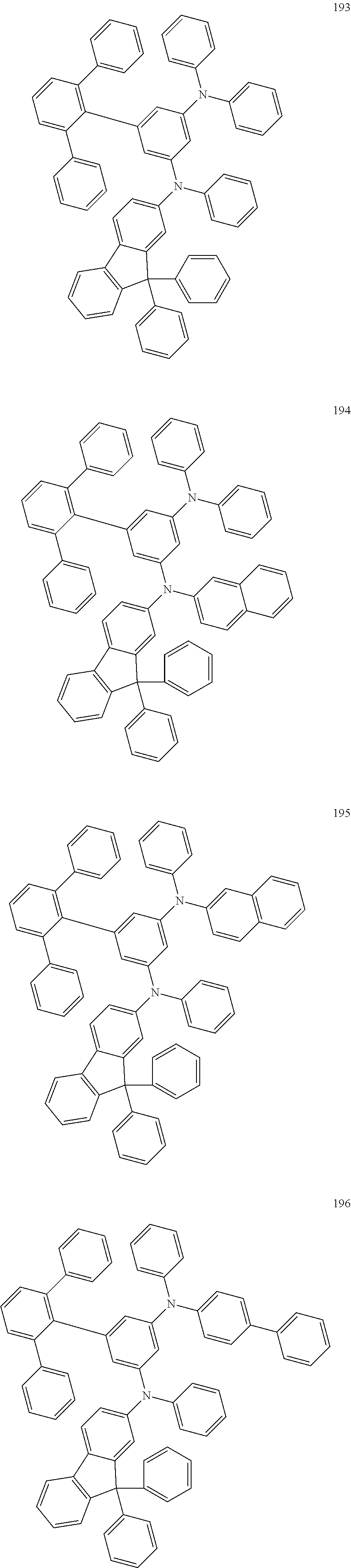 [SMILES: c1ccc(-c2cccc(-c3ccccc3)c2-c2cc(N(c3ccccc3)c3ccccc3)cc(N(c3ccccc3)c3ccc4c(c3)C(c3ccccc3)(c3ccccc3)c3ccccc3-4)c2)cc1 ; c1ccc(-c2cccc(-c3ccccc3)c2-c2cc(N(c3ccccc3)c3ccccc3)cc(N(c3ccc4c(c3)C(c3ccccc3)(c3ccccc3)c3ccccc3-4)c3ccc4ccccc4c3)c2)cc1 ; c1ccc(-c2cccc(-c3ccccc3)c2-c2cc(N(c3ccccc3)c3ccc4c(c3)C(c3ccccc3)(c3ccccc3)c3ccccc3-4)cc(N(c3ccccc3)c3ccc4ccccc4c3)c2)cc1 ; c1ccc(-c2ccc(N(c3ccccc3)c3cc(-c4c(-c5ccccc5)cccc4-c4ccccc4)cc(N(c4ccccc4)c4ccc5c(c4)C(c4ccccc4)(c4ccccc4)c4ccccc4-5)c3)cc2)cc1]

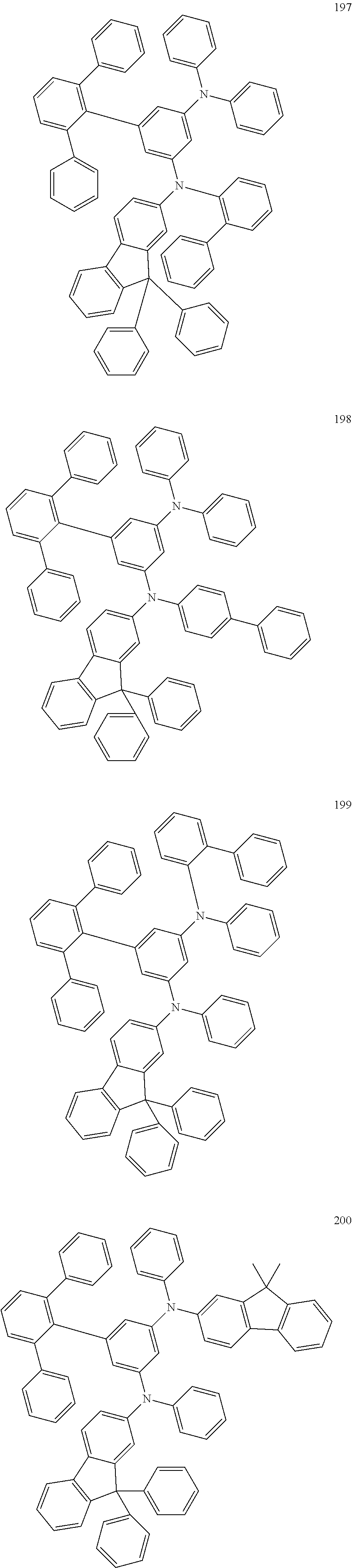

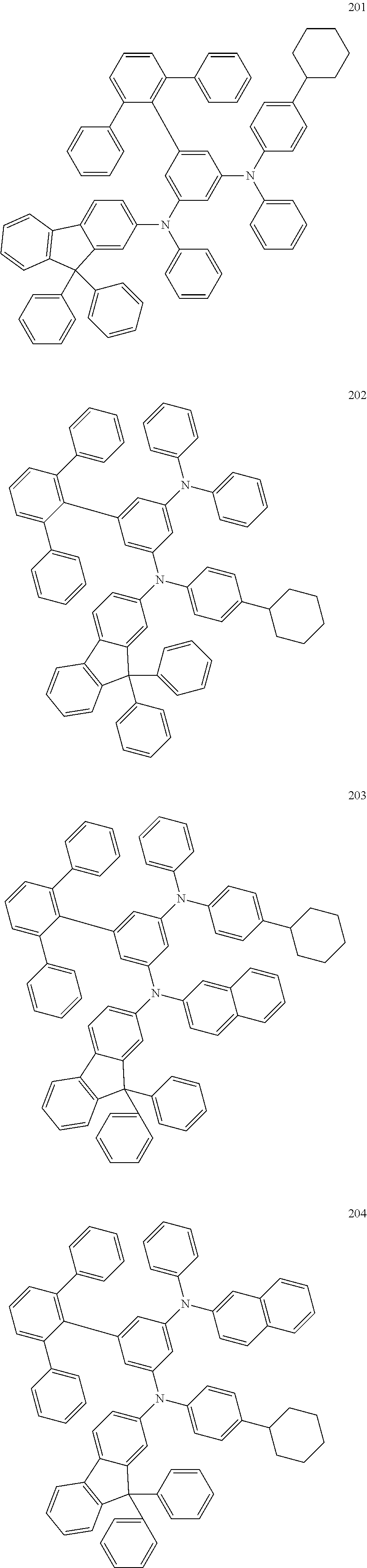

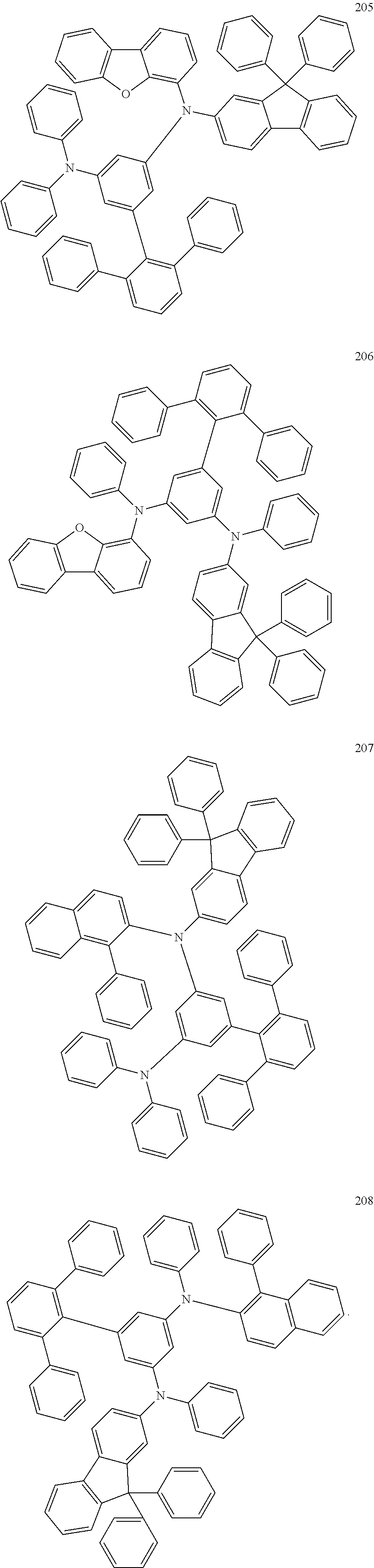

In one or more embodiments, the electron-transporting host may be any one of (e.g., selected from among) Compounds ETH1 to ETH102:

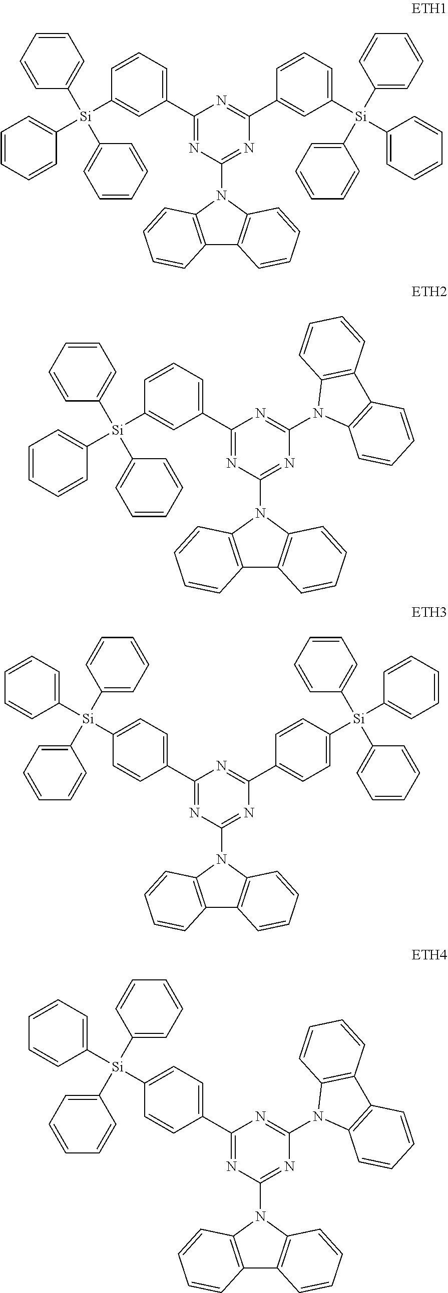 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1]

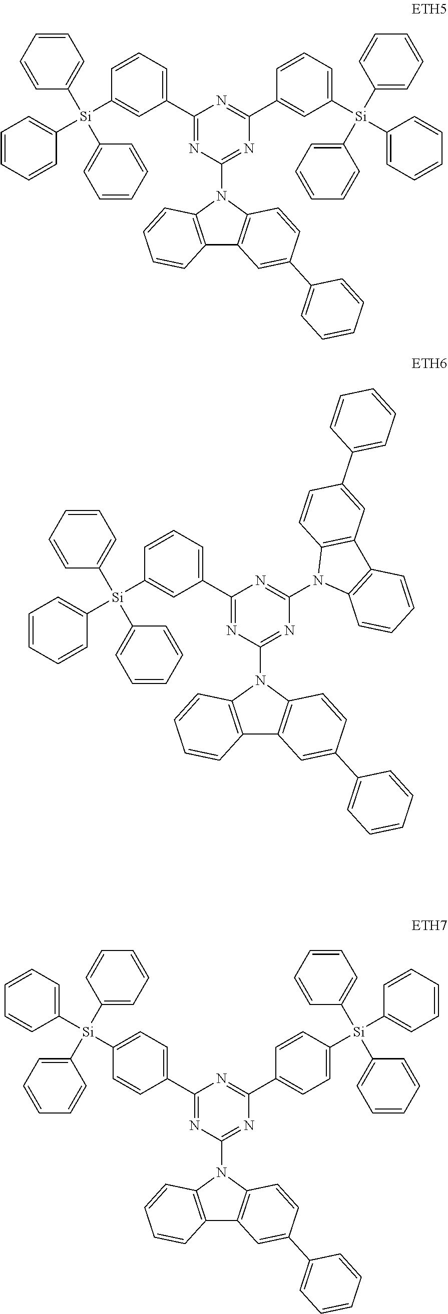 [SMILES: c1ccc(-c2ccc3c(c2)c2ccccc2n3-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; c1ccc(-c2ccc3c(c2)c2ccccc2n3-c2nc(-c3cccc([Si](c4ccccc4)(c4ccccc4)c4ccccc4)c3)nc(-n3c4ccccc4c4cc(-c5ccccc5)ccc43)n2)cc1 ; c1ccc(-c2ccc3c(c2)c2ccccc2n3-c2nc(-c3ccc([Si](c4ccccc4)(c4ccccc4)c4ccccc4)cc3)nc(-c3ccc([Si](c4ccccc4)(c4ccccc4)c4ccccc4)cc3)n2)cc1]

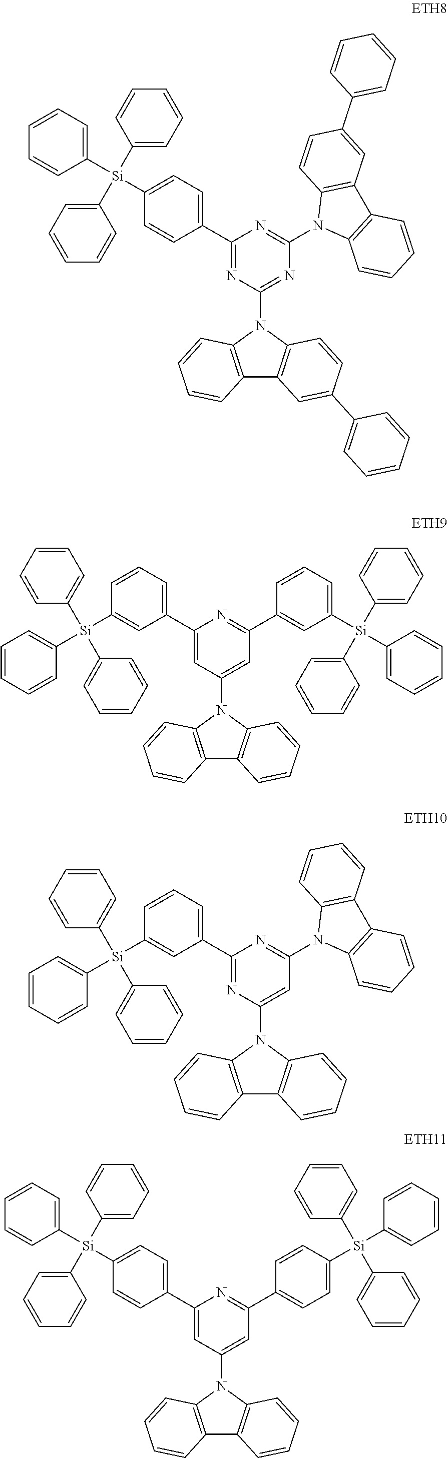 [SMILES: c1ccc(-c2ccc3c(c2)c2ccccc2n3-c2nc(-c3ccc([Si](c4ccccc4)(c4ccccc4)c4ccccc4)cc3)nc(-n3c4ccccc4c4cc(-c5ccccc5)ccc43)n2)cc1 ; c1ccc([Si](c2ccccc2)(c2ccccc2)c2cccc(-c3cc(-n4c5ccccc5c5ccccc54)cc(-c4cccc([Si](c5ccccc5)(c5ccccc5)c5ccccc5)c4)n3)c2)cc1 ; c1ccc([Si](c2ccccc2)(c2ccccc2)c2cccc(-c3nc(-n4c5ccccc5c5ccccc54)cc(-n4c5ccccc5c5ccccc54)n3)c2)cc1 ; c1ccc([Si](c2ccccc2)(c2ccccc2)c2ccc(-c3cc(-n4c5ccccc5c5ccccc54)cc(-c4ccc([Si](c5ccccc5)(c5ccccc5)c5ccccc5)cc4)n3)cc2)cc1]

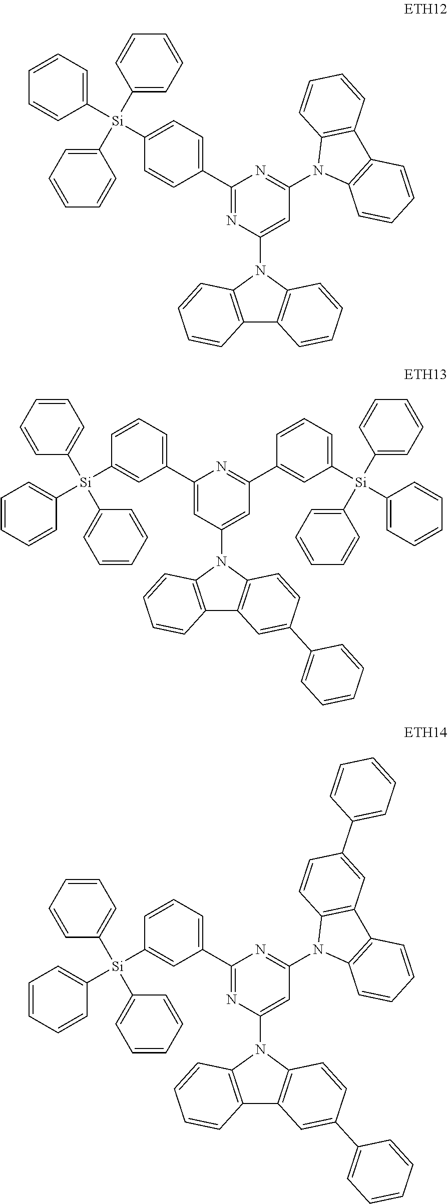 [SMILES: c1ccc([Si](c2ccccc2)(c2ccccc2)c2ccc(-c3nc(-n4c5ccccc5c5ccccc54)cc(-n4c5ccccc5c5ccccc54)n3)cc2)cc1 ; c1ccc(-c2ccc3c(c2)c2ccccc2n3-c2cc(-c3cccc([Si](c4ccccc4)(c4ccccc4)c4ccccc4)c3)nc(-c3cccc([Si](c4ccccc4)(c4ccccc4)c4ccccc4)c3)c2)cc1 ; c1ccc(-c2ccc3c(c2)c2ccccc2n3-c2cc(-n3c4ccccc4c4cc(-c5ccccc5)ccc43)nc(-c3cccc([Si](c4ccccc4)(c4ccccc4)c4ccccc4)c3)n2)cc1]

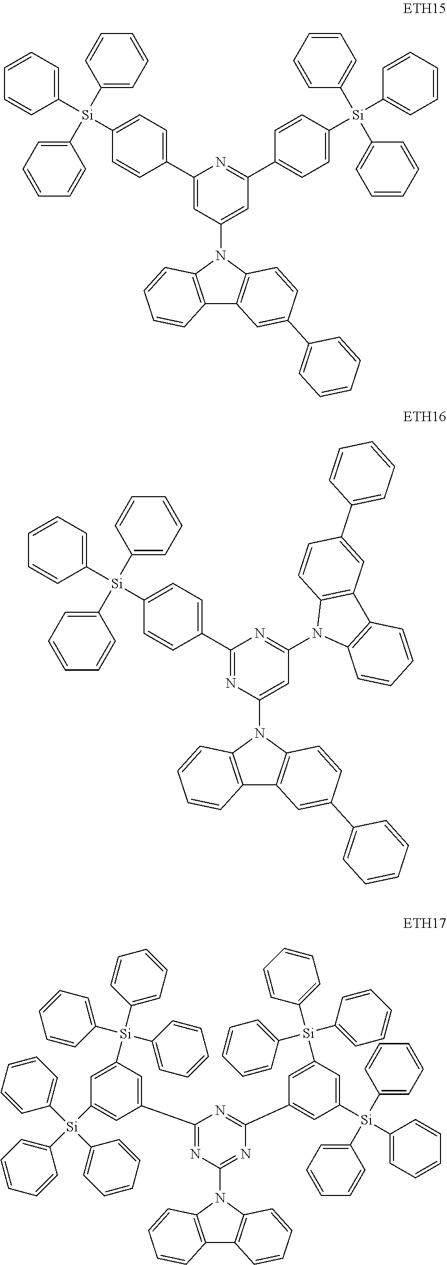 [SMILES: c1ccc(-c2ccc3c(c2)c2ccccc2n3-c2cc(-c3ccc([Si](c4ccccc4)(c4ccccc4)c4ccccc4)cc3)nc(-c3ccc([Si](c4ccccc4)(c4ccccc4)c4ccccc4)cc3)c2)cc1 ; c1ccc(-c2ccc3c(c2)c2ccccc2n3-c2cc(-n3c4ccccc4c4cc(-c5ccccc5)ccc43)nc(-c3ccc([Si](c4ccccc4)(c4ccccc4)c4ccccc4)cc3)n2)cc1 ; c1ccc([Si](c2ccccc2)(c2ccccc2)c2cc(-c3nc(-c4cc([Si](c5ccccc5)(c5ccccc5)c5ccccc5)cc([Si](c5ccccc5)(c5ccccc5)c5ccccc5)c4)nc(-n4c5ccccc5c5ccccc54)n3)cc([Si](c3ccccc3)(c3ccccc3)c3ccccc3)c2)cc1]

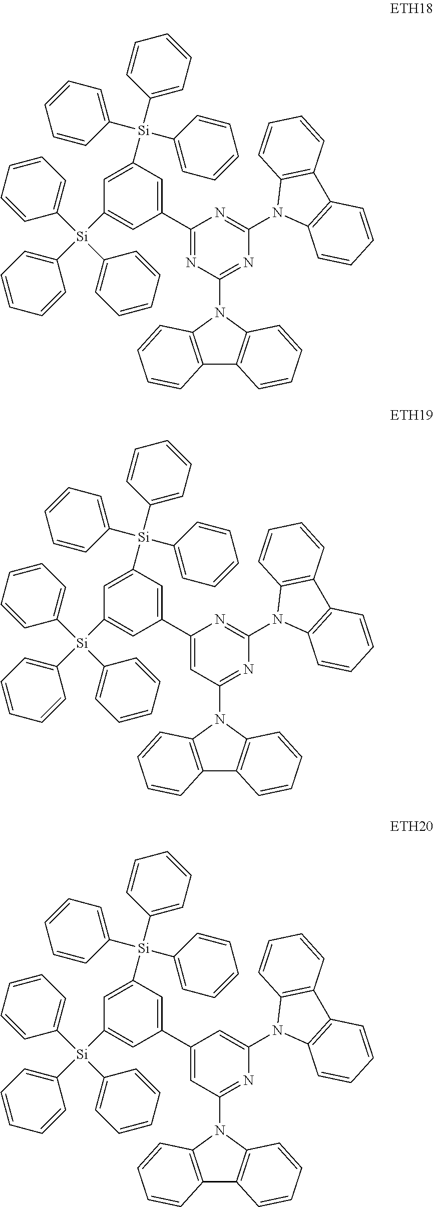 [SMILES: c1ccc([Si](c2ccccc2)(c2ccccc2)c2cc(-c3nc(-n4c5ccccc5c5ccccc54)nc(-n4c5ccccc5c5ccccc54)n3)cc([Si](c3ccccc3)(c3ccccc3)c3ccccc3)c2)cc1 ; c1ccc([Si](c2ccccc2)(c2ccccc2)c2cc(-c3cc(-n4c5ccccc5c5ccccc54)nc(-n4c5ccccc5c5ccccc54)n3)cc([Si](c3ccccc3)(c3ccccc3)c3ccccc3)c2)cc1 ; c1ccc([Si](c2ccccc2)(c2ccccc2)c2cc(-c3cc(-n4c5ccccc5c5ccccc54)nc(-n4c5ccccc5c5ccccc54)c3)cc([Si](c3ccccc3)(c3ccccc3)c3ccccc3)c2)cc1]

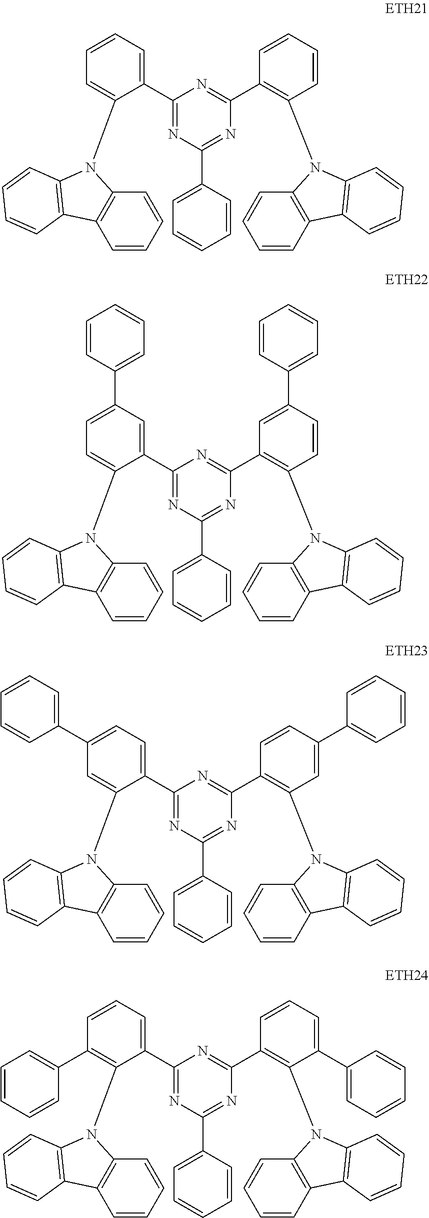 [SMILES: c1ccc(-c2nc(-c3ccccc3-n3c4ccccc4c4ccccc43)nc(-c3ccccc3-n3c4ccccc4c4ccccc43)n2)cc1 ; c1ccc(-c2ccc(-n3c4ccccc4c4ccccc43)c(-c3nc(-c4ccccc4)nc(-c4cc(-c5ccccc5)ccc4-n4c5ccccc5c5ccccc54)n3)c2)cc1 ; c1ccc(-c2ccc(-c3nc(-c4ccccc4)nc(-c4ccc(-c5ccccc5)cc4-n4c5ccccc5c5ccccc54)n3)c(-n3c4ccccc4c4ccccc43)c2)cc1 ; c1ccc(-c2nc(-c3cccc(-c4ccccc4)c3-n3c4ccccc4c4ccccc43)nc(-c3cccc(-c4ccccc4)c3-n3c4ccccc4c4ccccc43)n2)cc1]

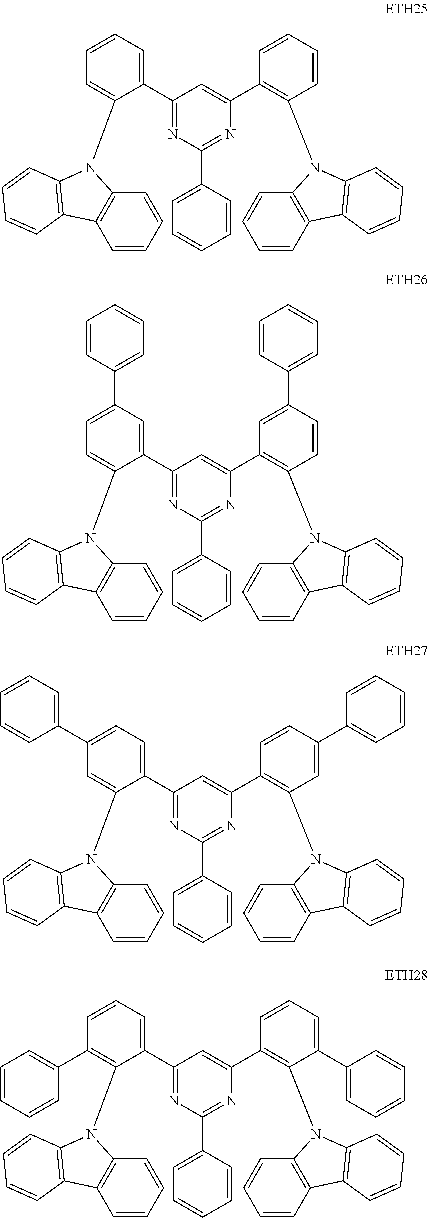 [SMILES: c1ccc(-c2nc(-c3ccccc3-n3c4ccccc4c4ccccc43)cc(-c3ccccc3-n3c4ccccc4c4ccccc43)n2)cc1 ; c1ccc(-c2ccc(-n3c4ccccc4c4ccccc43)c(-c3cc(-c4cc(-c5ccccc5)ccc4-n4c5ccccc5c5ccccc54)nc(-c4ccccc4)n3)c2)cc1 ; c1ccc(-c2ccc(-c3cc(-c4ccc(-c5ccccc5)cc4-n4c5ccccc5c5ccccc54)nc(-c4ccccc4)n3)c(-n3c4ccccc4c4ccccc43)c2)cc1 ; c1ccc(-c2nc(-c3cccc(-c4ccccc4)c3-n3c4ccccc4c4ccccc43)cc(-c3cccc(-c4ccccc4)c3-n3c4ccccc4c4ccccc43)n2)cc1]

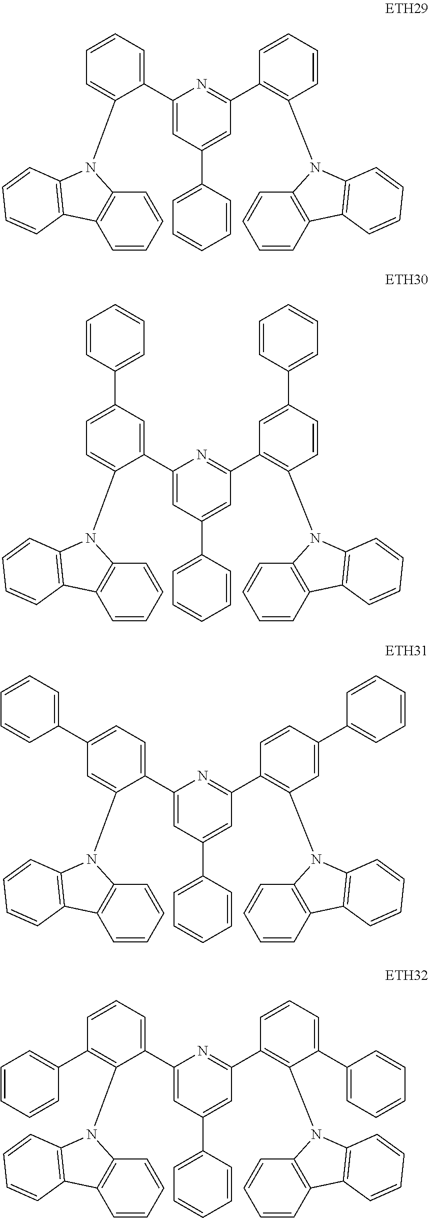 [SMILES: c1ccc(-c2cc(-c3ccccc3-n3c4ccccc4c4ccccc43)nc(-c3ccccc3-n3c4ccccc4c4ccccc43)c2)cc1 ; c1ccc(-c2cc(-c3cc(-c4ccccc4)ccc3-n3c4ccccc4c4ccccc43)nc(-c3cc(-c4ccccc4)ccc3-n3c4ccccc4c4ccccc43)c2)cc1 ; c1ccc(-c2cc(-c3ccc(-c4ccccc4)cc3-n3c4ccccc4c4ccccc43)nc(-c3ccc(-c4ccccc4)cc3-n3c4ccccc4c4ccccc43)c2)cc1 ; c1ccc(-c2cc(-c3cccc(-c4ccccc4)c3-n3c4ccccc4c4ccccc43)nc(-c3cccc(-c4ccccc4)c3-n3c4ccccc4c4ccccc43)c2)cc1]

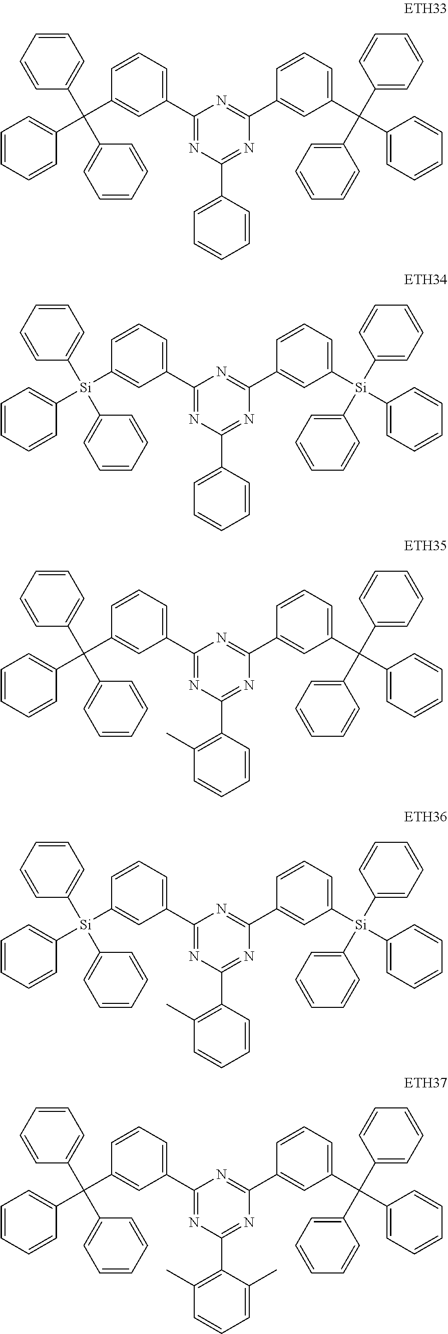

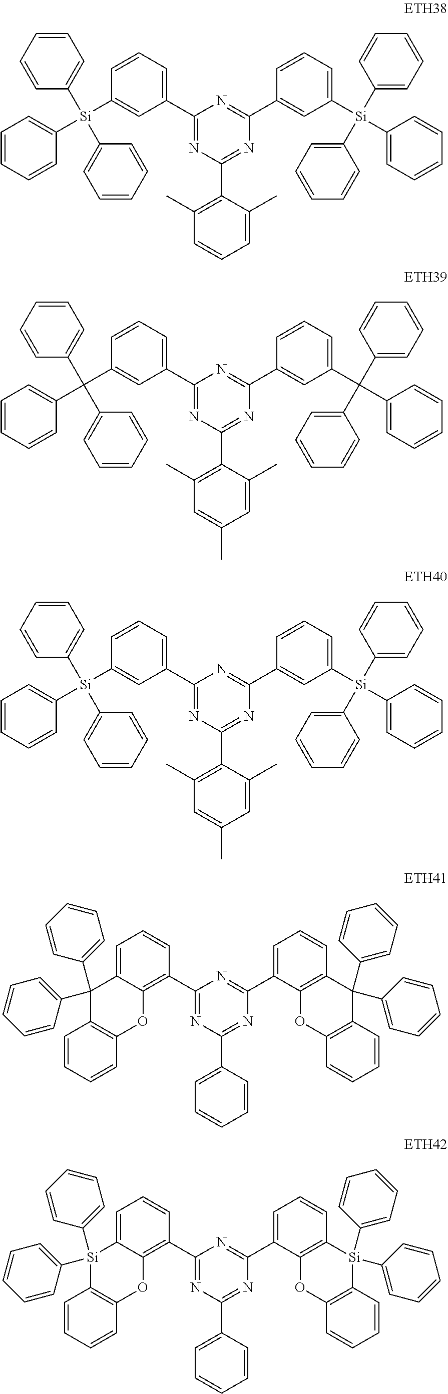

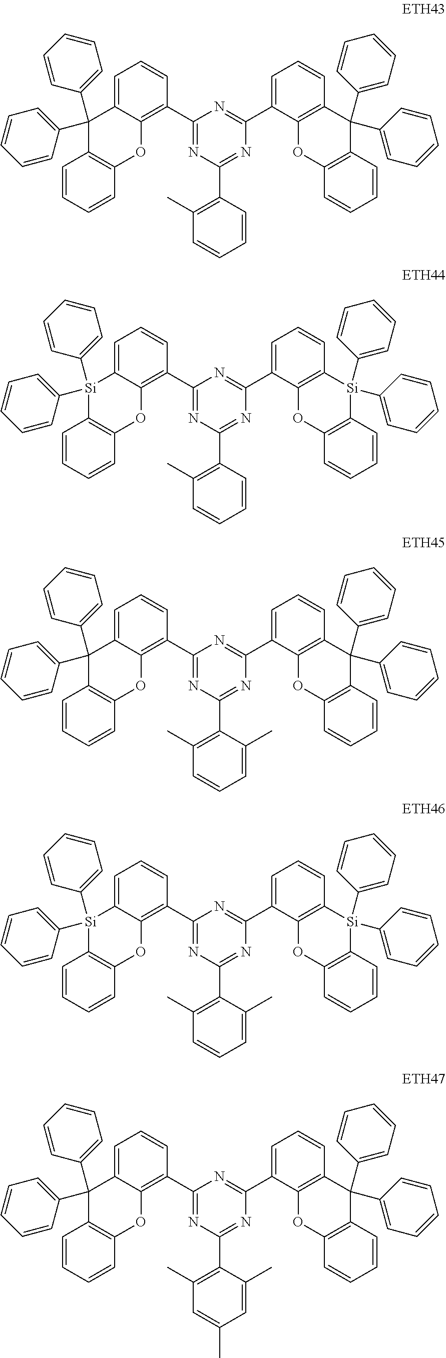

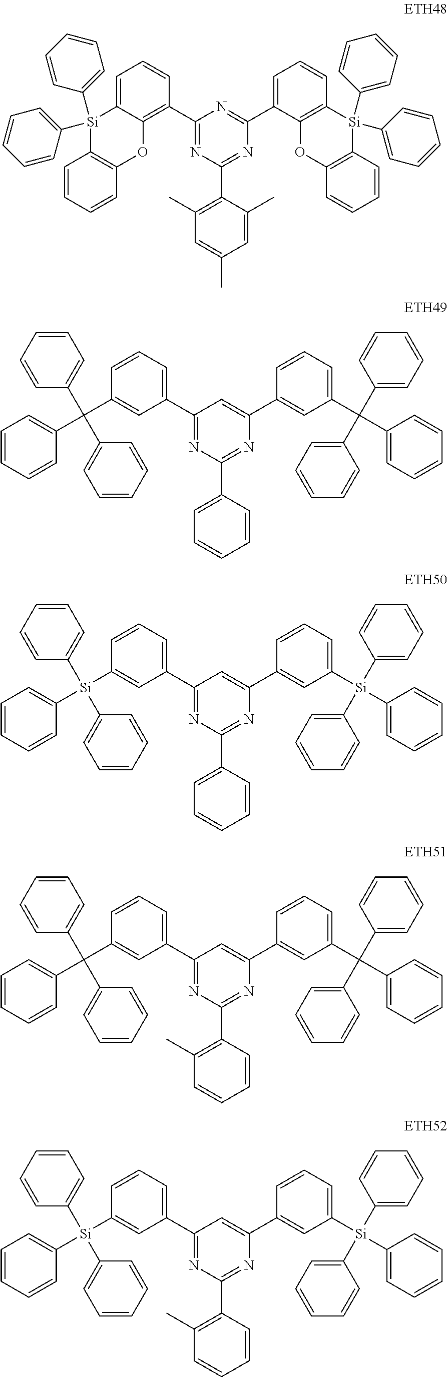

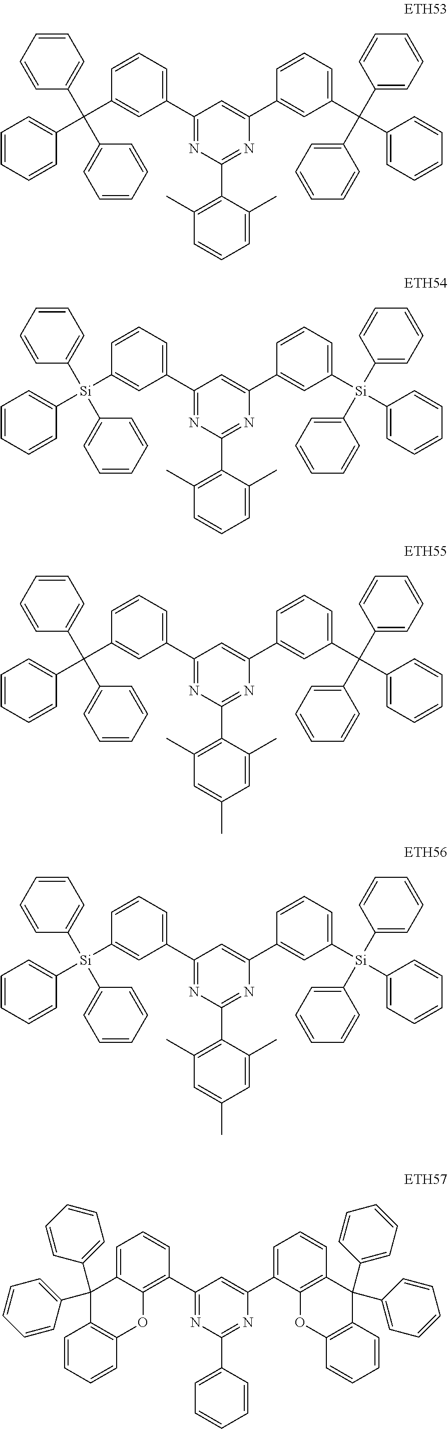

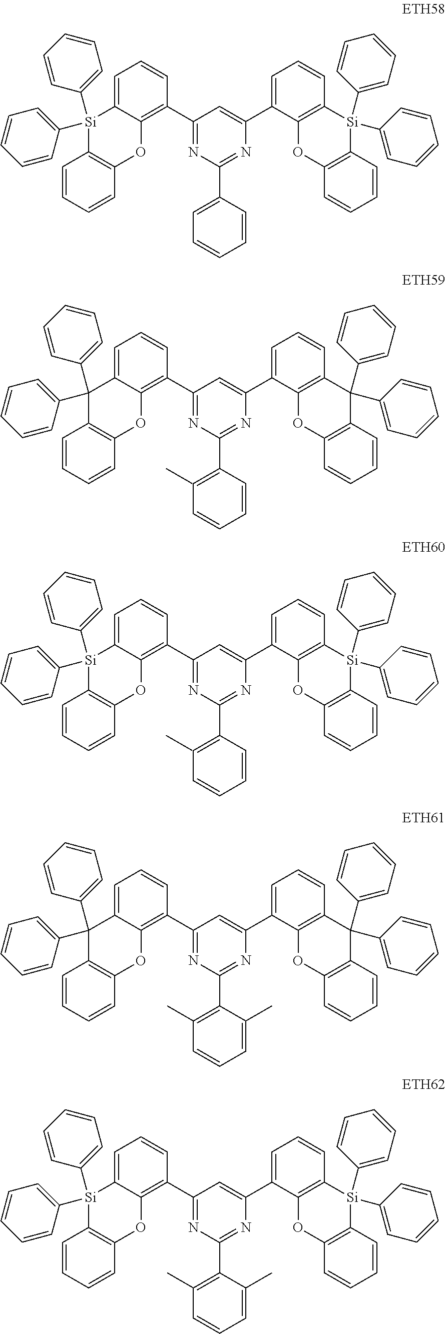

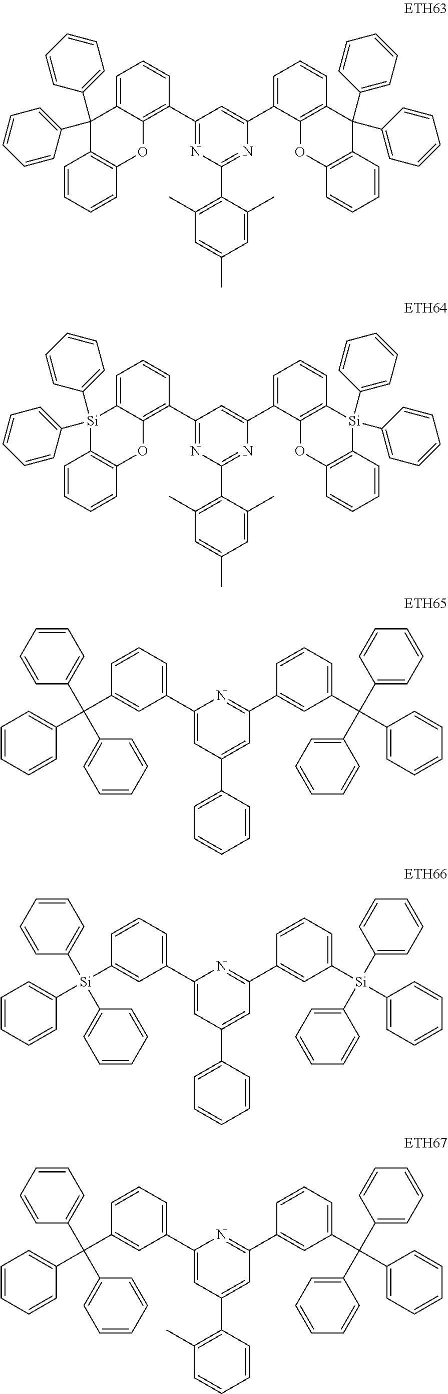

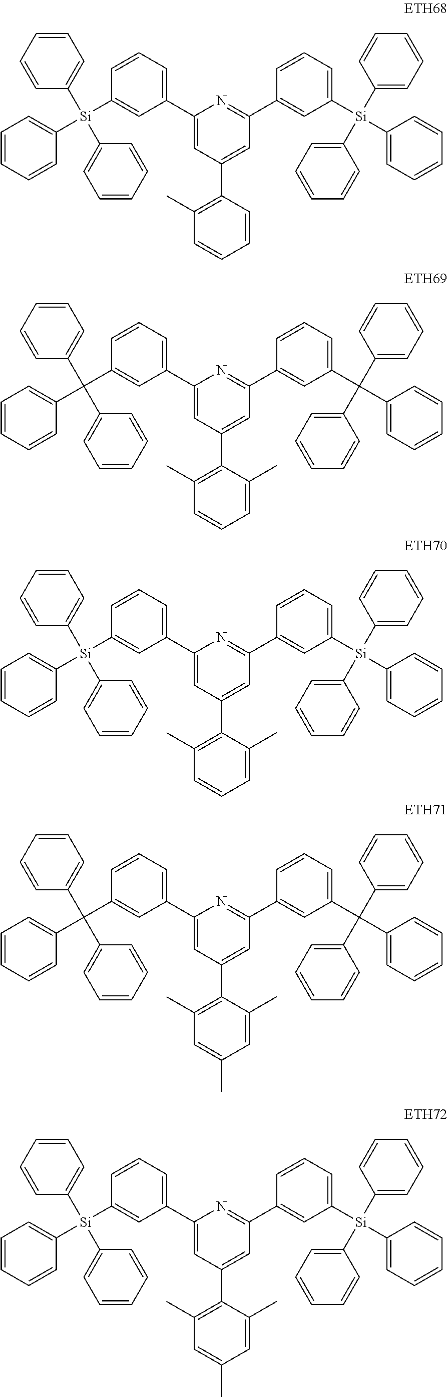

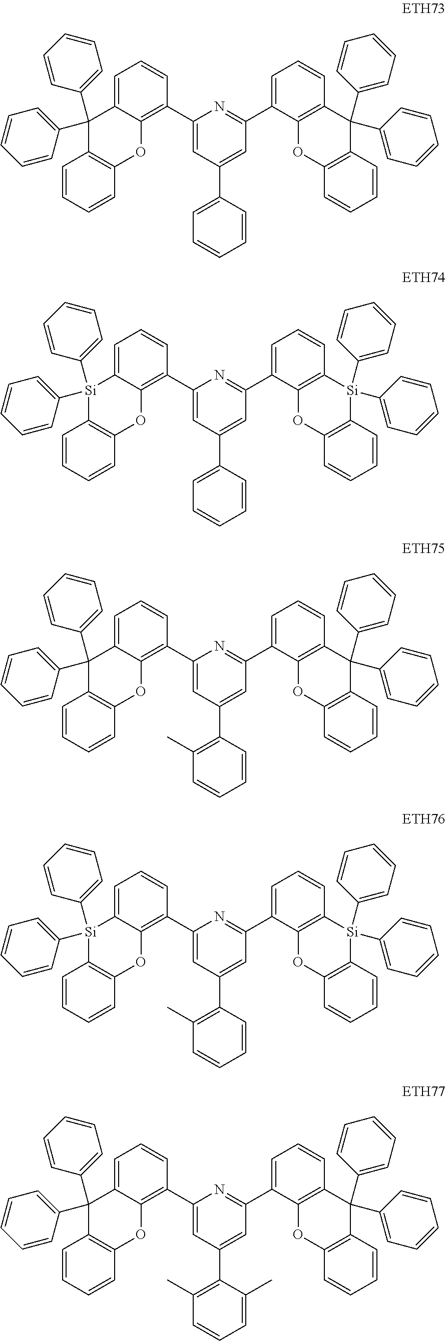

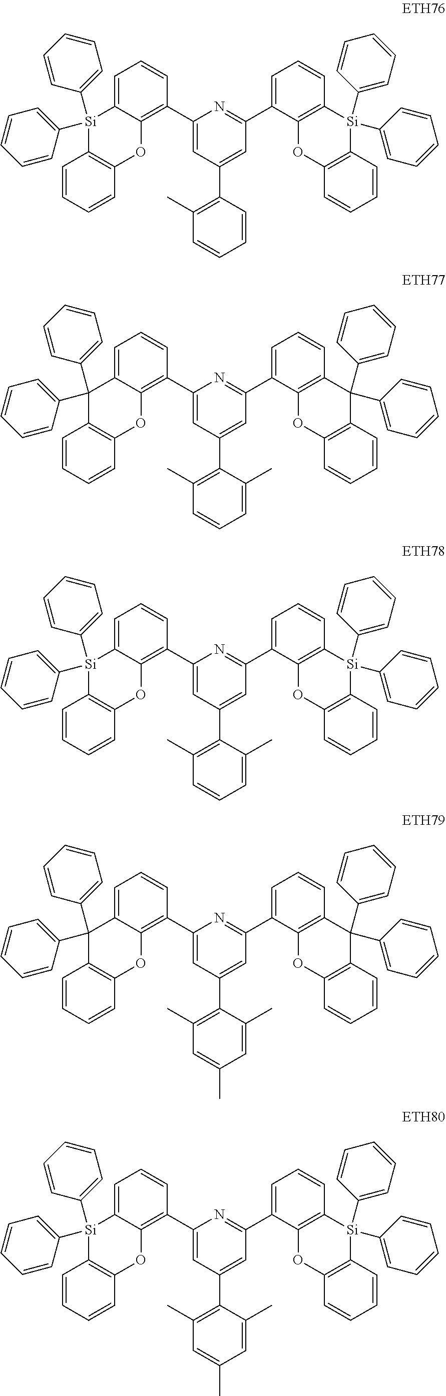

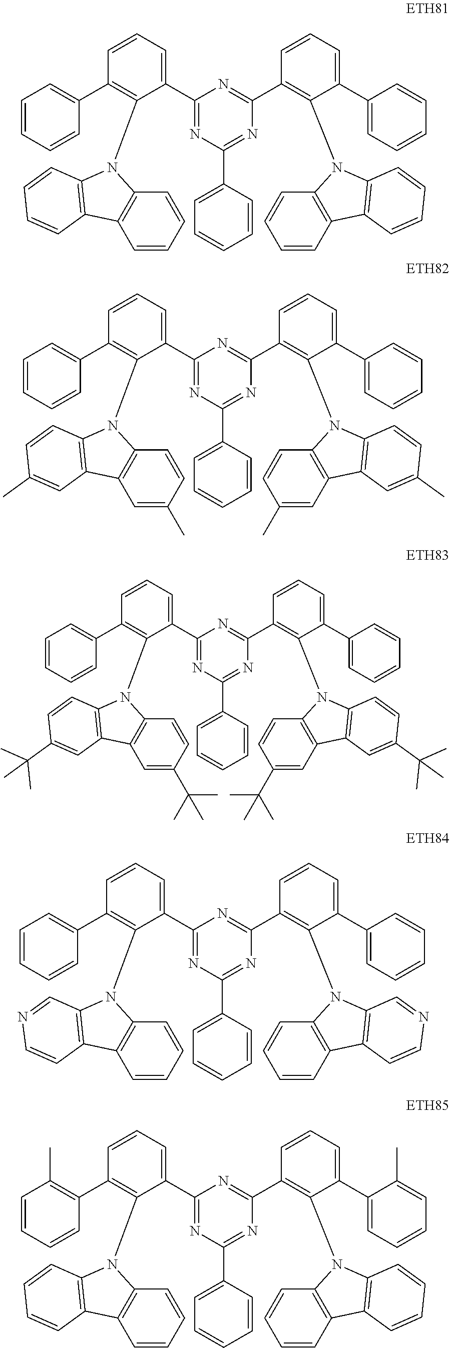

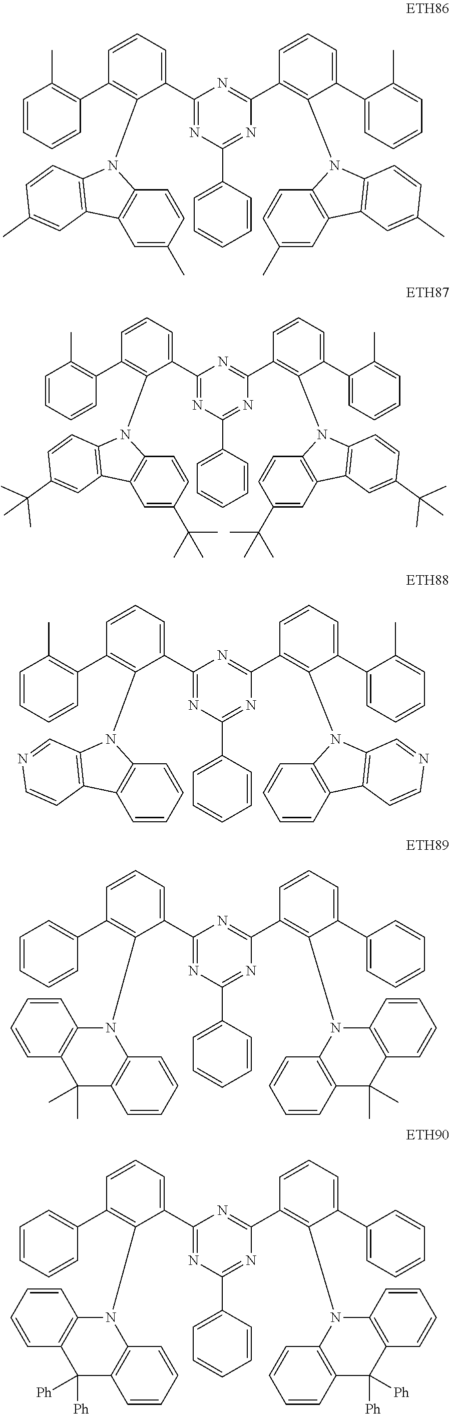

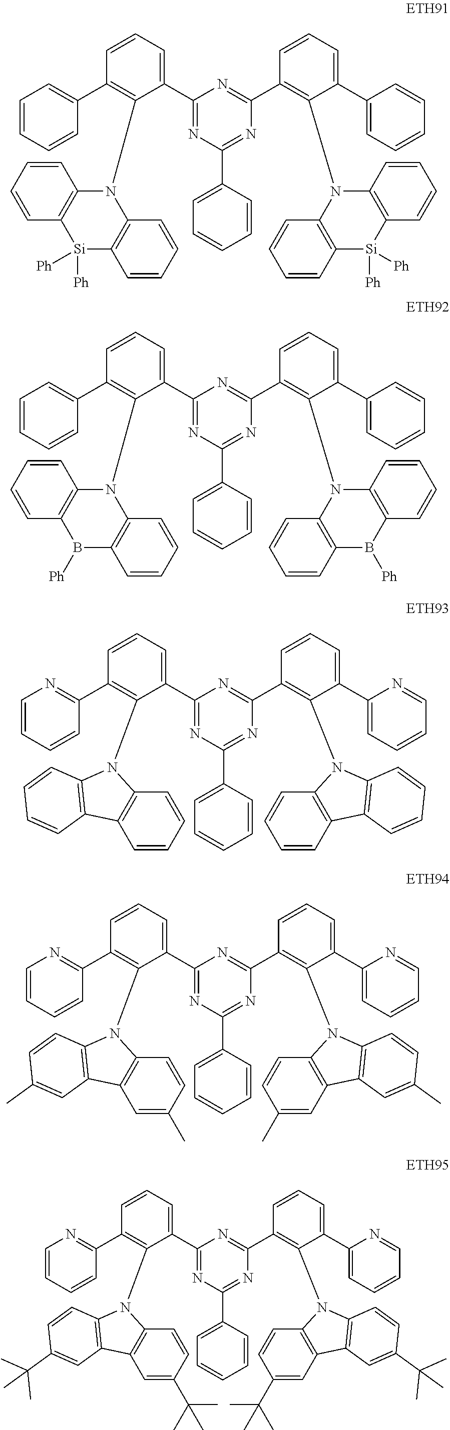

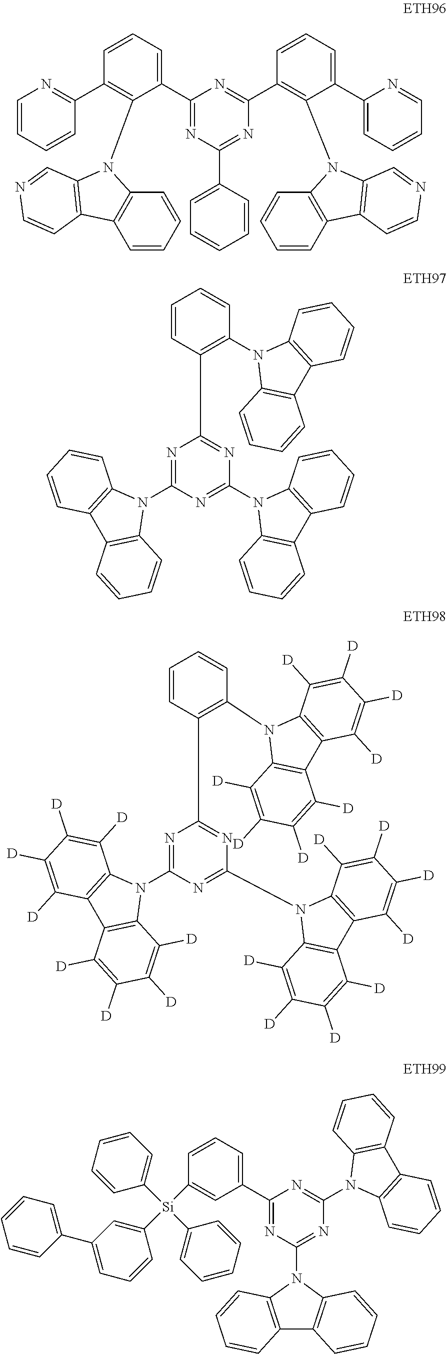

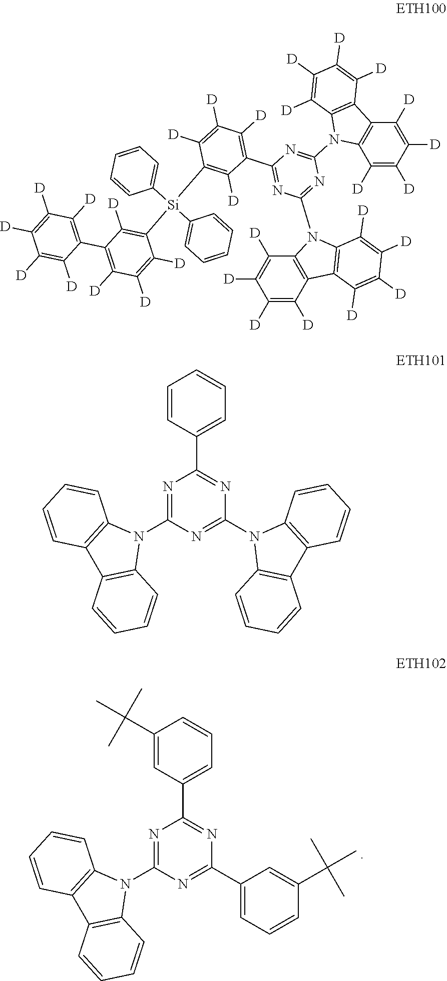 [SMILES: [2H]c1c([2H])c([2H])c(-c2c([2H])c([2H])c([2H])c([Si](c3ccccc3)(c3ccccc3)c3c([2H])c([2H])c([2H])c(-c4nc(-n5c6c([2H])c([2H])c([2H])c([2H])c6c6c([2H])c([2H])c([2H])c([2H])c65)nc(-n5c6c([2H])c([2H])c([2H])c([2H])c6c6c([2H])c([2H])c([2H])c([2H])c65)n4)c3[2H])c2[2H])c([2H])c1[2H] ; c1ccc(-c2nc(-n3c4ccccc4c4ccccc43)nc(-n3c4ccccc4c4ccccc43)n2)cc1 ; CC(C)(C)c1cccc(-c2nc(-c3cccc(C(C)(C)C)c3)nc(-n3c4ccccc4c4ccccc43)n2)c1]

In one or more embodiments, the hole-transporting host maybe anyone of (e.g., selected from among) Compounds HTH1 to HTH50:

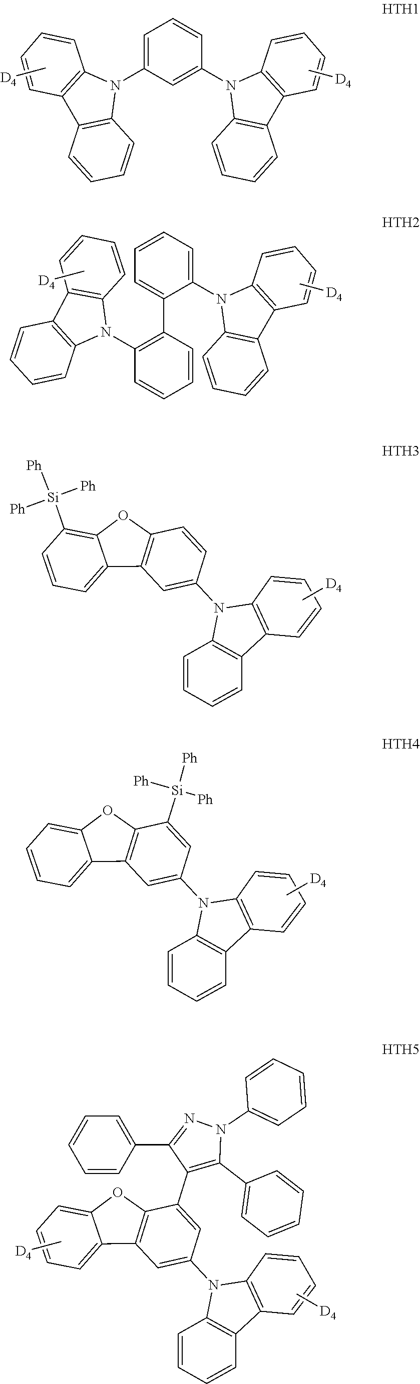

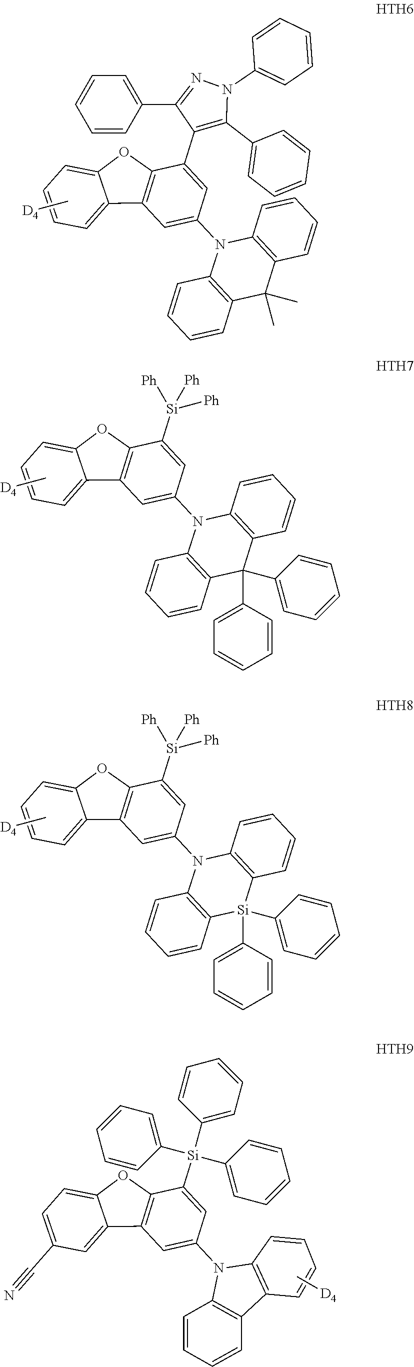

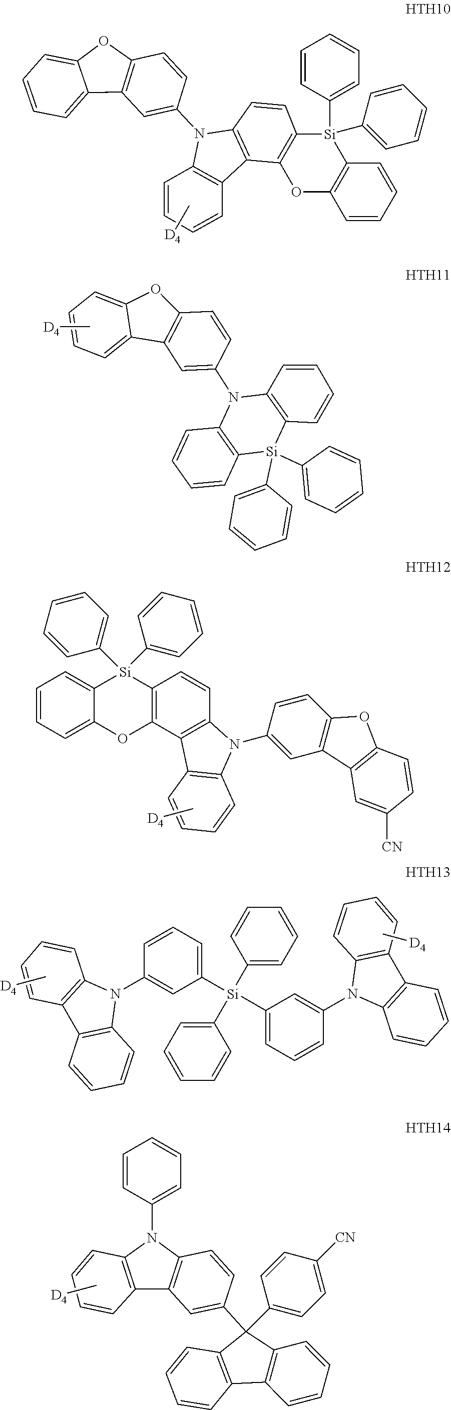

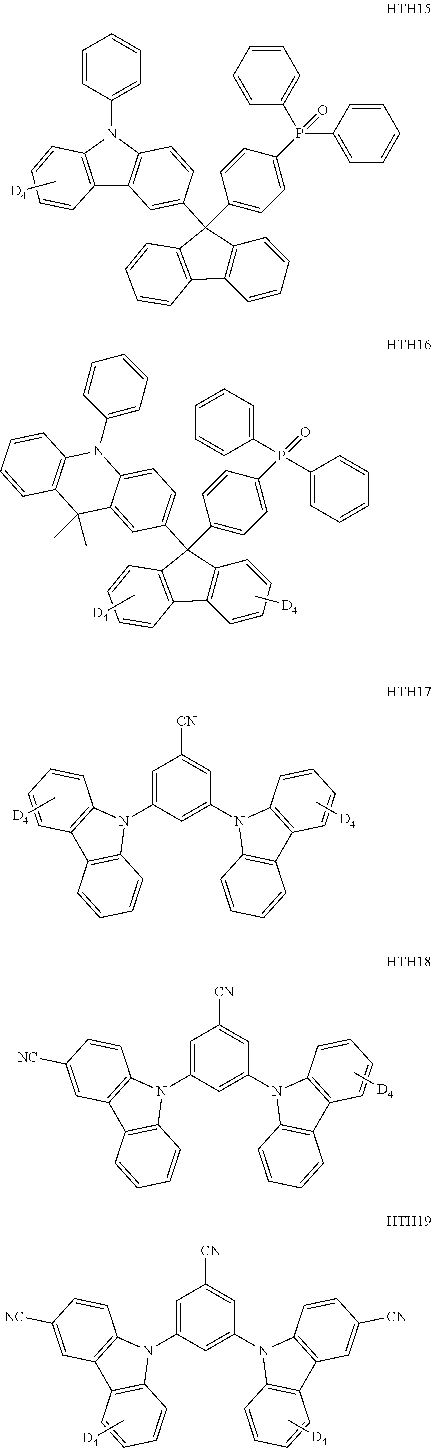

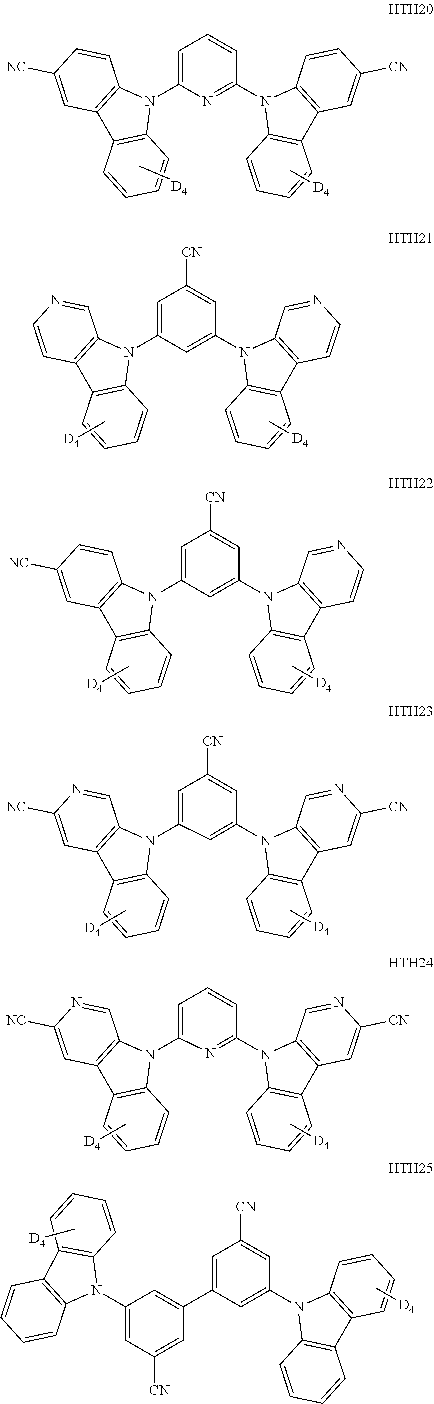

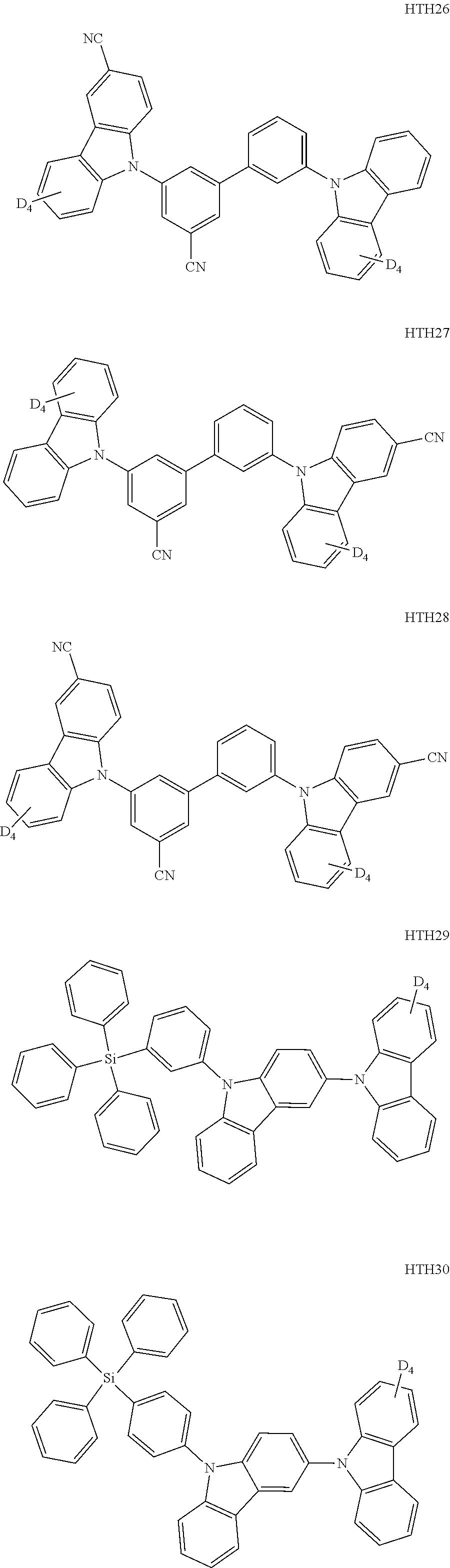

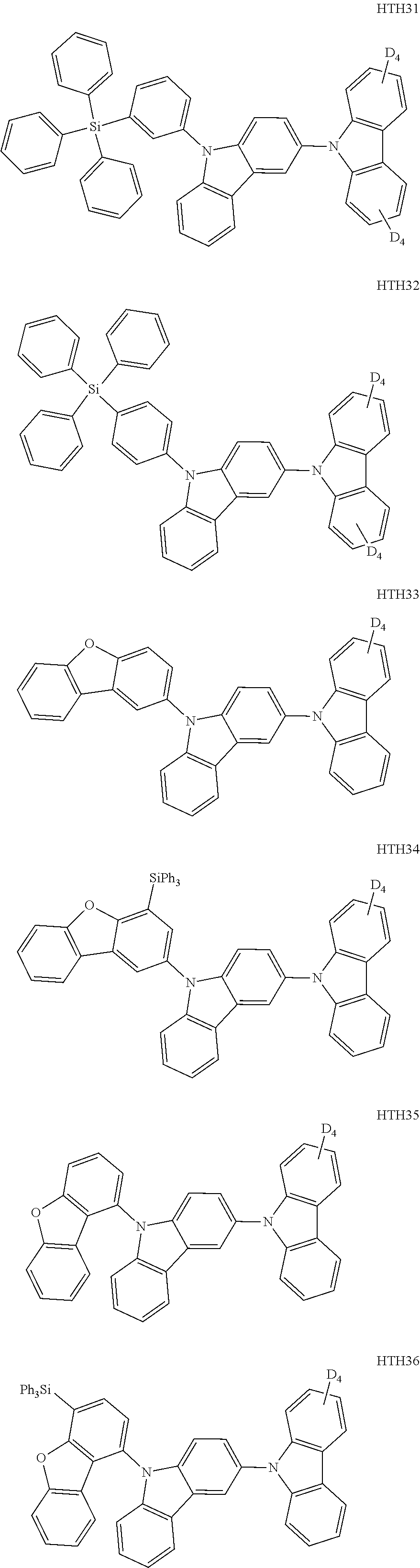

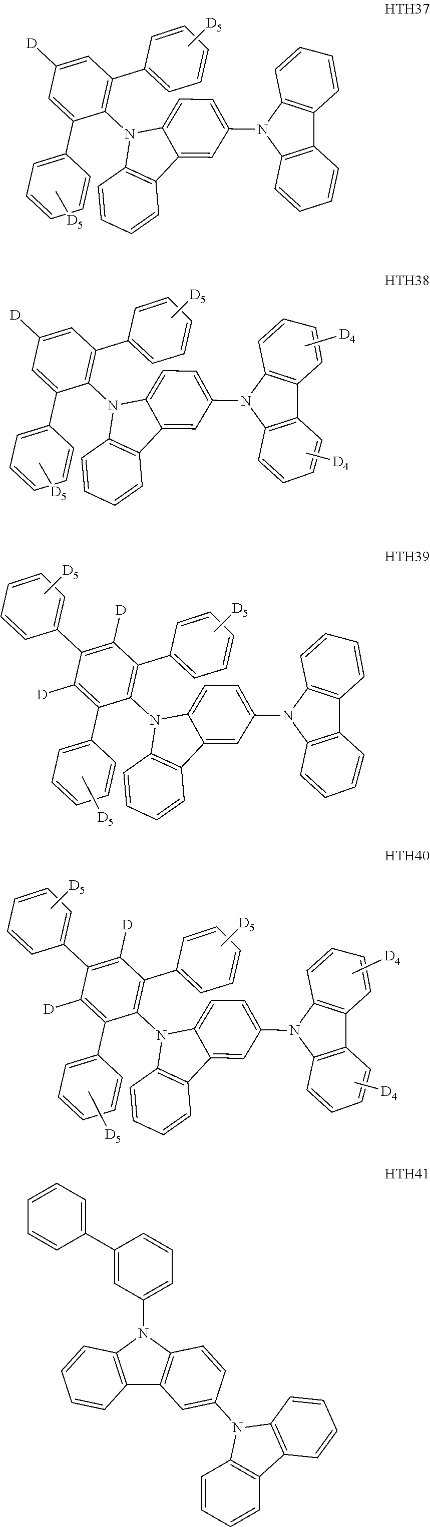

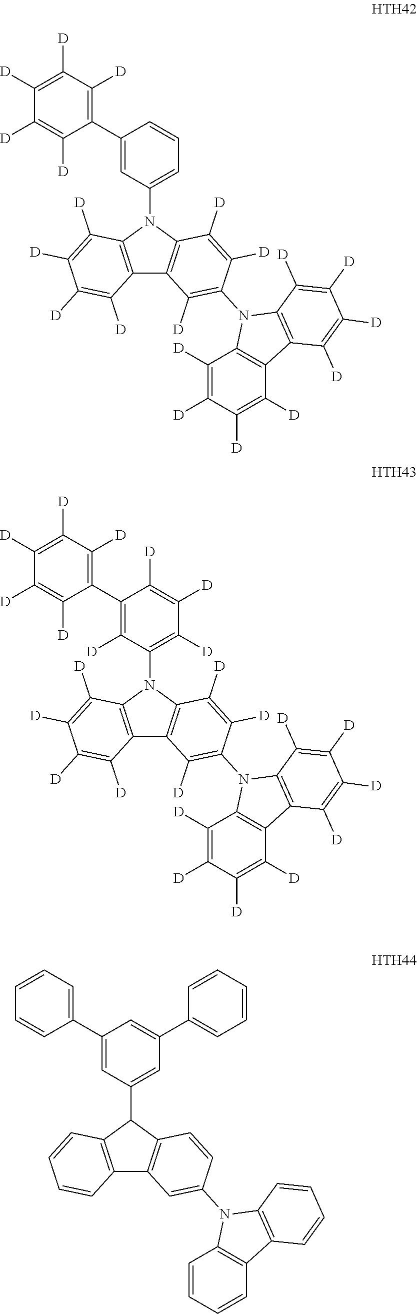 [SMILES: [2H]c1c([2H])c([2H])c(-c2cccc(-n3c4c([2H])c([2H])c([2H])c([2H])c4c4c([2H])c(-n5c6c([2H])c([2H])c([2H])c([2H])c6c6c([2H])c([2H])c([2H])c([2H])c65)c([2H])c([2H])c43)c2)c([2H])c1[2H] ; [2H]c1c([2H])c([2H])c(-c2c([2H])c([2H])c([2H])c(-n3c4c([2H])c([2H])c([2H])c([2H])c4c4c([2H])c(-n5c6c([2H])c([2H])c([2H])c([2H])c6c6c([2H])c([2H])c([2H])c([2H])c65)c([2H])c([2H])c43)c2[2H])c([2H])c1[2H] ; c1ccc(-c2cc(-c3ccccc3)cc(C3c4ccccc4-c4cc(-n5c6ccccc6c6ccccc65)ccc43)c2)cc1]

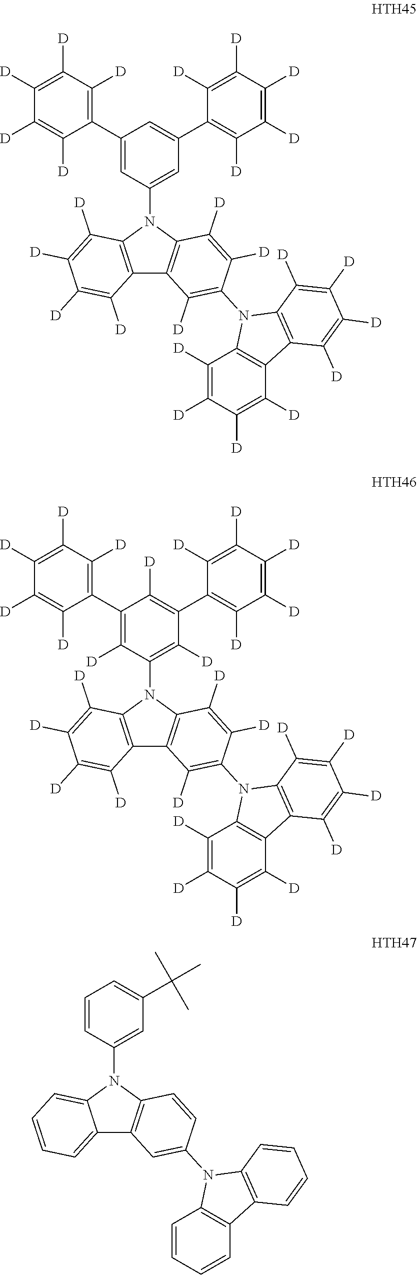

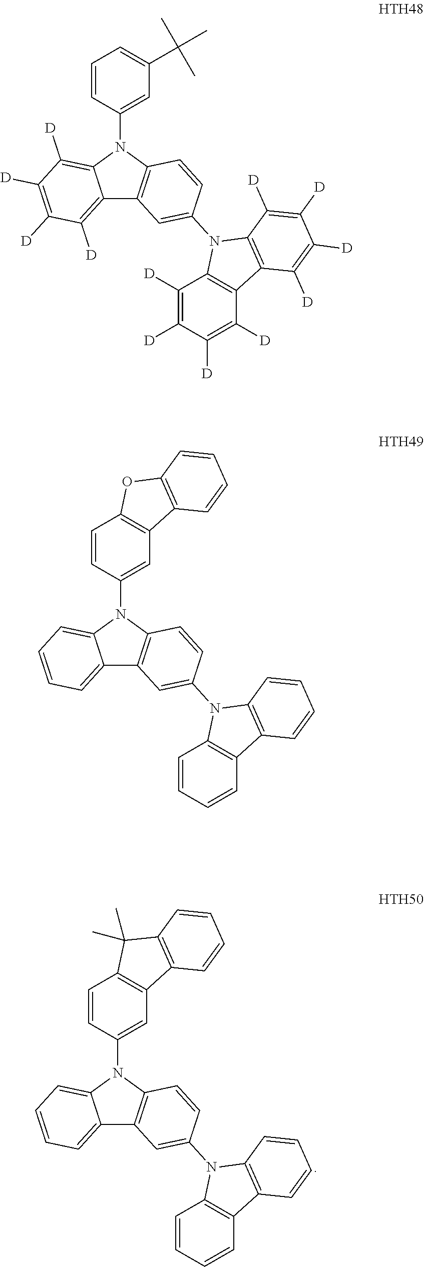 [SMILES: [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12 ; CC1(C)c2ccccc2-c2cc(-n3c4ccccc4c4cc(-n5c6ccccc6c6ccccc65)ccc43)ccc21]

In one or more embodiments, the sensitizer maybe anyone of (e.g., selected from among) Compounds D1 to D10 and PS-1 to PS-3, or may be a compound in which at least one hydrogen included in one of (e.g., selected from among) Compounds D1 to D10 and PS-1 to PS-3 is substituted with deuterium:

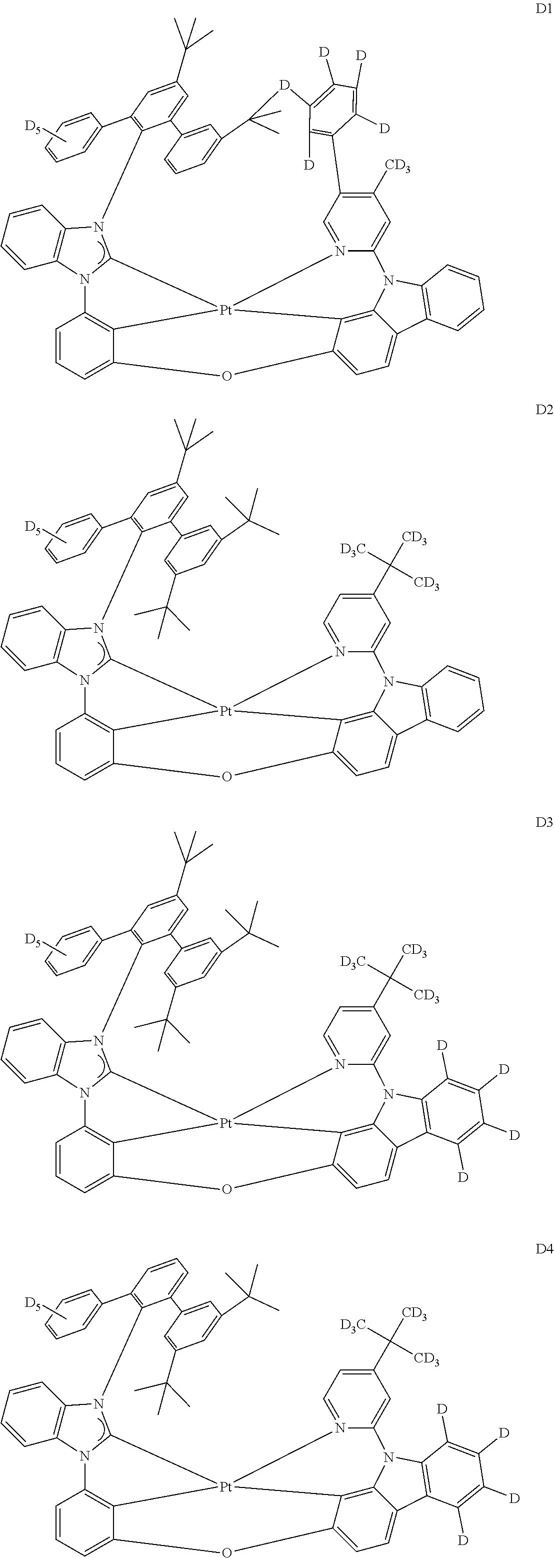

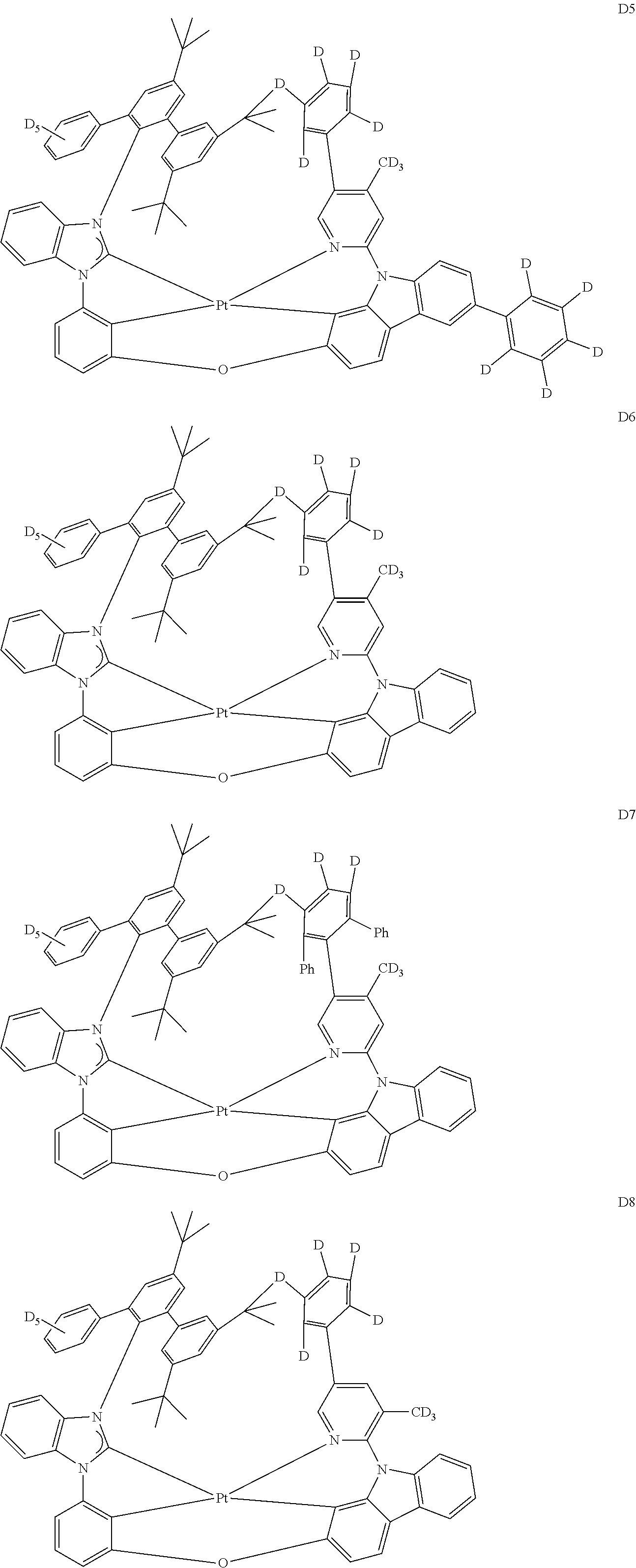

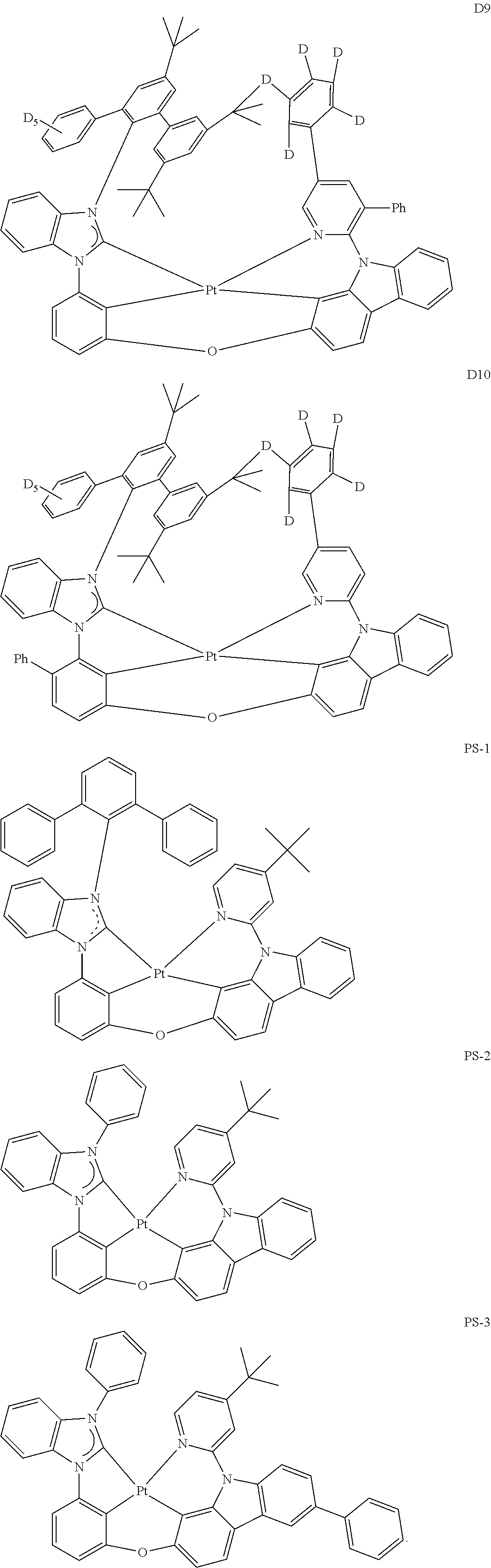

In one or more embodiments, the dopant may be any one of (e.g., selected from among Compounds DFD1 to DFD31:

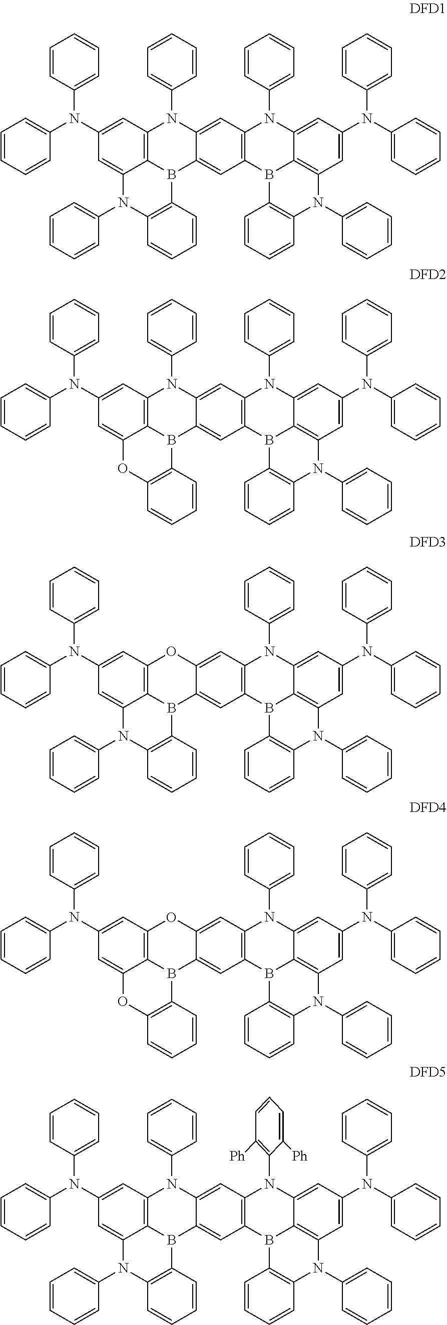

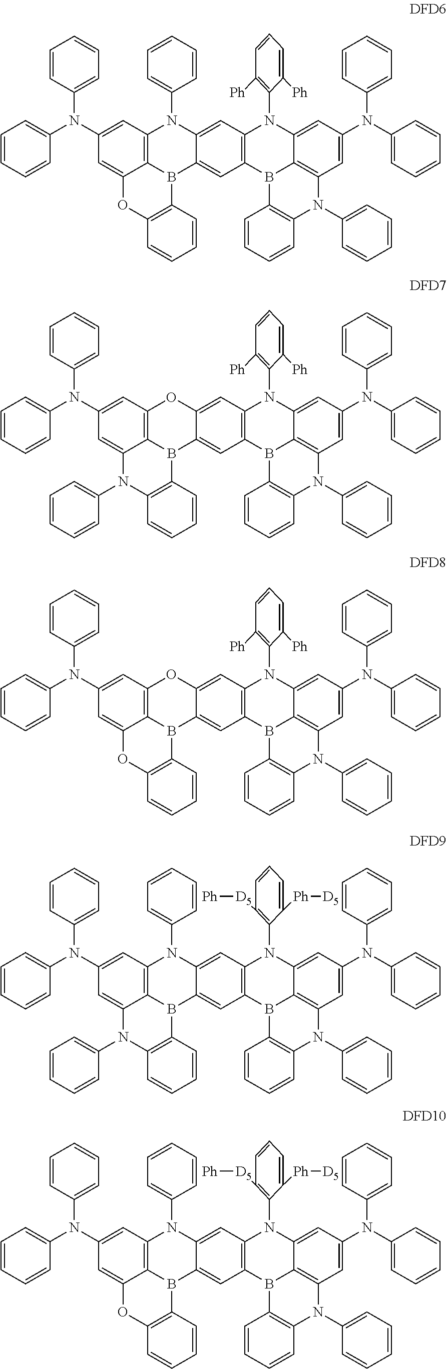

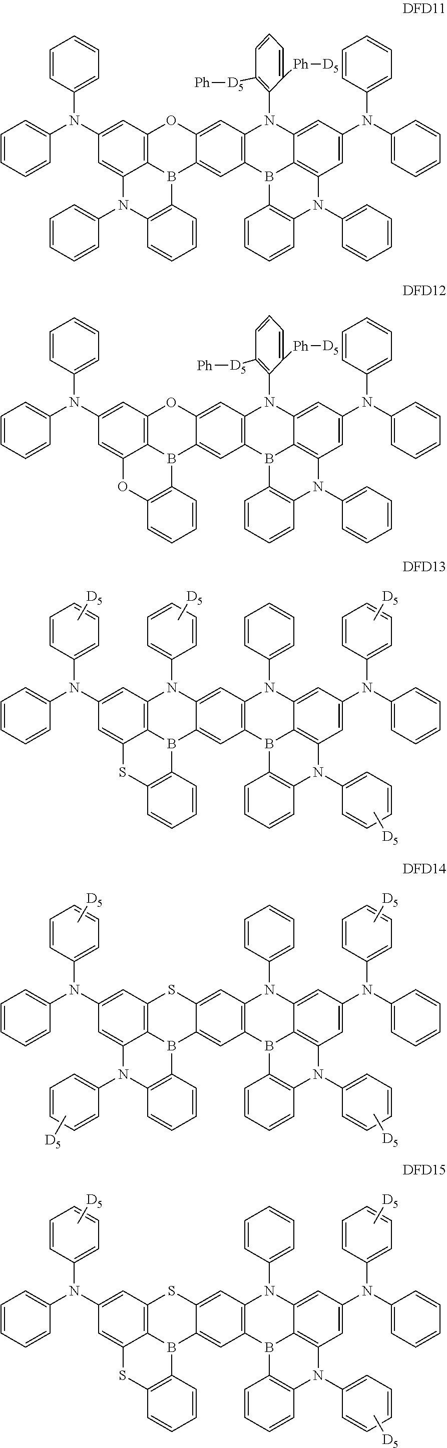

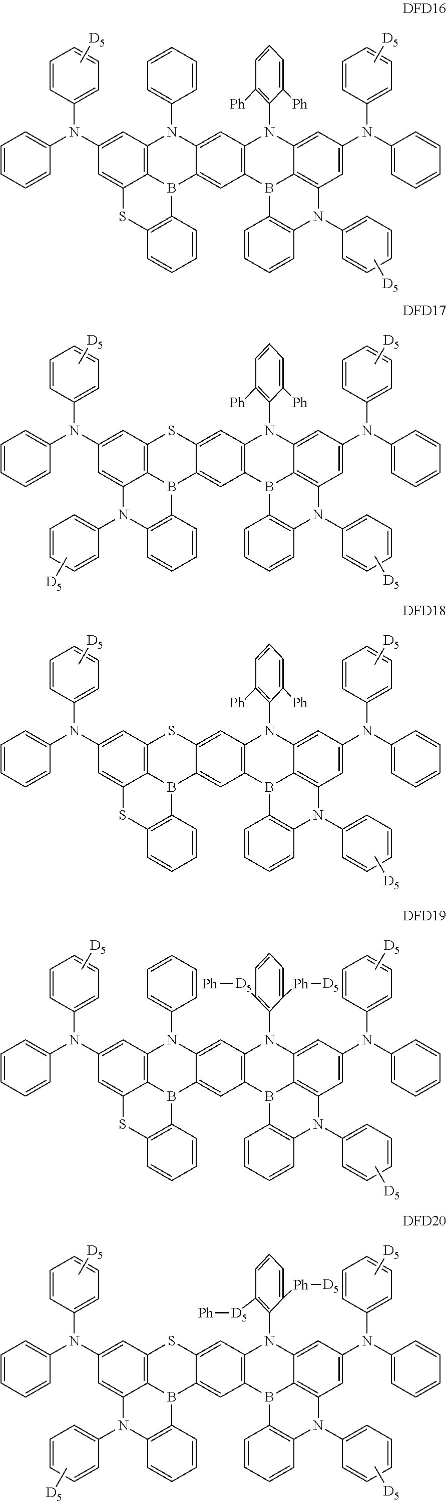

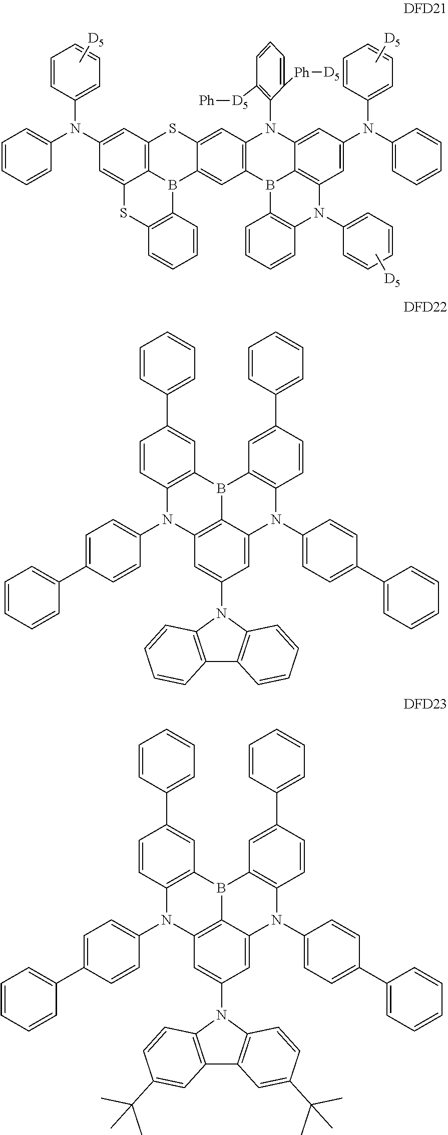

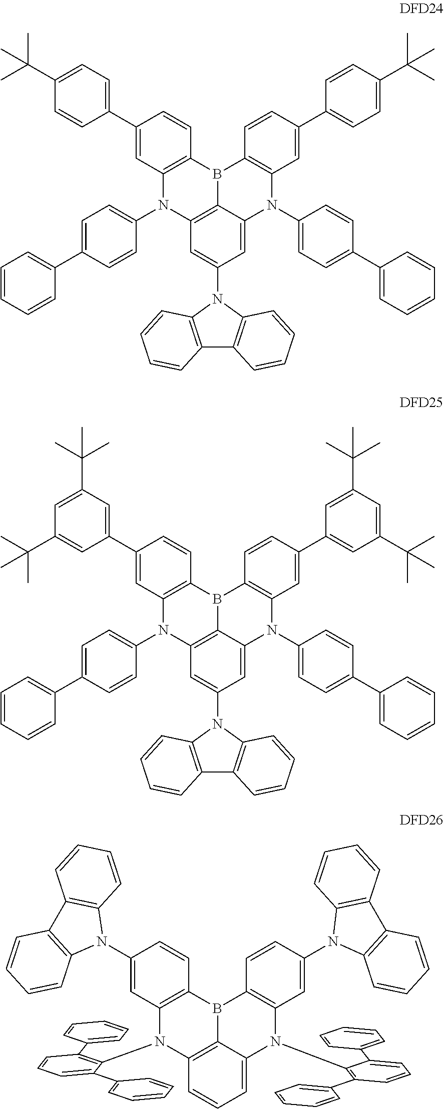 [SMILES: CC(C)(C)c1ccc(-c2ccc3c(c2)N(c2ccc(-c4ccccc4)cc2)c2cc(-n4c5ccccc5c5ccccc54)cc4c2B3c2ccc(-c3ccc(C(C)(C)C)cc3)cc2N4c2ccc(-c3ccccc3)cc2)cc1 ; CC(C)(C)c1cc(-c2ccc3c(c2)N(c2ccc(-c4ccccc4)cc2)c2cc(-n4c5ccccc5c5ccccc54)cc4c2B3c2ccc(-c3cc(C(C)(C)C)cc(C(C)(C)C)c3)cc2N4c2ccc(-c3ccccc3)cc2)cc(C(C)(C)C)c1 ; c1ccc(-c2cccc(-c3ccccc3)c2N2c3cc(-n4c5ccccc5c5ccccc54)ccc3B3c4ccc(-n5c6ccccc6c6ccccc65)cc4N(c4c(-c5ccccc5)cccc4-c4ccccc4)c4cccc2c43)cc1]

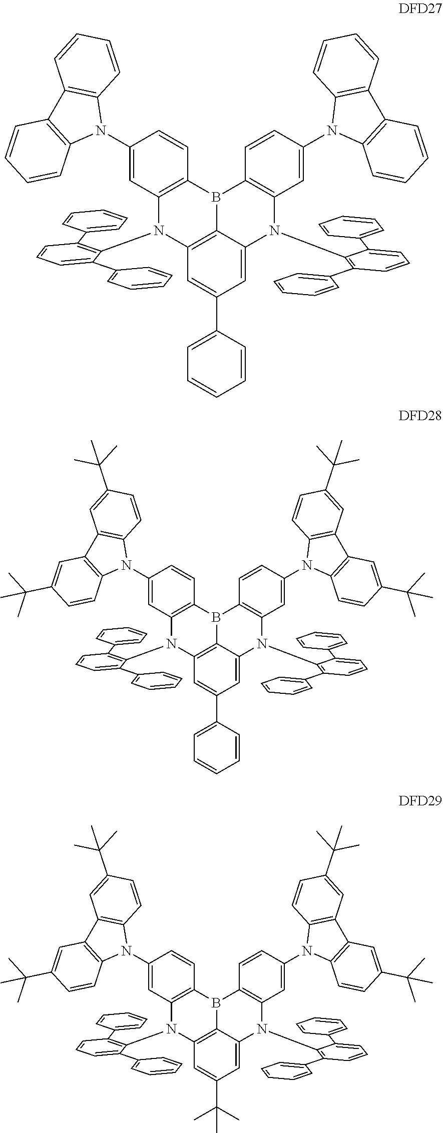

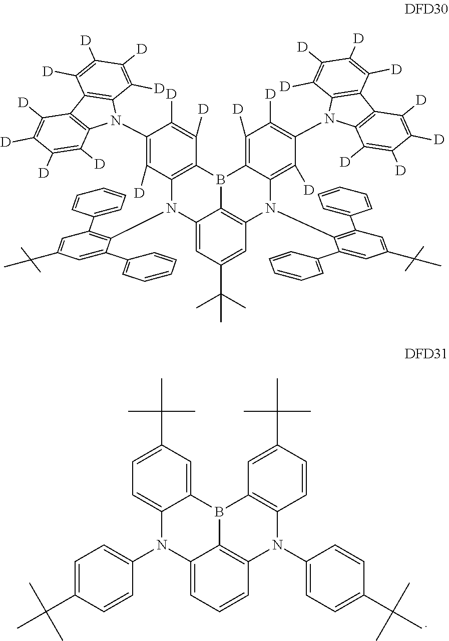

In the compounds described above, “Ph” represents a phenyl group, “D” represents deuterium, and “D₅” represents substitution with five deuterium atoms. For example, a group represented by

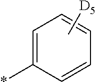

may be identical to a group represented by

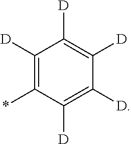

The organic compound of one or more embodiments is represented by Formula 1 and is a diamine-based compound to which a fluorenyl group is bonded. A linking group (L₁)ₐ₁ is bonded to a specific position of a benzene group linking two amine groups. An aryl group substituted with a phenyl group is linked to the linking group (L₁)ₐ₁. The phenyl group is in an ortho position with respect to the linking group (L₁)ₐ₁. In addition, if (e.g., when) the organic compound satisfies Condition 1, the group formed of the aryl group substituted with the phenyl group at the ortho position together with the phenyl may not be a biphenyl group and may be a group in which at least three rings are bonded to each other, and if (e.g., when) the organic compound satisfies Condition 2, the aryl group substituted with the phenyl group at the ortho position may not be a benzene group and may be a naphthalene group. The organic compound satisfying at least one of Condition 1 or Condition 2 may have desired electrical stability and high charge-transporting ability, and thus, a light-emitting device including the organic compound may have a low driving voltage, high luminescence efficiency, and a long lifespan.

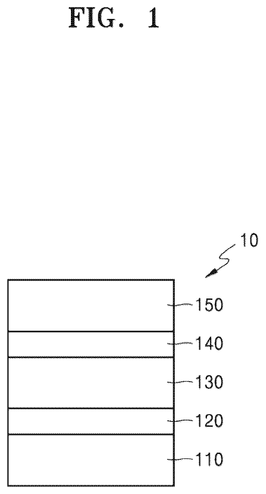

### Description of FIG. 1

FIG. 1 is a schematic cross-sectional view of a light-emitting device 10 according to one or more embodiments. The light-emitting device 10 may include a first electrode 110, an interlayer, and a second electrode 150. The interlayer may include a hole transport region 120, an emission layer 130, and an electron transport region 140.

Hereinafter, the structure of the light-emitting device 10 according to one or more embodiments and a method of manufacturing the light-emitting device 10 will be described with reference to FIG. 1.

### First Electrode 110

In FIG. 1, in one or more embodiments, a substrate may be additionally provided and arranged under the first electrode 110 and/or on the second electrode 150. As the substrate, a glass substrate or a plastic substrate may be used. In one or more embodiments, the substrate may be a flexible substrate. For example, the substrate may include plastics with excellent or suitable heat resistance and durability, such as polyimide, polyethylene terephthalate (PET), polycarbonate, polyethylene naphthalate, polyarylate (PAR), polyetherimide, or any combination thereof.

The first electrode 110 may be formed by depositing or sputtering a material for forming the first electrode 110 on the substrate. When the first electrode 110 is an anode, a high-work function material that facilitates injection of holes may be used as a material for forming the first electrode 110.

The first electrode 110 may be a reflective electrode, a transflective electrode, or a transmissive electrode. In one or more embodiments, if (e.g., when) the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In one or more embodiments, if (e.g., when) the first electrode 110 is a transflective electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layer structure including (e.g., consisting of) a single layer or a multi-layer structure including multiple layers. For example, in some embodiments, the first electrode 110 may have a three-layer structure of ITO/Ag/ITO.

### Interlayer

The interlayer may be arranged on the first electrode 110. The interlayer may include the hole transport region 120, the emission layer 130, and the electron transport region 140.

The interlayer may include one or more suitable organic materials, for example, the organic compound of the present disclosure, a metal-containing compound such as an organometallic compound, an inorganic material such as quantum dots, and/or the like.

In one or more embodiments, the interlayer may include i) two or more emitting units sequentially stacked between the first electrode 110 and the second electrode 150 and ii) a charge generation layer arranged between the two or more emitting units. When the interlayer includes the emitting units and the charge generation layer as described above, the light-emitting device 10 may be a tandem light-emitting device.

### Hole Transport Region 120

The hole transport region 120 may have i) a single-layer structure including (e.g., consisting of) a single layer including (e.g., consisting of) a single material, ii) a single-layer structure including (e.g., consisting of) a single layer including multiple materials that are different from each other, or iii) a multi-layer structure including multiple layers including multiple materials that are different from each other.

The hole transport region 120 may include a hole injection layer, a hole transport layer, an emission auxiliary layer, an electron-blocking layer, or any combination thereof.

In one or more embodiments, the hole transport region 120 may have a multi-layer structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron-blocking layer structure, wherein constituent layers of each structure are stacked sequentially from the first electrode 110 in the stated order.

In one or more embodiments, the hole transport region 120 may include a compound represented by Formula 201, a compound represented by Formula 202, or any combination thereof:

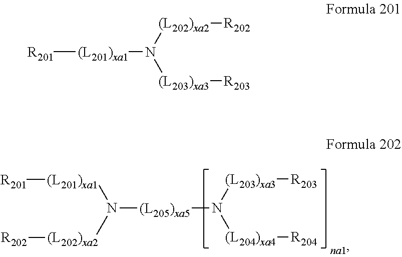

wherein, in Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group (for example, a carbazole group and/or the like) unsubstituted or substituted with at least one R₁₀ₐ (for example, see Compound HT16 and/or the like),
R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and
na1 may be an integer from 1 to 4.

In one or more embodiments, each of Formulae 201 and 202 may include at least one selected from among groups represented by Formulae CY201 to CY217:

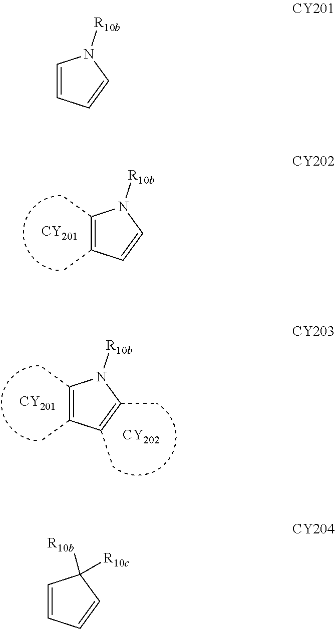

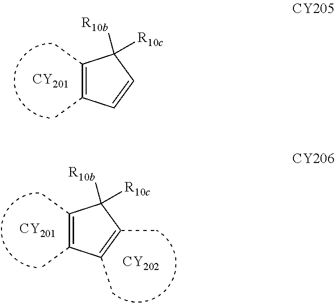

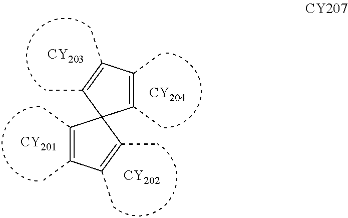

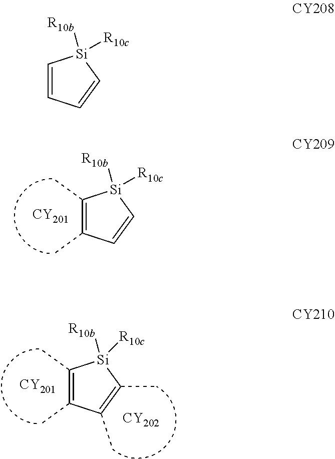

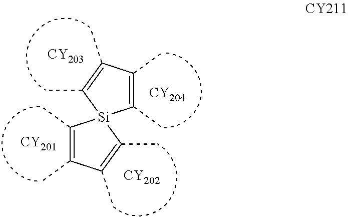

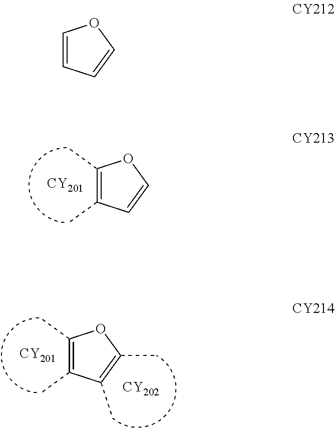

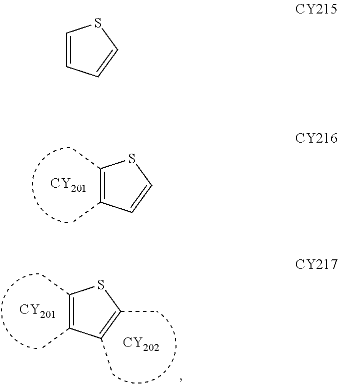

wherein, in Formulae CY201 to CY217, R_{10b} and R_{10c} may each independently be the same as described with respect to R₁₀ₐ, ring CY201 to ring CY204 may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

In one or more embodiments, ring CY201 to ring CY204 in Formulae CY201 to CY217 may each independently be a benzene group, a naphthalene group, a phenanthrene group, or an anthracene group.

In one or more embodiments, each of Formulae 201 and 202 may include at least one selected from among groups represented by Formulae CY201 to CY203.

In one or more embodiments, Formula 201 may include at least one selected from among groups represented by Formulae CY201 to CY203 and at least one selected from among groups represented by Formulae CY204 to CY217.

In one or more embodiments, in Formula 201, xa1 may be 1, R₂₀₁ may be a group represented by any one selected from among Formulae CY201 to CY203, xa2 may be 0, and R₂₀₂ may be a group represented by any one selected from among Formulae CY204 to CY207.

In one or more embodiments, each of Formulae 201 and 202 may not include (e.g., may exclude any of) groups represented by Formulae CY201 to CY203.

In one or more embodiments, each of Formulae 201 and 202 may not include (e.g., may exclude any of) groups represented by Formulae CY201 to CY203, and may include at least one selected from among groups represented by Formulae CY204 to CY217.

In one or more embodiments, each of Formulae 201 and 202 may not include (e.g., may exclude any of) groups represented by Formulae CY201 to CY217.

In one or more embodiments, the hole transport region 120 may include: at least one of (e.g., selected from among) Compounds HT1 to HT46; 4,4′,4″-[tris(3-methylphenyl)phenylamino]triphenylamine (m-MTDATA); 4,4′,4″-tris(N,N-diphenylamino)triphenylamine (TDATA); 4,4′,4″-tris[N-(2-naphthyl)-N-phenylamino]-triphenylamine (2-TNATA); N,N′-di(naphthalen-1-yl)-N,N′-diphenyl-benzidine (NPB(NPD)); β-NPB; N,N′-bis(3-methylphenyl)-N,N′-diphenyl-[1,1′-biphenyl]-4,4′-diamine (TPD); spiro-TPD; spiro-NPB; methylated NPB; 4,4′-cyclohexylidene bis[N,N-bis(4-methylphenyl)benzenamine](TAPC); 4,4′-bis[N,N′-(3-tolyl)amino]-3,3′-dimethylbiphenyl (HMTPD); 4,4′,4″-tris(N-carbazolyl)triphenylamine (TCTA); polyaniline/dodecylbenzenesulfonic acid (PANI/DBSA); poly(3,4-ethylenedioxythiophene)/poly(4-styrenesulfonate) (PEDOT/PSS); polyaniline/camphor sulfonic acid (PANI/CSA); polyaniline/poly(4-styrenesulfonate) (PANI/PSS); or any combination thereof:

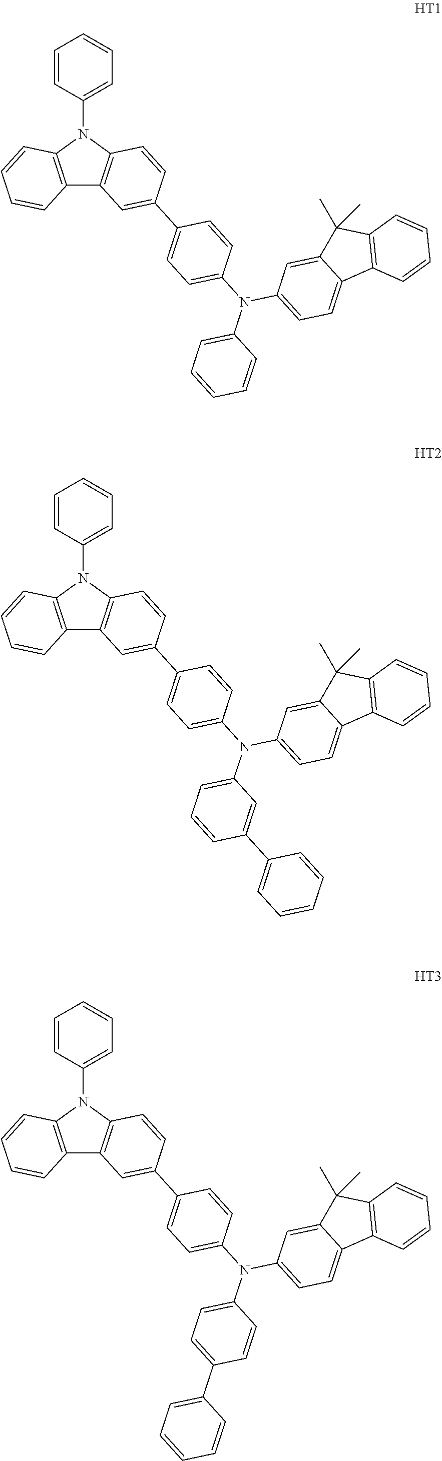 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

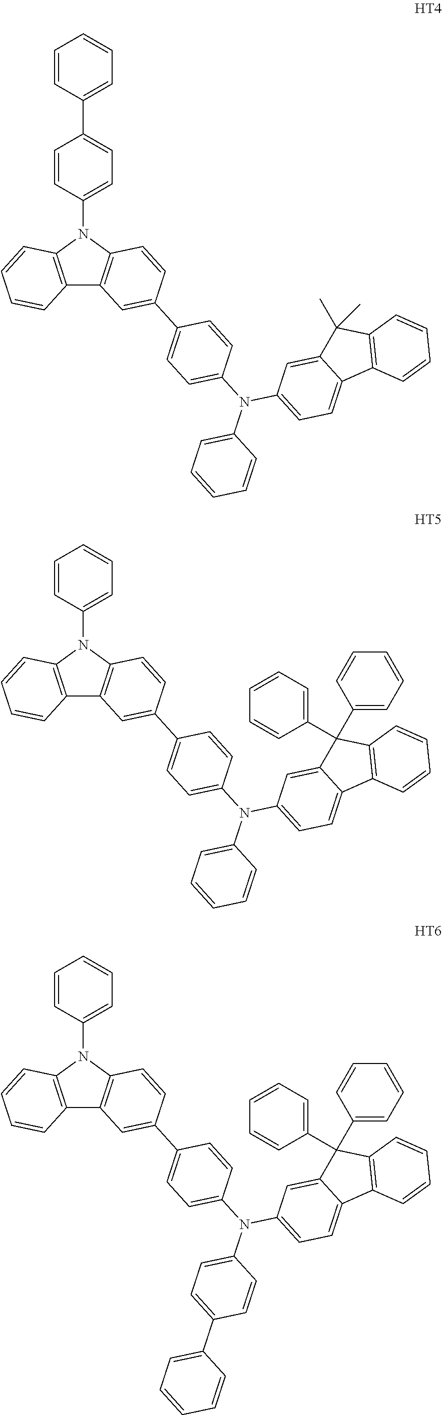 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

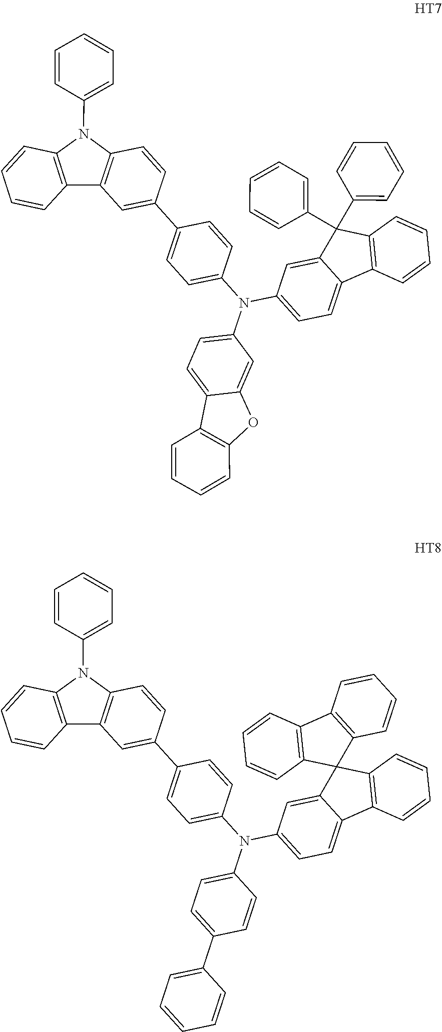 [SMILES: c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

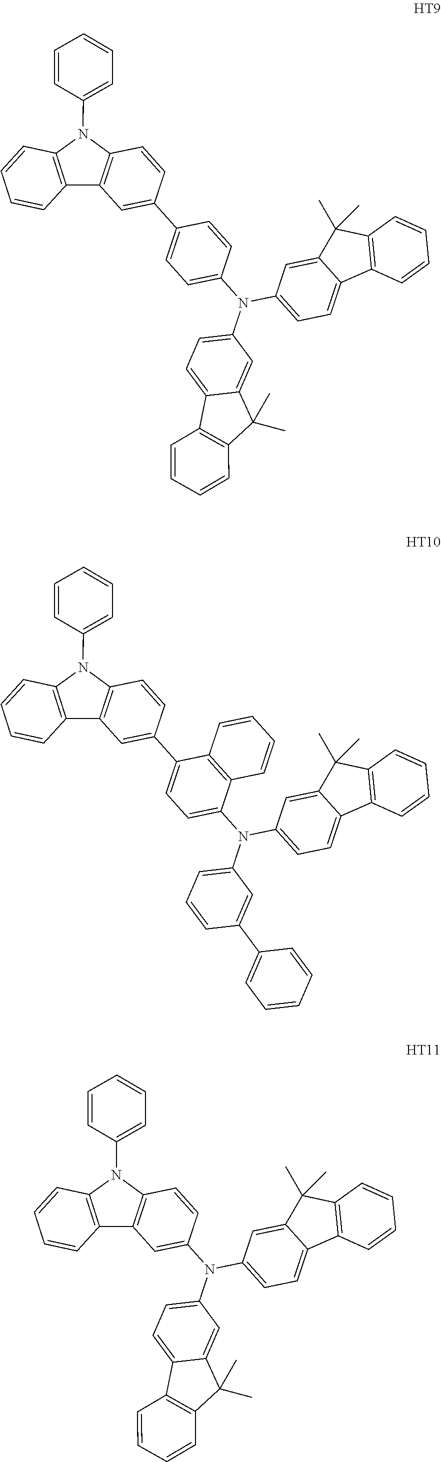 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21]

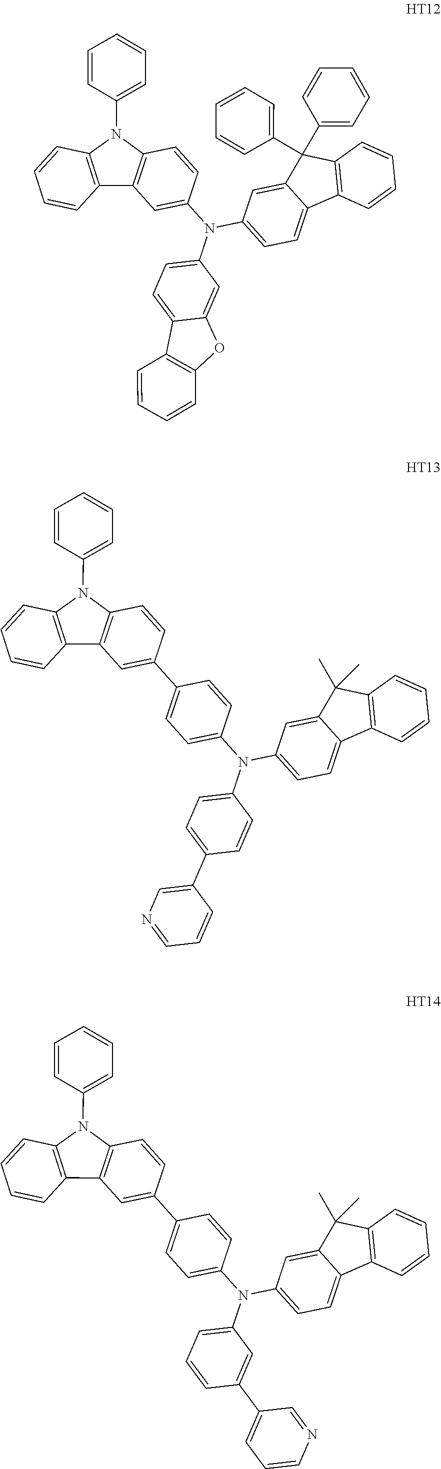 [SMILES: c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1 ; CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21]

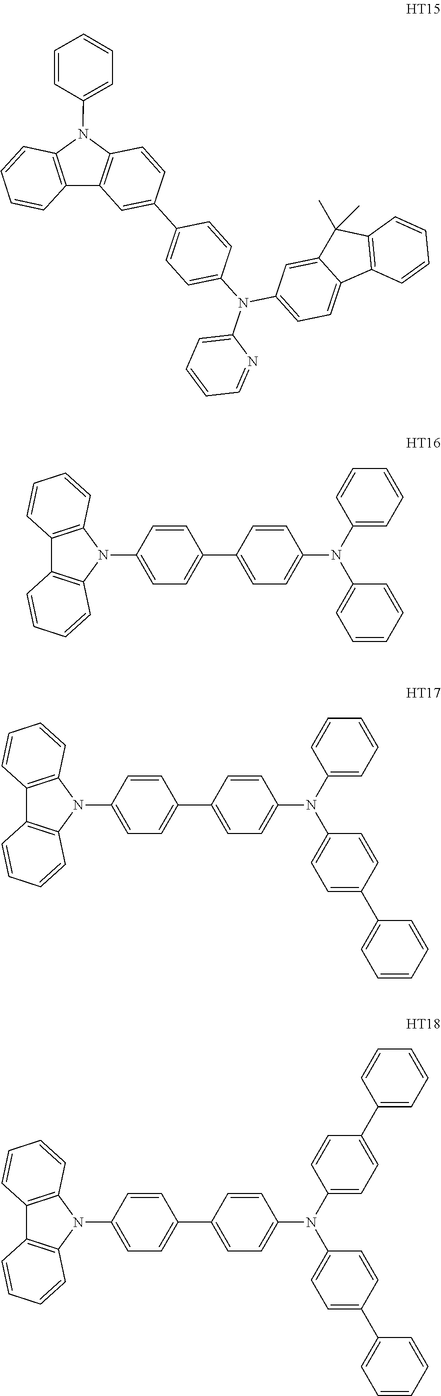 [SMILES: CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1 ; c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

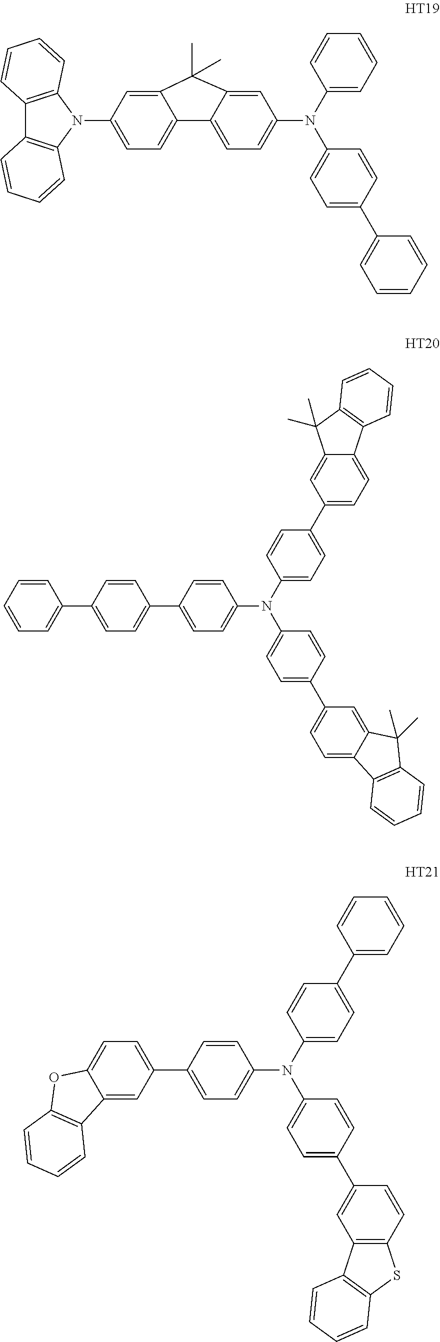 [SMILES: CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21 ; c1ccc(-c2ccc(N(c3ccc(-c4ccc5oc6ccccc6c5c4)cc3)c3ccc(-c4ccc5sc6ccccc6c5c4)cc3)cc2)cc1]

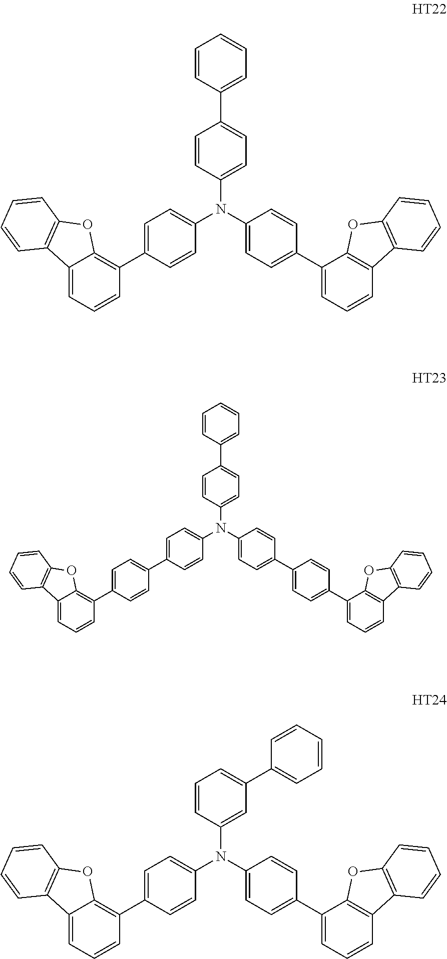 [SMILES: c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1 ; c1ccc(-c2cccc(N(c3ccc(-c4cccc5c4oc4ccccc45)cc3)c3ccc(-c4cccc5c4oc4ccccc45)cc3)c2)cc1]

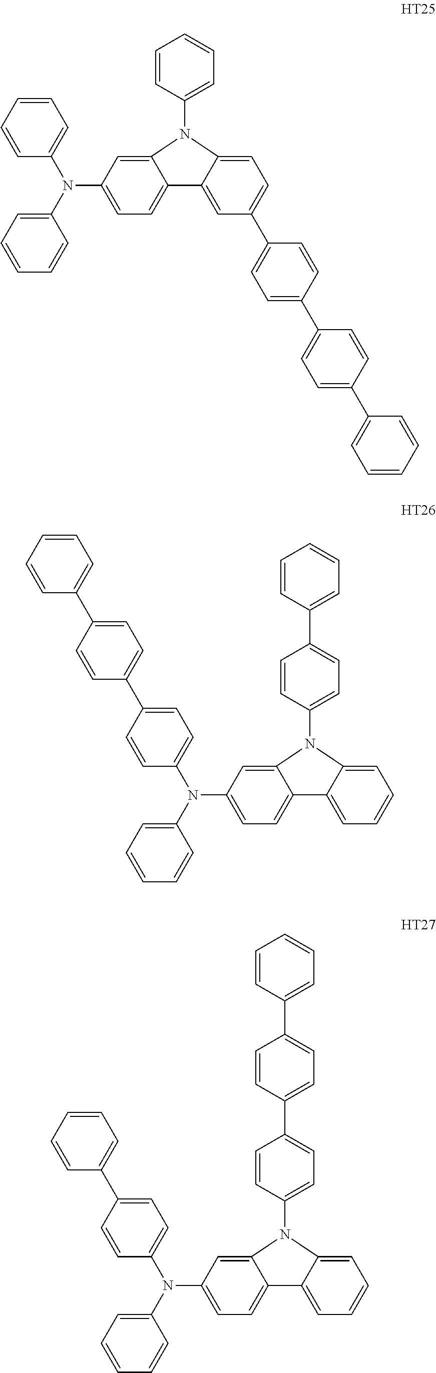 [SMILES: c1ccc(-c2ccc(-c3ccc(-c4ccc5c(c4)c4ccc(N(c6ccccc6)c6ccccc6)cc4n5-c4ccccc4)cc3)cc2)cc1 ; c1ccc(-c2ccc(-c3ccc(N(c4ccccc4)c4ccc5c6ccccc6n(-c6ccc(-c7ccccc7)cc6)c5c4)cc3)cc2)cc1 ; c1ccc(-c2ccc(-c3ccc(-n4c5ccccc5c5ccc(N(c6ccccc6)c6ccc(-c7ccccc7)cc6)cc54)cc3)cc2)cc1]

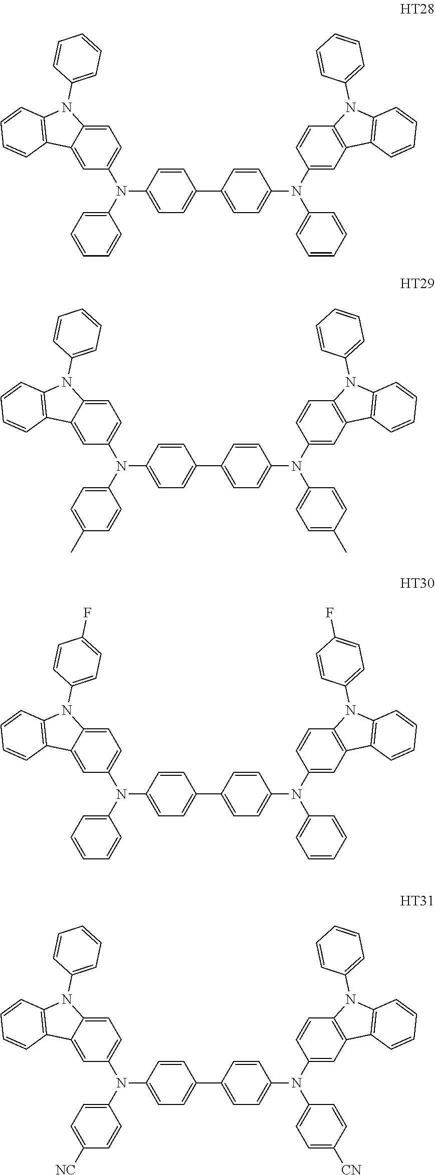 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1 ; N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1]

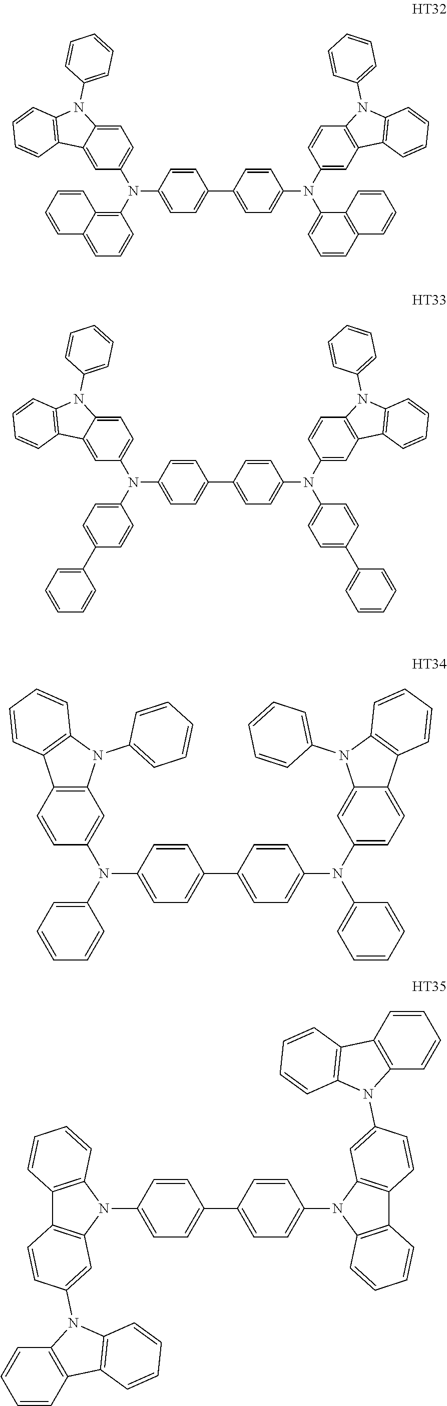 [SMILES: c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(N(c2ccc(-c3ccc(N(c4ccccc4)c4ccc5c6ccccc6n(-c6ccccc6)c5c4)cc3)cc2)c2ccc3c4ccccc4n(-c4ccccc4)c3c2)cc1 ; c1ccc2c(c1)c1ccccc1n2-c1ccc2c3ccccc3n(-c3ccc(-c4ccc(-n5c6ccccc6c6ccc(-n7c8ccccc8c8ccccc87)cc65)cc4)cc3)c2c1]

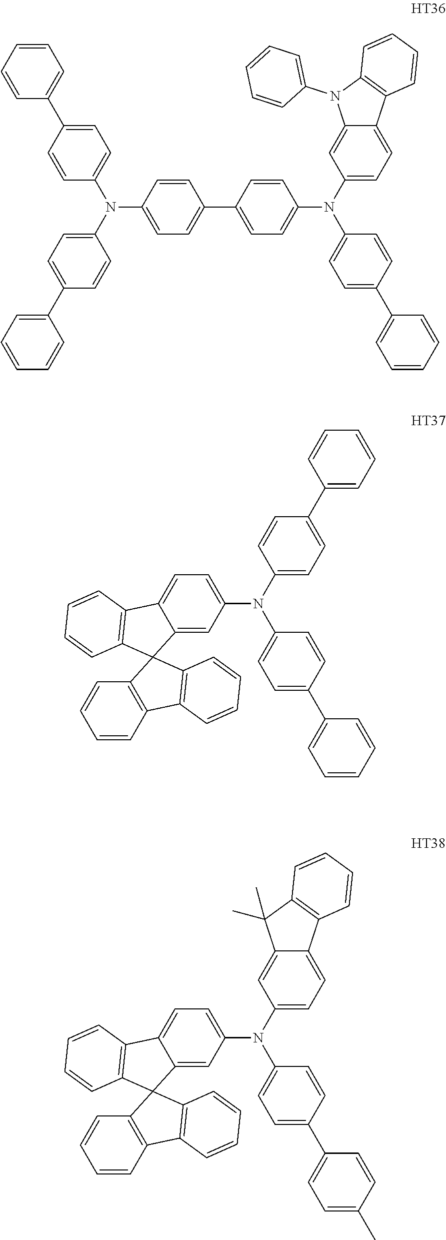 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1]

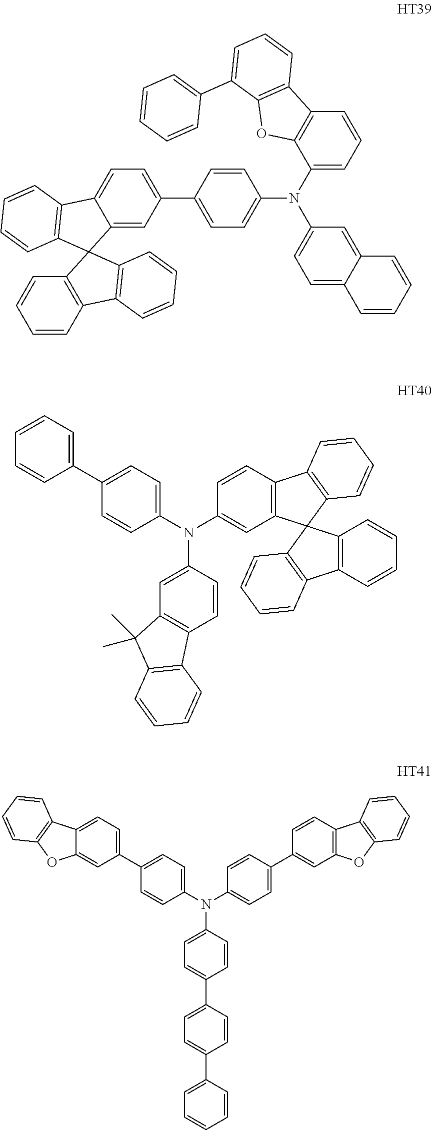 [SMILES: c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1 ; CC1(C)c2ccccc2-c2ccc(N(c3ccc(-c4ccccc4)cc3)c3ccc4c(c3)C3(c5ccccc5-c5ccccc53)c3ccccc3-4)cc21 ; c1ccc(-c2ccc(-c3ccc(N(c4ccc(-c5ccc6c(c5)oc5ccccc56)cc4)c4ccc(-c5ccc6c(c5)oc5ccccc56)cc4)cc3)cc2)cc1]

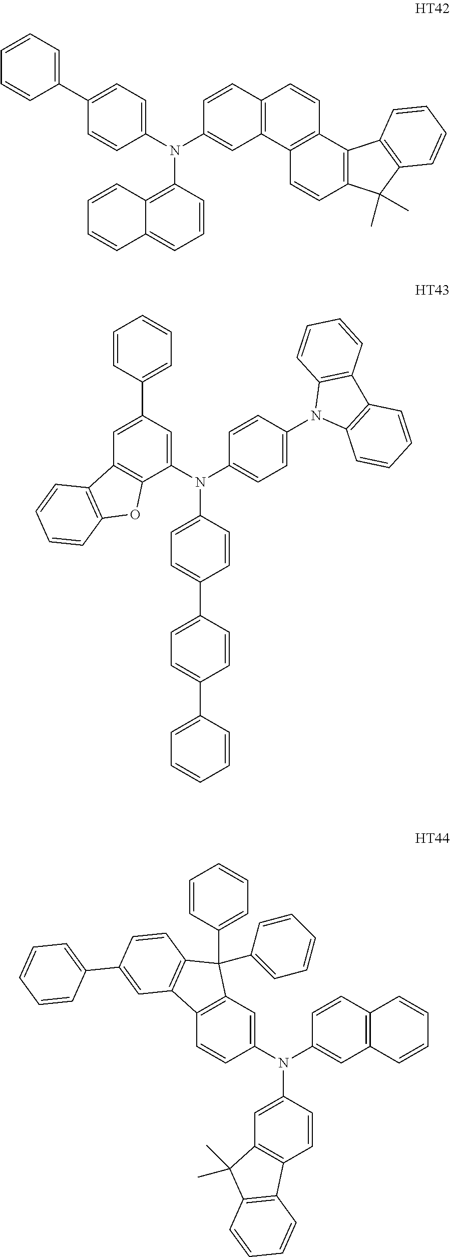 [SMILES: CC1(C)c2ccccc2-c2c1ccc1c2ccc2ccc(N(c3ccc(-c4ccccc4)cc3)c3cccc4ccccc34)cc21 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; CC1(C)c2ccccc2-c2ccc(N(c3ccc4c(c3)C(c3ccccc3)(c3ccccc3)c3ccc(-c5ccccc5)cc3-4)c3ccc4ccccc4c3)cc21]

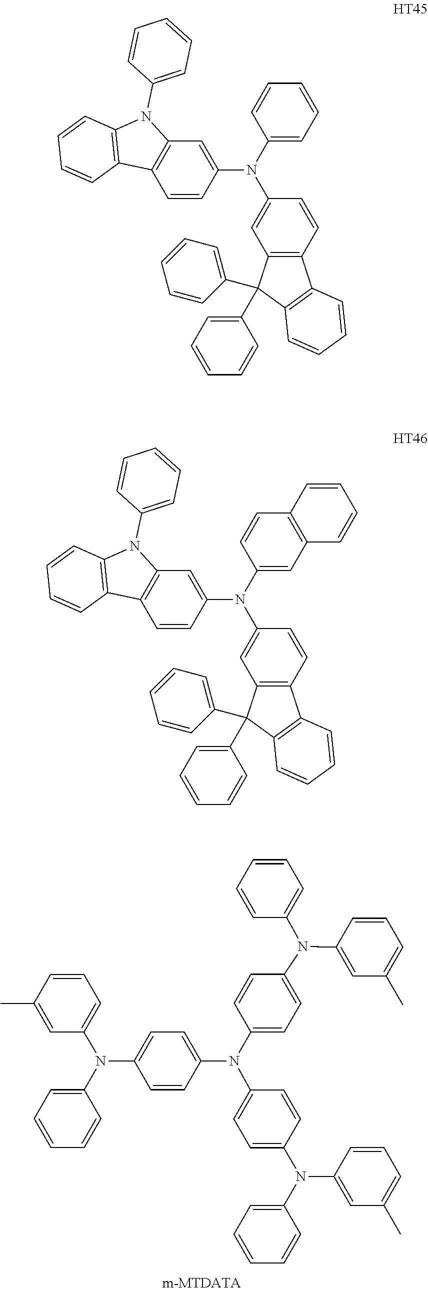

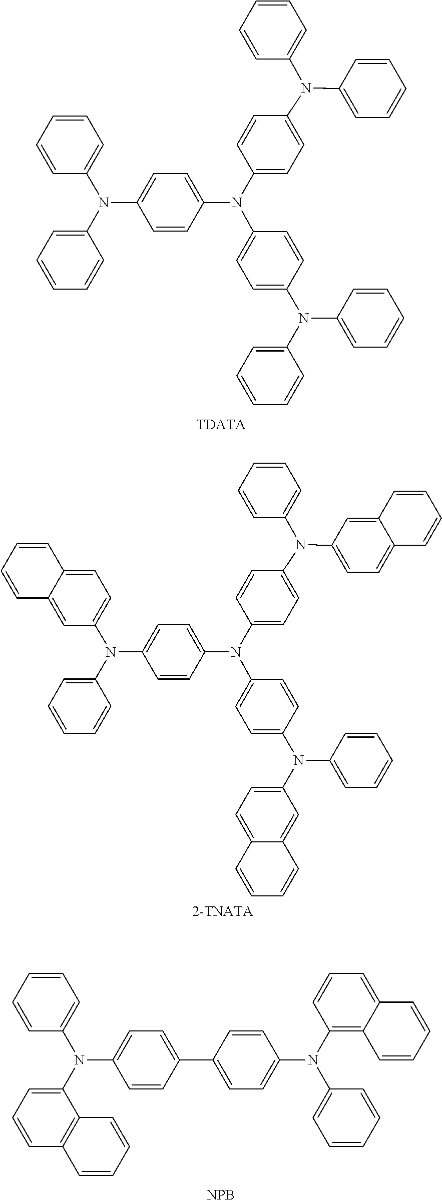 [SMILES: c1ccc(N(c2ccccc2)c2ccc(N(c3ccc(N(c4ccccc4)c4ccccc4)cc3)c3ccc(N(c4ccccc4)c4ccccc4)cc3)cc2)cc1 ; c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; "TDATA" ; "2-TNATA" ; c1ccc(N(c2ccc(-c3ccc(N(c4ccccc4)c4cccc5ccccc45)cc3)cc2)c2cccc3ccccc23)cc1 ; "NPB"]

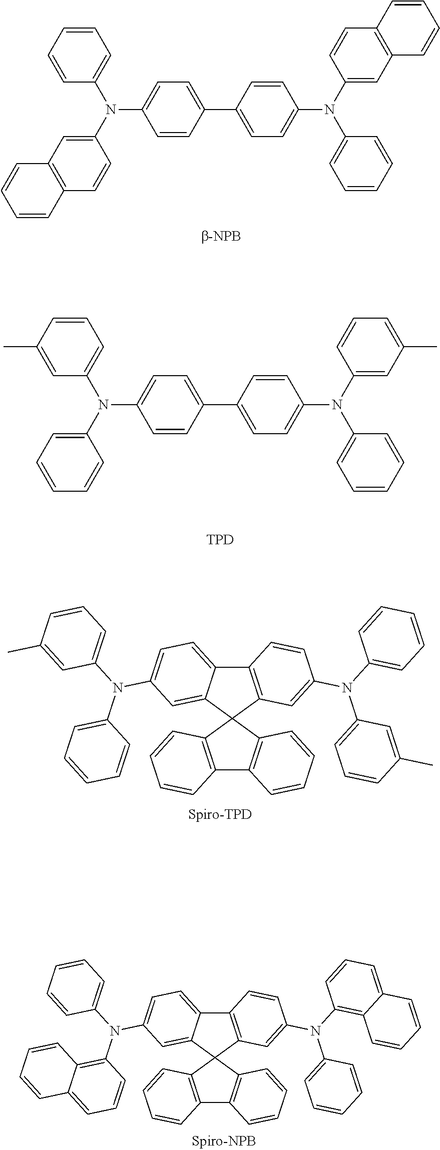

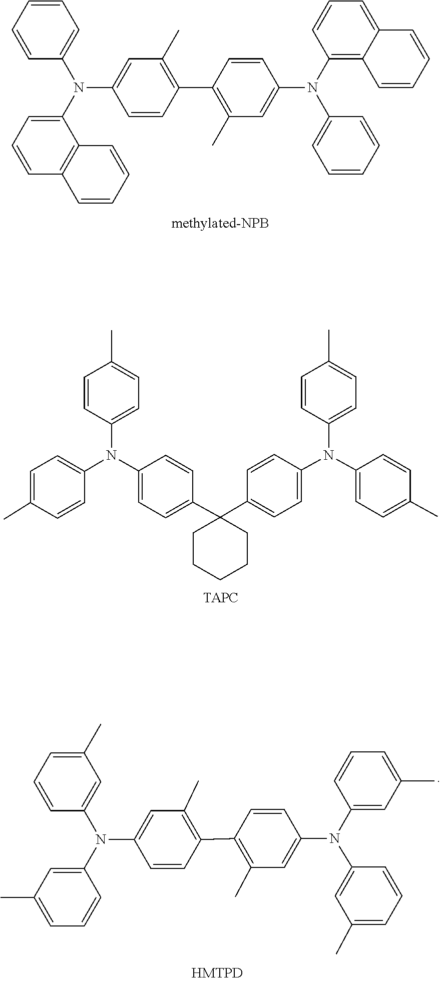

A thickness of the hole transport region 120 may be in a range of about 50 Angstroms (Å) to about 10,000 Å, for example, about 100 Å to about 4,000 Å. When the hole transport region 120 includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region 120, the hole injection layer, and the hole transport layer are within the ranges described above, satisfactory hole-transporting characteristics may be obtained without a substantial increase in driving voltage.

The emission auxiliary layer may serve to increase light-emission efficiency by compensating for an optical resonance distance according to the wavelength of light emitted by the emission layer 130. The electron-blocking layer may serve to prevent or reduce electron leakage from the emission layer 130 to the hole transport region 120. Materials that may be included in the hole transport region 120 may be included in the emission auxiliary layer and the electron-blocking layer.

p-dopant

In one or more embodiments, the hole transport region 120 may include, in addition to one or more of the materials described above, a charge-generation material for the improvement of conductive properties. The charge-generation material may be uniformly (e.g., substantially uniformly) or non-uniformly dispersed in the hole transport region 120 (for example, in the form of a single layer including (e.g., consisting of) a charge-generation material).

The charge-generation material may be, for example, a p-dopant.

In one or more embodiments, the p-dopant may have a lowest unoccupied molecular orbital (LUMO) energy level of about −3.5 eV or less.

In one or more embodiments, the p-dopant may include a quinone derivative, a cyano group-containing compound, a compound including element EL1 and element EL2, or any combination thereof.

Non-limiting examples of the quinone derivative may include tetracyanoquinodimethane (TCNQ), 2,3,5,6-tetrafluoro-7,7,8,8-tetracyanoquinodimethane (F₄-TCNQ), and/or the like.

Non-limiting examples of the cyano group-containing compound may include dipyrazino[2,3-f: 2′, 3′-h]quinoxaline-2,3,6,7,10,11-hexacarbonitrile (HAT-CN), a compound represented by Formula 221, and/or the like:

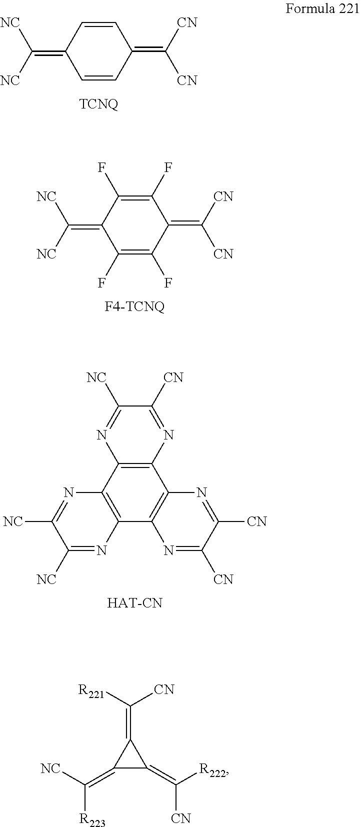

wherein, in Formula 221,
R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and
at least one selected from among R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

In the compound including element EL1 and element EL2, element EL1 may be a metal, a metalloid, or a (e.g., any suitable) combination thereof, and element EL2 may be a non-metal, a metalloid, or a (e.g., any suitable) combination thereof.

Non-limiting examples of the metal may include: an alkali metal (for example, lithium (Li), sodium (Na), potassium (K), rubidium (Rb), cesium (Cs), and/or the like); an alkaline earth metal (for example, beryllium (Be), magnesium (Mg), calcium (Ca), strontium (Sr), barium (Ba), and/or the like); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), and/or the like); a post-transition metal (for example, zinc (Zn), indium (In), tin (Sn), and/or the like); a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), and/or the like); and/or the like.

Non-limiting examples of the metalloid may include silicon (Si), antimony (Sb), tellurium (Te), and/or the like.

Non-limiting examples of the non-metal may include oxygen (O), halogen (for example, F, Cl, Br, I, and/or the like), and/or the like.

Non-limiting examples of the compound including element EL1 and element EL2 may include a metal oxide, a metal halide (for example, a metal fluoride, a metal chloride, a metal bromide, a metal iodide, and/or the like), a metalloid halide (for example, a metalloid fluoride, a metalloid chloride, a metalloid bromide, a metalloid iodide, and/or the like), a metal telluride, or any combination thereof.

Non-limiting examples of the metal oxide may include a tungsten oxide (for example, WO, W₂O₃, WO₂, WO₃, W₂O₅, and/or the like), a vanadium oxide (for example, VO, V₂O₃, VO₂, V₂O₅, and/or the like), a molybdenum oxide (for example, MoO, Mo₂O₃, MoO₂, MoO₃, Mo₂O₅, and/or the like), a rhenium oxide (for example, ReO₃ and/or the like), and/or the like.

Non-limiting examples of the metal halide may include an alkali metal halide, an alkaline earth metal halide, a transition metal halide, a post-transition metal halide, a lanthanide metal halide, and/or the like.

Non-limiting examples of the alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, CsI, and/or the like.

Non-limiting examples of the alkaline earth metal halide may include BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, SrI₂, BaI₂, and/or the like.

Non-limiting examples of the transition metal halide may include a titanium halide (for example, TiF₄, TiCl₄, TiBr₄, TiI₄, and/or the like), a zirconium halide (for example, ZrF₄, ZrCl₄, ZrBr₄, ZrI₄, and/or the like), a hafnium halide (for example, HfF₄, HfCl₄, HfBr₄, HfI₄, and/or the like), a vanadium halide (for example, VF₃, VCl₃, VBr₃, VI₃, and/or the like), a niobium halide (for example, NbF₃, NbCl₃, NbBr₃, NbI₃, and/or the like), a tantalum halide (for example, TaF₃, TaCl₃, TaBr₃, TaI₃, and/or the like), a chromium halide (for example, CrF₃, CrCl₃, CrBr₃, CrI₃, and/or the like), a molybdenum halide (for example, MoF₃, MoCl₃, MoBr₃, MoI₃, and/or the like), a tungsten halide (for example, WF₃, WCl₃, WBr₃, WI₃, and/or the like), a manganese halide (for example, MnF₂, MnCl₂, MnBr₂, MnI₂, and/or the like), a technetium halide (for example, TcF₂, TcCl₂, TcBr₂, TcI₂, and/or the like), a rhenium halide (for example, ReF₂, ReCl₂, ReBr₂, ReI₂, and/or the like), an iron(II) halide (for example, FeF₂, FeCl₂, FeBr₂, FeI₂, and/or the like), a ruthenium halide (for example, RuF₂, RuCl₂, RuBr₂, RuI₂, and/or the like), an osmium halide (for example, OsF₂, OsCl₂, OsBr₂, OsI₂, and/or the like), a cobalt halide (for example, CoF₂, CoCl₂, CoBr₂, CoI₂, and/or the like), a rhodium halide (for example, RhF₂, RhCl₂, RhBr₂, RhI₂, and/or the like), an iridium halide (for example, IrF₂, IrCl₂, IrBr₂, IrI₂, and/or the like), a nickel halide (for example, NiF₂, NiCl₂, NiBr₂, NiI₂, and/or the like), a palladium halide (for example, PdF₂, PdCl₂, PdBr₂, PdI₂, and/or the like), a platinum halide (for example, PtF₂, PtCl₂, PtBr₂, PtI₂, and/or the like), a copper(I) halide (for example, CuF, CuCl, CuBr, CuI, and/or the like), a silver halide (for example, AgF, AgCl, AgBr, AgI, and/or the like), a gold halide (for example, AuF, AuCl, AuBr, AuI, and/or the like), and/or the like.

Non-limiting examples of the post-transition metal halide may include a zinc halide (for example, ZnF₂, ZnCl₂, ZnBr₂, ZnI₂, and/or the like), an indium halide (for example, InI₃ and/or the like), a tin halide (for example, SnI₂ and/or the like), and/or the like.

Non-limiting examples of the lanthanide metal halide may include YbF, YbF₂, YbF₃, SmF₃, YbCl, YbCl₂, YbCl₃, SmCl₃, YbBr, YbBr₂, YbBr₃, SmBr₃, Ybl, YbI₂, YbI₃, SmI₃, and/or the like.

Non-limiting examples of the metalloid halide may include an antimony halide (for example, SbCl₅ and/or the like) and/or the like.

Non-limiting examples of the metal telluride may include an alkali metal telluride (for example, Li₂Te, Na₂Te, K₂Te, Rb₂Te, Cs₂Te, and/or the like), an alkaline earth metal telluride (for example, BeTe, MgTe, CaTe, SrTe, BaTe, and/or the like), a transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, and/or the like), a post-transition metal telluride (for example, ZnTe and/or the like), a lanthanide metal telluride (for example, LaTe, CeTe, PrTe, NdTe, PmTe, EuTe, GdTe, TbTe, DyTe, HoTe, ErTe, TmTe, YbTe, LuTe, and/or the like), and/or the like.

### Emission Layer 130

When the light-emitting device 10 is a full-color light-emitting device, the emission layer 130 may be patterned into a red emission layer, a green emission layer, and/or a blue emission layer, according to a subpixel. In one or more embodiments, the emission layer 130 may have a stacked structure of two or more layers selected from among a red emission layer, a green emission layer, and a blue emission layer, in which the two or more layers contact each other or are separated from each other, to emit white light (e.g., combined white light). In one or more embodiments, the emission layer 130 may include two or more materials selected from among a red light-emitting material, a green light-emitting material, and a blue light-emitting material, in which the two or more materials are mixed with each other in a single layer, to emit white light (e.g., combined white light).

The emission layer 130 may include a host and a dopant. The dopant may include a phosphorescent dopant, a fluorescent dopant, or any combination thereof.

An amount of the dopant in the emission layer 130 may be in a range of about 0.01 part by weight to about 15 parts by weight based on 100 parts by weight of the host.

In one or more embodiments, the emission layer 130 may include quantum dots.

In one or more embodiments, the emission layer 130 may include a delayed fluorescence material. The delayed fluorescence material may act as a host or a dopant in the emission layer 130.

A thickness of the emission layer 130 may be in a range of about 100 Å to about 1,000 Å, for example, about 200 Å to about 600 Å. When the thickness of the emission layer 130 is within the range described above, excellent or suitable luminescence characteristics may be obtained without a substantial increase in driving voltage.

### Host

In one or more embodiments, the host may include a compound represented by Formula 301:

[Ar₃₀₁]_{xb11}-[(L₃₀₁)_{xb1}-R₃₀₁]_{xb21}, Formula 301
wherein, in Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ may each independently be the same as described with respect to Q₁.

In one or more embodiments, if (e.g., when) xb11 in Formula 301 is 2 or more, two or more of Ar₃₀₁(s) may be linked to each other via a single bond.

In one or more embodiments, the host may include a compound represented by Formula 301-1, a compound represented by Formula 301-2, or any combination thereof:

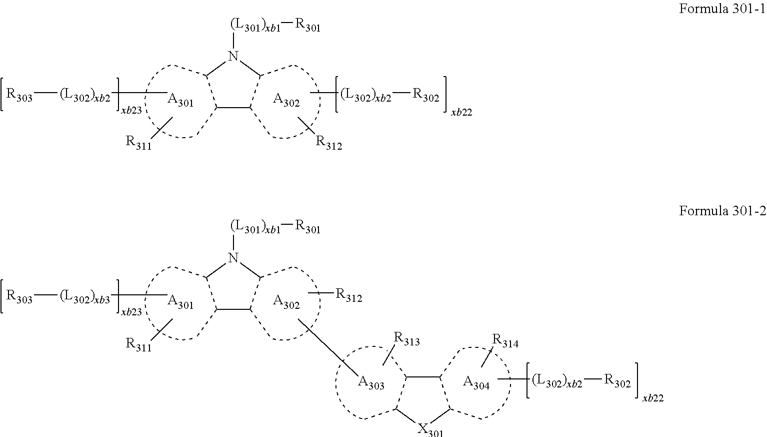

wherein, in Formulae 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ may each be the same as described herein,

L₃₀₂ to L₃₀₄ may each independently be the same as described with respect to L₃₀₁,
xb2 to xb4 may each independently be the same as described with respect to xb1, and
R₃₀₂ to R₃₀₅ and R₃₁₁ to R₃₁₄ may each independently be the same as described with respect to R₃₀₁.

In one or more embodiments, the host may include an alkaline earth metal complex, a post-transition metal complex, or any combination thereof. For example, in some embodiments, the host may include a Be complex (for example, Compound H55), a Mg complex, a Zn complex, or any combination thereof.

In one or more embodiments, the host may include: at least one of (e.g., selected from among) Compounds H1 to H128; 9,10-di(2-naphthyl)anthracene (ADN); 2-methyl-9,10-bis(naphthalen-2-yl)anthracene (MADN); 9,10-di-(2-naphthyl)-2-t-butyl-anthracene (TBADN); 4,4′-bis(N-carbazolyl)-1,1′-biphenyl (CBP); 1,3-di(9H-carbazol-9-yl)benzene (mCP); 1,3,5-tri(carbazol-9-yl)benzene (TCP); or any combination thereof:

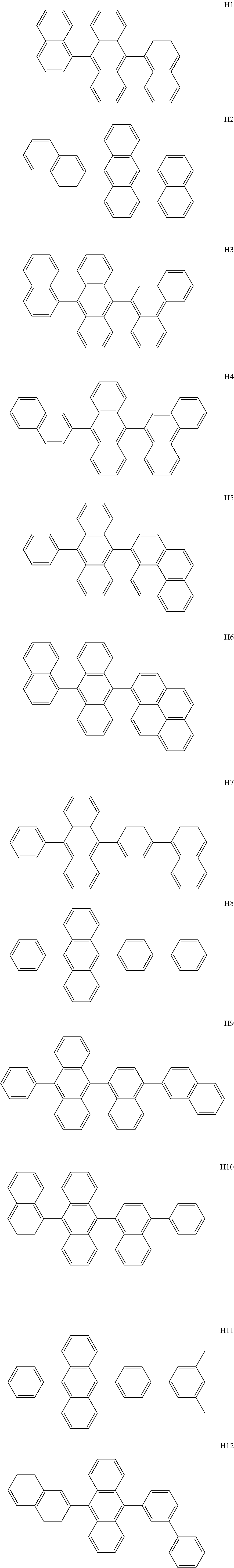

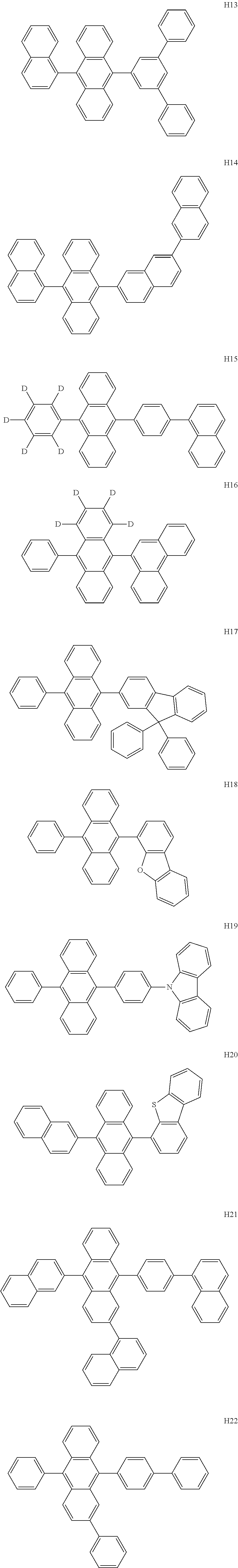

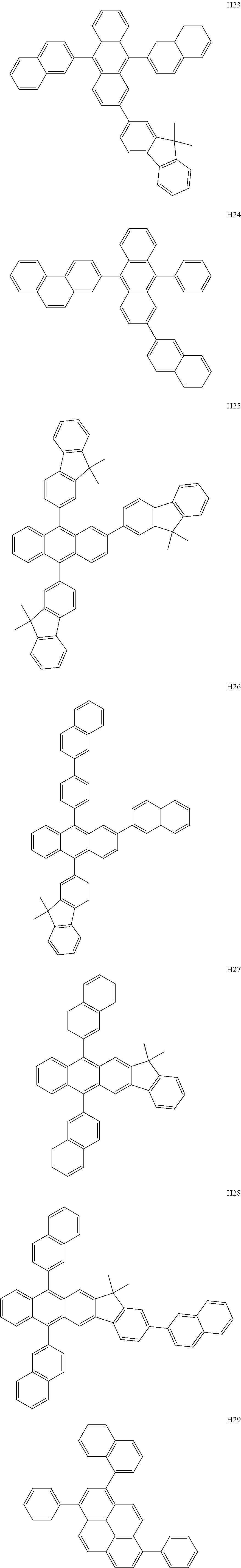

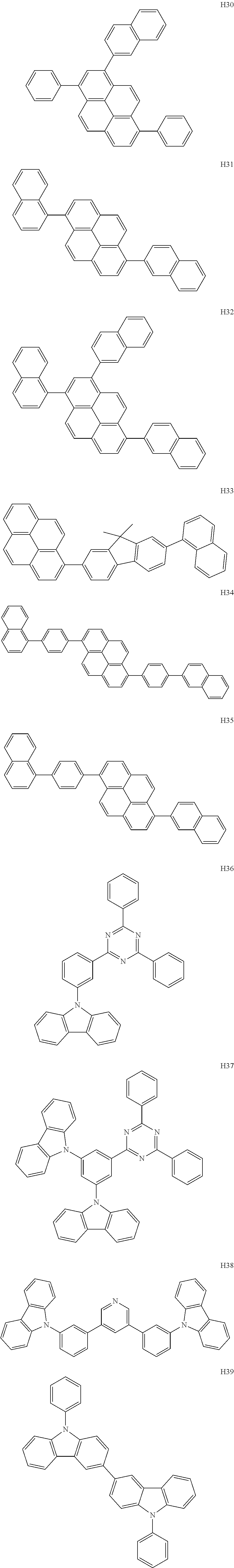

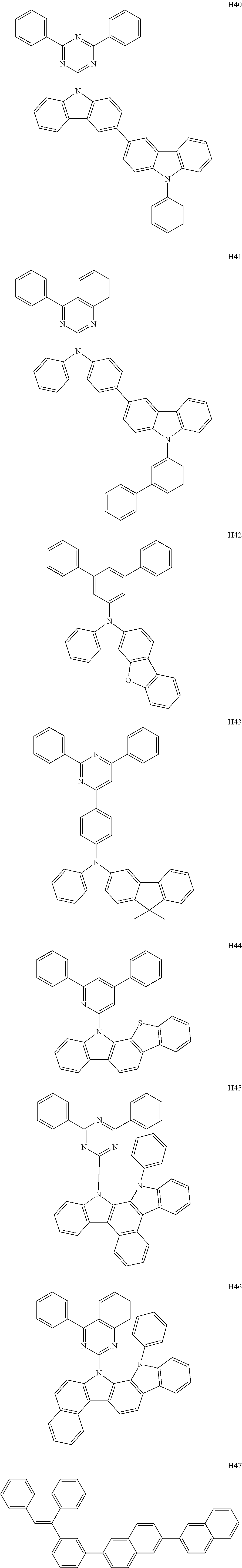

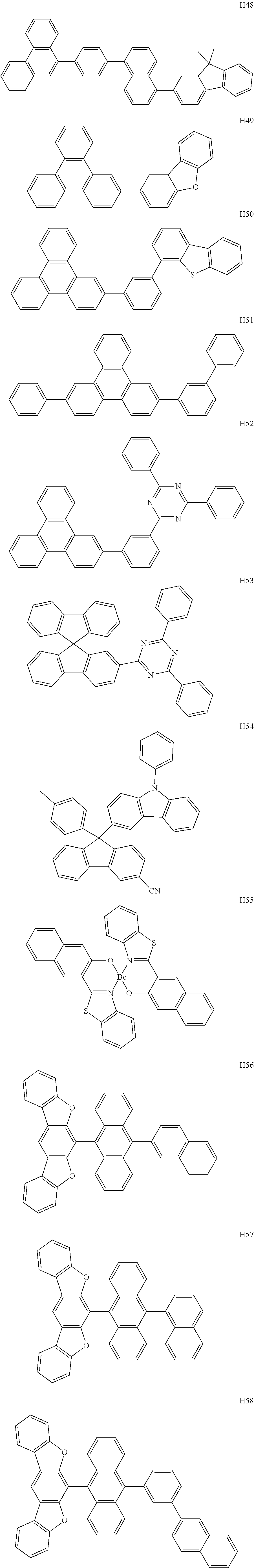

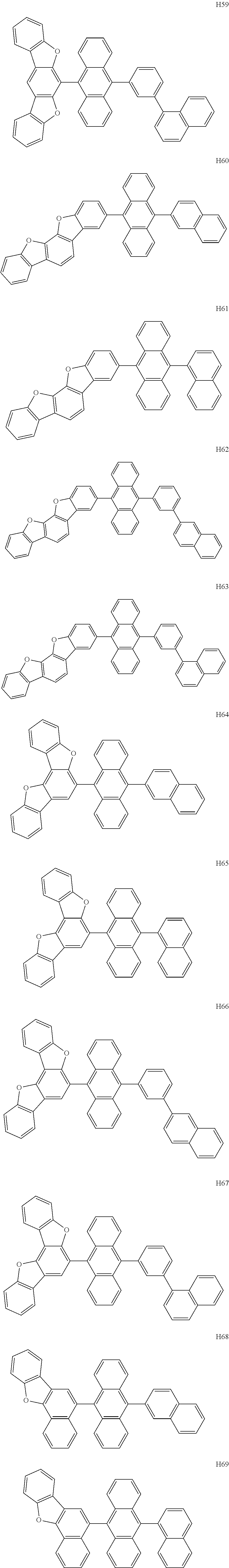

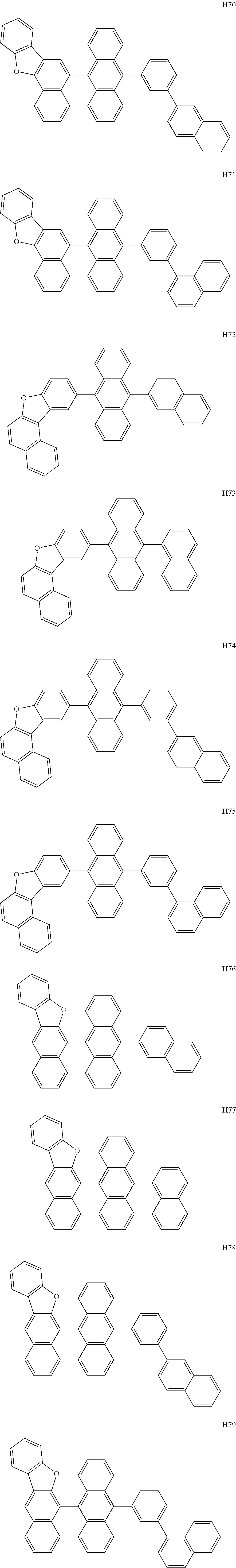

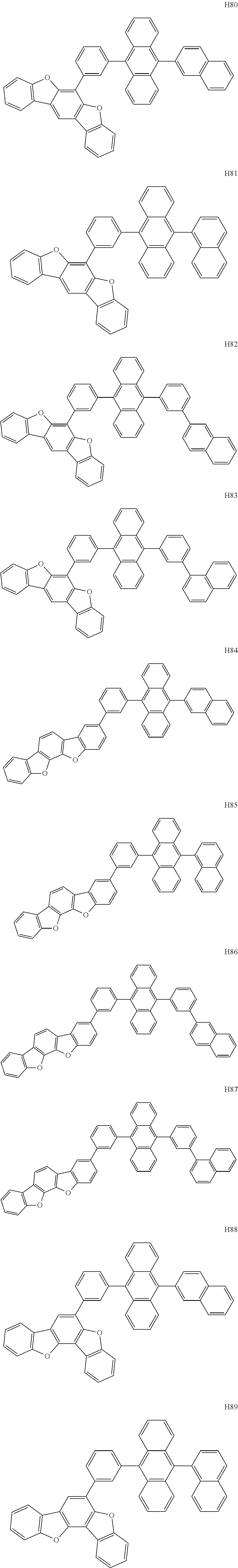

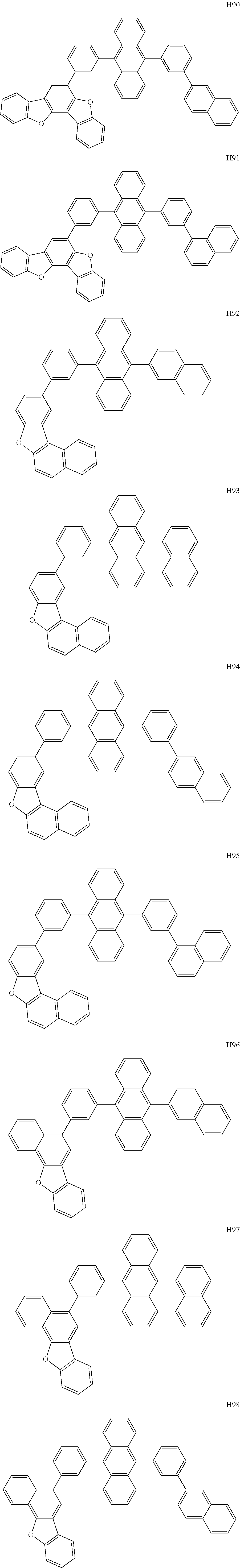

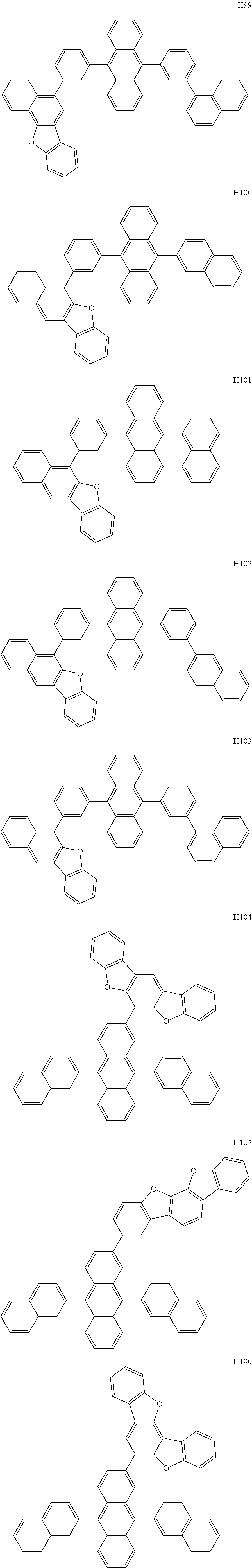

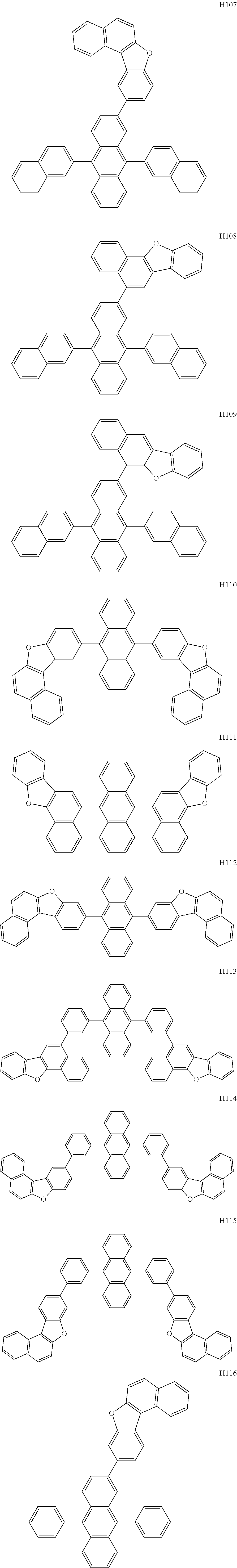

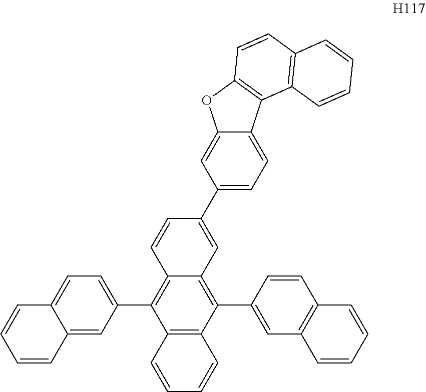 [SMILES: c1ccc2cc(-c3c4ccccc4c(-c4ccc5ccccc5c4)c4cc(-c5ccc6c(c5)oc5ccc7ccccc7c56)ccc34)ccc2c1]

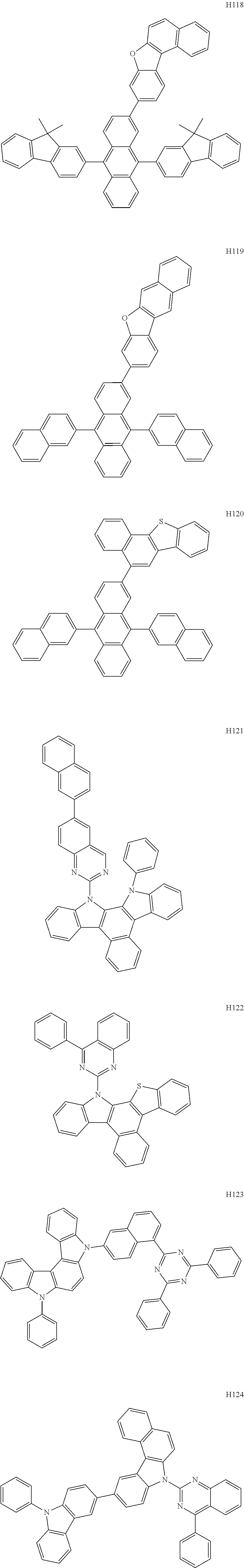

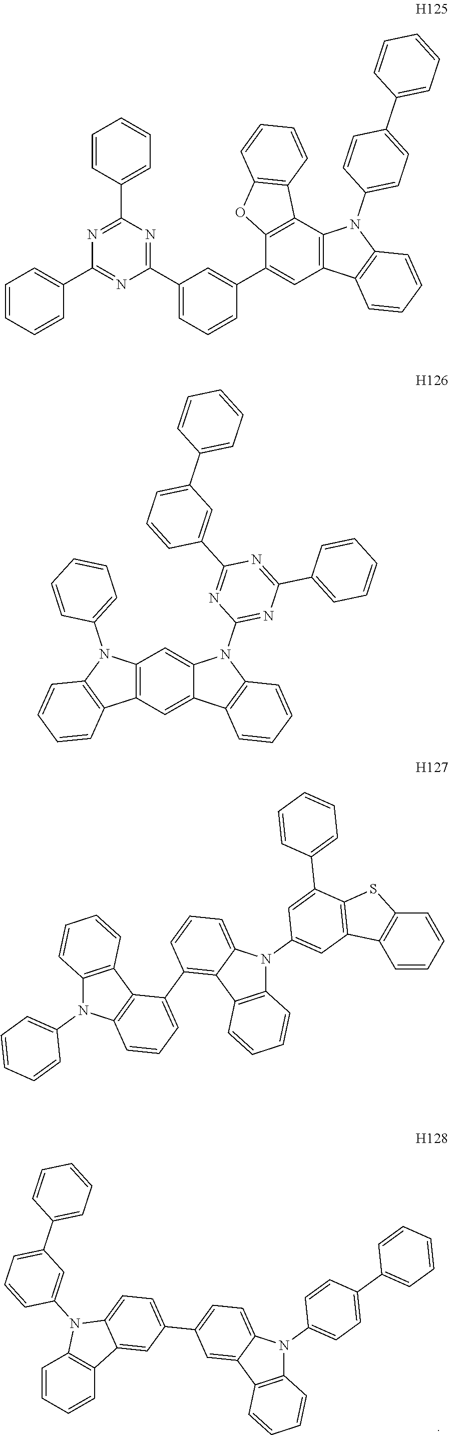 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1]

### Phosphorescent Dopant

The phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

In one or more embodiments, the phosphorescent dopant may include an organometallic compound represented by Formula 401:

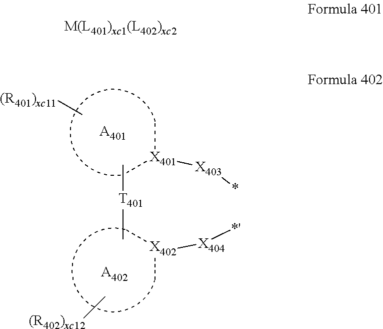

wherein, in Formulae 401 and 402,
M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, wherein, if (e.g., when) xc1 is 2 or more, two or more of L₄₀₁(s) may be identical to or different from each other,
L₄₀₂ may be an organic ligand, and xc2 may be 0, 1, 2, 3, or 4, wherein, if (e.g., when) xc2 is 2 or more, two or more of L₄₀₂(s) may be identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*′, *—C(═O)—*′, *—N(Q₄₁₁)-*′, *—C(Q₄₁₁)(Q₄₁₂)*′, *—C(Q₄₁₁)=C(Q₄₁₂)-*′, *—C(Q₄₁₁)=*′, or *═C═*′,
X₄₀₃ and X₄₀₄ may each independently be a chemical bond (for example, a covalent bond or a coordinate bond), O, S, N(Q₄₁₃), B(Q₄₁₃), P(Q₄₁₃), C(Q₄₁₃)(Q₄₁₄), or Si(Q₄₁₃)(Q₄₁₄),
Q₄₁₁ to Q₄₁₄ may each independently be the same as described with respect to Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ may each independently be the same as described with respect to Q₁,
xc11 and xc12 may each independently be an integer from 0 to 10, and
* and *′ in Formula 402 each indicate a binding site to M in Formula 401.

In one or more embodiments, in Formula 402, i) X₄₀₁ may be nitrogen, and X₄₀₂ may be carbon, or ii) each of X₄₀₁ and X₄₀₂ may be nitrogen.

In one or more embodiments, if (e.g., when) xc1 in Formula 401 is 2 or more, two of ring A₄₀₁ (s) among two or more of L₄₀₁ (s) may optionally be linked to each other via T₄₀₂, which is a linking group, and/or two of ring A₄₀₂(s) among two or more of L₄₀₁(s) may optionally be linked to each other via T₄₀₃, which is a linking group (see Compounds PD1 to PD4 and PD7). T₄₀₂ and T₄₀₃ may each independently be the same as described with respect to T₄₀₁.

L₄₀₂ in Formula 401 may be an organic ligand. For example, L₄₀₂ may include a halogen, a diketone group (for example, an acetylacetonate group), a carboxylic acid group (for example, a picolinate group), —C(═O), an isonitrile group, a —CN group, a phosphorus-containing group (for example, a phosphine group, a phosphite group, and/or the like), or any combination thereof.

In one or more embodiments, the phosphorescent dopant may include, for example, at least one (e.g., may be any one) of (e.g., selected from among) Compounds PD1 to PD39, or any combination thereof:

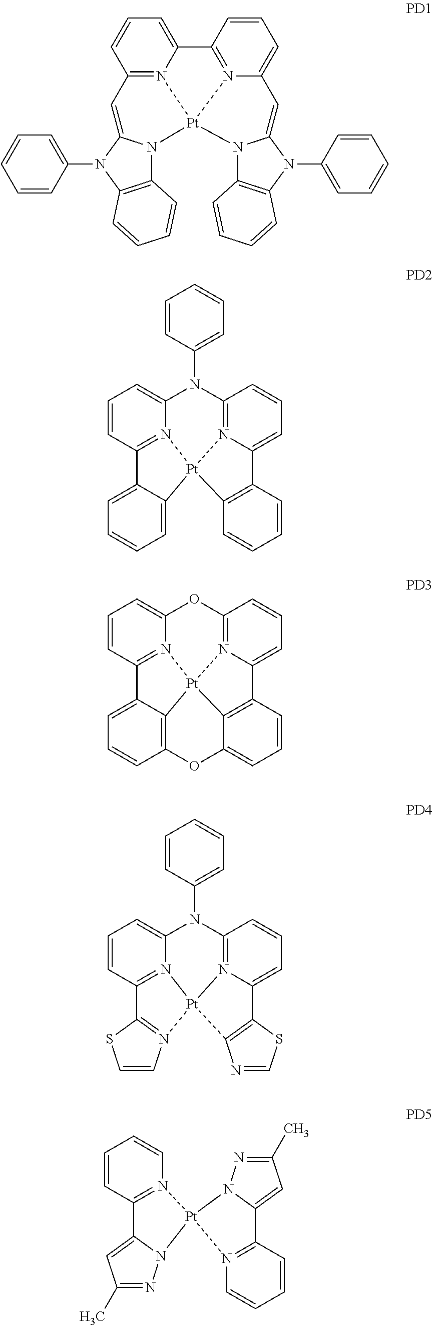

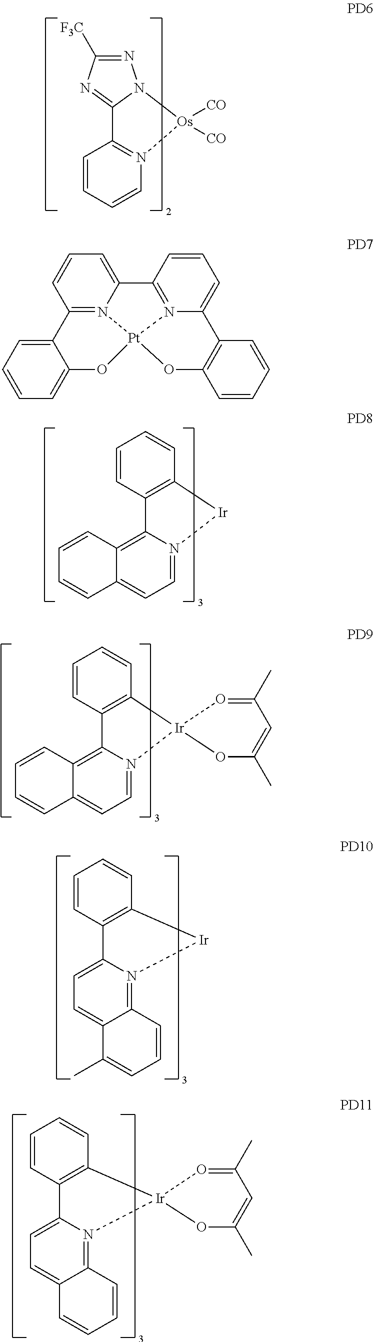

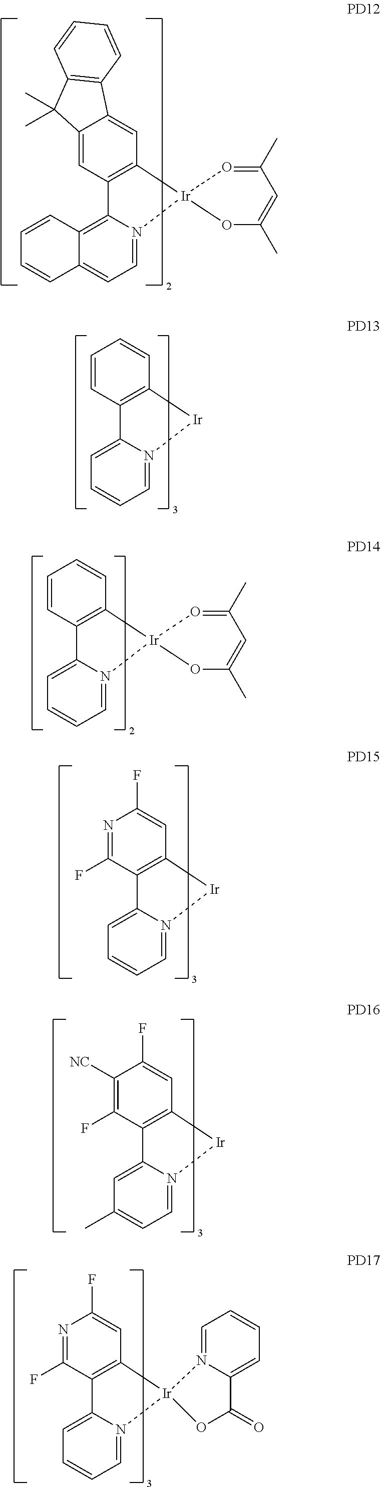

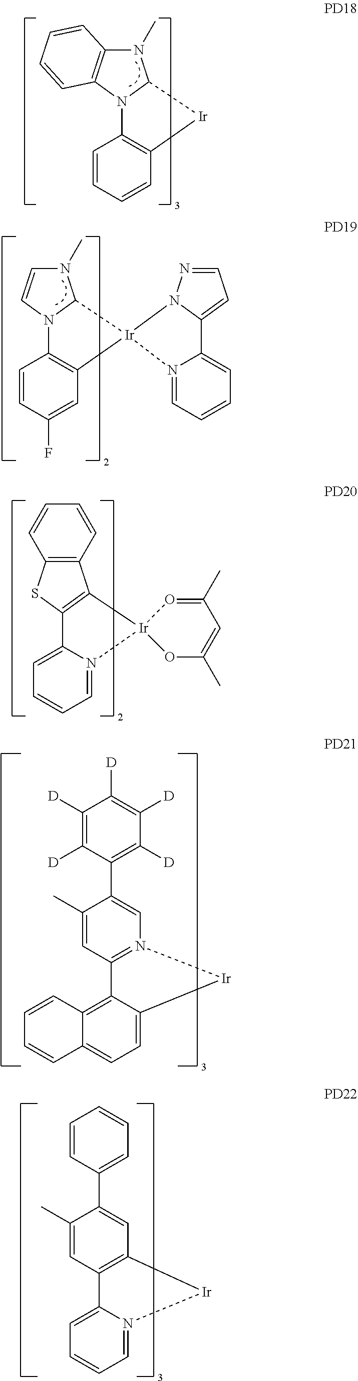

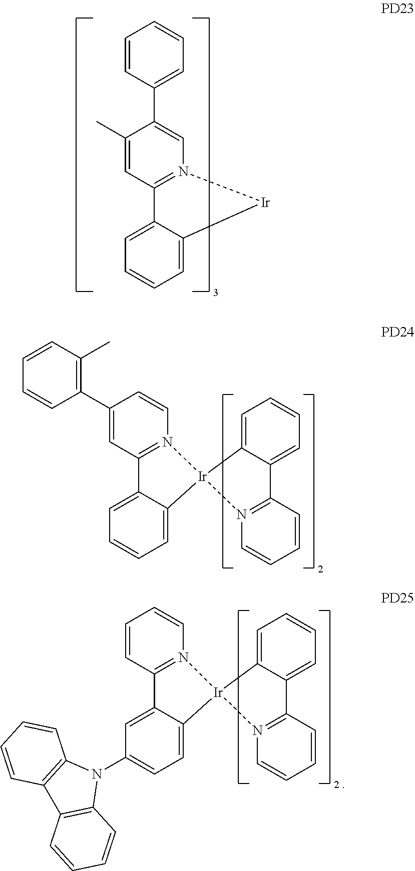

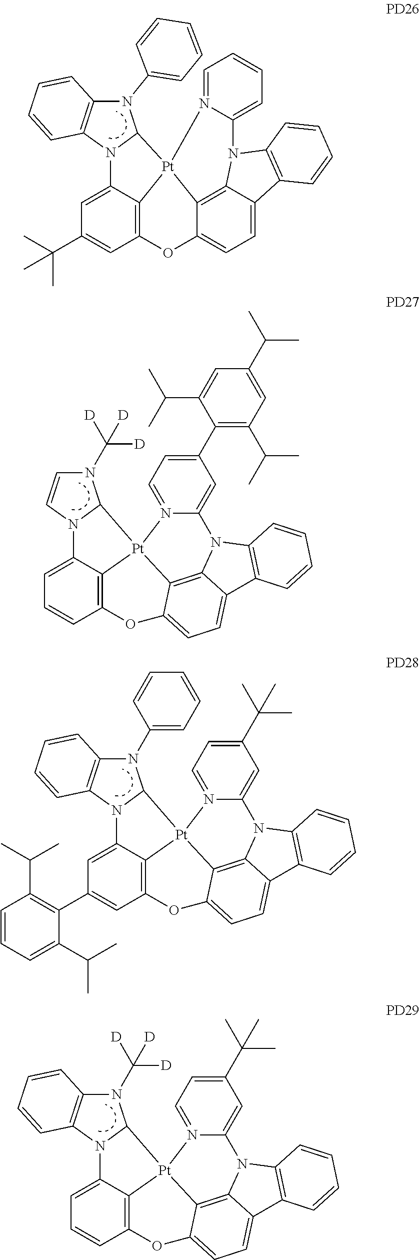

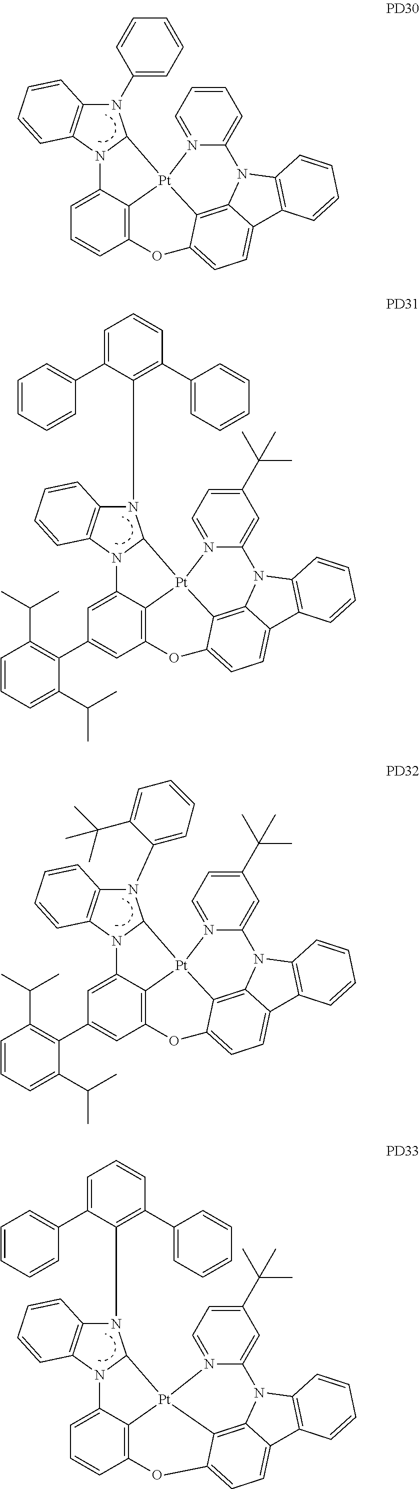

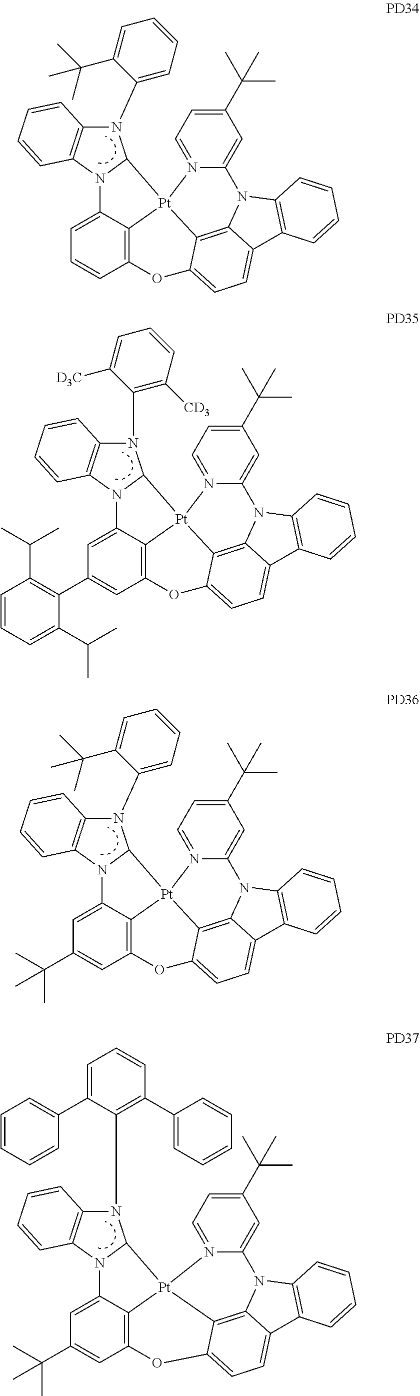

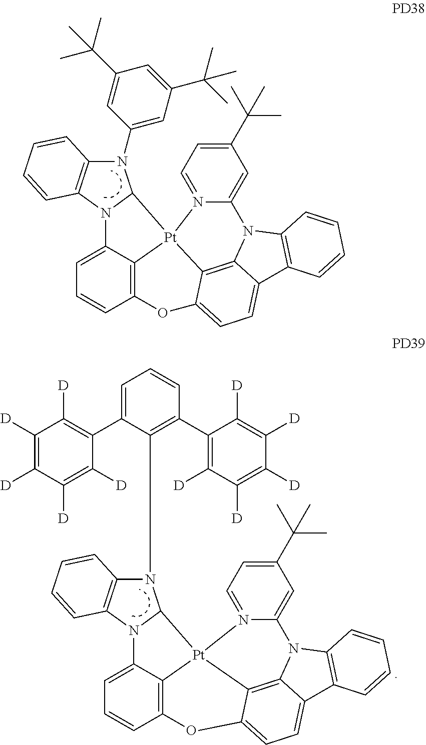

### Fluorescent Dopant

The fluorescent dopant may include an amine group-containing compound, a styryl group-containing compound, or any combination thereof.

In one or more embodiments, the fluorescent dopant may include a compound represented by Formula 501:

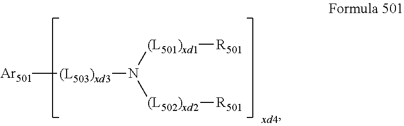

wherein, in Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

In one or more embodiments, Ar₅₀₁ in Formula 501 may be a condensed cyclic group (for example, an anthracene group, a chrysene group, a pyrene group, and/or the like) in which three or more monocyclic groups are condensed with each other.

In one or more embodiments, xd4 in Formula 501 may be 2.

In one or more embodiments, the fluorescent dopant may include: at least one of (e.g., selected from among) Compounds FD1 to FD37; 4,4′-bis(2,2-diphenylvinyl)-1,1′-biphenyl (DPVBi); 4,4′-bis[4-(N,N-diphenylamino)styryl]biphenyl (DPAVBi); or any combination thereof:

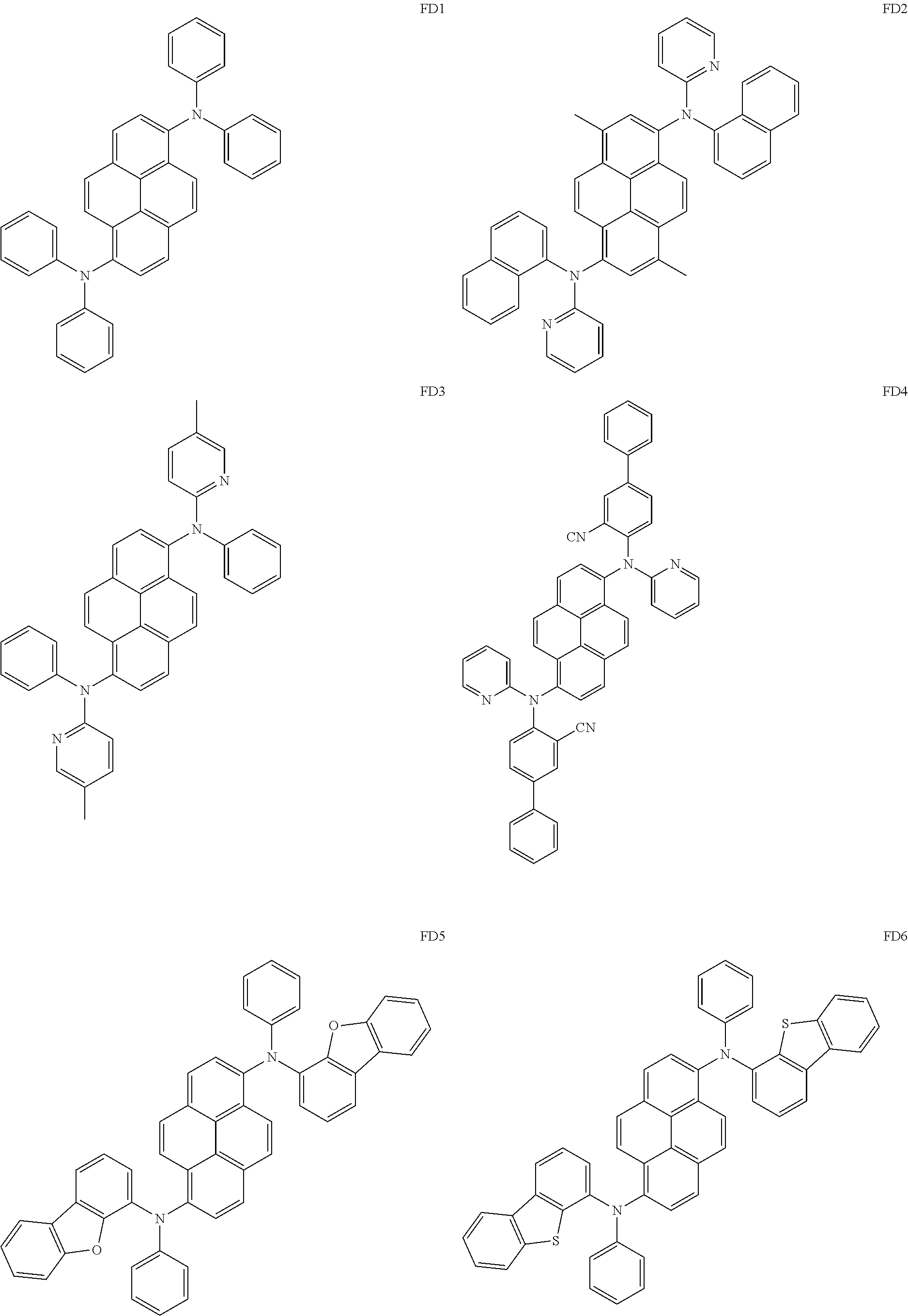

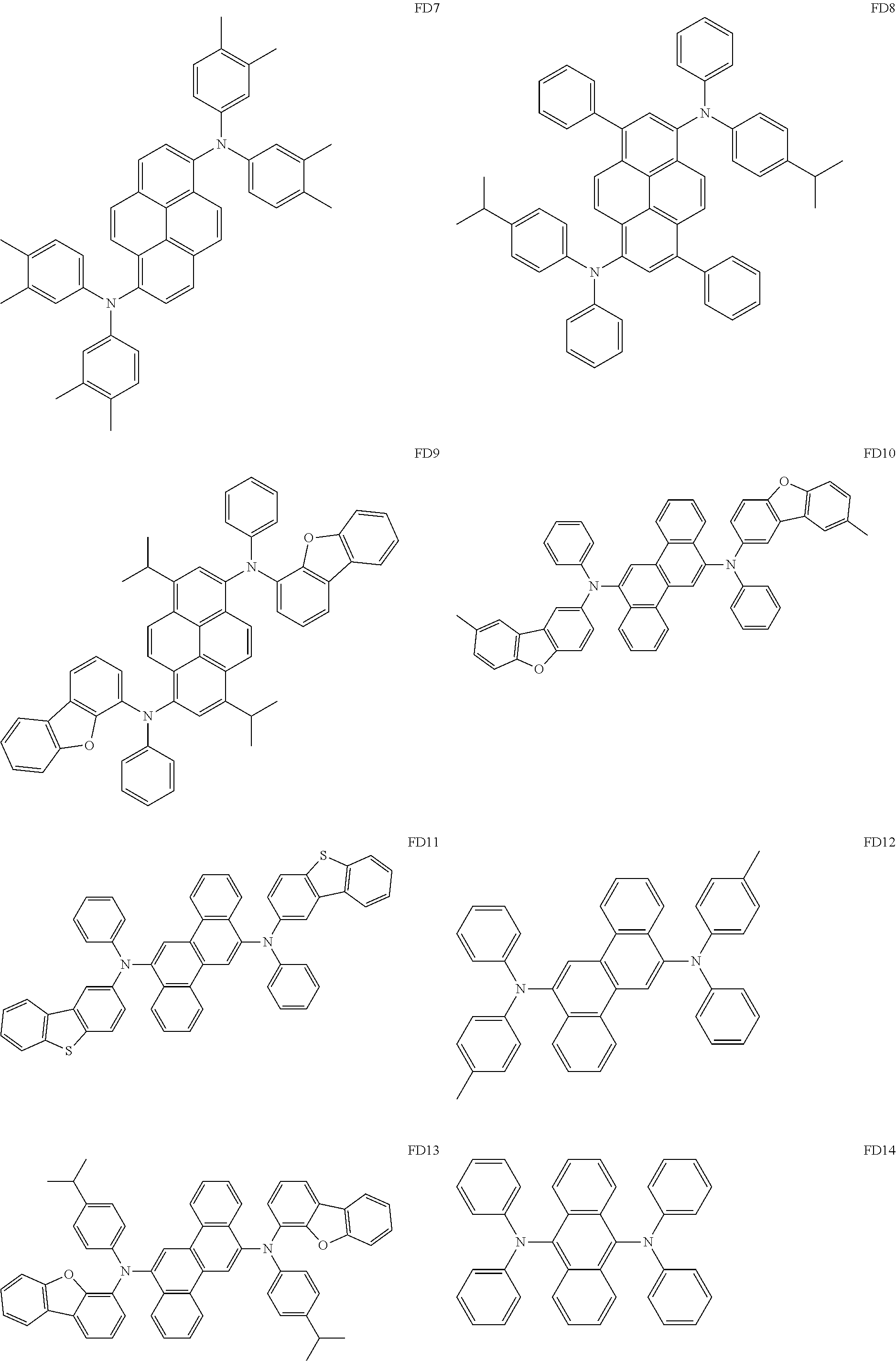

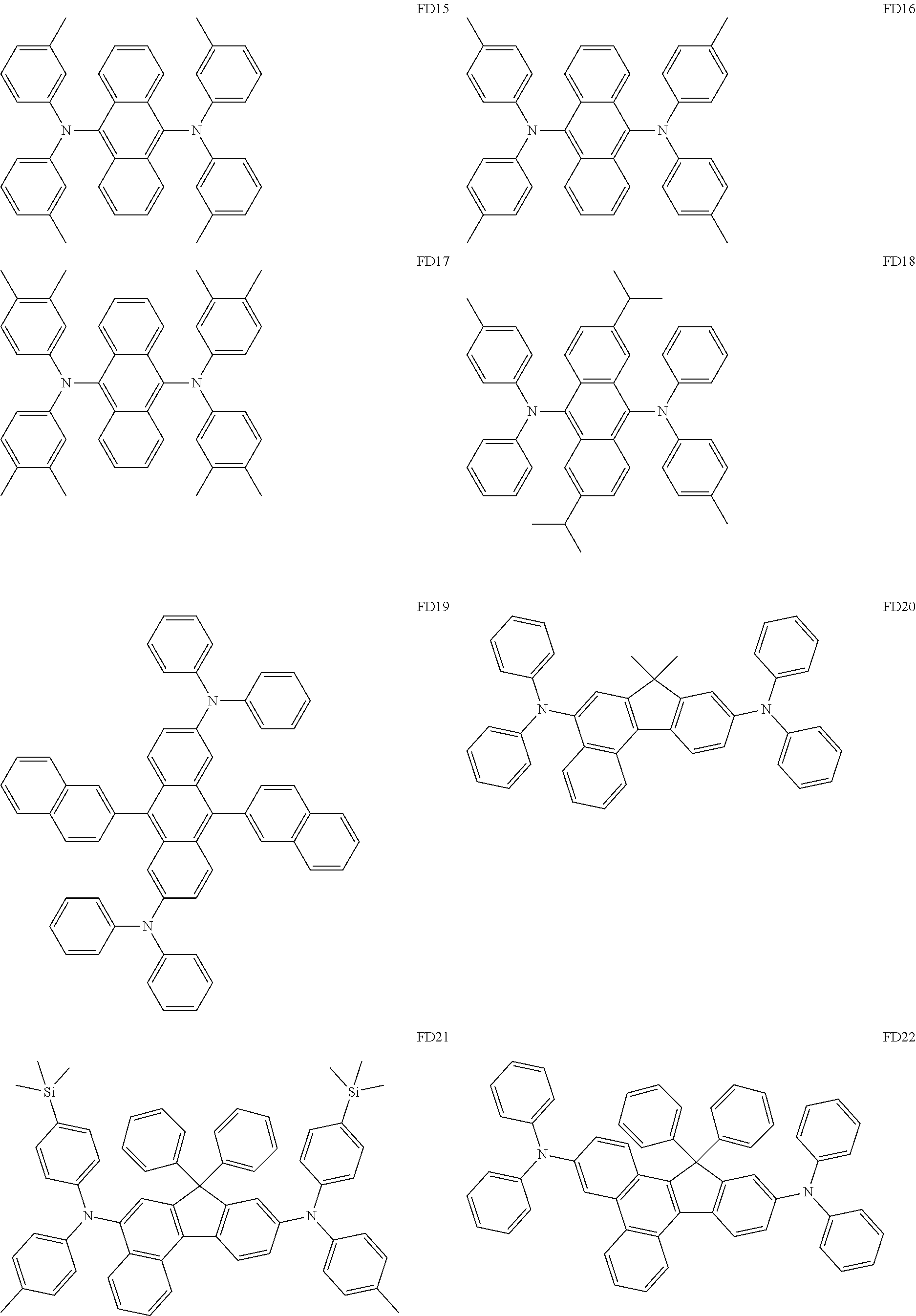

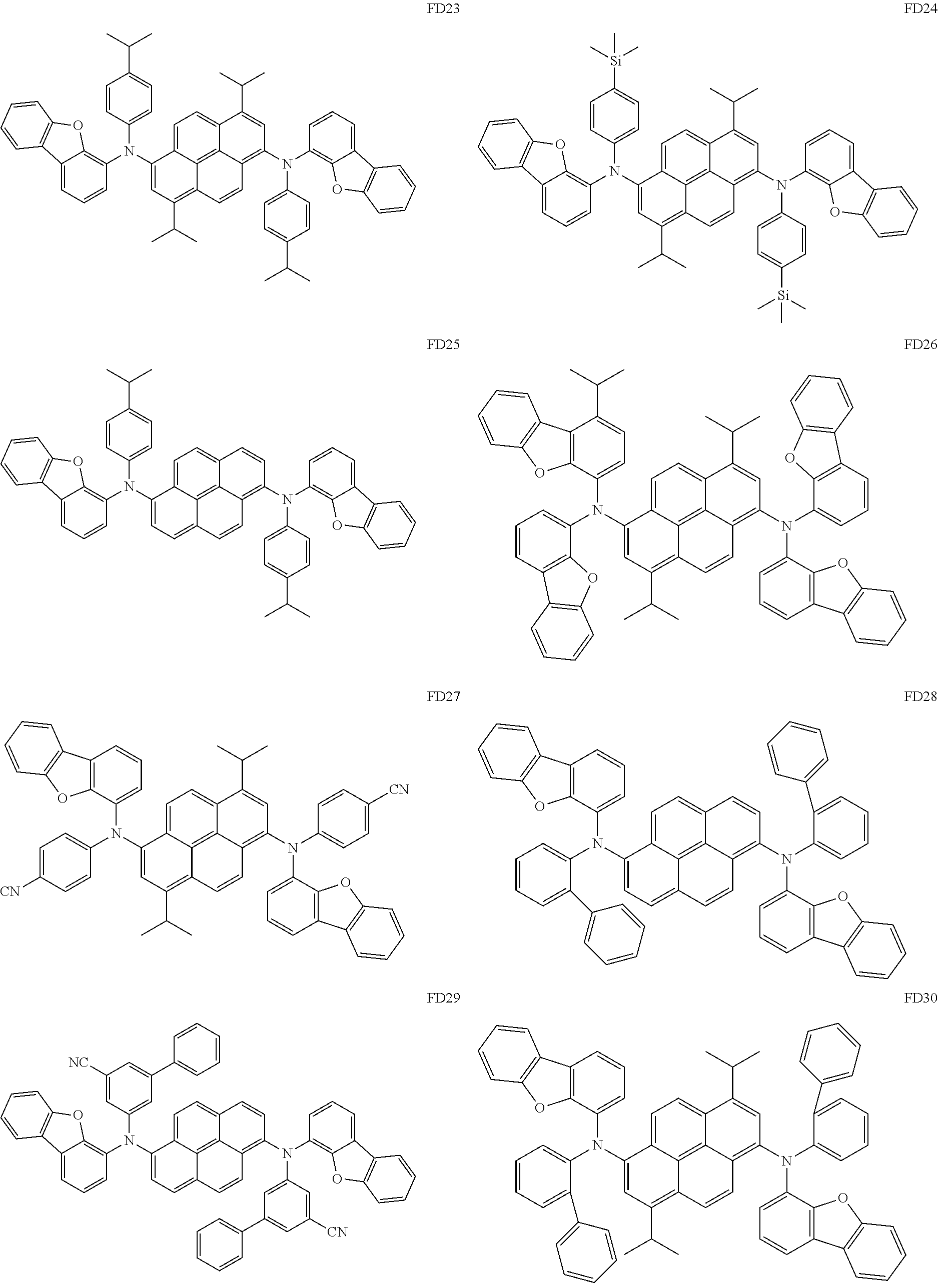

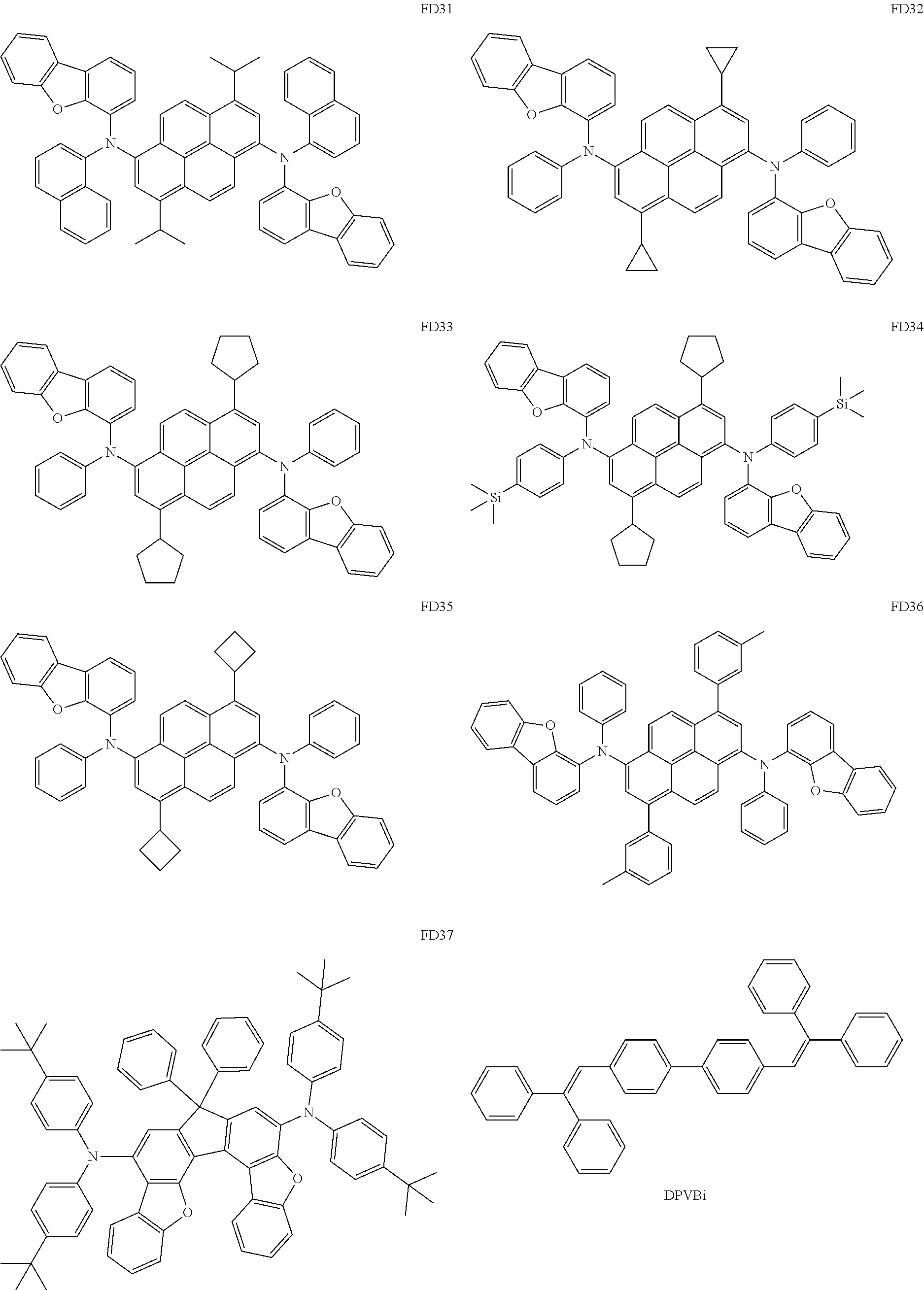

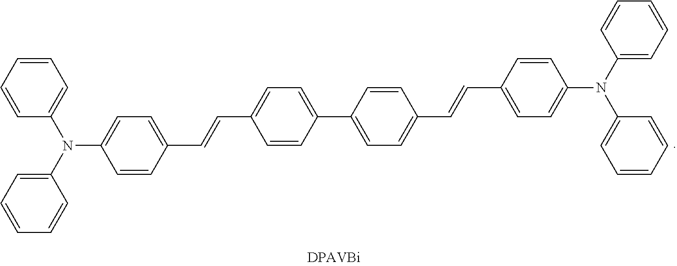 [SMILES: C(=C\c1ccc(N(c2ccccc2)c2ccccc2)cc1)/c1ccc(-c2ccc(/C=C/c3ccc(N(c4ccccc4)c4ccccc4)cc3)cc2)cc1]

### Delayed Fluorescence Material

In one or more embodiments, the emission layer 130 may include a delayed fluorescence material.

The delayed fluorescence material described herein may be selected from among compounds capable of emitting delayed fluorescence based on a delayed fluorescence emission mechanism.

The delayed fluorescence material included in the emission layer 130 may act as a host or a dopant depending on the type or kind of other materials included in the emission layer 130.

In one or more embodiments, a difference (e.g., an absolute value of the difference) between a triplet energy level (eV) of the delayed fluorescence material and a singlet energy level (eV) of the delayed fluorescence material may be in a range of about 0 eV to about 0.5 eV. When the difference between the triplet energy level (eV) of the delayed fluorescence material and the singlet energy level (eV) of the delayed fluorescence material is within the above range, up-conversion from the triplet state to the singlet state of the delayed fluorescence materials may effectively occur, and thus, the light-emitting device 10 may have improved luminescence efficiency.

In one or more embodiments, the delayed fluorescence material may include i) a material including at least one electron donor (for example, a π electron-rich C₃-C₆₀ cyclic group, such as a carbazole group, and/or the like) and at least one electron acceptor (for example, a sulfoxide group, a cyano group, a π electron-deficient nitrogen-containing C₁-C₆₀ cyclic group, and/or the like), and/or ii) a material including a C₈-C₆₀ polycyclic group including two or more cyclic groups that are condensed with each other while sharing boron (B).

Non-limiting examples of the delayed fluorescence material may include at least one of (e.g., selected from among) Compounds DF1 to DF14:

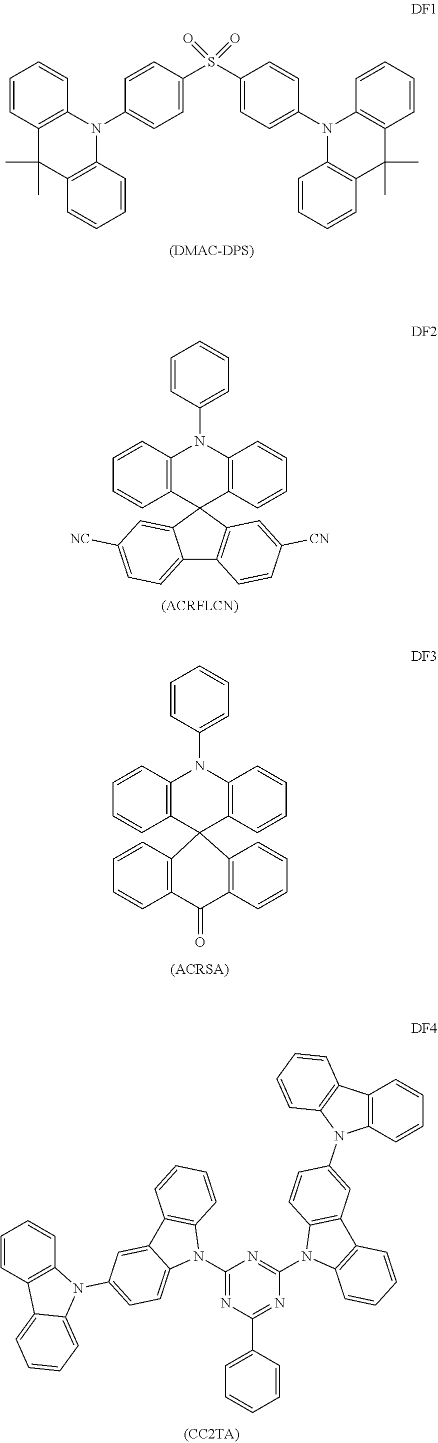

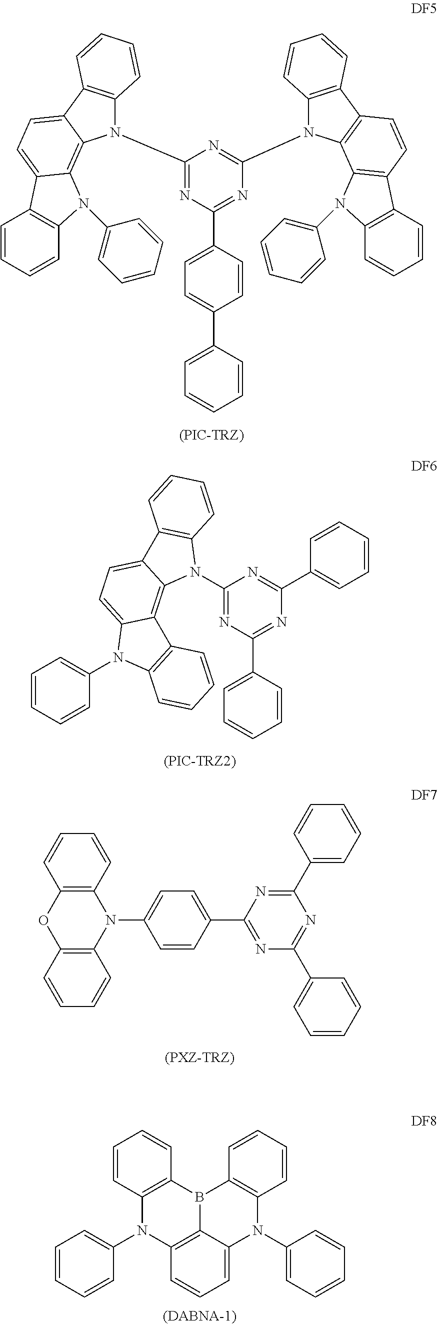

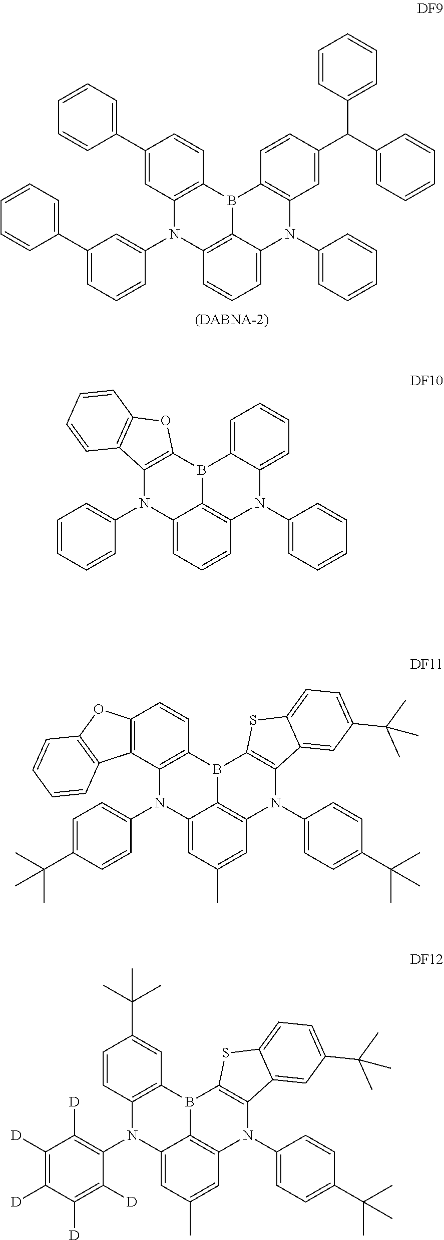

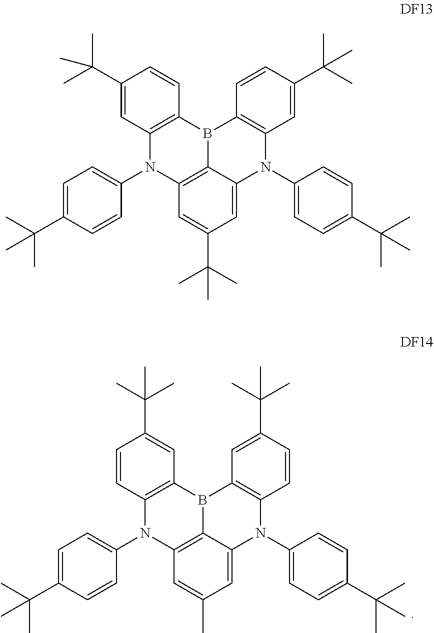

### Quantum Dots

In one or more embodiments, the emission layer 130 may include quantum dots.

The term “quantum dot” as used herein refers to a crystal of a semiconductor compound. Quantum dots may be to emit light of one or more suitable emission wavelengths according to the size of the crystal. Quantum dots may also emit light of one or more suitable emission wavelengths by adjusting the ratio of elements constituting the quantum dots.

The diameter of the quantum dots may be, for example, in a range of about 1 nm to about 10 nm. In the present disclosure, when dot, dots, or dot particles are spherical, “diameter” indicates a particle diameter or an average particle diameter, and when the particles are non-spherical, the “diameter” indicates a major axis length or an average major axis length. The diameter of the particles may be measured utilizing a scanning electron microscope or a particle size analyzer. As the particle size analyzer, for example, HORIBA, LA-950 laser particle size analyzer, may be utilized. When the size of the particles is measured utilizing a particle size analyzer, the average particle diameter is referred to as D50. D50 refers to the average diameter of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

The quantum dots may be synthesized by a wet chemical process, a metal organic chemical vapor deposition process, a molecular beam epitaxy process, or any process similar thereto.

The wet chemical process is a method including mixing a precursor material of a quantum dot with an organic solvent and then growing quantum dot particle crystals. When the crystal grows, the organic solvent naturally acts as a dispersant coordinated on the surface of the quantum dot crystal and controls the growth of the crystal so that the growth of quantum dot particles may be controlled or selected through a process which costs less, and is easier than vapor deposition methods, such as metal organic chemical vapor deposition (MOCVD) or molecular beam epitaxy (MBE).

The quantum dots may include: a Group II-VI semiconductor compound; a Group III-V semiconductor compound; a Group III-VI semiconductor compound; a Group I-III-VI semiconductor compound; a Group IV-VI semiconductor compound; a Group IV element or compound; or any combination thereof.

Non-limiting examples of the Group II-VI semiconductor compound may include: a binary compound, such as CdS, CdSe, CdTe, ZnS, ZnSe, ZnTe, ZnO, HgS, HgSe, HgTe, MgSe, MgS, and/or the like; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and/or the like; a quaternary compound, such as CdZnSeS, CdZnSeTe, CdZnSTe, CdHgSeS, CdHgSeTe, CdHgSTe, HgZnSeS, HgZnSeTe, HgZnSTe, and/or the like; or any combination thereof.

Non-limiting examples of the Group III-V semiconductor compound may include: a binary compound, such as GaN, GaP, GaAs, GaSb, AlN, AlP, AlAs, AlSb, InN, InP, InAs, InSb, and/or the like; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, InPSb, and/or the like; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and/or the like; or any combination thereof. In one or more embodiments, the Group III-V semiconductor compound may further include a Group II element. Non-limiting examples of the Group III-V semiconductor compound further including a Group II element may include InZnP, InGaZnP, InAlZnP, and/or the like.

Non-limiting examples of the Group III-VI semiconductor compound may include: a binary compound, such as GaS, GaSe, Ga₂Se₃, GaTe, InS, InSe, In₂S₃, In₂Se₃, InTe, and/or the like; a ternary compound, such as InGaS₃, InGaSe₃, and/or the like; or any combination thereof.

Non-limiting examples of the Group I-III-VI semiconductor compound may include: a ternary compound, such as AgInS, AgInS₂, AgInSe₂, AgGaS, AgGaS₂, AgGaSe₂, CuInS, CuInS₂, CuInSe₂, CuGaS₂, CuGaSe₂, CuGaO₂, AgGaO₂, AgAlO₂, and/or the like; a ternary compound, such as AgInGaS₂, AgInGaSe₂, and/or the like; or any combination thereof.

Non-limiting examples of the Group IV-VI semiconductor compound may include: a binary compound, such as SnS, SnSe, SnTe, PbS, PbSe, PbTe, and/or the like; a ternary compound, such as SnSeS, SnSeTe, SnSTe, PbSeS, PbSeTe, PbSTe, SnPbS, SnPbSe, SnPbTe, and/or the like; a quaternary compound, such as SnPbSSe, SnPbSeTe, SnPbSTe, and/or the like; or any combination thereof.

The Group IV element or compound may include: a single element, such as Si, Ge, and/or the like; a binary compound, such as SiC, SiGe, and/or the like; or any combination thereof.

Each element included in a multi-element compound, such as the binary compound, the ternary compound, and the quaternary compound, may be present at a substantially uniform concentration or non-uniform concentration in a particle. For example, the formulae above refer to the types (kinds) of elements included in the compound, wherein the ratio of elements in the compound may vary. For example, AgInGaS₂ refers to AgIn_{X}Ga₁₋ₓS₂ (wherein 0<x<1).

In one or more embodiments, the quantum dots may have a single structure in which the concentration of each element in the quantum dots is substantially uniform, or a core-shell dual structure. For example, a material included in the core and a material included in the shell may be different from each other.

The shell of the quantum dots may act as a protective layer that prevents chemical degeneration of the core to maintain semiconductor characteristics, and/or as a charging layer that imparts electrophoretic characteristics to the quantum dots. The shell may be a single layer or a multi-layer. An interface between the core and the shell may have a concentration gradient in which the concentration of an element existing in the shell decreases toward the center of the core.

Examples of the shell of the quantum dots may include an oxide of a metal or non-metal, a semiconductor compound, and/or a (e.g., any suitable) combination thereof. Non-limiting examples of the oxide of a metal or non-metal may include: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, NiO, and/or the like; a ternary compound, such as MgAl₂O₄, CoFe₂O₄, NiFe₂O₄, CoMn₂O₄, and/or the like; or any combination thereof. Examples of the semiconductor compound may include, as described herein: a Group III-VI semiconductor compound; a Group II-VI semiconductor compound; a Group III-V semiconductor compound; a Group III-VI semiconductor compound; a Group I-III-VI semiconductor compound; a Group IV-VI semiconductor compound; or any combination thereof. For example, the semiconductor compound suitable as a shell may include CdS, CdSe, CdTe, ZnS, ZnSe, ZnTe, ZnSeS, ZnTeS, GaAs, GaP, GaS, GaSe, AgGaS, AgGaS₂, GaSb, HgS, HgSe, HgTe, InAs, InP, InGaP, InSb, AlAs, AlP, AlSb, or any combination thereof.

Each element included in a multi-element compound, such as the binary compound and the ternary compound, may be present at a substantially uniform concentration or non-uniform concentration in a particle. For example, the formulae above refer to the types (kinds) of elements included in the compound, wherein the ratio of elements in the compound may vary.

The quantum dots may have a full width at half maximum (FWHM) of an emission spectrum of about 45 nm or less, about 40 nm or less, or for example, about 30 nm or less. When the FWHM of the emission spectrum of the quantum dots is within these ranges, the quantum dots may have improved color purity or improved color reproducibility. In addition, because light emitted through the quantum dots is emitted in all directions, the wide viewing angle may be improved.

In addition, the quantum dots may be, for example, in the form of spherical nanoparticles, pyramidal nanoparticles, multi-arm or cubic nanoparticles, nanotubes, nanowires, nanofibers, or nanoplate particles.

Because the energy band gap of the quantum dot may be controlled or selected by adjusting the size of the quantum dots or the ratio of elements in a quantum dot compound, light of one or more suitable wavelengths may be obtained from a quantum dot emission layer. Accordingly, by using the quantum dots described above (by using quantum dots of different sizes or by varying the ratio of elements in the quantum dot compound), a light-emitting device emitting light of one or more suitable wavelengths may be implemented. For example, the control of the size of the quantum dots or the ratio of elements in the quantum dot compound may enable the quantum dots to emit red light, green light, and/or blue light. In addition, the quantum dots with suitable sizes may be configured to emit white light by combination of light of one or more suitable colors.

### Electron Transport Region 140

The electron transport region 140 may have i) a single-layer structure including (e.g., consisting of) a single layer including (e.g., consisting of) a single material, ii) a single-layer structure including (e.g., consisting of) a single layer including multiple materials that are different from each other, or iii) a multi-layer structure including multiple layers including multiple different materials that are different from each other.

The electron transport region 140 may include a buffer layer, a hole-blocking layer, an electron control layer, an electron transport layer, an electron injection layer, or any combination thereof.

In one or more embodiments, the electron transport region 140 may have an electron transport layer/electron injection layer structure, a hole-blocking layer/electron transport layer/electron injection layer structure, an electron control layer/electron transport layer/electron injection layer structure, or a buffer layer/electron transport layer/electron injection layer structure, wherein constituent layers of each structure are stacked sequentially from the emission layer 130 in the stated order.

In one or more embodiments, the electron transport region 140 (for example, the buffer layer, the hole-blocking layer, the electron control layer, or the electron transport layer in the electron transport region 140) may include a metal-free compound including at least one π electron-deficient nitrogen-containing C₁-C₆₀ cyclic group.

In one or more embodiments, the electron transport region 140 may include a compound represented by Formula 601:

[Ar₆₀₁]ₓₑ₁₁-[(L₆₀₁)ₓₑ₁-R₆₀₁]ₓₑ₂₁, Formula 601
wherein, in Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each independently be the same as described with respect to Q₁,
xe21 may be 1, 2, 3, 4, or 5, and
at least one selected from among A₆₀₁, L₆₀₁, and R₆₀₁ may each independently be a π electron-deficient nitrogen-containing C₁-C₆₀ cyclic group unsubstituted or substituted with at least one R₁₀ₐ.

In one or more embodiments, if (e.g., when) xe11 in Formula 601 is 2 or more, two or more of Ar₆₀₁(s) may be linked to each other via a single bond.

In one or more embodiments, Ar₆₀₁ in Formula 601 may be an anthracene group unsubstituted or substituted with at least one R₁₀ₐ.

In one or more embodiments, the electron transport region 140 may include a compound represented by Formula 601-1:

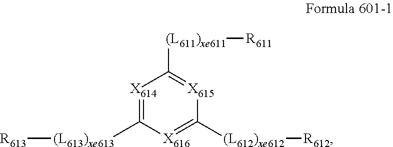

wherein, in Formula 601-1,
X₆₁₄ may be N or C(R₆₁₄), X₆₁₅ may be N or C(R₆₁₅), X₆₁₆ may be N or C(R₆₁₆), and at least one selected from among X₆₁₄ to X₆₁₆ may be N,
L₆₁₁ to L₆₁₃ may each independently be the same as described with L₆₀₁,
xe611 to xe613 may each independently be the same as described with respect to xe1,
R₆₁₁ to R₆₁₃ may each independently be the same as described with respect to R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

In one or more embodiments, xe1 and xe611 to xe613 in Formulae 601 and 601-1 may each independently be 0, 1, or 2.

In one or more embodiments, the electron transport region 140 may include: at least one of (e.g., selected from among) Compounds ET1 to ET45; 2,9-dimethyl-4,7-diphenyl-1,10-phenanthroline (BCP); 4,7-diphenyl-1,10-phenanthroline (Bphen); tris(8-hydroxyquinolinato)aluminum (Alq₃); bis(2-methyl-8-quinolinolato-N1,O8)-(1,1′-biphenyl-4-olato)aluminum (BAlq); 3-(4-biphenyl)-4-phenyl-5-tert-butylphenyl-1,2,4-triazole (TAZ); 4-(naphthalen-1-yl)-3,5-diphenyl-4H-1,2,4-triazole (NTAZ); or any combination thereof:

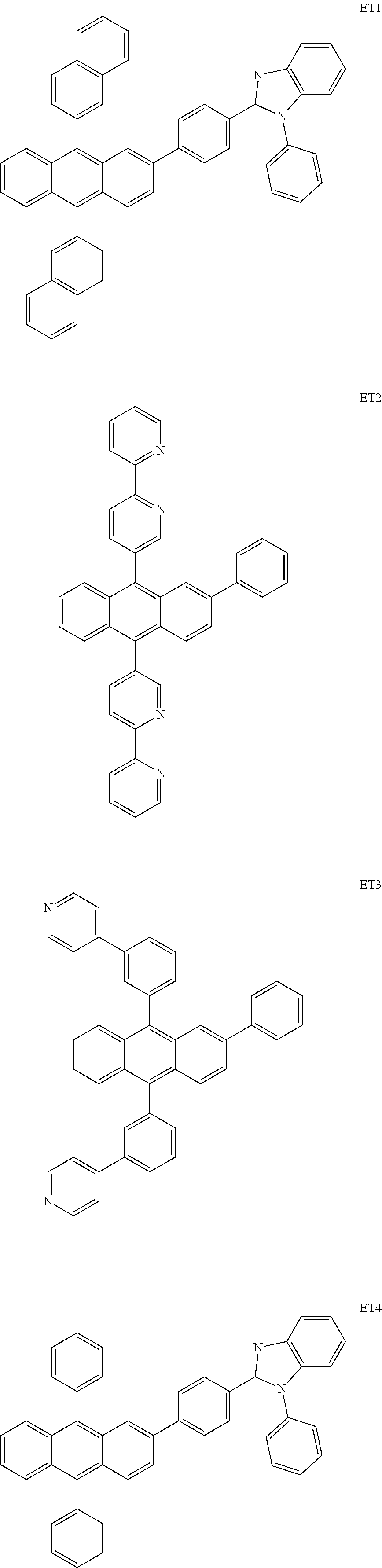

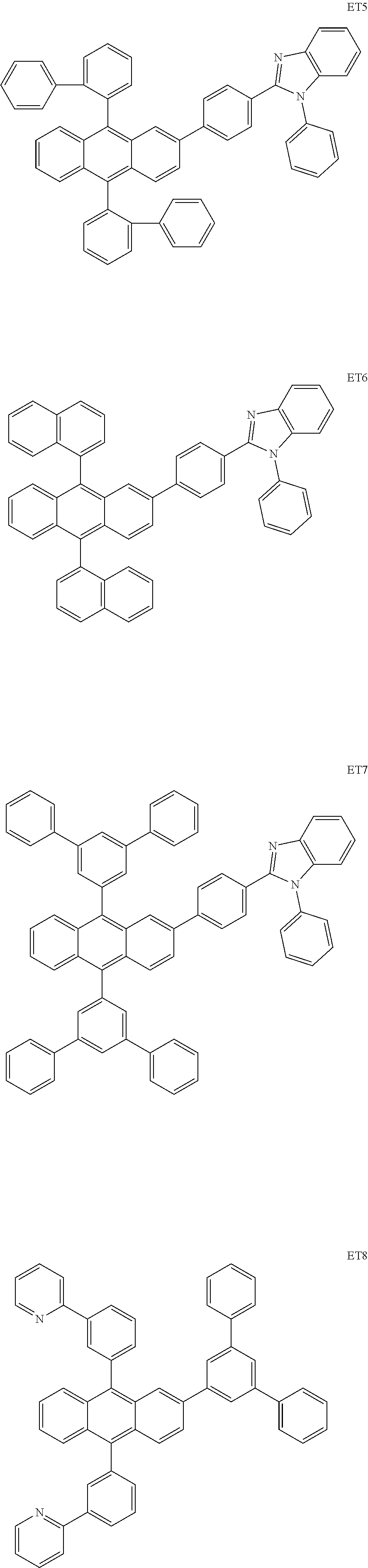 [SMILES: c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1]

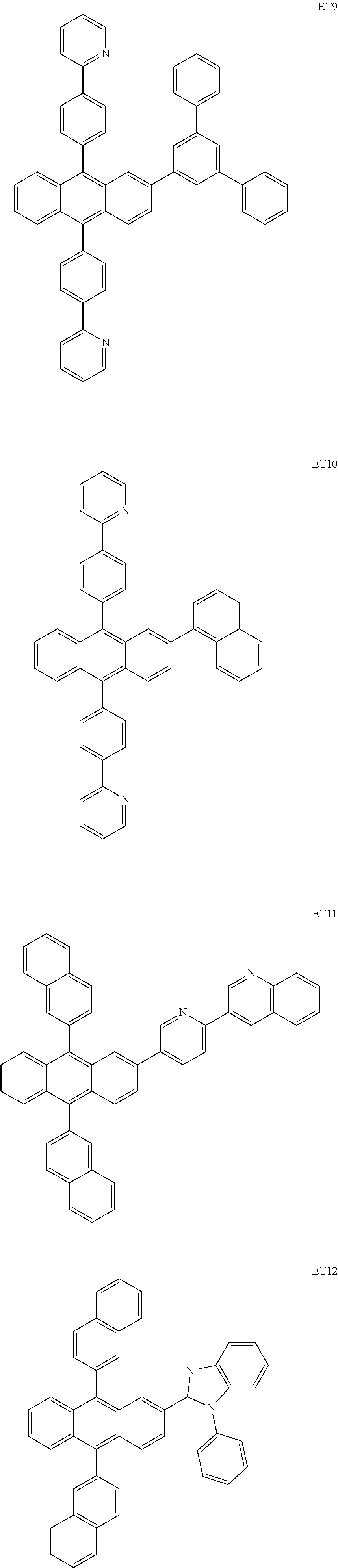

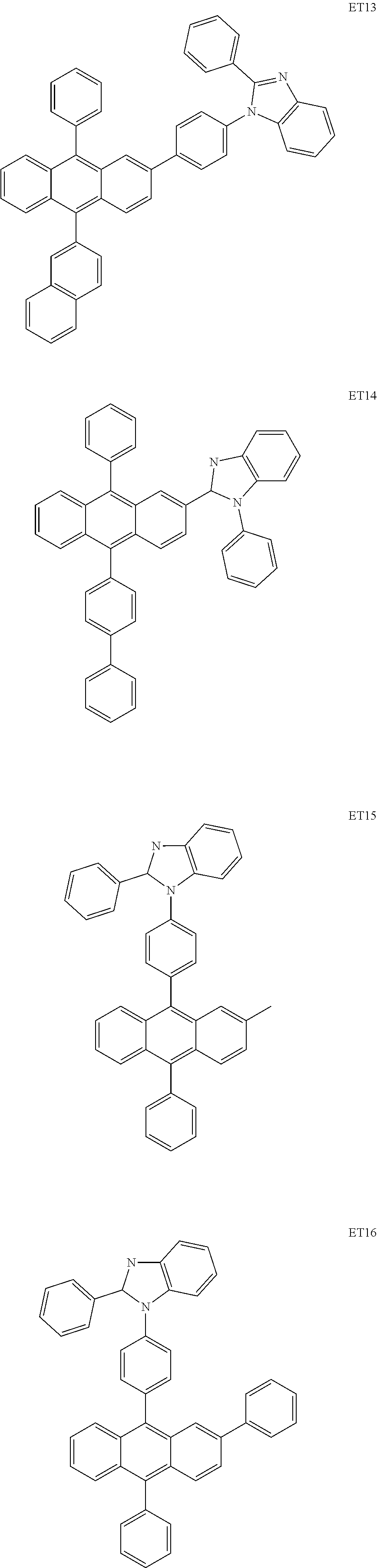

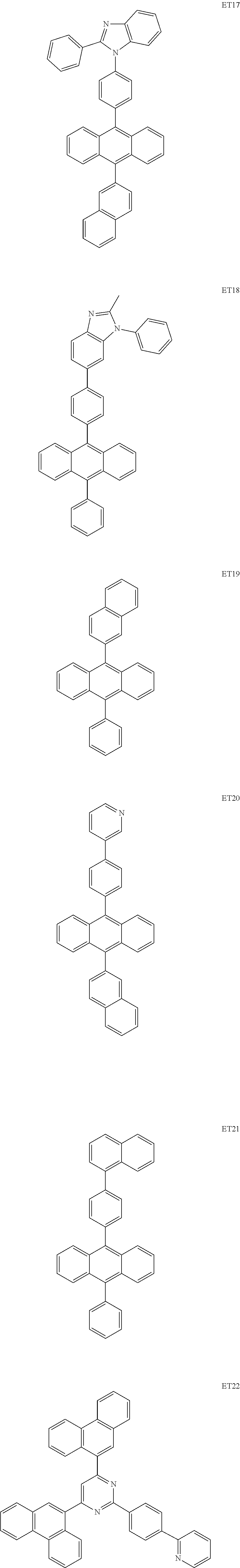

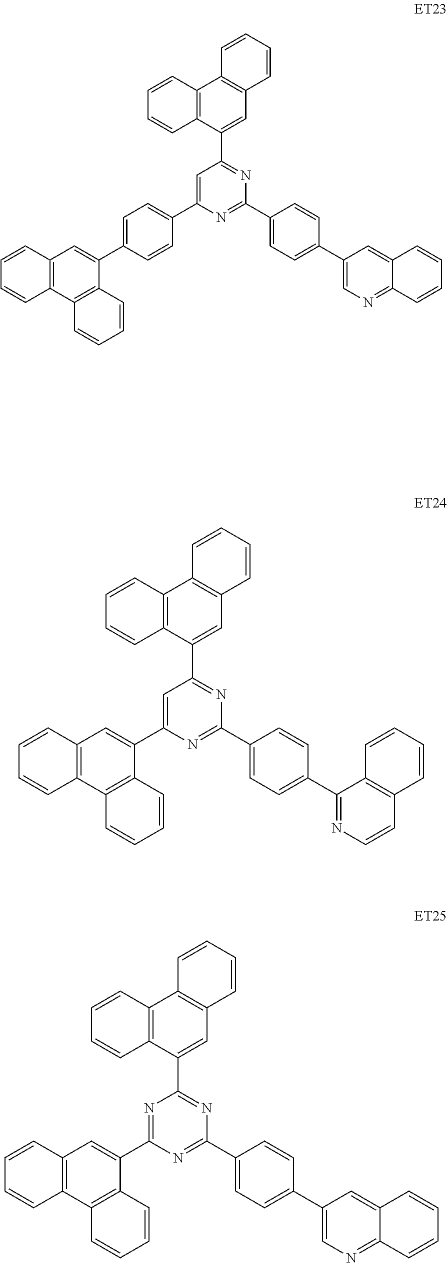 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

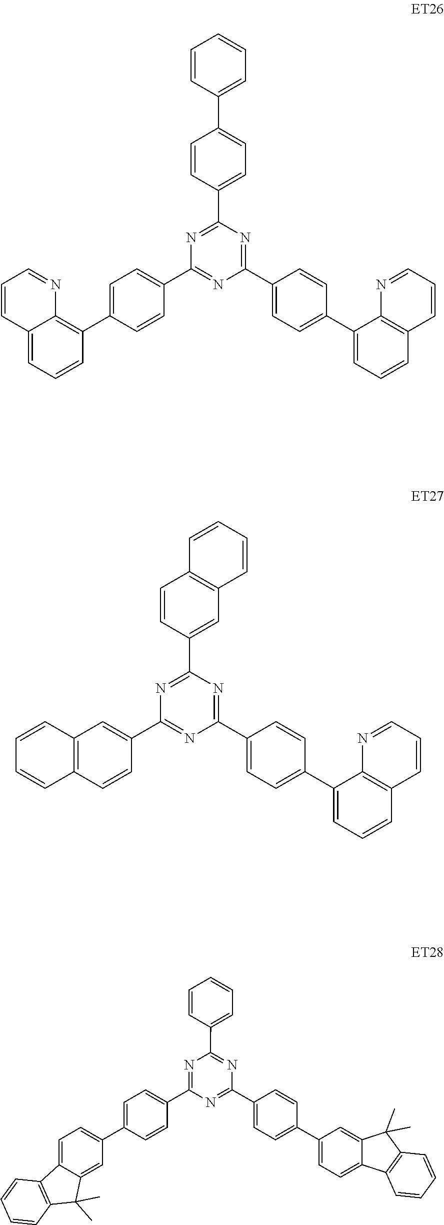 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

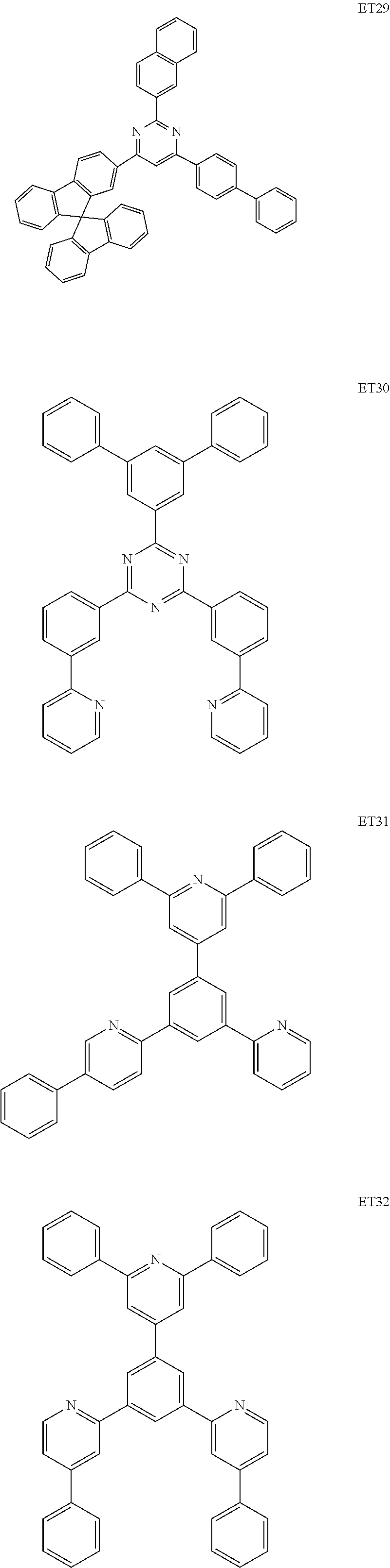 [SMILES: c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1]

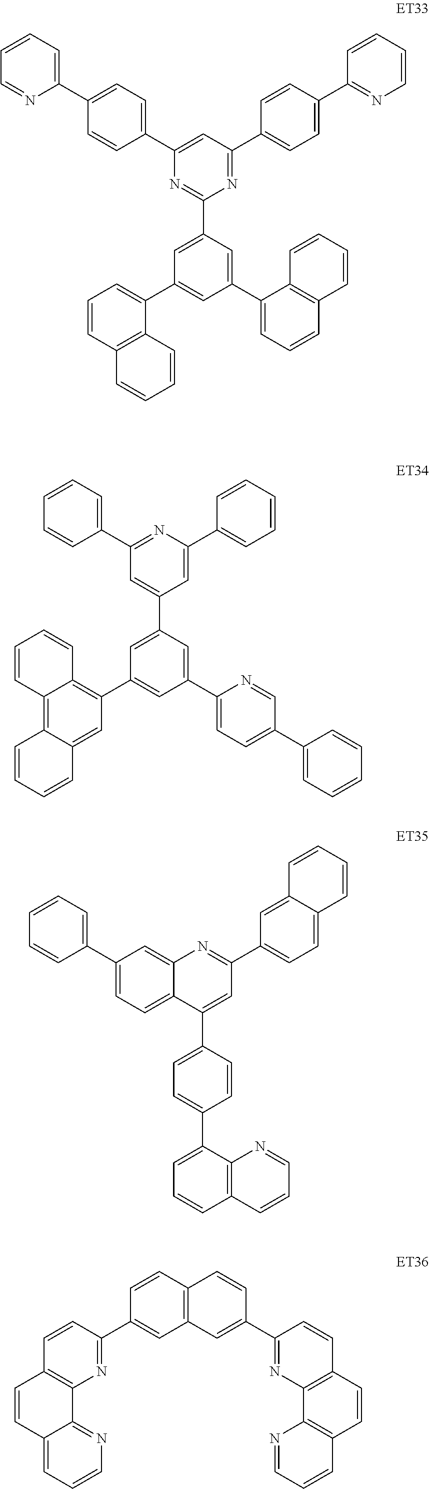 [SMILES: c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

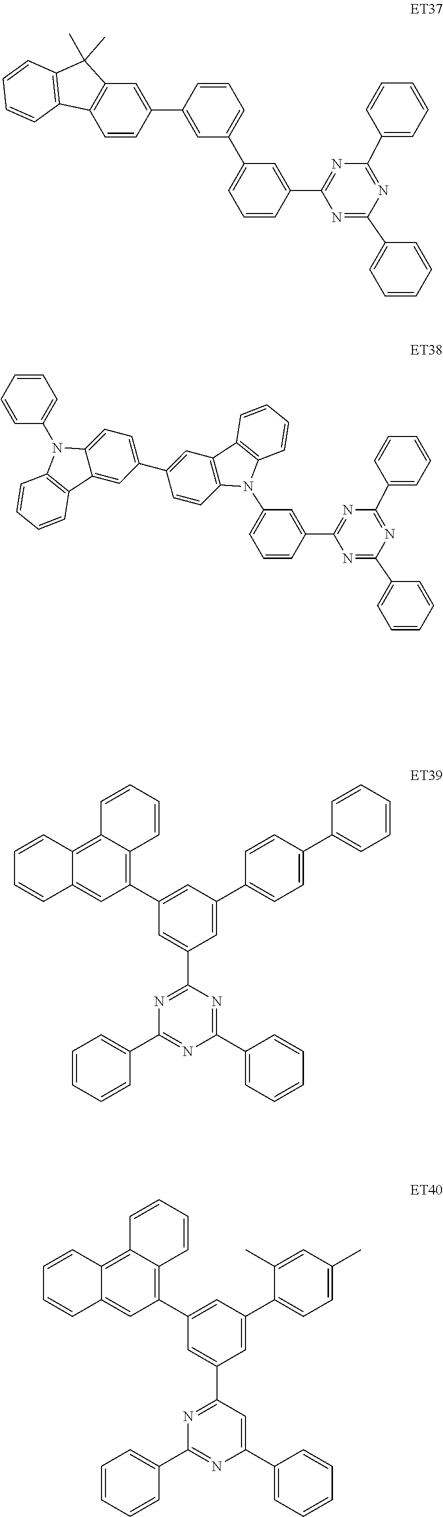 [SMILES: CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(-c2cc(-c3cc(-c4ccccc4)nc(-c4ccccc4)n3)cc(-c3cc4ccccc4c4ccccc34)c2)c(C)c1]

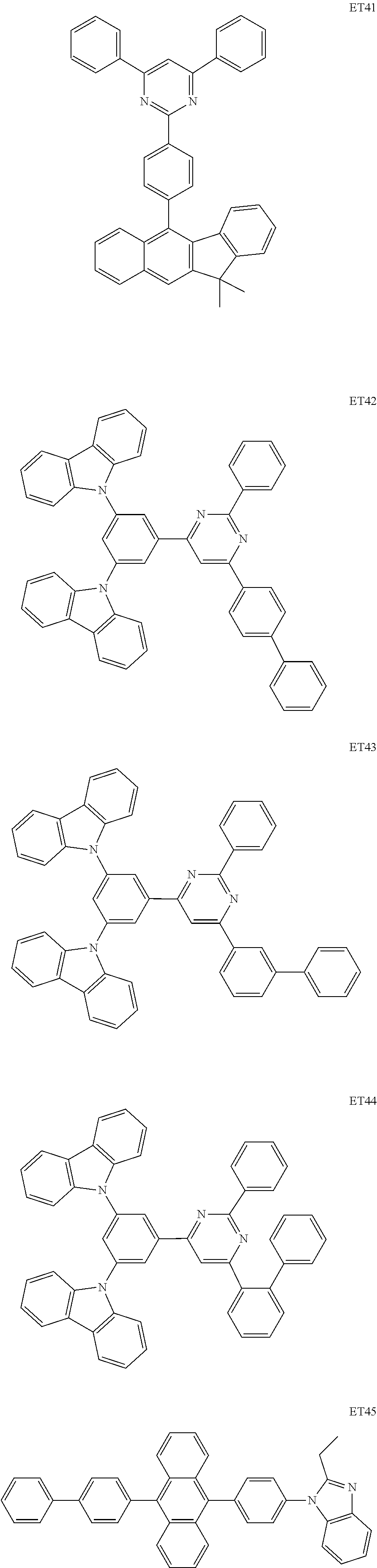 [SMILES: C=C/C(=C\C=C/C)c1cc(-c2ccccc2)nc(-c2ccc(-c3c4c(cc5ccccc35)C(C)(C)c3ccccc3-4)cc2)n1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1]

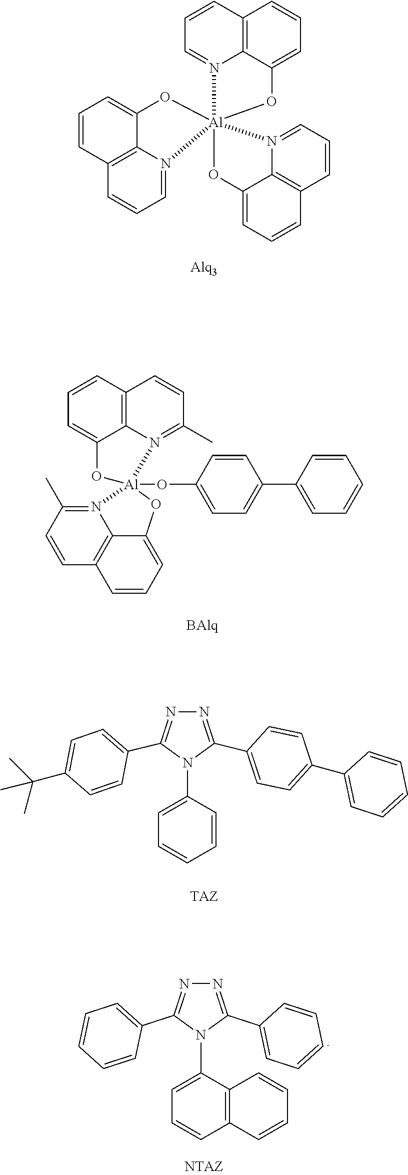

A thickness of the electron transport region 140 may be in a range of about 100 Å to about 5,000 Å, for example, about 160 Å to about 4,000 Å. When the electron transport region 140 includes a buffer layer, a hole-blocking layer, an electron control layer, an electron transport layer, or any combination thereof, a thickness of the buffer layer, the hole-blocking layer, or the electron control layer may be in a range of about 20 Å to about 1,000 Å, for example, about 30 Å to about 300 Å, and a thickness of the electron transport layer may be in a range of about 100 Å to about 1,000 Å, for example, about 150 Å to about 500 Å. When the thicknesses of the buffer layer, the hole-blocking layer, the electron control layer, the electron transport layer, and/or the electron transport region 140 are within the ranges described above, satisfactory electron-transporting characteristics may be obtained without a substantial increase in driving voltage.

In one or more embodiments, the electron transport region 140 (for example, the electron transport layer in the electron transport region 140) may further include, in addition to one or more of the materials described above, a metal-containing material.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the metal ion of the alkaline earth-metal complex may include hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenylbenzimidazole, hydroxyphenylbenzothiazole, bipyridine, phenanthroline, cyclopentadiene, or any combination thereof.

In one or more embodiments, the metal-containing material may include a Li complex. The Li complex may include, for example, Compound ET-D1 (LiQ) or ET-D2:

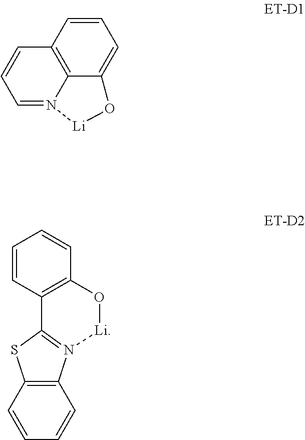

In one or more embodiments, the electron transport region 140 may include an electron injection layer that facilitates injection of electrons from the second electrode 150. The electron injection layer may directly contact the second electrode 150.

The electron injection layer may have i) a single-layer structure including (e.g., consisting of) a single layer including (e.g., consisting of) a single material, ii) a single-layer structure including (e.g., consisting of) a single layer including multiple materials that are different from each other, or iii) a multi-layer structure including multiple layers including multiple materials that are different from each other.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include scandium (Sc), yttrium (Y), cerium (Ce), terbium (Tb), ytterbium (Yb), gadolinium (Gd), or any combination thereof.

The alkali metal-containing compound, the alkaline earth metal-containing compound, and the rare earth metal-containing compound may include oxides, halides (for example, fluorides, chlorides, bromides, iodides, and/or the like), or tellurides of the alkali metal, the alkaline earth metal, and the rare earth metal, respectively, or any combination thereof.

The alkali metal-containing compound may include: an alkali metal oxide, such as Li₂O, Cs₂O, K₂O, and/or the like; an alkali metal halide, such as LiF, NaF, CsF, KF, LiI, NaI, CsI, KI, and/or the like; or any combination thereof. The alkaline earth metal-containing compound may include an alkaline earth metal compound, such as BaO, SrO, CaO, BaₓSr₁₋ₓO (wherein x is a real number satisfying 0<x<1), BaₓCa₁₋ₓO (wherein x is a real number satisfying 0<x<1), and/or the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include a lanthanide metal telluride. Non-limiting examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, Lu₂Te₃, and/or the like.

The alkali metal complex, the alkaline earth-metal complex, and the rare earth metal complex may include i) one of metal ions of the alkali metal, one of metal ions of the alkaline earth metal, and one of metal ions of the rare earth metal, respectively, and ii) a ligand bonded to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

In one or more embodiments, the electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (for example, a compound represented by Formula 601).

In one or more embodiments, the electron injection layer may include (e.g., consist of) i) an alkali metal-containing compound (for example, an alkali metal halide), or ii) a) an alkali metal-containing compound (for example, an alkali metal halide), and b) an alkali metal, an alkaline earth metal, a rare earth metal, or any combination thereof. In one or more embodiments, the electron injection layer may be a KI:Yb co-deposited layer, an RbI:Yb co-deposited layer, a LiF:Yb co-deposited layer, and/or the like.

When the electron injection layer further includes an organic material, the alkali metal, the alkaline earth metal, the rare earth metal, the alkali metal-containing compound, the alkaline earth metal-containing compound, the rare earth metal-containing compound, the alkali metal complex, the alkaline earth-metal complex, the rare earth metal complex, or any combination thereof may be uniformly (e.g., substantially uniformly) or non-uniformly dispersed in a matrix including the organic material.

A thickness of the electron injection layer may be in a range of about 1 Å to about 100 Å, for example, about 3 Å to about 90 Å. When the thickness of the electron injection layer is within the range described above, satisfactory electron injection characteristics may be obtained without a substantial increase in driving voltage.

### Second Electrode 150

The second electrode 150 may be arranged on the electron transport region 140. The second electrode 150 may be a cathode, which is an electron injection electrode, and as a material for forming the second electrode 150, a metal, an alloy, an electrically conductive compound, or any combination thereof, each having a low-work function, may be used.

The second electrode 150 may include lithium (Li), silver (Ag), magnesium (Mg), aluminum (Al), aluminum-lithium (Al-Li), calcium (Ca), magnesium-indium (Mg-In), magnesium-silver (Mg—Ag), ytterbium (Yb), silver-ytterbium (Ag—Yb), ITO, IZO, or any combination thereof. The second electrode 150 may be a transmissive electrode, a transflective electrode, or a reflective electrode.

The second electrode 150 may have a single-layer structure or a multi-layer structure including multiple layers.

### Capping Layer

A first capping layer may be arranged outside (e.g., on) the first electrode 110, and/or a second capping layer may be arranged outside (e.g., on) the second electrode 150. For example, in one or more embodiments, the light-emitting device 10 may have a structure in which the first capping layer, the first electrode 110, the interlayer, and the second electrode 150 are sequentially stacked in the stated order, a structure in which the first electrode 110, the interlayer, the second electrode 150, and the second capping layer are sequentially stacked in the stated order, or a structure in which the first capping layer, the first electrode 110, the interlayer, the second electrode 150, and the second capping layer are sequentially stacked in the stated order.

In one or more embodiments, light generated in the emission layer 130 of the light-emitting device 10 may pass through the first electrode 110, which is a semi-transmissive electrode or a transmissive electrode, and through the first capping layer to the outside. In one or more embodiments, light generated in the emission layer 130 of the light-emitting device 10 may pass through the second electrode 150, which is a semi-transmissive electrode or a transmissive electrode, and through the second capping layer to the outside.

The first capping layer and the second capping layer may increase external emission efficiency according to the principle of constructive interference. Accordingly, the light extraction efficiency of the light-emitting device 10 may be increased, and thus, the luminescence efficiency of the light-emitting device 10 may be improved.

Each of the first capping layer and the second capping layer may include a material having a refractive index of about 1.2 or more (e.g., at 460 nm).

The first capping layer and the second capping layer may each independently be an organic capping layer including an organic material, an inorganic capping layer including an inorganic material, or an organic-inorganic composite capping layer including an organic material and an inorganic material.

At least one of the first capping layer and/or the second capping layer may (e.g., the first capping layer and the second capping layer may each independently) include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may each optionally be substituted with a substituent including O, N, S, Se, Si, F, Cl, Br, I, or any combination thereof. In one or more embodiments, at least one of the first capping layer and/or the second capping layer may (e.g., the first capping layer and the second capping layer may each independently) include an amine group-containing compound.

In one or more embodiments, at least one of the first capping layer and/or the second capping layer may (e.g., the first capping layer and the second capping layer may each independently) include a compound represented by Formula 201, a compound represented by Formula 202, or any combination thereof.

In one or more embodiments, at least one of the first capping layer and/or the second capping layer may (e.g., the first capping layer and the second capping layer may each independently) include: at least one of (e.g., selected from among) Compounds HT28 to HT33; at least one of (e.g., selected from among) Compounds CP1 to CP6; β-NPB; or any combination thereof:

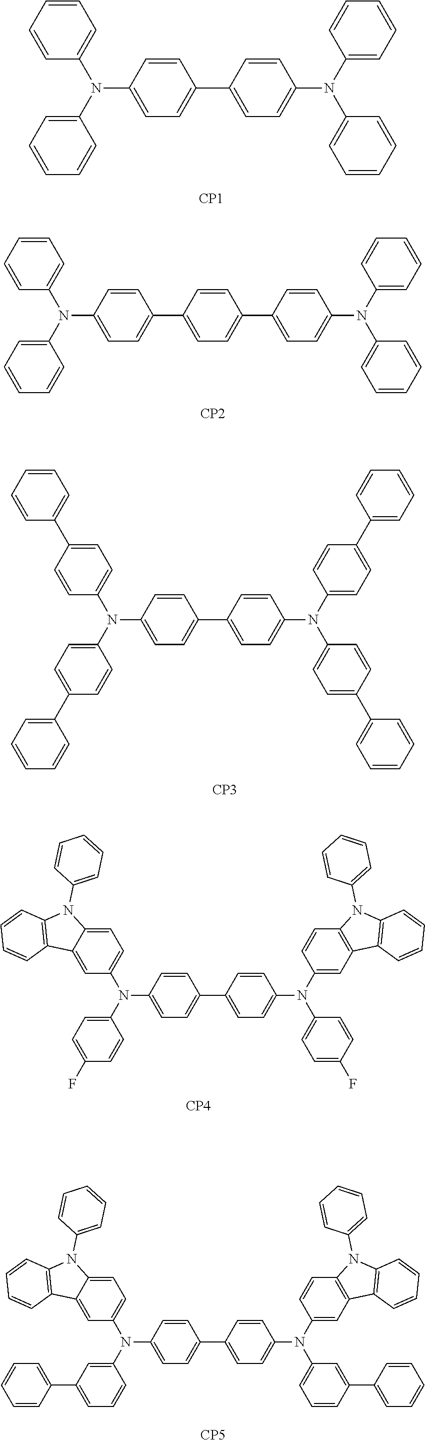

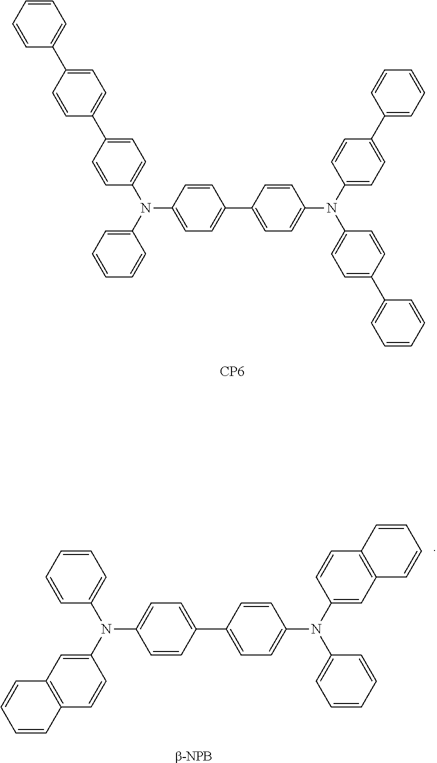 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; "CP6" ; c1ccc(N(c2ccc(-c3ccc(N(c4ccccc4)c4ccc5ccccc5c4)cc3)cc2)c2ccc3ccccc3c2)cc1]

### Film

In one or more embodiments, the electronic apparatus may further include a film. The film may be, for example, an optical member (or a light control element) (for example, a color filter, a color conversion member, a capping layer, a light-extraction efficiency enhancement layer, a selective light-absorbing layer, a polarizing layer, a quantum dot-containing layer, and/or the like), a light-blocking member (for example, a light reflective layer, a light-absorbing layer, and/or the like), a protective member (for example, an insulating layer, a dielectric layer, and/or the like), and/or the like.

### Electronic Apparatus

The light-emitting device 10 may be included in one or more suitable electronic apparatuses. For example, the electronic apparatus including the light-emitting device 10 may be a display apparatus, an authentication apparatus, and/or the like.

In one or more embodiments, the electronic apparatus (for example, a display apparatus) may further include, in addition to the light-emitting device 10, i) a color filter, ii) a color conversion layer, or iii) a color filter and a color conversion layer. The color filter and/or the color conversion layer may be arranged in at least one direction of travel of light emitted from the light-emitting device 10. For example, in one or more embodiments, the light emitted from the light-emitting device 10 may be blue light or white light (e.g., combined white light). Details on the light-emitting device 10 may be the same as described above. In one or more embodiments, the color conversion layer may include quantum dots. The quantum dots may be, for example, the quantum dots described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be arranged among the subpixel areas to define each of the subpixel areas.

In one or more embodiments, the color filter may further include a plurality of color filter areas and light-shielding patterns arranged among the color filter areas, and the color conversion layer may further include a plurality of color conversion areas and light-shielding patterns arranged among the color conversion areas.

The plurality of color filter areas (or the plurality of color conversion areas) may include a first area configured to emit first color light, a second area configured to emit second color light, and/or a third area configured to emit third color light, wherein the first color light, the second color light, and/or the third color light may have different maximum emission wavelengths. For example, in one or more embodiments, the first color light may be red light, the second color light may be green light, and the third color light may be blue light. For example, in one or more embodiments, the plurality of color filter areas (or the plurality of color conversion areas) may include quantum dots. For example, the first area may include red quantum dots to emit red light, the second area may include green quantum dots to emit green light, and the third area may not include (e.g., may exclude any of) quantum dots. Details on the quantum dot may be the same as described herein. The first area, the second area, and/or the third area may each further include a scatter.

In one or more embodiments, the light-emitting device may be to emit first light, the first area may be to absorb the first light to emit first-first color light, the second area may be to absorb the first light to emit second-first color light, and the third area may be to absorb the first light to emit third-first color light. In this regard, the first-first color light, the second-first color light, and the third-first color light may have different maximum emission wavelengths. For example, the first light may be blue light, the first-first color light may be red light, the second-second color light may be green light, and the third-first color light may be blue light.

In one or more embodiments, the electronic apparatus may further include a thin-film transistor, in addition to the light-emitting device 10. The thin-film transistor may include a source electrode, a drain electrode, and an active layer, wherein one selected from among the source electrode and the drain electrode may be electrically connected to the first electrode or the second electrode of the light-emitting device.

The thin-film transistor may further include a gate electrode, a gate insulating film, and/or the like.

The active layer may include crystalline silicon, amorphous silicon, an organic semiconductor, an oxide semiconductor, and/or the like.

In one or more embodiments, the electronic apparatus may further include a sealing portion for sealing the light-emitting device. The sealing portion may be arranged between the color filter and/or the color conversion layer and the light-emitting device. The sealing portion may allow light from the light-emitting device to be extracted to the outside, and may concurrently (e.g., simultaneously) prevent or reduce ambient air and moisture from penetrating into the light-emitting device. The sealing portion may be a sealing substrate including a transparent glass substrate or a plastic substrate. The sealing portion may be a thin-film encapsulation layer including at least one layer of an organic layer and/or an inorganic layer. When the sealing portion is a thin-film encapsulation layer, the electronic apparatus may be flexible.

In one or more embodiments, various functional layers may be additionally arranged on the sealing portion, in addition to the color filter and/or the color conversion layer, according to the use of the electronic apparatus. Non-limiting examples of the functional layers may include a touch screen layer, a polarizing layer, and/or the like. The touch screen layer may be a pressure-sensitive touch screen layer, a capacitive touch screen layer, or an infrared touch screen layer.

The authentication apparatus may be, for example, a biometric authentication apparatus that authenticates an individual by using biometric information of a living body (for example, fingertips, pupils, and/or the like). The authentication apparatus may further include, in addition to the light-emitting device described above, a biometric information collector.

The electronic apparatus may be applied to one or more of displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, one or more suitable measuring instruments, meters (for example, meters for a vehicle, an aircraft, and a vessel), projectors, and/or the like.

### Electronic Equipment

The light-emitting device 10 may be included in one or more suitable electronic equipment.

In one or more embodiments, the electronic equipment including the light-emitting device 10 may be at least one of a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a PDA, a wearable device, a laptop computer, a digital camera, a camcorder, a viewfinder, a micro display, a 3D display, a virtual reality display, an augmented reality display, a vehicle, a video wall with multiple displays tiled together, a theater screen, a stadium screen, a phototherapy device, or a signboard.

Because the light-emitting device 10 has improved luminescence efficiency and improved lifespan, the electronic equipment including the light-emitting device 10 may have characteristics, such as high luminance, high resolution, and/or low power consumption.

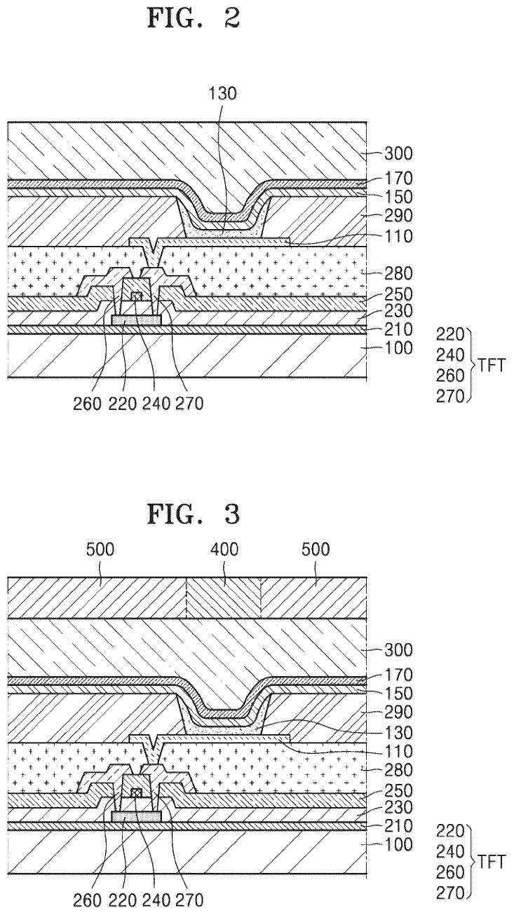

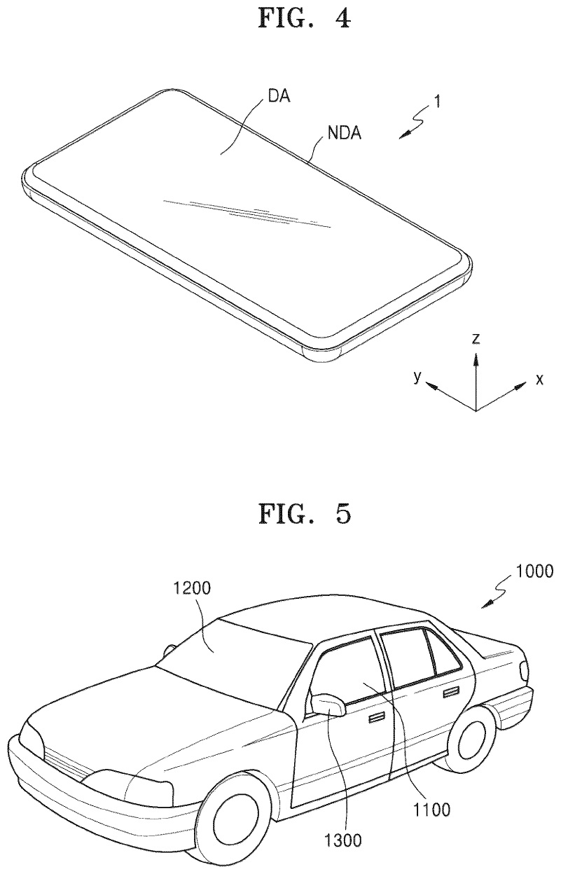

### Descriptions of FIGS. 2 and 3

FIG. 2 is a cross-sectional view of an electronic apparatus according to one or more embodiments of the present disclosure.

The electronic apparatus of FIG. 2 may include a substrate 100, a thin-film transistor (TFT), a light-emitting device, and an encapsulation portion 300.

The substrate 100 may be a flexible substrate, a glass substrate, or a metal substrate. A buffer layer 210 may be on the substrate 100. The buffer layer 210 may prevent or reduce penetration of impurities through the substrate 100, and may provide a flat surface on the substrate 100.

The TFT may be on the buffer layer 210. The TFT may include an active layer 220, a gate electrode 240, a source electrode 260, and a drain electrode 270.

The active layer 220 may include an inorganic semiconductor, such as silicon or polysilicon, an organic semiconductor, or an oxide semiconductor, and may include a source region, a drain region, and a channel region.

A gate insulating film 230 for insulating the active layer 220 from the gate electrode 240 may be on the active layer 220, and the gate electrode 240 may be on the gate insulating film 230.

An interlayer insulating film 250 may be on the gate electrode 240. The interlayer insulating film 250 may be arranged between the gate electrode 240 and the source electrode 260 and between the gate electrode 240 and the drain electrode 270, to insulate from one another.

The source electrode 260 and the drain electrode 270 may be on the interlayer insulating film 250. The interlayer insulating film 250 and the gate insulating film 230 may be formed to expose the source region and the drain region of the active layer 220, and the source electrode 260 and the drain electrode 270 may be arranged in contact with the exposed portions of the source region and the drain region of the active layer 220, respectively.

The TFT may be electrically connected to the light-emitting device to drive the light-emitting device, and may be covered and protected by a passivation layer 280. The passivation layer 280 may include an inorganic insulating film, an organic insulating film, or any combination thereof. The light-emitting device may be provided on the passivation layer 280. The light-emitting device may include a first electrode 110, an interlayer, and a second electrode 150.

The first electrode 110 may be on the passivation layer 280. The passivation layer 280 may be arranged to expose a portion of the drain electrode 270, not fully covering the drain electrode 270, and the first electrode 110 may be arranged to be connected to the exposed portion of the drain electrode 270.

A pixel-defining film 290 including an insulating material may be on the first electrode 110. The pixel-defining film 290 may expose a certain region of the first electrode 110, and the interlayer may be formed in the exposed region of the first electrode 110. The pixel-defining film 290 may be a polyimide-based organic film or a polyacrylic organic film. Although not shown in FIG. 2, in one or more embodiments, at least some layers of the interlayer may extend beyond the upper portion of the pixel-defining film 290 to be arranged in the form of a common layer.

The second electrode 150 may be on the interlayer, and a capping layer 170 may be further formed on the second electrode 150. The capping layer 170 may be formed to cover the second electrode 150.

The encapsulation portion 300 may be on the capping layer 170. The encapsulation portion 300 may be arranged on the light-emitting device to protect the light-emitting device from moisture and/or oxygen. The encapsulation portion 300 may include: an inorganic film including silicon nitride (SiNₓ), silicon oxide (SiOₓ), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic-based resin (for example, polymethyl methacrylate, polyacrylic acid, and/or the like), an epoxy-based resin (for example, aliphatic glycidyl ether (AGE), and/or the like), or any combination thereof; and/or a (e.g., any suitable) combination of the inorganic film and the organic film.

FIG. 3 is a cross-sectional view of an electronic apparatus according to one or more embodiments of the present disclosure.

The electronic apparatus of FIG. 3 is substantially the same as the electronic apparatus of FIG. 2, except that a light-shielding pattern 500 and a functional region 400 are additionally arranged on the encapsulation portion 300. The functional region 400 may be i) a color filter area, ii) a color conversion area, or iii) a combination of the color filter area and the color conversion area. In one or more embodiments, the light-emitting device included in the electronic apparatus of FIG. 3 may be a tandem light-emitting device.

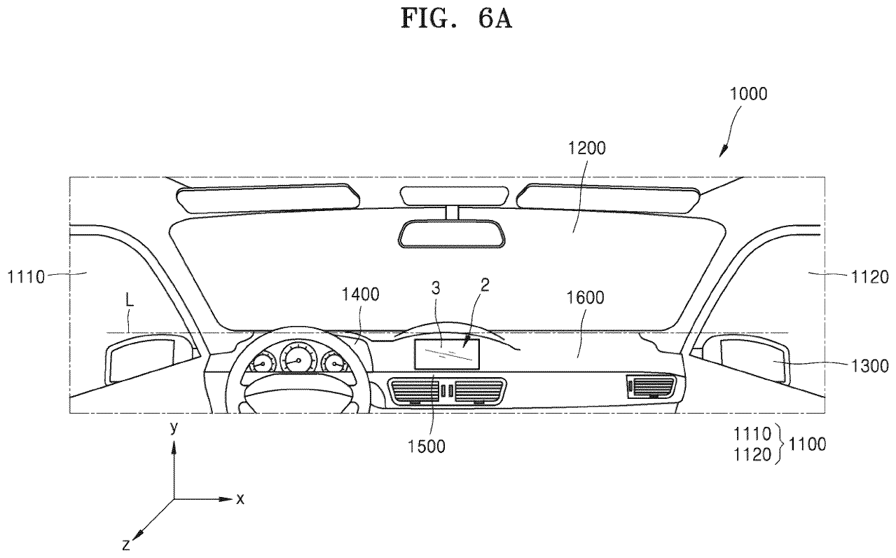

### Description of FIG. 4

FIG. 4 is a schematic perspective view of electronic equipment 1 including a light-emitting device according to one or more embodiments of the present disclosure. The electronic equipment 1 may be, as an apparatus that displays a moving image or a still image, a portable electronic equipment, such as a mobile phone, a smartphone, a tablet personal computer (PC), a mobile communication terminal, an electronic notebook, an electronic book, a portable multimedia player (PMP), a navigation, or a ultra-mobile PC (UMPC), as well as one or more suitable products, such as a television, a laptop, a monitor, a billboard, or an Internet of things (IoT) device. The electronic equipment 1 may be such a product above or a part thereof. In one or more embodiments, the electronic equipment 1 may be a wearable device, such as a smart watch, a watch phone, a glasses-type or kind display, or a head mounted display (HMD), or a part of the wearable device. However, embodiments of the present disclosure are not limited thereto. For example, in one or more embodiments, the electronic equipment 1 may be a center information display (CID) on an instrument panel and a center fascia or dashboard of a vehicle, a room mirror display instead of a side mirror of a vehicle, an entertainment display for a rear seat of a vehicle or a display arranged on the back of a front seat thereof, a head up display (HUD) installed in the front of a vehicle or projected on a front window glass thereof, or a computer generated hologram augmented reality head up display (CGH AR HUD). FIG. 4 illustrates an embodiment in which the electronic equipment 1 is a smartphone for convenience of explanation.

The electronic equipment 1 may include a display area DA and a non-display area NDA outside the display area DA. The electronic equipment 1 may implement an image through an array of a plurality of pixels that are two-dimensionally arranged in the display area DA.

The non-display area NDA is an area that does not display an image, and may entirely be around (e.g., surround) the display area DA. On the non-display area NDA, a driver for providing electrical signals or power to display devices arranged on the display area DA may be arranged. On the non-display area NDA, a pad, which is an area to which an electronic element or a printed circuit board may be electrically connected, may be arranged.

In the electronic equipment 1, a length in an x-axis direction and a length (e.g., a width) in a y-axis direction may be different from each other. In one or more embodiments, as shown in FIG. 4, the length in the x-axis direction may be less than the length (e.g., the width) in the y-axis direction. In one or more embodiments, the length in the x-axis direction may be substantially the same as the length (e.g., the width) in the y-axis direction. In one or more embodiments, the length in the x-axis direction may be greater than the length (e.g., the width) in the y-axis direction.

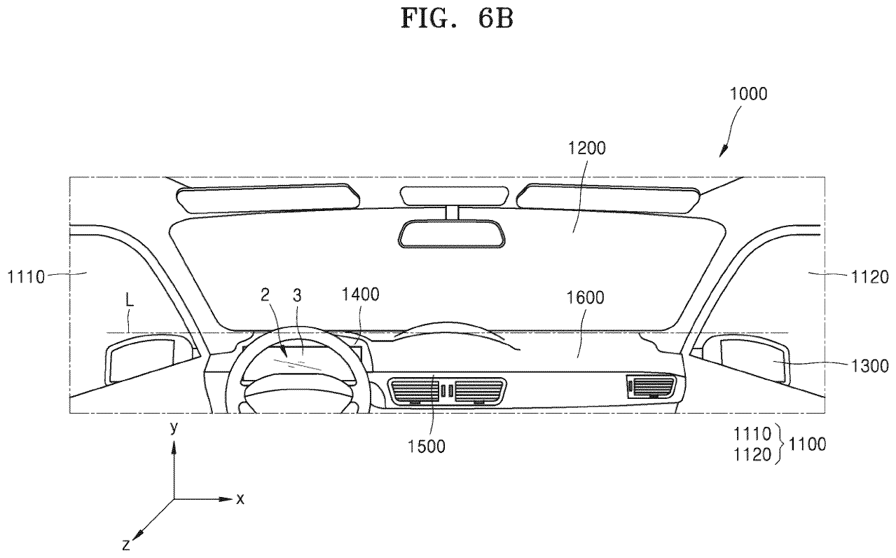

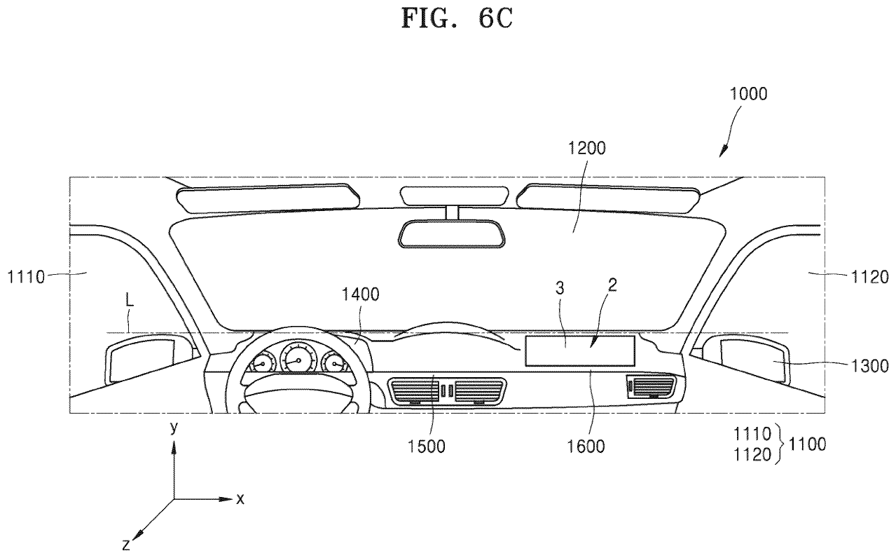

### Descriptions of FIGS. 5 and 6A to 6C

FIG. 5 is a schematic view of an exterior of a vehicle 1000 as electronic equipment including a light-emitting device according to one or more embodiments. FIGS. 6A to 6C are each a schematic view of an interior of the vehicle 1000 according to one or more embodiments.

Referring to FIGS. 5, 6A, 6B, and 6C, the vehicle 1000 may refer to one or more suitable apparatuses for moving an object to be transported, such as a human, an object, or an animal, from a departure point to a destination point. The vehicle 1000 may include a vehicle traveling on a road or a track, a vessel moving over the sea or a river, an airplane flying in the sky using the action of air, and/or the like.

In one or more embodiments, the vehicle 1000 may travel on a road or a track. The vehicle 1000 may move in a certain direction according to rotation of at least one wheel thereof. For example, the vehicle 1000 may include a three-wheeled or four-wheeled vehicle, a construction machine, a two-wheeled vehicle, a prime mover device, a bicycle, or a train running on a track.

The vehicle 1000 may include a body of the vehicle 1000 having an interior and an exterior, and a chassis in which mechanical apparatuses necessary for driving are installed as other parts except for the body of the vehicle 1000. The exterior of the body of the vehicle 1000 may include a front panel, a bonnet, a roof panel, a rear panel, a trunk, a pillar provided at a boundary between doors, and/or the like. The chassis of the vehicle 1000 may include a power generating device, a power transmitting device, a driving device, a steering device, a braking device, a suspension device, a transmission device, a fuel device, front and rear wheels, left and right wheels, and/or the like.

The vehicle 1000 may include a side window glass 1100, a front window glass 1200, a side mirror 1300, a cluster 1400, a center fascia 1500, a passenger seat dashboard 1600, and a display apparatus 2.

The side window glass 1100 and the front window glass 1200 may be partitioned by a pillar arranged between the side window glass 1100 and the front window glass 1200.

The side window glass 1100 may be installed on a side of the vehicle 1000. In one or more embodiments, the side window glass 1100 may be installed on a door of the vehicle 1000. A plurality of side window glasses 1100 may be provided and may face each other. In one or more embodiments, the side window glass 1100 may include a first side window glass 1110 and a second side window glass 1120. In one or more embodiments, the first side window glass 1110 may be arranged adjacent to the cluster 1400. The second side window glass 1120 may be arranged adjacent to the passenger seat dashboard 1600.

In one or more embodiments, the side window glasses 1100 may be spaced and/or apart (e.g., spaced apart or separated) from each other in the x-direction or the −x-direction (the direction opposite the x-direction). For example, the first side window glass 1110 and the second side window glass 1120 may be spaced and/or apart (e.g., spaced apart or separated) from each other in the x-direction or the −x-direction. For example, an imaginary straight line L connecting the side window glasses 1100 may extend in the x-direction or the −x-direction. For example, an imaginary straight line L connecting the first side window glass 1110 and the second side window glass 1120 to each other may extend in the x-direction or the −x-direction.

The front window glass 1200 may be installed in the front of the vehicle 1000. The front window glass 1200 may be arranged between the side window glasses 1100 opposite to (e.g., facing) each other.

The side mirror 1300 may provide a rear view of the vehicle 1000. The side mirror 1300 may be installed on the exterior of the body of the vehicle 1000. In one or more embodiments, a plurality of side mirrors 1300 may be provided. Any one of the plurality of side mirrors 1300 may be arranged outside the first side window glass 1110. The other one of the plurality of side mirrors 1300 may be arranged outside the second side window glass 1120.

The cluster 1400 may be arranged in front of a steering wheel. The cluster 1400 may include a tachometer, a speedometer, a coolant thermometer, a fuel gauge, a turn signal indicator, a high beam indicator, a warning light, a seat belt warning light, an odometer, a tachograph, an automatic shift selector indicator, a door open warning light, an engine oil warning light, and/or a low fuel warning light.

The center fascia 1500 may include a control panel on which a plurality of buttons for adjusting an audio device, an air conditioning device, and/or a seat heater are arranged. The center fascia 1500 may be arranged on one side of the cluster 1400.

The passenger seat dashboard 1600 may be spaced and/or apart (e.g., spaced apart or separated) from the cluster 1400, and the center fascia 1500 may be arranged between the cluster 1400 and the passenger seat dashboard 1600. In one or more embodiments, the cluster 1400 may be arranged to correspond to a driver seat, and the passenger seat dashboard 1600 may be arranged to correspond to a passenger seat. In one or more embodiments, the cluster 1400 may be adjacent to the first side window glass 1110, and the passenger seat dashboard 1600 may be adjacent to the second side window glass 1120.

In one or more embodiments, the display apparatus 2 may include a display panel 3, and the display panel 3 may display an image. The display apparatus 2 may be arranged inside the vehicle 1000. In one or more embodiments, the display apparatus 2 may be arranged between the side window glasses 1100 opposite to (e.g., facing) each other. The display apparatus 2 may be arranged on at least one of the cluster 1400, the center fascia 1500, or the passenger seat dashboard 1600.

The display apparatus 2 may include an organic light-emitting display apparatus, an inorganic light-emitting display apparatus, a quantum dot display apparatus, and/or the like. Hereinafter, as the display apparatus 2 according to one or more embodiments, an organic light-emitting display apparatus including the light-emitting device according to the disclosure will be described as an example, but one or more suitable types (kinds) of display apparatuses as described above may be used in embodiments.

Referring to FIG. 6A, in one or more embodiments, the display apparatus 2 may be arranged on the center fascia 1500. In one or more embodiments, the display apparatus 2 may display navigation information. In one or more embodiments, the display apparatus 2 may display information regarding audio settings, video settings, or vehicle settings.

Referring to FIG. 6B, in one or more embodiments, the display apparatus 2 may be arranged on the cluster 1400. In these embodiments, the cluster 1400 may display driving information and/or the like through the display apparatus 2. For example, the cluster 1400 may digitally implement driving information and/or the like. The cluster 1400 may digitally display vehicle information and driving information as images. For example, in one or more embodiments, a needle and a gauge of a tachometer and one or more suitable warning light icons may be displayed by a digital signal.

Referring to FIG. 6C, in one or more embodiments, the display apparatus 2 may be arranged on the passenger seat dashboard 1600. The display apparatus 2 may be embedded in the passenger seat dashboard 1600 or arranged on the passenger seat dashboard 1600. In one or more embodiments, the display apparatus 2 arranged on the passenger seat dashboard 1600 may display an image related to information displayed on the cluster 1400 and/or information displayed on the center fascia 1500. In one or more embodiments, the display apparatus 2 arranged on the passenger seat dashboard 1600 may display information different from information displayed on the cluster 1400 and/or information displayed on the center fascia 1500.

### Manufacturing Method

The layers constituting the hole transport region 120, the emission layer 130, and the layers constituting the electron transport region 140 may each be formed in a certain region by using one or more suitable methods such as vacuum deposition, spin coating, casting, Langmuir-Blodgett (LB) deposition, ink-jet printing, laser-printing, laser-induced thermal imaging, and/or the like.

When the layers constituting the hole transport region 120, the emission layer 130, and the layers constituting the electron transport region 140 are each formed by vacuum deposition, the deposition may be performed at a deposition temperature of about 100° C. to about 500° C., a vacuum degree of about 10⁻⁸ torr to about 10-3 torr, and a deposition speed of about 0.01 Å/sec to about 100 Å/sec, depending on a material to be included in a layer to be formed and the structure of a layer to be formed.

### DEFINITION OF TERMS

The term “C₃-C₆₀ carbocyclic group” as used herein refers to a cyclic group including (e.g., consisting of) carbon atoms as the only ring-forming atoms and having 3 to 60 carbon atoms.

The term “C₁-C₆₀ heterocyclic group” as used herein refers to a cyclic group that has 1 to 60 carbon atoms and further has, in addition to carbon, a heteroatom as a ring-forming atom.

The C₃-C₆₀ carbocyclic group and the C₁-C₆₀ heterocyclic group may each be a monocyclic group including (e.g., consisting of) one (e.g., exactly one) ring or a polycyclic group in which two or more rings are condensed with each other. For example, the number of ring-forming atoms of the C₁-C₆₀ heterocyclic group may be from 3 to 61.

The term “cyclic group” as used herein may include both (e.g., simultaneously) the C₃-C₆₀ carbocyclic group and the C₁-C₆₀ heterocyclic group.

The term “π electron-rich C₃-C₆₀ cyclic group” as used herein refers to a cyclic group that has 3 to 60 carbon atoms and does not include *—N═*′ as a ring-forming moiety.

The term “π electron-deficient nitrogen-containing C₁-C₆₀ cyclic group” as used herein refers to a heterocyclic group that has 1 to 60 carbon atoms and includes *—N═*′ as a ring-forming moiety.

For example,
the C₃-C₆₀ carbocyclic group may be i) Group T1 or ii) a condensed cyclic group in which two or more of Group T1 are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group), and
the C₁-C₆₀ heterocyclic group may be i) Group T2, ii) a condensed cyclic group in which two or more of Group T2 are condensed with each other, or iii) a condensed cyclic group in which at least one Group T2 and at least one Group T1 are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, a xanthene group, and/or the like).

The π electron-rich C₃-C₆₀ cyclic group may be i) Group T1, ii) a condensed cyclic group in which two or more of Group T1 are condensed with each other, iii) Group T3, iv) a condensed cyclic group in which two or more of Group T3 are condensed with each other, or v) a condensed cyclic group in which at least one Group T3 and at least one Group T1 are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, and/or the like).

The π electron-deficient nitrogen-containing C₁-C₆₀ cyclic group may be i) Group T4, ii) a condensed cyclic group in which two or more of Group T4 are condensed with each other, iii) a condensed cyclic group in which at least one Group T4 and at least one Group T1 are condensed with each other, iv) a condensed cyclic group in which at least one Group T4 and at least one Group T3 are condensed with each other, or v) a condensed cyclic group in which at least one Group T4, at least one Group T1, and at least one Group T3 are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like).

Group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or bicyclo[2.2.1]heptane) group, a norbornene group, a bicyclo[1.1.1]pentane group, a bicyclo[2.1.1]hexane group, a bicyclo[2.2.2]octane group, or a benzene group.

Group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group.

Group T3 may be a furan group, a thiophene group, a 1H-pyrrole group, a silole group, or a borole group.

Group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “cyclic group,” “C₃-C₆₀ carbocyclic group,” “C₁-C₆₀ heterocyclic group,” “π electron-rich C₃-C₆₀ cyclic group,” or “π electron-deficient nitrogen-containing C₁-C₆₀ cyclic group,” as used herein, refer to a monovalent or polyvalent group (for example, a divalent group, a trivalent group, a tetravalent group, or the like) that is condensed with (e.g., combined together with) a cyclic group according to the structure of a formula for which the corresponding term is used.

For example, the “benzene group” may be a benzo group, a phenyl group, a phenylene group, and/or the like, which may be easily understood by one of ordinary skill in the art according to the structure of a formula including the “benzene group.”

Non-limiting examples of the monovalent C₃-C₆₀ carbocyclic group and the monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

Non-limiting examples of the divalent C₃-C₆₀ carbocyclic group and the divalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as used herein refers to a linear or branched aliphatic hydrocarbon monovalent group that has 1 to 60 carbon atoms, and non-limiting examples thereof may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, a tert-decyl group, and/or the like.

The term “C₁-C₆₀ alkylene group” as used herein refers to a divalent group having substantially the same structure as the C₁-C₆₀ alkyl group.

The term “C₂-C₆₀ alkenyl group” as used herein refers to a monovalent hydrocarbon group having at least one carbon-carbon double bond in the middle or at the terminus of a C₂-C₆₀ alkyl group, and non-limiting examples thereof may include an ethenyl group, a propenyl group, a butenyl group, and/or the like.

The term “C₂-C₆₀ alkenylene group” as used herein refers to a divalent group having substantially the same structure as the C₂-C₆₀ alkenyl group.

The term “C₂-C₆₀ alkynyl group” as used herein refers to a monovalent hydrocarbon group having at least one carbon-carbon triple bond in the middle or at the terminus of a C₂-C₆₀ alkyl group, and non-limiting examples thereof may include an ethynyl group, a propynyl group, and/or the like.

The term “C₂-C₆₀ alkynylene group” as used herein refers to a divalent group having substantially the same structure as the C₂-C₆₀ alkynyl group.

The term “C₁-C₆₀ alkoxy group” as used herein refers to a monovalent group represented by —OA₁₀₁ (wherein A₁₀₁ is a C₁-C₆₀ alkyl group), and non-limiting examples thereof may include a methoxy group, an ethoxy group, an isopropyloxy group, and/or the like.

The term “C₃-C₁₀ cycloalkyl group” as used herein refers to a monovalent saturated hydrocarbon cyclic group having 3 to 10 carbon atoms, and non-limiting examples thereof may include a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, a bicyclo[2.2.2]octyl group, and/or the like.

The term “C₃-C₁₀ cycloalkylene group” as used herein refers to a divalent group having substantially the same structure as the C₃-C₁₀ cycloalkyl group.

The term “C₁-C₁₀ heterocycloalkyl group” as used herein refers to a monovalent cyclic group of 1 to 10 carbon atoms, further including, in addition to a carbon atom, at least one heteroatom as a ring-forming atom, and non-limiting examples thereof may include a 1,2,3,4-oxatriazolidinyl group, a tetrahydrofuranyl group, a tetrahydrothiophenyl group, and/or the like.

The term “C₁-C₁₀ heterocycloalkylene group” as used herein refers to a divalent group having substantially the same structure as the C₁-C₁₀ heterocycloalkyl group.

The term “C₃-C₁₀ cycloalkenyl group” as used herein refers to a monovalent cyclic group that has 3 to 10 carbon atoms and at least one carbon-carbon double bond in the ring thereof and no aromaticity, and non-limiting examples thereof may include a cyclopentenyl group, a cyclohexenyl group, a cycloheptenyl group, and/or the like.

The term “C₃-C₁₀ cycloalkenylene group” as used herein refers to a divalent group having substantially the same structure as the C₃-C₁₀ cycloalkenyl group.

The term “C₁-C₁₀ heterocycloalkenyl group” as used herein refers to a monovalent cyclic group that has 1 to 10 carbon atoms, further includes, in addition to a carbon atom, at least one heteroatom as a ring-forming atom, and has at least one double bond in the ring thereof. Non-limiting examples of the C₁-C₁₀ heterocycloalkenyl group may include a 4,5-dihydro-1,2,3,4-oxatriazolyl group, a 2,3-dihydrofuranyl group, a 2,3-dihydrothiophenyl group, and/or the like.

The term “C₁-C₁₀ heterocycloalkenylene group” as used herein refers to a divalent group having substantially the same structure as the C₁-C₁₀ heterocycloalkenyl group.

The term “C₆-C₆₀ aryl group” as used herein refers to a monovalent group having a carbocyclic aromatic system of 6 to 60 carbon atoms.

The term “C₆-C₆₀ arylene group” as used herein refers to a divalent group having a carbocyclic aromatic system of 6 to 60 carbon atoms.

Non-limiting examples of the C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, and/or the like.

When the C₆-C₆₀ aryl group and the C₆-C₆₀ arylene group each include two or more rings, the two or more rings may be condensed with each other.

The term “C₁-C₆₀ heteroaryl group” as used herein refers to a monovalent group having a heterocyclic aromatic system of 1 to 60 carbon atoms, further including, in addition to carbon atoms, at least one heteroatom, as ring-forming atoms.

The term “C₁-C₆₀ heteroarylene group” as used herein refers to a divalent group having a heterocyclic aromatic system of 1 to 60 carbon atoms, further including, in addition to carbon atoms, at least one heteroatom, as ring-forming atoms.

Non-limiting examples of the C₁-C₆₀ heteroaryl group may include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a naphthyridinyl group, and/or the like.

When the C₁-C₆₀ heteroaryl group and the C₁-C₆₀ heteroarylene group each include two or more rings, the two or more rings may be condensed with each other.

The term “monovalent non-aromatic condensed polycyclic group” as used herein refers to a monovalent group (for example, having 8 to 60 carbon atoms) having two or more rings condensed to each other, only carbon atoms as ring-forming atoms, and non-aromaticity in the entire molecular structure as a whole. Non-limiting examples of the monovalent non-aromatic condensed polycyclic group may include an indenyl group, a fluorenyl group, a spiro-bifluorenyl group, a benzofluorenyl group, an indenophenanthrenyl group, an indenoanthracenyl group, and/or the like.

The term “divalent non-aromatic condensed polycyclic group” as used herein refers to a divalent group having substantially the same structure as the monovalent non-aromatic condensed polycyclic group.

The term “monovalent non-aromatic condensed heteropolycyclic group” as used herein refers to a monovalent group (for example, having 1 to 60 carbon atoms) having two or more rings condensed to each other, further including, in addition to carbon atoms, at least one heteroatom, as ring-forming atoms, and having non-aromaticity in its entire molecular structure as a whole. Non-limiting examples of the monovalent non-aromatic condensed heteropolycyclic group may include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, a benzothienodibenzothiophenyl group, and/or the like.

The term “divalent non-aromatic condensed heteropolycyclic group” as used herein refers to a divalent group having substantially the same structure as the monovalent non-aromatic condensed heteropolycyclic group.

The term “C₆-C₆₀ aryloxy group” as used herein refers to —OA₁₀₂ (wherein A₁₀₂ is a C₆-C₆₀ aryl group).

The term “C₆-C₆₀ arylthio group” as used herein refers to —SA₁₀₃ (wherein A₁₀₃ is a C₆-C₆₀ aryl group).

The term “C₇-C₆₀ arylalkyl group” as used herein refers to -A₁₀₄A₁₀₅ (wherein A₁₀₄ is a C₁-C₅₄ alkylene group, and A₁₀₅ is a C₆-C₅₉ aryl group).

The term “C₂-C₆₀ heteroarylalkyl group” as used herein refers to -A₁₀₆A₁₀₇ (wherein A₁₀₆ is a C₁-C₅₉ alkylene group, and A₁₀₇ is a C₁-C₅₉ heteroaryl group).

The term “R₁₀ₐ” as used herein may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Q₁ to Q₃, Q₁₁ to Q₁₃, Q₂₁ to Q₂₃, and Q₃₁ to Q₃₃ as used herein may each independently be: hydrogen; deuterium; —F; —Cl; —Br; —I; a hydroxyl group; a cyano group; a nitro group; C₁-C₆₀ alkyl group; C₂-C₆₀ alkenyl group; C₂-C₆₀ alkynyl group; C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

The term “heteroatom” as used herein refers to any atom other than a carbon atom. Non-limiting examples of the heteroatom may include O, S, N, P, Si, B, Ge, Se, or any combination thereof.

The term “transition metal” as used herein may include hafnium (Hf), tantalum (Ta), tungsten (W), rhenium (Re), osmium (Os), iridium (Ir), platinum (Pt), gold (Au), and/or the like.

As used herein, “D” refers to deuterium, “Ph” refers to a phenyl group, “Me” refers to a methyl group, “Et” refers to an ethyl group, “tert-Bu,” “^{t}Bu,” or “Bu^{t}” refers to a tert-butyl group, and “OMe” refers to a methoxy group.

The term “biphenyl group” as used herein refers to “a phenyl group substituted with a phenyl group.” For example, the “biphenyl group” may be a substituted phenyl group having a C₆-C₆₀ aryl group as a substituent.

The term “terphenyl group” as used herein refers to “a phenyl group substituted with a biphenyl group.” The “terphenyl group” may belong to i) “a substituent phenyl group” in which a substituent is “a C₆-C₆₀ aryl group substituted with a C₆-C₆₀ aryl group”, or ii) “a substituted phenyl group” having two substituents, each of which is “a C₆-C₆₀ aryl group.”

* and *′ as used herein, unless defined otherwise, each refer to a binding site to a neighboring atom in a corresponding formula or moiety.

The terms “x-axis,” “y-axis,” and “z-axis” as used herein are not limited to three axes in an orthogonal coordinate system, and may be interpreted in a broader sense than the aforementioned three axes in an orthogonal coordinate system. For example, the x-axis, y-axis, and z-axis may refer to those orthogonal to each other, or may refer to those in different directions that are not orthogonal to each other.

Hereinafter, organic compounds according to one or more embodiments and light-emitting devices including the organic compounds will be described in more detail with reference to Synthesis Examples and Examples.

### Synthesis Example 1 (Synthesis of Compound 1)

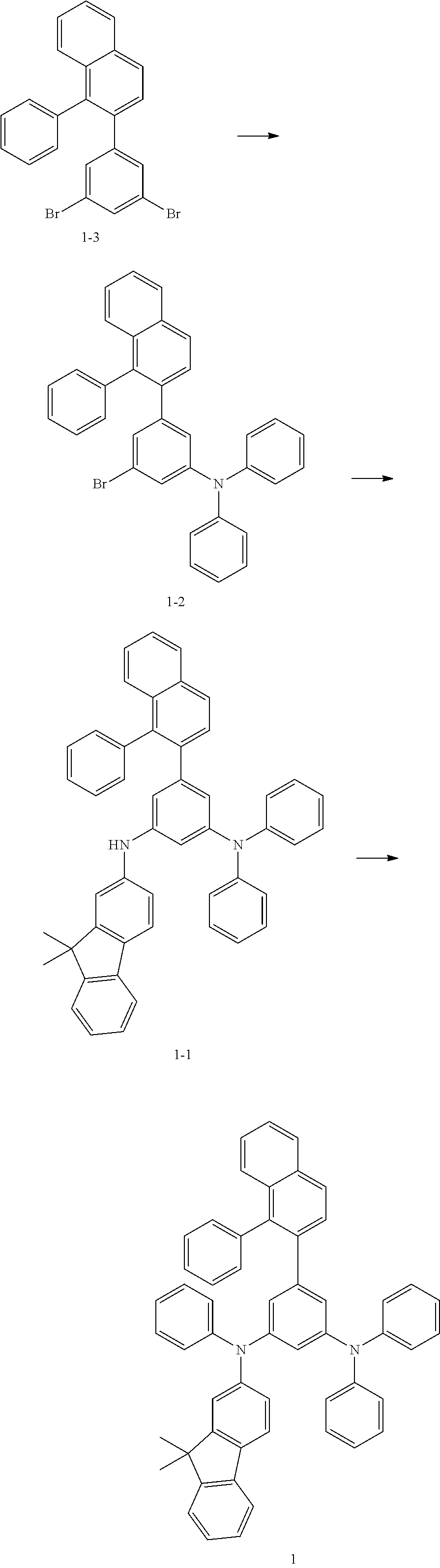 [SMILES: Brc1cc(Br)cc(-c2ccc3ccccc3c2-c2ccccc2)c1 ; Brc1cc(-c2ccc3ccccc3c2-c2ccccc2)cc(N(c2ccccc2)c2ccccc2)c1 ; CC1(C)c2ccccc2-c2ccc(Nc3cc(-c4ccc5ccccc5c4-c4ccccc4)cc(N(c4ccccc4)c4ccccc4)c3)cc21 ; CC1(C)c2ccccc2-c2ccc(N(c3ccccc3)c3cc(-c4ccc5ccccc5c4-c4ccccc4)cc(N(c4ccccc4)c4ccccc4)c3)cc21]

### Synthesis of Compound 1-2

2-(3,5-dibromophenyl)-1-phenylnaphthalene (Compound 1-3, 46 mmol, 2.3 equiv.), diphenylamine (20 mmol, 1 equiv.), tris(dibenzylideneacetone)dipalladium(0) (Pd₂(dba)₃) (1 mmol, 0.05 equiv.), t-BuONa (40 mmol, 2 equiv.), 2,2′-bis(diphenylphosphino)-1,1′-binaphthyl (BINAP) (2 mmol, 0.1 equiv.), and 300 mL of toluene were added to a 1-neck-round flask, and the mixture was stirred at 110° C. for 1 hour. After completion of the reaction, the product was worked up with ether/water and then purified by column chromatography using dichloromethane/hexane (volume ratio=1/5) as an eluent to obtain 16 mmol of Compound 1-2 (yield: 80%).

### Synthesis of Compound 1-1

Compound 1-2 (16 mmol, 1 equiv.), 9,9-dimethyl-9H-fluoren-2-amine (24 mmol, 1.5 equiv.), Pd₂(dba)₃ (0.48 mmol, 0.03 equiv.), t-BuONa (32 mmol, 2 equiv.), t-Bu₃P (0.8 mmol, 0.05 equiv.), and 150 mL of toluene were added to a 1-neck-round flask, and the mixture was stirred at 110° C. for 1 hour. After completion of the reaction, the product was worked up with ether/water and then purified by column chromatography using dichloromethane/hexane (volume ratio=1/8) as an eluent to obtain 12 mmol of Compound 1-1 (yield: 75%).

### Synthesis of Compound 1

Compound 1-1 (12 mmol, 1 equiv.), iodobenzene (15.6 mmol, 1.3 equiv.), Pd₂(dba)₃ (0.36 mmol, 0.03 equiv.), t-BuONa (24 mmol, 2 equiv.), t-Bu₃P (0.6 mmol, 0.05 equiv.), and 150 mL of toluene were added to a 1-neck-round flask, and the mixture was stirred at 110° C. for 1 hour. After completion of the reaction, the product was worked up with ether/water and then purified by column chromatography using dichloromethane/hexane (volume ratio=1/5) as an eluent to obtain 10 mmol of Compound 1 (yield: 83%).

### Synthesis Example 2 (Synthesis of Compound 2)

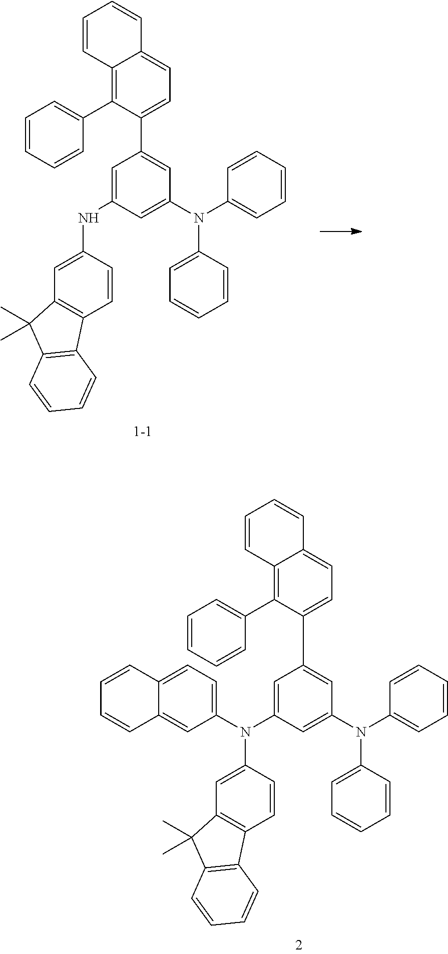 [SMILES: CC1(C)c2ccccc2-c2ccc(Nc3cc(-c4ccc5ccccc5c4-c4ccccc4)cc(N(c4ccccc4)c4ccccc4)c3)cc21 ; CC1(C)c2ccccc2-c2ccc(N(c3cc(-c4ccc5ccccc5c4-c4ccccc4)cc(N(c4ccccc4)c4ccccc4)c3)c3ccc4ccccc4c3)cc21]

Compound 1-1 (12 mmol, 1 equiv.), 2-bromonaphthalene (15.6 mmol, 1.3 equiv.), Pd₂(dba)₃ (0.36 mmol, 0.03 equiv.), t-BuONa (24 mmol, 2 equiv.), t-Bu₃P (0.6 mmol, 0.05 equiv.), and 150 mL of toluene were added to a 1-neck-round flask, and the mixture was stirred at 110° C. for 1 hour. After completion of the reaction, the product was worked up with ether/water and then purified by column chromatography using dichloromethane/hexane (volume ratio=1/5) as an eluent to obtain 8 mmol of Compound 2 (yield: 67%).

### Synthesis Example 3 (Synthesis of Compound 3)

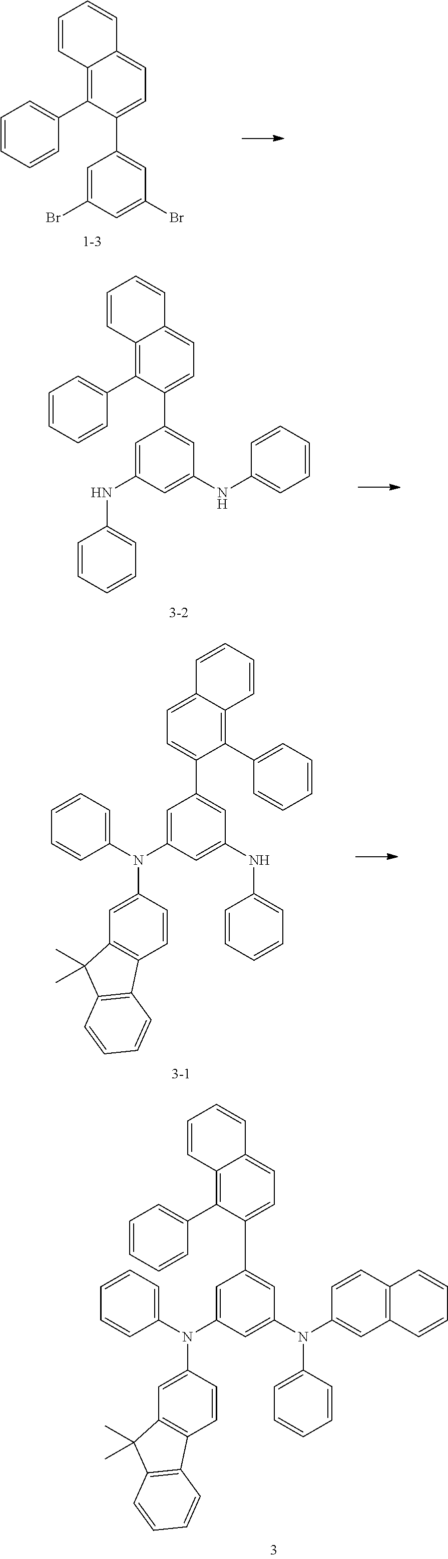 [SMILES: Brc1cc(Br)cc(-c2ccc3ccccc3c2-c2ccccc2)c1 ; c1ccc(Nc2cc(Nc3ccccc3)cc(-c3ccc4ccccc4c3-c3ccccc3)c2)cc1 ; CC1(C)c2ccccc2-c2ccc(N(c3ccccc3)c3cc(Nc4ccccc4)cc(-c4ccc5ccccc5c4-c4ccccc4)c3)cc21 ; CC1(C)c2ccccc2-c2ccc(N(c3ccccc3)c3cc(-c4ccc5ccccc5c4-c4ccccc4)cc(N(c4ccccc4)c4ccc5ccccc5c4)c3)cc21]

### Synthesis of Compound 3-2

Compound 1-3 (20 mmol, 1 equiv.), aniline (46 mmol, 2.3 equiv.), Pd₂(dba)₃ (1 mmol, 0.05 equiv.), t-BuONa (40 mmol, 2 equiv.), BINAP (2 mmol, 0.1 equiv.), and 300 mL of toluene were added to a 1-neck-round flask, and the mixture was stirred at 110° C. for 1 hour. After completion of the reaction, the product was worked up with ether/water and then purified by column chromatography using dichloromethane/hexane (volume ratio=1/5) as an eluent to obtain 14 mmol of Compound 3-2 (yield: 70%).

### Synthesis of Compound 3-1

Compound 3-2 (16.8 mmol, 1.4 equiv.), 2-bromo-9,9-dimethyl-9H-fluorene (12 mmol, 1 equiv.), Pd₂(dba)₃ (0.36 mmol, 0.03 equiv.), t-BuONa (24 mmol, 2 equiv.), t-Bu₀P (0.6 mmol, 0.05 equiv.), and 150 mL of toluene were added to a 1-neck-round flask, and the mixture was stirred at 110° C. for 1 hour. After completion of the reaction, the product was worked up with ether/water and then purified by column chromatography using dichloromethane/hexane (volume ratio=1/5) as an eluent to obtain 10 mmol of Compound 3-1 (yield: 83%).

### Synthesis of Compound 3

Compound 3-1 (10 mmol, 1 equiv.), 2-bromonaphthalene (13 mmol, 1.3 equiv.), Pd₂(dba)₃ (0.3 mmol, 0.03 equiv.), t-BuONa (20 mmol, 2 equiv.), t-Bu₃P (0.5 mmol, 0.05 equiv.), and 150 mL of toluene were added to a 1-neck-round flask, and the mixture was stirred at 110° C. for 1 hour. After completion of the reaction, the product was worked up with ether/water and then purified by column chromatography using dichloromethane/hexane (volume ratio=1/8) as an eluent to obtain 8 mmol of Compound 3 (yield: 80%).

### Synthesis Example 4 (Synthesis of Compound 4)

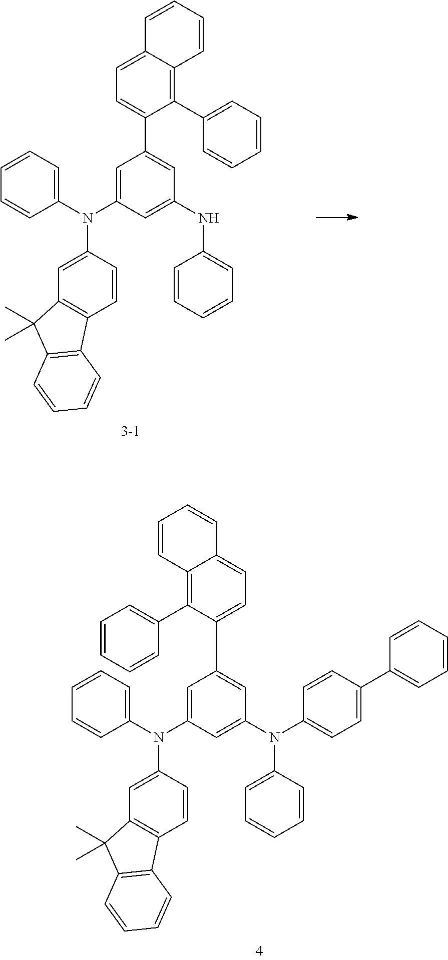 [SMILES: CC1(C)c2ccccc2-c2ccc(N(c3ccccc3)c3cc(Nc4ccccc4)cc(-c4ccc5ccccc5c4-c4ccccc4)c3)cc21 ; CC1(C)c2ccccc2-c2ccc(N(c3ccccc3)c3cc(-c4ccc5ccccc5c4-c4ccccc4)cc(N(c4ccccc4)c4ccc(-c5ccccc5)cc4)c3)cc21]

Compound 3-1 (10 mmol, 1 equiv.), 4-iodobiphenyl (13 mmol, 1.3 equiv.), Pd₂(dba)₃ (0.3 mmol, 0.03 equiv.), t-BuONa (20 mmol, 2 equiv.), t-Bu₃P (0.5 mmol, 0.05 equiv.), and 150 mL of toluene were added to a 1-neck-round flask, and the mixture was stirred at 110° C. for 1 hour. After completion of the reaction, the product was worked up with ether/water and then purified by column chromatography using dichloromethane/hexane (volume ratio=1/8) as an eluent to obtain 8.2 mmol of Compound 4 (yield: 82%).

### Synthesis Example 5 (Synthesis of Compound 5)

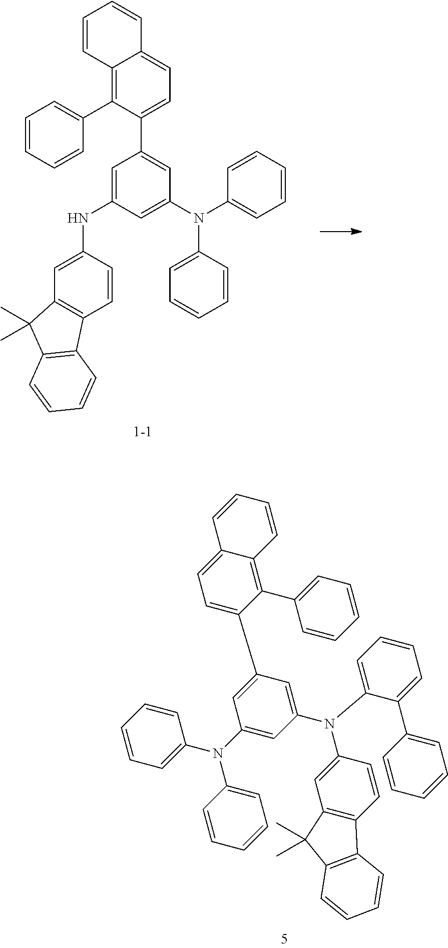 [SMILES: CC1(C)c2ccccc2-c2ccc(Nc3cc(-c4ccc5ccccc5c4-c4ccccc4)cc(N(c4ccccc4)c4ccccc4)c3)cc21 ; CC1(C)c2ccccc2-c2ccc(N(c3cc(-c4ccc5ccccc5c4-c4ccccc4)cc(N(c4ccccc4)c4ccccc4)c3)c3ccccc3-c3ccccc3)cc21]

Compound 1-1 (12 mmol, 1 equiv.), 2-bromobiphenyl (15.6 mmol, 1.3 equiv.), Pd₂(dba)₃ (0.36 mmol, 0.03 equiv.), t-BuONa (24 mmol, 2 equiv.), t-Bu₃P (0.6 mmol, 0.05 equiv.), and 150 mL of toluene were added to a 1-neck-round flask, and the mixture was stirred at 110° C. for 1 hour. After completion of the reaction, the product was worked up with ether/water and then purified by column chromatography using dichloromethane/hexane (volume ratio=1/5) as an eluent to obtain 6 mmol of Compound 5 (yield: 50%).

### Synthesis Example 6 (Synthesis of Compound 6)

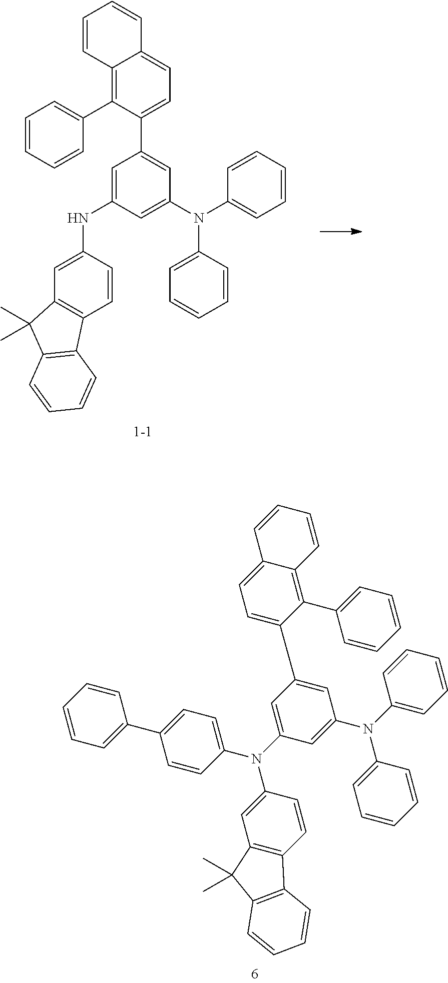 [SMILES: CC1(C)c2ccccc2-c2ccc(Nc3cc(-c4ccc5ccccc5c4-c4ccccc4)cc(N(c4ccccc4)c4ccccc4)c3)cc21 ; CC1(C)c2ccccc2-c2ccc(N(c3ccc(-c4ccccc4)cc3)c3cc(-c4ccc5ccccc5c4-c4ccccc4)cc(N(c4ccccc4)c4ccccc4)c3)cc21]

Compound 1-1 (12 mmol, 1 equiv.), 4-iodobiphenyl (15.6 mmol, 1.3 equiv.), Pd₂(dba)₃ (0.36 mmol, 0.03 equiv.), t-BuONa (24 mmol, 2 equiv.), t-Bu₃P (0.6 mmol, 0.05 equiv.), and 150 mL of toluene were added to a 1-neck-round flask, and the mixture was stirred at 110° C. for 1 hour. After completion of the reaction, the product was worked up with ether/water and then purified by column chromatography using dichloromethane/hexane (volume ratio=1/5) as an eluent to obtain 8 mmol of Compound 6 (yield: 67%).

### Synthesis Example 7 (Synthesis of Compound 9)

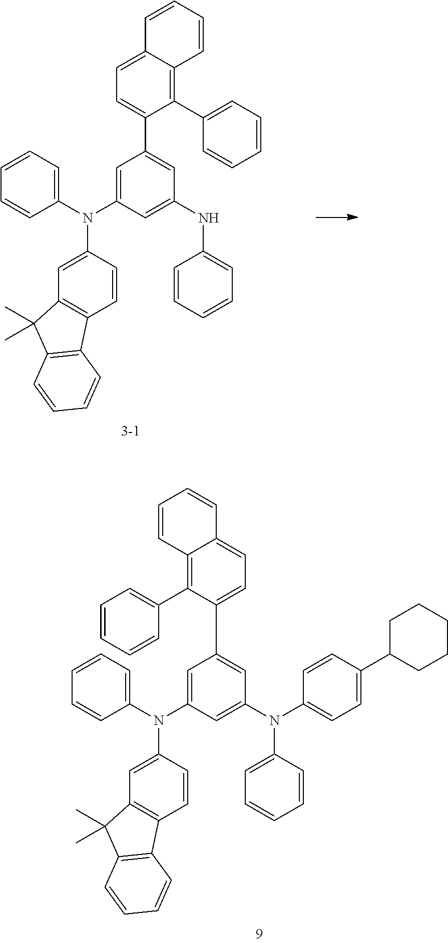 [SMILES: CC1(C)c2ccccc2-c2ccc(N(c3ccccc3)c3cc(Nc4ccccc4)cc(-c4ccc5ccccc5c4-c4ccccc4)c3)cc21 ; CC1(C)c2ccccc2-c2ccc(N(c3ccccc3)c3cc(-c4ccc5ccccc5c4-c4ccccc4)cc(N(c4ccccc4)c4ccc(C5CCCCC5)cc4)c3)cc21]

Compound 3-1 (10 mmol, 1 equiv.), 4-bromo-cyclohexylbenzene (13 mmol, 1.3 equiv.), Pd₂(dba)₃ (0.3 mmol, 0.03 equiv.), t-BuONa (20 mmol, 2 equiv.), t-Bu₃P (0.5 mmol, 0.05 equiv.), and 150 mL of toluene were added to a 1-neck-round flask, and the mixture was stirred at 110° C. for 1 hour. After completion of the reaction, the product was worked up with ether/water and then purified by column chromatography using dichloromethane/hexane (volume ratio=1/8) as an eluent to obtain 7.6 mmol of Compound 9 (yield: 76%).

### Synthesis Example 8 (Synthesis of Compound 10)

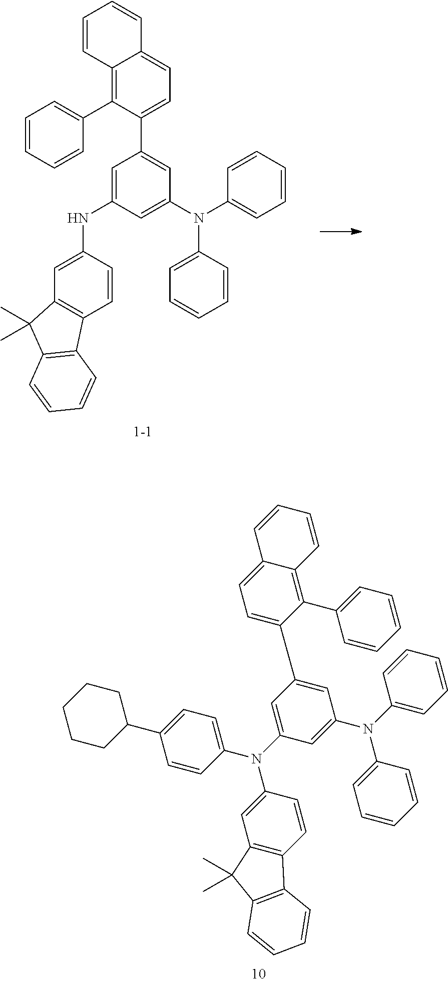 [SMILES: CC1(C)c2ccccc2-c2ccc(Nc3cc(-c4ccc5ccccc5c4-c4ccccc4)cc(N(c4ccccc4)c4ccccc4)c3)cc21 ; CC1(C)c2ccccc2-c2ccc(N(c3ccc(C4CCCCC4)cc3)c3cc(-c4ccc5ccccc5c4-c4ccccc4)cc(N(c4ccccc4)c4ccccc4)c3)cc21]

Compound 1-1 (12 mmol, 1 equiv.), 4-bromo-cyclohexylbenzene (15.6 mmol, 1.3 equiv.), Pd₂(dba)₃ (0.36 mmol, 0.03 equiv.), t-BuONa (24 mmol, 2 equiv.), t-Bu₃P (0.6 mmol, 0.05 equiv.), and 150 mL of toluene were added to a 1-neck-round flask, and the mixture was stirred at 110° C. for 1 hour. After completion of the reaction, the product was worked up with ether/water and then purified by column chromatography using dichloromethane/hexane (volume ratio=1/5) as an eluent to obtain 7 mmol of Compound 10 (yield: 58%).

### Synthesis Example 9 (Synthesis of Compound 65)

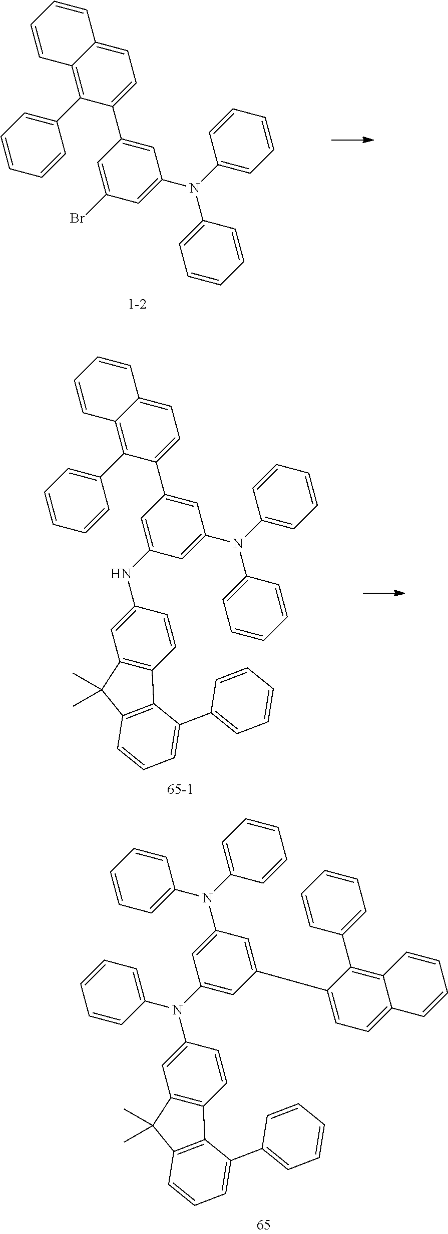 [SMILES: Brc1cc(-c2ccc3ccccc3c2-c2ccccc2)cc(N(c2ccccc2)c2ccccc2)c1 ; CC1(C)c2cc(Nc3cc(-c4ccc5ccccc5c4-c4ccccc4)cc(N(c4ccccc4)c4ccccc4)c3)ccc2-c2c(-c3ccccc3)cccc21 ; CC1(C)c2cc(N(c3ccccc3)c3cc(-c4ccc5ccccc5c4-c4ccccc4)cc(N(c4ccccc4)c4ccccc4)c3)ccc2-c2c(-c3ccccc3)cccc21]

### Synthesis of Compound 65-1

Compound 1-2 (16 mmol, 1 equiv.), 9,9-dimethyl-5-phenyl-9H-fluoren-2-amine (24 mmol, 1.5 equiv.), Pd₂(dba)₃ (0.48 mmol, 0.03 equiv.), t-BuONa (32 mmol, 2 equiv.), t-Bu₃P (0.8 mmol, 0.05 equiv.), and 150 mL of toluene were added to a 1-neck-round flask, and the mixture was stirred at 110° C. for 1 hour. After completion of the reaction, the product was worked up with ether/water and then purified by column chromatography using dichloromethane/hexane (volume ratio=1/5) as an eluent to obtain 12 mmol of Compound 65-1 (yield: 75%).

### Synthesis of Compound 65

Compound 65-1 (12 mmol, 1 equiv.), iodobenzene (15.6 mmol, 1.3 equiv.), Pd₂(dba)₃ (0.36 mmol, 0.03 equiv.), t-BuONa (24 mmol, 2 equiv.), t-Bu₃P (0.6 mmol, 0.05 equiv.), and 150 mL of toluene were added to a 1-neck-round flask, and the mixture was stirred at 110° C. for 1 hour. After completion of the reaction, the product was worked up with ether/water and then purified by column chromatography using dichloromethane/hexane (volume ratio=1/5) as an eluent to obtain 10 mmol of Compound 65 (yield: 83%).

### Synthesis Example 10 (Synthesis of Compound 81)

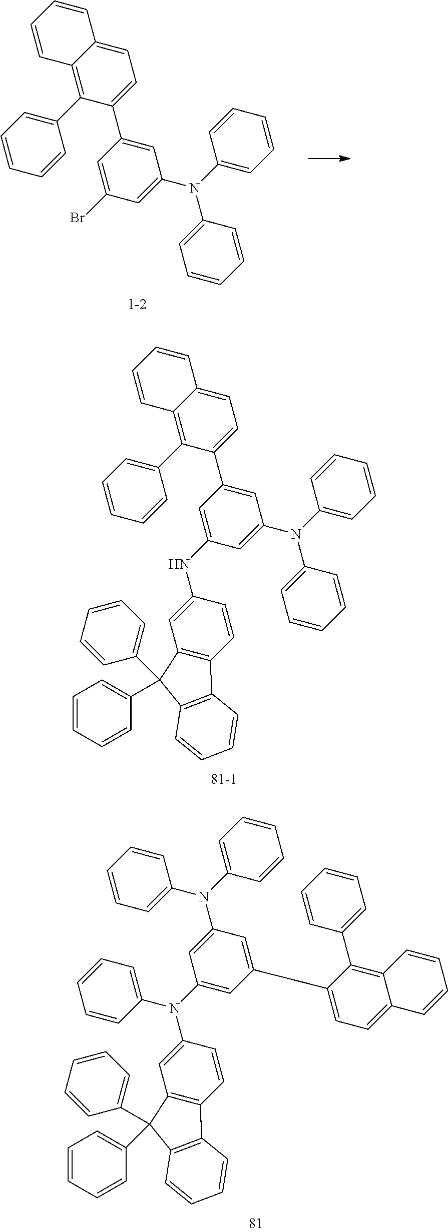 [SMILES: Brc1cc(-c2ccc3ccccc3c2-c2ccccc2)cc(N(c2ccccc2)c2ccccc2)c1 ; c1ccc(-c2c(-c3cc(Nc4ccc5c(c4)C(c4ccccc4)(c4ccccc4)c4ccccc4-5)cc(N(c4ccccc4)c4ccccc4)c3)ccc3ccccc23)cc1 ; c1ccc(-c2c(-c3cc(N(c4ccccc4)c4ccccc4)cc(N(c4ccccc4)c4ccc5c(c4)C(c4ccccc4)(c4ccccc4)c4ccccc4-5)c3)ccc3ccccc23)cc1]

### Synthesis of Compound 81-1

Compound 1-2 (16 mmol, 1 equiv.), 9,9-diphenyl-9H-fluoren-2-amine (24 mmol, 1.5 equiv.), Pd₂(dba)₃ (0.48 mmol, 0.03 equiv.), t-BuONa (32 mmol, 2 equiv.), t-Bu₃P (0.8 mmol, 0.05 equiv.), and 150 mL of toluene were added to a 1-neck-round flask, and the mixture was stirred at 110° C. for 1 hour. After completion of the reaction, the product was worked up with ether/water and then purified by column chromatography using dichloromethane/hexane (volume ratio=1/5) as an eluent to obtain 13 mmol of Compound 81-1 (yield: 81%).

### Synthesis of Compound 81

Compound 81-1 (10 mmol, 1 equiv.), iodobenzene (13 mmol, 1.3 equiv.), Pd₂(dba)₃ (0.3 mmol, 0.03 equiv.), t-BuONa (20 mmol, 2 equiv.), t-Bu₃P (0.5 mmol, 0.05 equiv.), and 150 mL of toluene were added to a 1-neck-round flask, and the mixture was stirred at 110° C. for 1 hour. After completion of the reaction, the product was worked up with ether/water and then purified by column chromatography using dichloromethane/hexane (volume ratio=1/6) as an eluent to obtain 8 mmol of Compound 81 (yield: 80%).

### Synthesis Example 11 (Synthesis of Compound 129)

Compound 129 was obtained in substantially the same manner as in the synthesis of Compound 1, except that 3,5-dibromo-6′-phenyl-1,1′:2′,1″-terphenyl was used instead of 2-(3,5-dibromophenyl)-1-phenylnaphthalene (Compound 1-3) in the synthesis of Compound 1-2.

### Synthesis Example 12 (Synthesis of Compound 145)

Compound 145 was obtained in substantially the same manner as in the synthesis of Compound 1, except that 3′″,5′″-dibromo-3′-phenyl-1,1′:2′,1″: 4″,1′″-quarterphenyl was used instead of 2-(3,5-dibromophenyl)-1-phenylnaphthalene (Compound 1-3) in the synthesis of Compound 1-2.

### Comparative Example 1

As an anode, a glass substrate (product of Corning Inc.) with a 15 Ω/cm² (1,200 Å) ITO electrode formed thereon was cut to a size of 50 mm×50 mm×0.7 mm, sonicated with isopropyl alcohol and then pure water each for 5 minutes, cleaned by irradiation of ultraviolet rays and exposure of ozone thereto for 30 minutes, and then mounted on a vacuum deposition apparatus.

NPD was deposited on the anode to form a hole injection layer having a thickness of 300 Å. CE1 was deposited on the hole injection layer to form a hole transport layer having a thickness of 200 Å. CzSi was deposited on the hole transport layer to form an emission auxiliary layer having a thickness of 100 Å.

HTH49 as a hole-transporting host, ETH102 as an electron-transporting host, PS-3 as a phosphorescent sensitizer, and DFD31 as a dopant were co-deposited on the emission auxiliary layer at a weight ratio of 42:42:15:1 to form an emission layer having a thickness of 200 Å.

TSPO1 was deposited on the emission layer to form a hole-blocking layer having a thickness of 200 Å. TPBI was deposited on the hole-blocking layer to form an electron transport layer having a thickness of 300 Å. LiF was deposited on the electron transport layer to form an electron injection layer having a thickness of 10 Å, and Al was deposited on the electron injection layer to form a cathode having a thickness of 3,000 Å, thereby completing the manufacture of a light-emitting device.

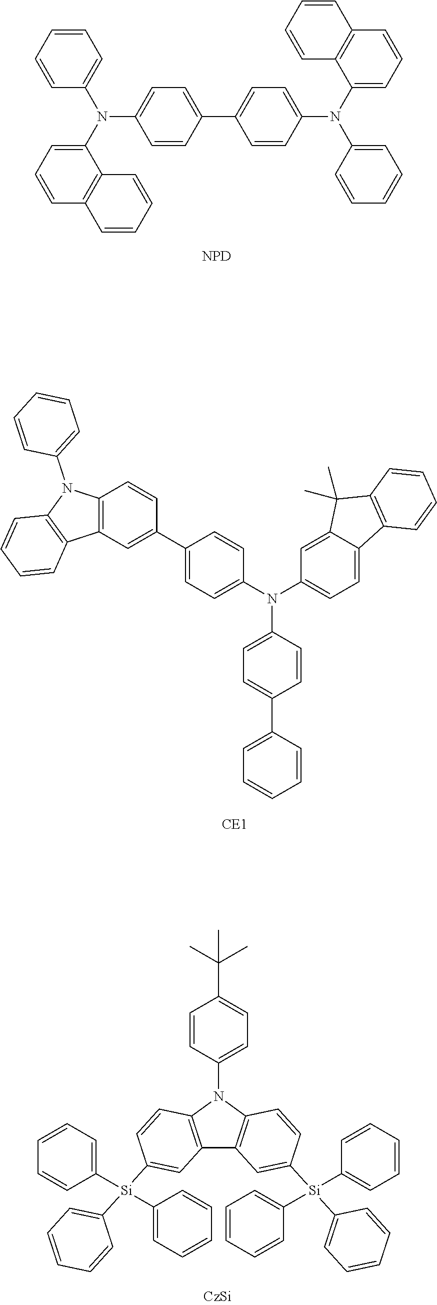

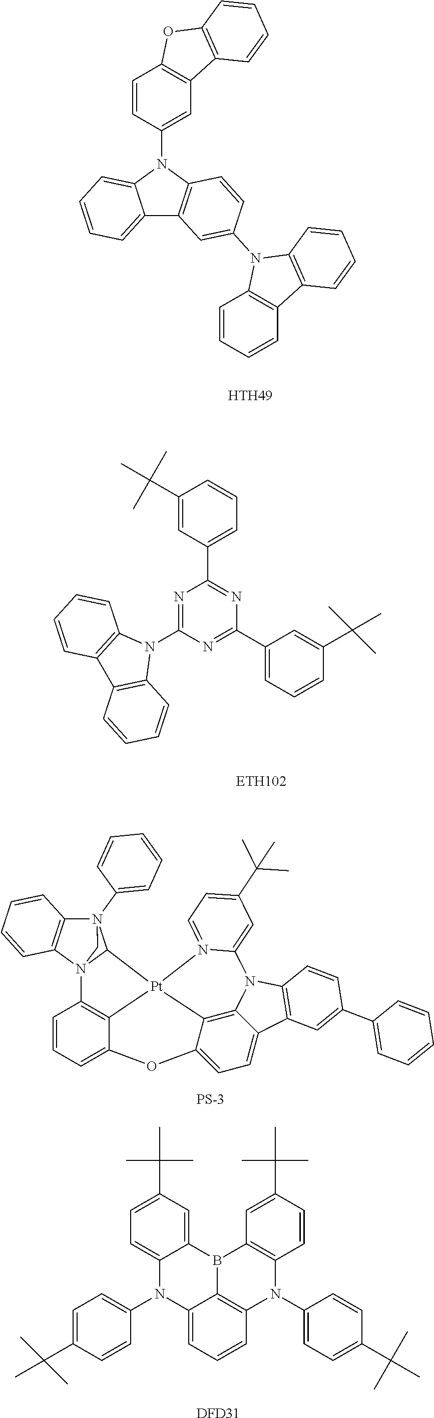

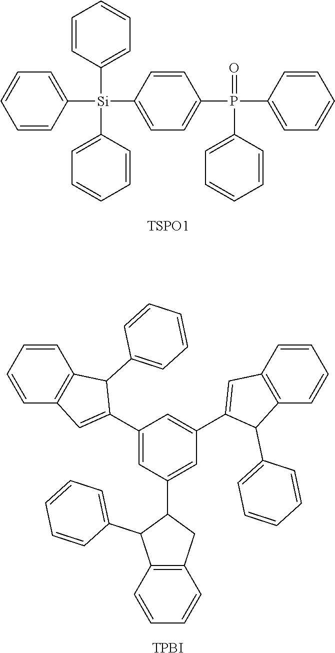

### Comparative Examples 2 to 14

Light-emitting devices were each manufactured in substantially the same manner as in Comparative Example 1, except that, in forming the hole transport layer, compounds shown in Table 1 were each respectively used instead of Compound CE1.

### Examples 1 to 32

Light-emitting devices were each manufactured in substantially the same manner as in Comparative Example 1, except that, in forming the hole transport layer, compounds shown in Table 2 were each respectively used instead of Compound CE.

### Evaluation Example

The driving voltage at 1,000 cd/m², luminescence efficiency, maximum emission wavelength (wavelength of maximum emission peak), and lifespan (T₉₅) of each of the light-emitting devices of Comparative Examples 1 to 14 and Examples to 32 were measured by using Keithley MU 236 and luminance meter PR650, and results thereof are shown in Tables 1 and 2. The lifespan (T₉₅) represents the time (hr) taken until the luminance declines to 95% of the initial luminance.

TABLE 1

| | Hole | | | Maximum | Life- |
|---|---|---|---|---|---|
| | transport | Driving | Luminescence | emission | span |
| | layer | voltage | efficiency | wavelength | (T₉₅, |
| No. | material | (V) | (cd/A) | (nm) | hr) |
| Comparative | CE1 | 4.4 | 26.3 | 462 | 220 |
| Example 1 | | | | | |
| Comparative | CE2 | 4.18 | 27.5 | 461 | 240 |
| Example 2 | | | | | |
| Comparative | CE3 | 4.16 | 25.0 | 460 | 230 |
| Example 3 | | | | | |
| Comparative | CE4 | 4.20 | 25.0 | 460 | 200 |
| Example 4 | | | | | |
| Comparative | CE5 | 4.19 | 26.0 | 461 | 250 |
| Example 5 | | | | | |
| Comparative | CE6 | 4.22 | 25.0 | 461 | 230 |
| Example 6 | | | | | |
| Comparative | CE7 | 4.23 | 24.0 | 460 | 230 |
| Example 7 | | | | | |
| Comparative | CE8 | 4.03 | 27.0 | 460 | 285 |
| Example 8 | | | | | |
| Comparative | CE9 | 4.02 | 27.5 | 460 | 280 |
| Example 9 | | | | | |
| Comparative | CE10 | 4.02 | 27.0 | 462 | 270 |
| Example 10 | | | | | |
| Comparative | CE11 | 4.04 | 26.0 | 461 | 250 |
| Example 11 | | | | | |
| Comparative | CE12 | 4.03 | 27.0 | 460 | 265 |
| Example 12 | | | | | |
| Comparative | CE13 | 4.03 | 27.0 | 460 | 250 |
| Example 13 | | | | | |
| Comparative | CE14 | 4.42 | 20.0 | 460 | 215 |
| Example 14 | | | | | |

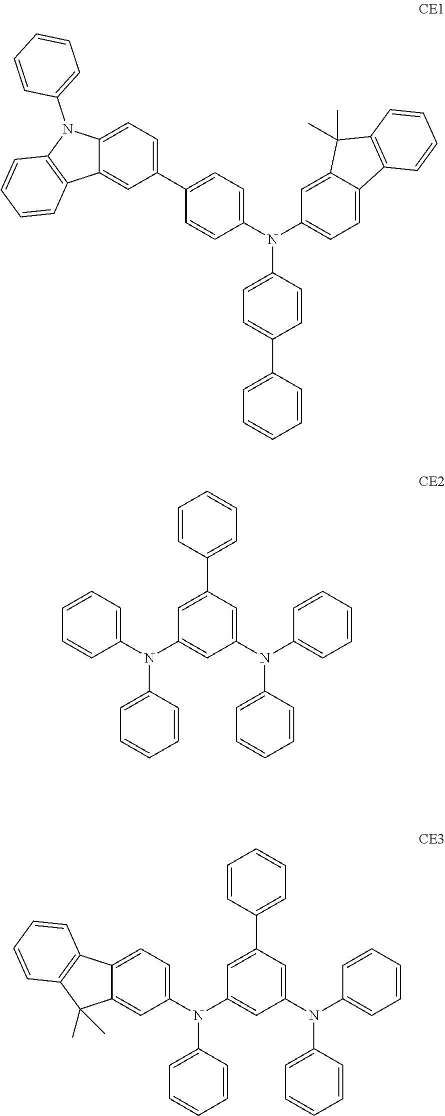 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2cc(N(c3ccccc3)c3ccccc3)cc(N(c3ccccc3)c3ccccc3)c2)cc1 ; CC1(C)c2ccccc2-c2ccc(N(c3ccccc3)c3cc(-c4ccccc4)cc(N(c4ccccc4)c4ccccc4)c3)cc21]

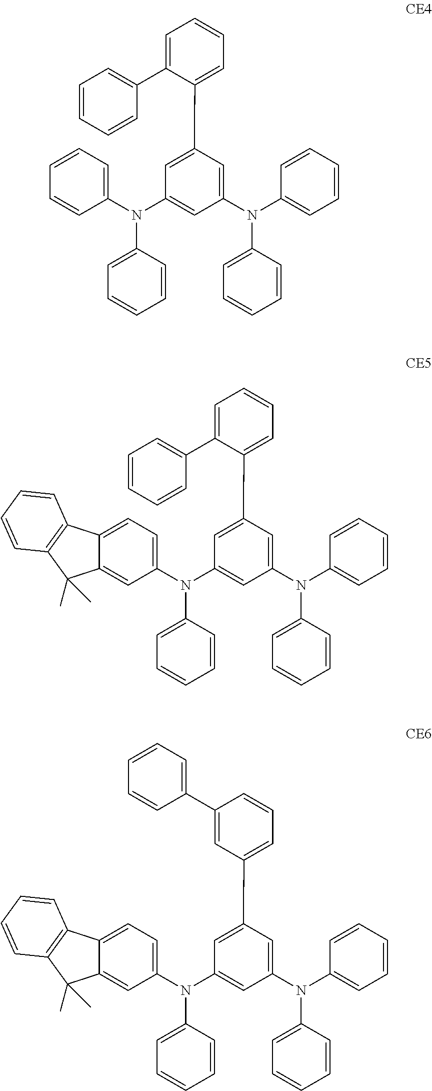 [SMILES: c1ccc(-c2ccccc2-c2cc(N(c3ccccc3)c3ccccc3)cc(N(c3ccccc3)c3ccccc3)c2)cc1 ; CC1(C)c2ccccc2-c2ccc(N(c3ccccc3)c3cc(-c4ccccc4-c4ccccc4)cc(N(c4ccccc4)c4ccccc4)c3)cc21 ; CC1(C)c2ccccc2-c2ccc(N(c3ccccc3)c3cc(-c4cccc(-c5ccccc5)c4)cc(N(c4ccccc4)c4ccccc4)c3)cc21]

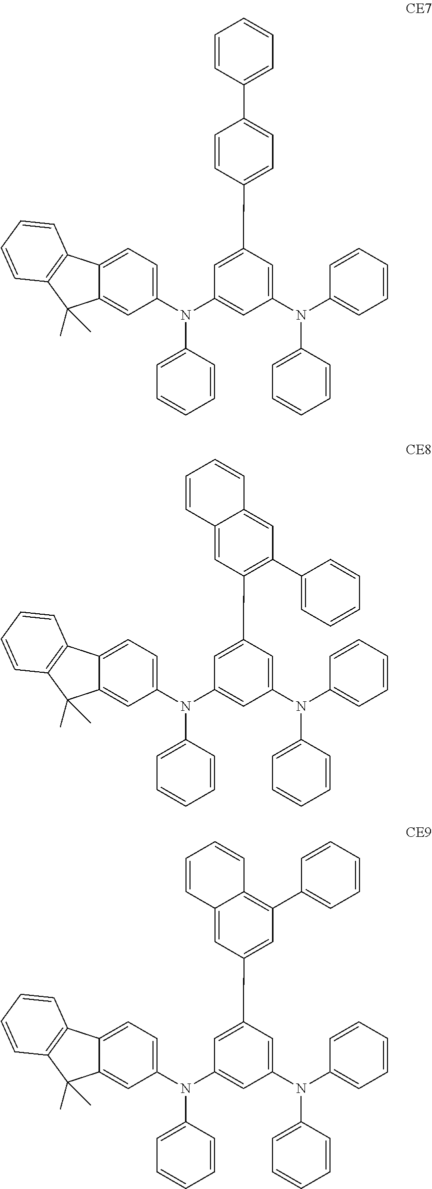

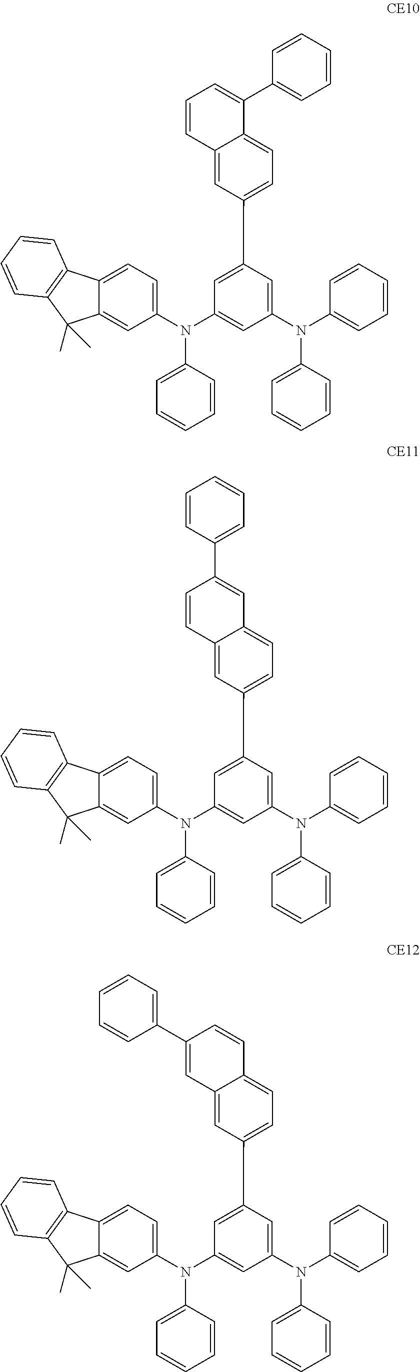 [SMILES: CC1(C)c2ccccc2-c2ccc(N(c3ccccc3)c3cc(-c4ccc5c(-c6ccccc6)cccc5c4)cc(N(c4ccccc4)c4ccccc4)c3)cc21 ; CC1(C)c2ccccc2-c2ccc(N(c3ccccc3)c3cc(-c4ccc5cc(-c6ccccc6)ccc5c4)cc(N(c4ccccc4)c4ccccc4)c3)cc21 ; CC1(C)c2ccccc2-c2ccc(N(c3ccccc3)c3cc(-c4ccc5ccc(-c6ccccc6)cc5c4)cc(N(c4ccccc4)c4ccccc4)c3)cc21]

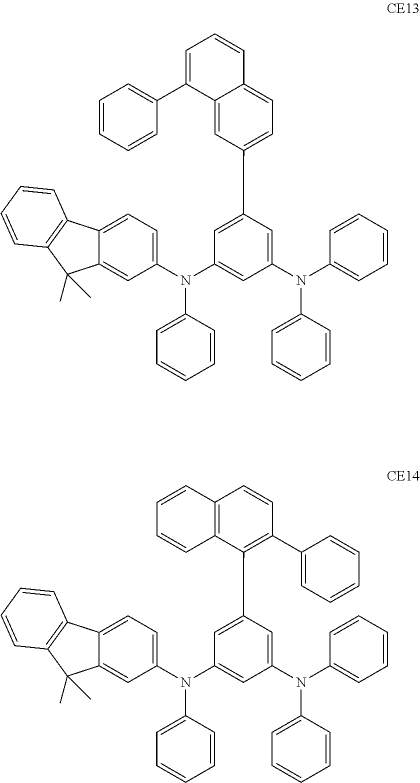 [SMILES: CC1(C)c2ccccc2-c2ccc(N(c3ccccc3)c3cc(-c4ccc5cccc(-c6ccccc6)c5c4)cc(N(c4ccccc4)c4ccccc4)c3)cc21 ; CC1(C)c2ccccc2-c2ccc(N(c3ccccc3)c3cc(-c4c(-c5ccccc5)ccc5ccccc45)cc(N(c4ccccc4)c4ccccc4)c3)cc21]

TABLE 2

| | Hole | | | Maximum | Life- |
|---|---|---|---|---|---|
| | transport | Driving | Luminescence | emission | span |
| | layer | voltage | efficiency | wavelength | (T₉₅, |
| No. | material | (V) | (cd/A) | (nm) | hr) |
| Example 1 | Compound | 3.55 | 30.5 | 460 | 360 |
| | 1 | | | | |
| Example 2 | Compound | 3.61 | 30.2 | 460 | 350 |
| | 2 | | | | |
| Example 3 | Compound | 3.65 | 32.2 | 460 | 365 |
| | 3 | | | | |
| Example 4 | Compound | 3.64 | 31.4 | 462 | 345 |
| | 4 | | | | |
| Example 5 | Compound | 3.57 | 30.6 | 459 | 340 |
| | 5 | | | | |
| Example 6 | Compound | 3.54 | 30.4 | 459 | 380 |
| | 6 | | | | |
| Example 7 | Compound | 3.54 | 30.5 | 459 | 360 |
| | 9 | | | | |
| Example 8 | Compound | 3.61 | 31.5 | 460 | 365 |
| | 10 | | | | |
| Example 9 | Compound | 3.64 | 31.6 | 460 | 370 |
| | 65 | | | | |
| Example | Compound | 3.59 | 33.0 | 461 | 350 |
| 10 | 66 | | | | |
| Example | Compound | 3.48 | 32.8 | 460 | 350 |
| 11 | 67 | | | | |
| Example | Compound | 3.52 | 32.7 | 460 | 345 |
| 12 | 68 | | | | |
| Example | Compound | 3.52 | 30.6 | 461 | 330 |
| 13 | 73 | | | | |
| Example | Compound | 3.55 | 31.7 | 462 | 360 |
| 14 | 74 | | | | |
| Example | Compound | 3.65 | 30.6 | 462 | 360 |
| 15 | 81 | | | | |
| Example | Compound | 3.57 | 31.3 | 460 | 360 |
| 16 | 82 | | | | |
| Example | Compound | 3.53 | 31.4 | 460 | 370 |
| 17 | 83 | | | | |
| Example | Compound | 3.54 | 31.5 | 460 | 360 |
| 18 | 84 | | | | |
| Example | Compound | 3.61 | 32.0 | 459 | 360 |
| 19 | 89 | | | | |
| Example | Compound | 3.60 | 30.9 | 460 | 355 |
| 20 | 90 | | | | |
| Example | Compound | 3.59 | 31.2 | 460 | 350 |
| 21 | 129 | | | | |
| Example | Compound | 3.48 | 30.8 | 462 | 380 |
| 22 | 130 | | | | |
| Example | Compound | 3.52 | 30.9 | 462 | 355 |
| 23 | 131 | | | | |
| Example | Compound | 3.52 | 33.0 | 461 | 360 |
| 24 | 132 | | | | |
| Example | Compound | 3.55 | 33.0 | 461 | 370 |
| 25 | 137 | | | | |
| Example | Compound | 3.57 | 31.3 | 460 | 360 |
| 26 | 138 | | | | |
| Example | Compound | 3.53 | 31.4 | 460 | 370 |
| 27 | 145 | | | | |
| Example | Compound | 3.61 | 31.5 | 460 | 365 |
| 28 | 146 | | | | |
| Example | Compound | 3.64 | 31.6 | 460 | 370 |
| 29 | 177 | | | | |
| Example | Compound | 3.59 | 33.0 | 461 | 355 |
| 30 | 178 | | | | |
| Example | Compound | 3.48 | 32.8 | 460 | 370 |
| 31 | 193 | | | | |
| Example | Compound | 3.52 | 32.7 | 460 | 375 |
| 32 | 194 | | | | |

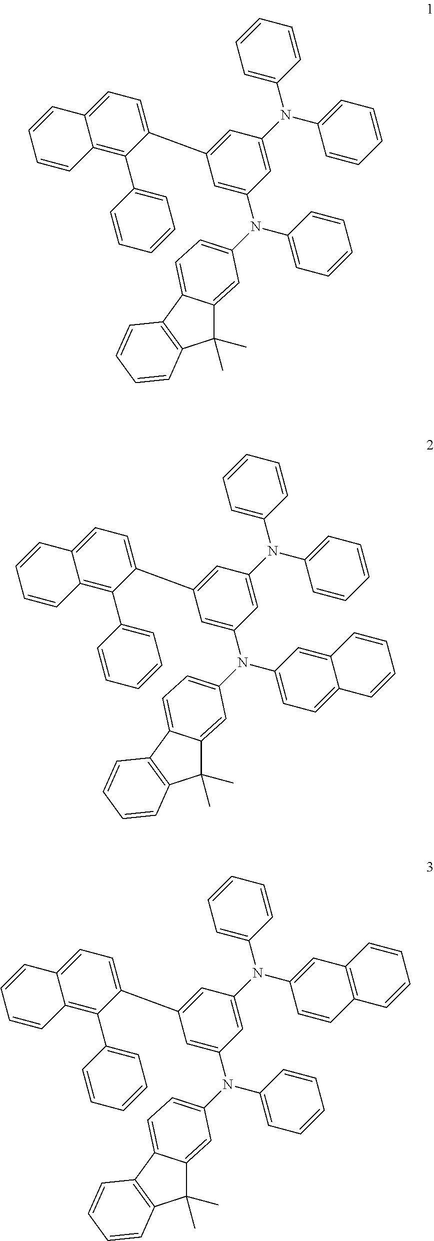 [SMILES: CC1(C)c2ccccc2-c2ccc(N(c3ccccc3)c3cc(-c4ccc5ccccc5c4-c4ccccc4)cc(N(c4ccccc4)c4ccccc4)c3)cc21 ; CC1(C)c2ccccc2-c2ccc(N(c3cc(-c4ccc5ccccc5c4-c4ccccc4)cc(N(c4ccccc4)c4ccccc4)c3)c3ccc4ccccc4c3)cc21 ; CC1(C)c2ccccc2-c2ccc(N(c3ccccc3)c3cc(-c4ccc5ccccc5c4-c4ccccc4)cc(N(c4ccccc4)c4ccc5ccccc5c4)c3)cc21]

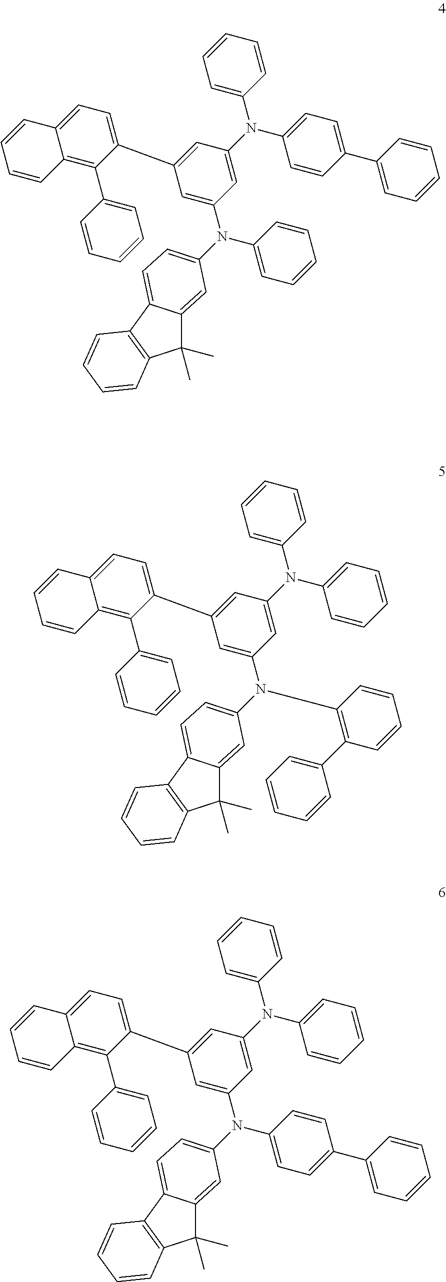 [SMILES: CC1(C)c2ccccc2-c2ccc(N(c3ccccc3)c3cc(-c4ccc5ccccc5c4-c4ccccc4)cc(N(c4ccccc4)c4ccc(-c5ccccc5)cc4)c3)cc21 ; CC1(C)c2ccccc2-c2ccc(N(c3cc(-c4ccc5ccccc5c4-c4ccccc4)cc(N(c4ccccc4)c4ccccc4)c3)c3ccccc3-c3ccccc3)cc21 ; CC1(C)c2ccccc2-c2ccc(N(c3ccc(-c4ccccc4)cc3)c3cc(-c4ccc5ccccc5c4-c4ccccc4)cc(N(c4ccccc4)c4ccccc4)c3)cc21]

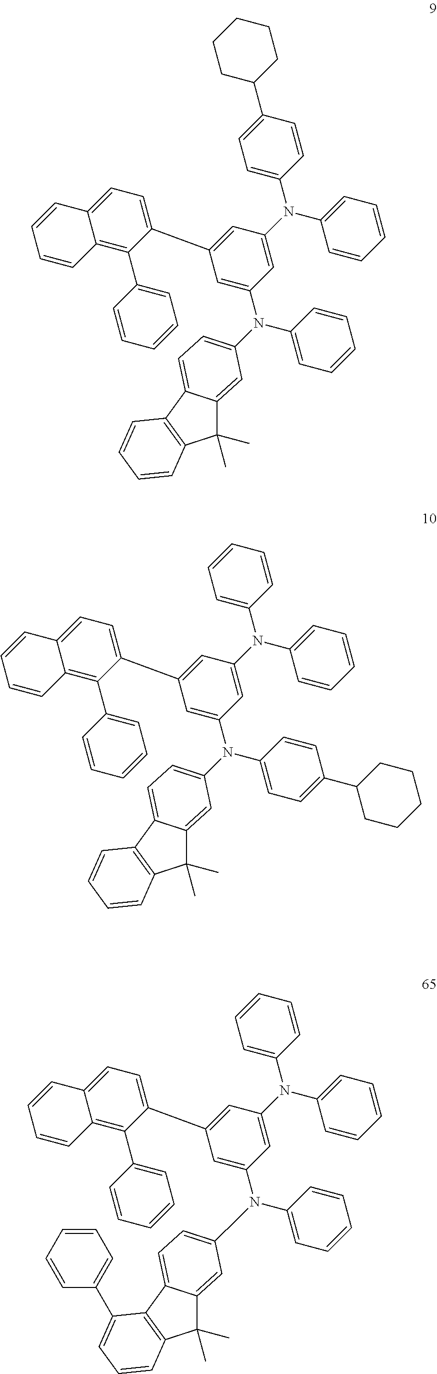 [SMILES: CC1(C)c2ccccc2-c2ccc(N(c3ccccc3)c3cc(-c4ccc5ccccc5c4-c4ccccc4)cc(N(c4ccccc4)c4ccc(C5CCCCC5)cc4)c3)cc21 ; CC1(C)c2ccccc2-c2ccc(N(c3ccc(C4CCCCC4)cc3)c3cc(-c4ccc5ccccc5c4-c4ccccc4)cc(N(c4ccccc4)c4ccccc4)c3)cc21 ; CC1(C)c2cc(N(c3ccccc3)c3cc(-c4ccc5ccccc5c4-c4ccccc4)cc(N(c4ccccc4)c4ccccc4)c3)ccc2-c2c(-c3ccccc3)cccc21]

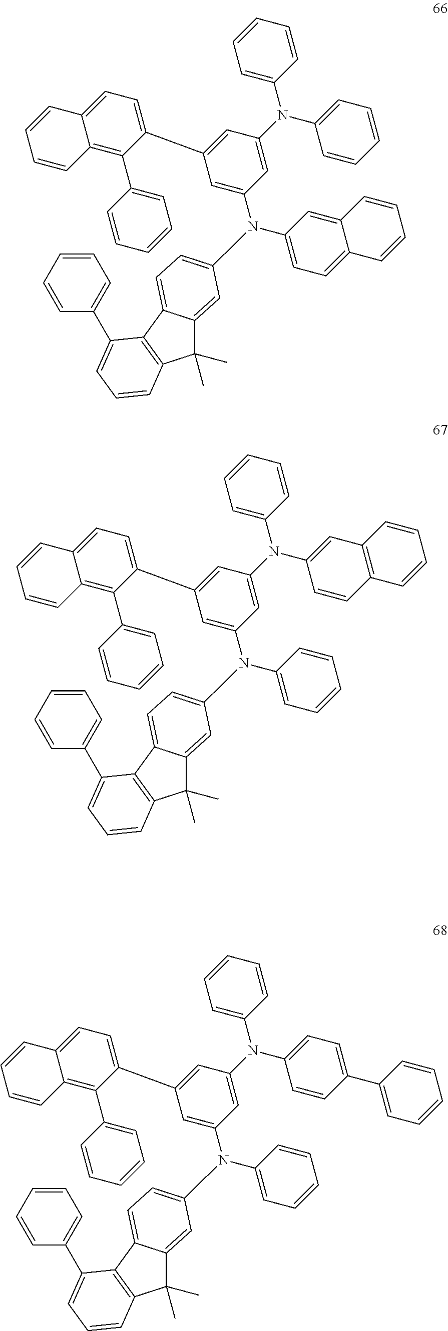 [SMILES: CC1(C)c2cc(N(c3cc(-c4ccc5ccccc5c4-c4ccccc4)cc(N(c4ccccc4)c4ccccc4)c3)c3ccc4ccccc4c3)ccc2-c2c(-c3ccccc3)cccc21 ; CC1(C)c2cc(N(c3ccccc3)c3cc(-c4ccc5ccccc5c4-c4ccccc4)cc(N(c4ccccc4)c4ccc5ccccc5c4)c3)ccc2-c2c(-c3ccccc3)cccc21 ; CC1(C)c2cc(N(c3ccccc3)c3cc(-c4ccc5ccccc5c4-c4ccccc4)cc(N(c4ccccc4)c4ccc(-c5ccccc5)cc4)c3)ccc2-c2c(-c3ccccc3)cccc21]

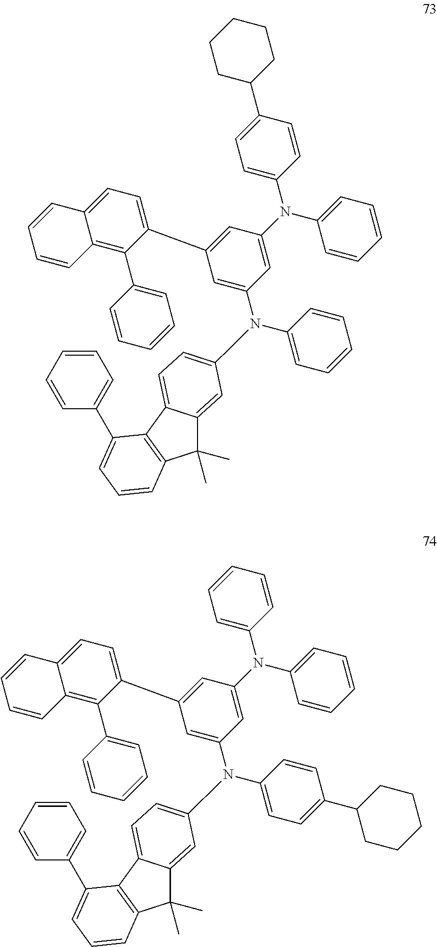 [SMILES: CC1(C)c2cc(N(c3ccccc3)c3cc(-c4ccc5ccccc5c4-c4ccccc4)cc(N(c4ccccc4)c4ccc(C5CCCCC5)cc4)c3)ccc2-c2c(-c3ccccc3)cccc21 ; CC1(C)c2cc(N(c3ccc(C4CCCCC4)cc3)c3cc(-c4ccc5ccccc5c4-c4ccccc4)cc(N(c4ccccc4)c4ccccc4)c3)ccc2-c2c(-c3ccccc3)cccc21]

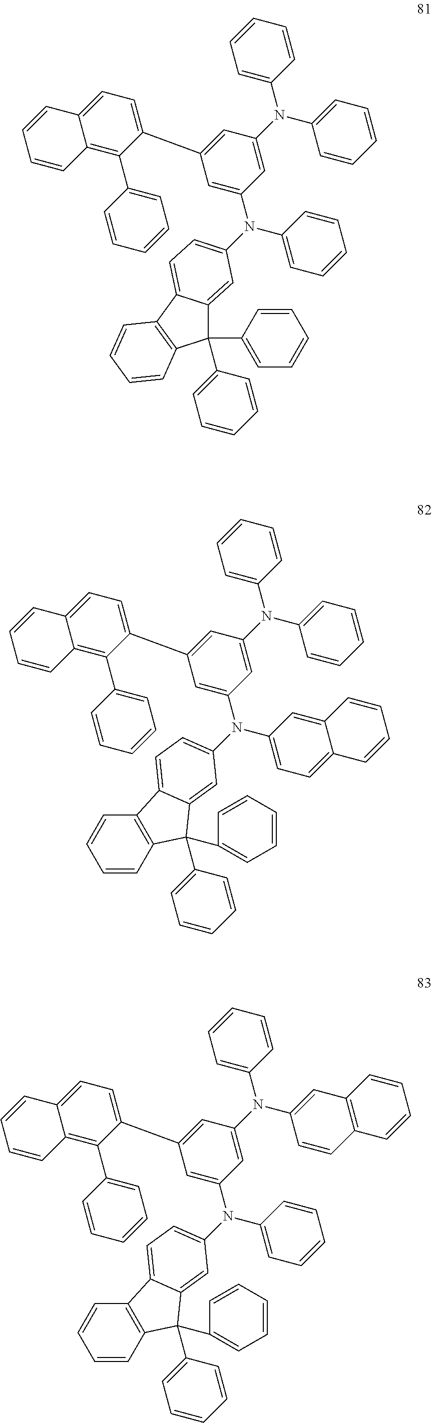 [SMILES: c1ccc(-c2c(-c3cc(N(c4ccccc4)c4ccccc4)cc(N(c4ccccc4)c4ccc5c(c4)C(c4ccccc4)(c4ccccc4)c4ccccc4-5)c3)ccc3ccccc23)cc1 ; c1ccc(-c2c(-c3cc(N(c4ccccc4)c4ccccc4)cc(N(c4ccc5c(c4)C(c4ccccc4)(c4ccccc4)c4ccccc4-5)c4ccc5ccccc5c4)c3)ccc3ccccc23)cc1 ; c1ccc(-c2c(-c3cc(N(c4ccccc4)c4ccc5c(c4)C(c4ccccc4)(c4ccccc4)c4ccccc4-5)cc(N(c4ccccc4)c4ccc5ccccc5c4)c3)ccc3ccccc23)cc1]

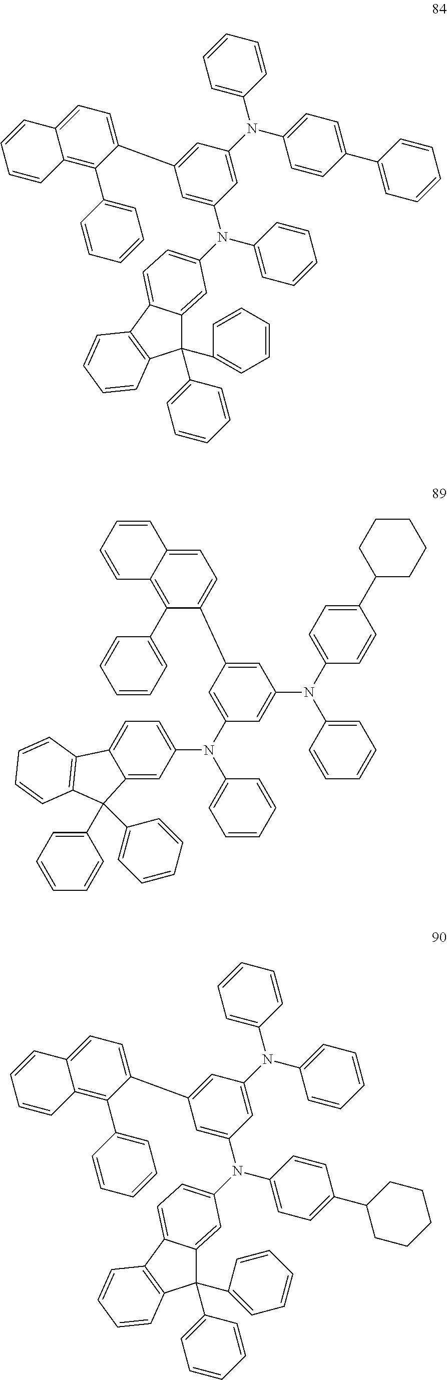 [SMILES: c1ccc(-c2ccc(N(c3ccccc3)c3cc(-c4ccc5ccccc5c4-c4ccccc4)cc(N(c4ccccc4)c4ccc5c(c4)C(c4ccccc4)(c4ccccc4)c4ccccc4-5)c3)cc2)cc1 ; c1ccc(-c2c(-c3cc(N(c4ccccc4)c4ccc(C5CCCCC5)cc4)cc(N(c4ccccc4)c4ccc5c(c4)C(c4ccccc4)(c4ccccc4)c4ccccc4-5)c3)ccc3ccccc23)cc1 ; c1ccc(-c2c(-c3cc(N(c4ccccc4)c4ccccc4)cc(N(c4ccc(C5CCCCC5)cc4)c4ccc5c(c4)C(c4ccccc4)(c4ccccc4)c4ccccc4-5)c3)ccc3ccccc23)cc1]

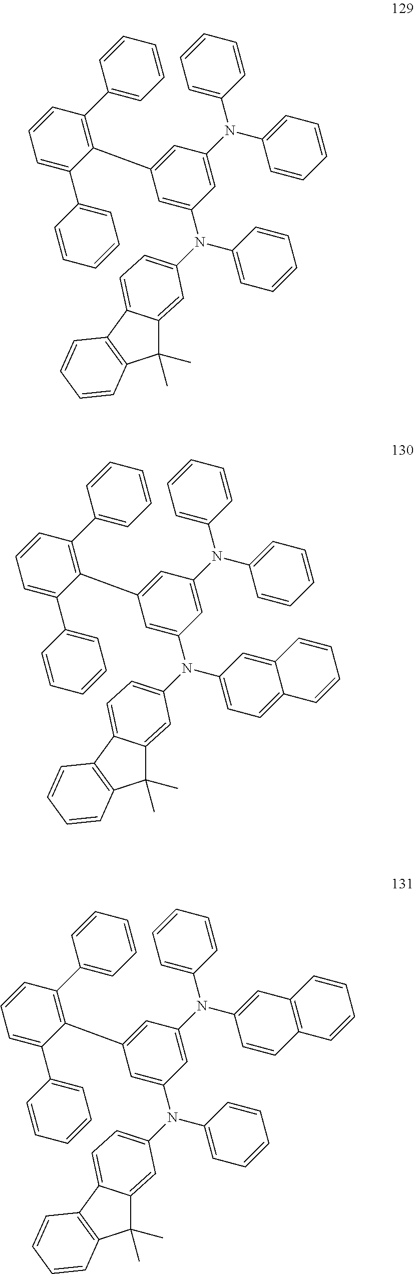 [SMILES: CC1(C)c2ccccc2-c2ccc(N(c3ccccc3)c3cc(-c4c(-c5ccccc5)cccc4-c4ccccc4)cc(N(c4ccccc4)c4ccccc4)c3)cc21 ; CC1(C)c2ccccc2-c2ccc(N(c3cc(-c4c(-c5ccccc5)cccc4-c4ccccc4)cc(N(c4ccccc4)c4ccccc4)c3)c3ccc4ccccc4c3)cc21 ; CC1(C)c2ccccc2-c2ccc(N(c3ccccc3)c3cc(-c4c(-c5ccccc5)cccc4-c4ccccc4)cc(N(c4ccccc4)c4ccc5ccccc5c4)c3)cc21]

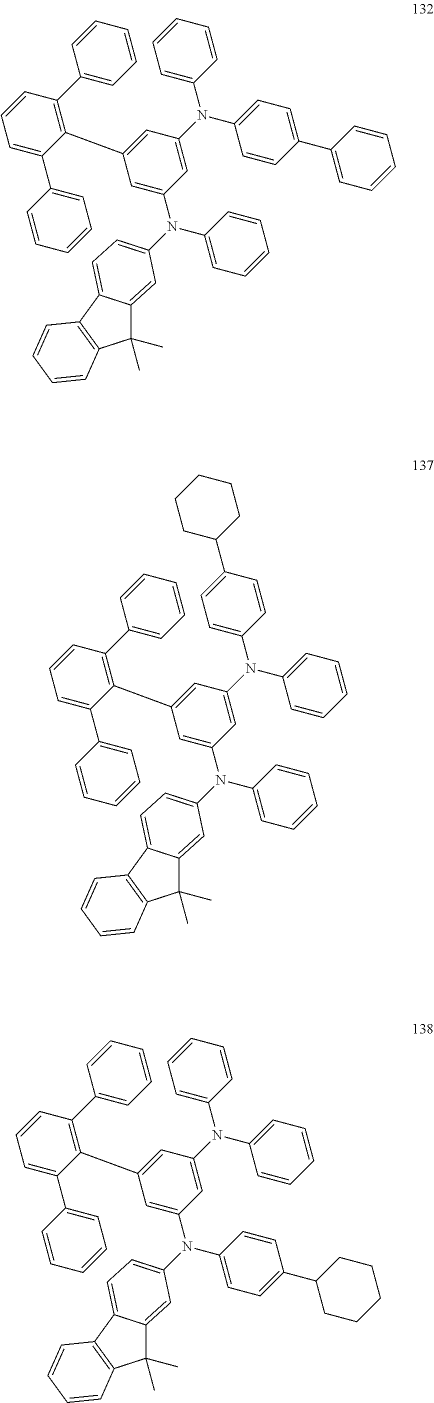 [SMILES: CC1(C)c2ccccc2-c2ccc(N(c3ccccc3)c3cc(-c4c(-c5ccccc5)cccc4-c4ccccc4)cc(N(c4ccccc4)c4ccc(-c5ccccc5)cc4)c3)cc21 ; CC1(C)c2ccccc2-c2ccc(N(c3ccccc3)c3cc(-c4c(-c5ccccc5)cccc4-c4ccccc4)cc(N(c4ccccc4)c4ccc(C5CCCCC5)cc4)c3)cc21 ; CC1(C)c2ccccc2-c2ccc(N(c3ccc(C4CCCCC4)cc3)c3cc(-c4c(-c5ccccc5)cccc4-c4ccccc4)cc(N(c4ccccc4)c4ccccc4)c3)cc21]

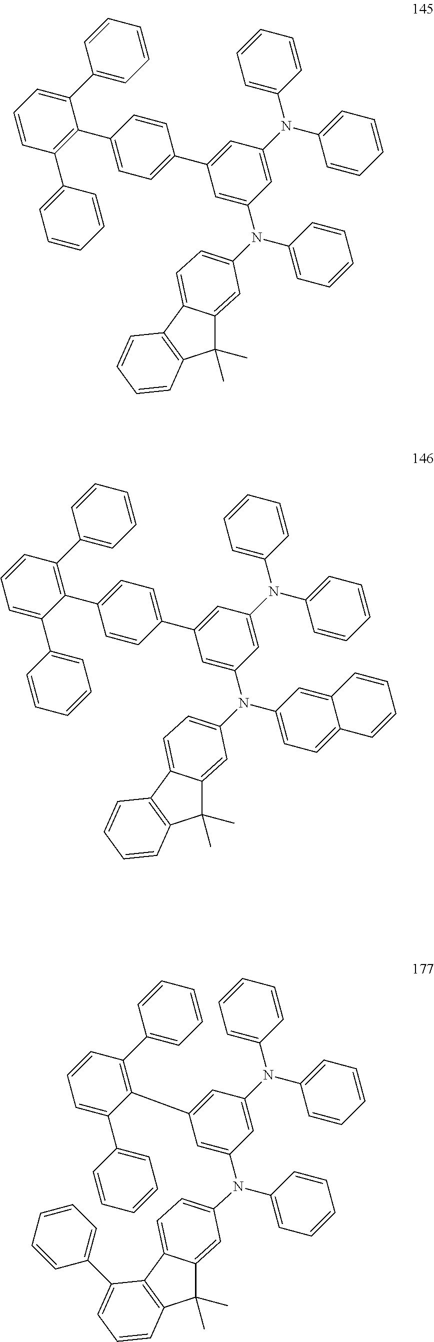 [SMILES: CC1(C)c2ccccc2-c2ccc(N(c3ccccc3)c3cc(-c4ccc(-c5c(-c6ccccc6)cccc5-c5ccccc5)cc4)cc(N(c4ccccc4)c4ccccc4)c3)cc21 ; CC1(C)c2ccccc2-c2ccc(N(c3cc(-c4ccc(-c5c(-c6ccccc6)cccc5-c5ccccc5)cc4)cc(N(c4ccccc4)c4ccccc4)c3)c3ccc4ccccc4c3)cc21 ; CC1(C)c2cc(N(c3ccccc3)c3cc(-c4c(-c5ccccc5)cccc4-c4ccccc4)cc(N(c4ccccc4)c4ccccc4)c3)ccc2-c2c(-c3ccccc3)cccc21]

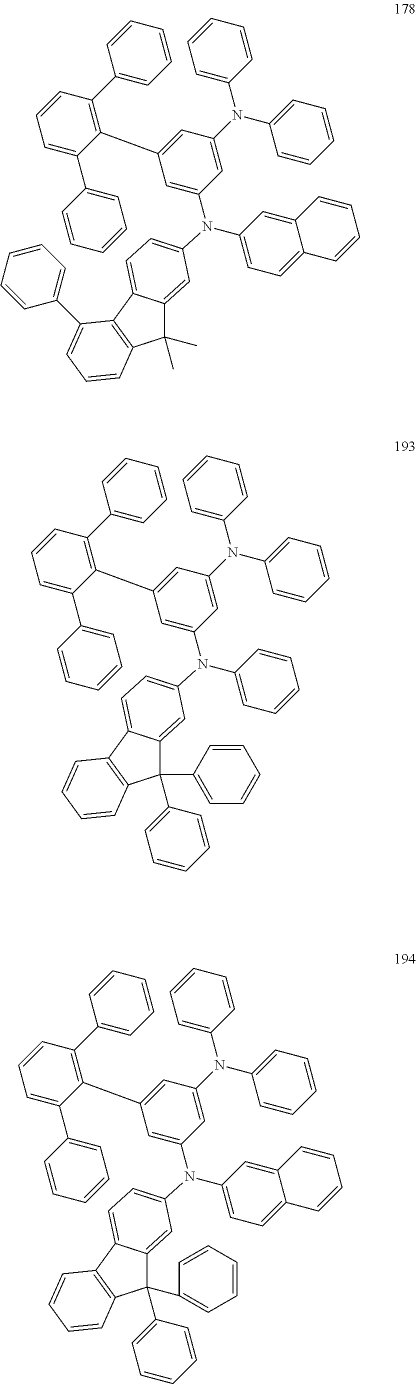 [SMILES: CC1(C)c2cc(N(c3cc(-c4c(-c5ccccc5)cccc4-c4ccccc4)cc(N(c4ccccc4)c4ccccc4)c3)c3ccc4ccccc4c3)ccc2-c2c(-c3ccccc3)cccc21 ; c1ccc(-c2cccc(-c3ccccc3)c2-c2cc(N(c3ccccc3)c3ccccc3)cc(N(c3ccccc3)c3ccc4c(c3)C(c3ccccc3)(c3ccccc3)c3ccccc3-4)c2)cc1 ; c1ccc(-c2cccc(-c3ccccc3)c2-c2cc(N(c3ccccc3)c3ccccc3)cc(N(c3ccc4c(c3)C(c3ccccc3)(c3ccccc3)c3ccccc3-4)c3ccc4ccccc4c3)c2)cc1]

Referring to Tables 1 and 2, it was confirmed that the light-emitting devices according to Examples 21 to 32, each of which included an organic compound represented by Formula 1 and satisfying Condition 1, and the light-emitting devices according to Examples 1 to 20, each of which included an organic compound represented by Formula 1 and satisfying Condition 2, emitted blue light and had a low driving voltage, high luminescence efficiency, and a long lifespan, as compared with the light-emitting devices according to Comparative Examples 1 to 14, each of which included a compound not represented by Formula 1 and/or not satisfying Conditions 1 and 2.

The organic compound described above is represented by Formula 1 and is a diamine-based compound to which a fluorenyl group is bonded. A linking group (L₁)ₐ₁ is bonded to a specific position of a benzene group linking two amine groups. An aryl group substituted with a phenyl group is linked to the linking group (L₁)ₐ₁. The phenyl group is in an ortho position with respect to the linking group (L₁)ₐ₁. In addition, if (e.g., when) the organic compound represented by Formula 1 satisfies Condition 1, the group formed of the aryl group substituted with the phenyl group at the ortho position together with the phenyl may not be a biphenyl group and may be a group in which at least three rings are bonded to each other, and if (e.g., when) the organic compound satisfies Condition 2, the aryl group substituted with the phenyl group at the ortho position may not be a benzene group and may be a naphthalene group. The organic compound satisfying at least one of Condition 1 or Condition 2 may have desired electrical stability and high charge-transporting ability, and thus, a light-emitting device including the organic compound may have a low driving voltage, high luminescence efficiency, and a long lifespan. In other words, the compound as represented by Formula 1, is a diamine derivative with a fluorenyl moiety. It features a linker (L₁)ₐ₁ attached to a benzene ring, which connects two amine groups. This benzene ring is further connected with an aryl group via the linker, the aryl group being substituted with a phenyl group at the ortho position relative to the linker. Depending on specific conditions, this phenyl-substituted aryl group can vary: under Condition 1, the group formed of it together with the phenyl cannot be a biphenyl but must be a tricyclic group, and under Condition 2, it cannot be a benzene but must be a naphthalene group. When either condition or both Conditions are met, the compound exhibits enhanced electrical stability and charge transport properties. Consequently, light-emitting devices incorporating this compound can benefit from reduced driving voltages, improved luminescence efficiency, and extended operational life.

In the present disclosure, it will be understood that the term “comprise(s),” “include(s),” or “have/has” specifies the presence of stated features, integers, steps, operations, elements, and/or components, but do not preclude the presence or addition of one or more other features, integers, steps, operations, elements, components, and/or groups thereof.

In the context of the present application and unless otherwise defined, the terms “use,” “using,” and “used” may be considered synonymous with the terms “utilize,” “utilizing,” and “utilized,” respectively.

Throughout the present disclosure, when a component such as a layer, a film, a region, or a plate is mentioned to be placed “on” another component, it will be understood that it may be directly on another component or that another component may be interposed therebetween. In some embodiments, “directly on” may refer to that there are no additional layers, films, regions, plates, etc., between a layer, a film, a region, a plate, etc. and the other part. For example, “directly on” may refer to two layers or two members are disposed without utilizing an additional member such as an adhesive member therebetween.

In the present disclosure, although the terms “first,” “second,” etc., may be utilized herein to describe one or more elements, components, regions, and/or layers, these elements, components, regions, and/or layers should not be limited by these terms. These terms are only utilized to distinguish one component from another component.

As utilized herein, the singular forms “a,” “an,” “one,” and “the” are intended to include the plural forms as well, unless the context clearly indicates otherwise. Further, the use of “may” when describing embodiments of the present disclosure refers to “one or more embodiments of the present disclosure”.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, or 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in the present disclosure is intended to include all higher numerical limitations subsumed therein. Accordingly, Applicant reserves the right to amend the disclosure, including the claims, to expressly recite any sub-range subsumed within the ranges expressly recited herein.

The light-emitting device, the electronic apparatus, the display device/apparatus, the electronic equipment, the electronic device, a device for manufacturing thereof, or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the drawings, it will be understood by those of ordinary skill in the art that one or more suitable changes in form and details may be made therein without departing from the spirit and scope as defined by the appended claims and equivalents thereof.

What is claimed is:

1. A light-emitting device comprising:
a first electrode;
a second electrode opposite to the first electrode;
an interlayer between the first electrode and the second electrode and comprising an emission layer; and
at least one organic compound represented by Formula 1 and satisfying at least one selected from among Condition 1 and Condition 2: 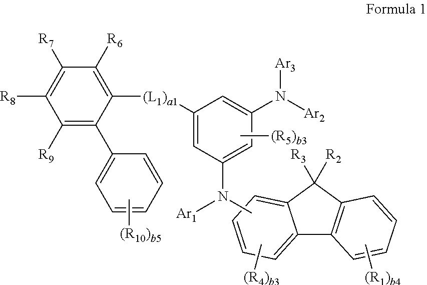
wherein, in Formula 1,
L₁ is a C₃-C₆₀ carbocyclic group unsubstituted or substituted with at least one R₁₀ₐ or a C₁-C₆₀ heterocyclic group unsubstituted or substituted with at least one R₁₀ₐ,
a1 is an integer from 0 to 2, wherein, when a1 is 0, then (L₁)ₐ₁ is a single bond,
Ar₁ to Ar₃ and R₁ to R₁₀ are each independently hydrogen, deuterium, —F, —Cl, -Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
b3 is an integer from 0 to 3,
b4 is an integer from 0 to 4,
b5 is an integer from 0 to 5,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, or a nitro group; or
a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof,
Condition 1
at least one selected from among R₆ to R₉ is a C₆-C₆₀ aryl group unsubstituted or substituted with at least one R₁₀ₐ or a C₁-C₆₀ heteroaryl group unsubstituted or substituted with at least one R₁₀ₐ
Condition 2
R₈ and R₉ are bonded to each other to form a C₃-C₆₀ carbocyclic group or a C₁-C₆₀ heterocyclic group.

2. The light-emitting device of claim 1, wherein the interlayer comprises the at least one organic compound.

3. The light-emitting device of claim 1, wherein the interlayer further comprises a hole transport region between the first electrode and the emission layer, and
the hole transport region comprises the at least one organic compound.

4. The light-emitting device of claim 3, wherein the hole transport region comprises:
a hole injection layer on the first electrode; and
a hole transport layer on the hole injection layer, and
the hole transport layer comprises the at least one organic compound.

5. The light-emitting device of claim 1, further comprising at least one of a first capping layer arranged outside the first electrode or a second capping layer arranged outside the second electrode,
wherein the at least one of the first capping layer or the second capping layer comprises the at least one organic compound.

6. The light-emitting device of claim 1, wherein the emission layer comprises:
a dopant comprising at least one cyclic group comprising both boron and nitrogen as ring-forming atoms; and
a sensitizer comprising a transition metal.

7. The light-emitting device of claim 1, wherein the emission layer is to emit blue light.

8. An electronic apparatus comprising:
the light-emitting device of claim 1; and
a thin-film transistor electrically connected to the light-emitting device.

9. An electronic equipment comprising the light-emitting device of claim 1, wherein the electronic equipment is at least one of a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality display, an augmented reality display, a vehicle, a video wall with multiple displays tiled together, a theater screen, a stadium screen, a phototherapy device, or a signboard.

10. An organic compound represented by Formula 1 and satisfying at least one selected from among Condition 1 and Condition 2: 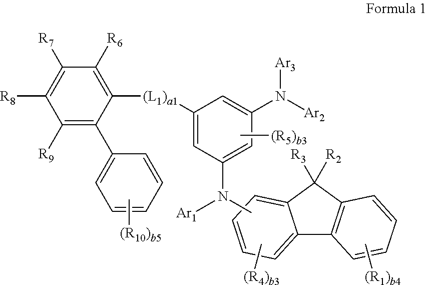
wherein, in Formula 1,
L₁ is a C₃-C₆₀ carbocyclic group unsubstituted or substituted with at least one R₁₀ₐ or a C₁-C₆₀ heterocyclic group unsubstituted or substituted with at least one R₁₀ₐ,
a1 is an integer from 0 to 2, wherein, when a1 is 0, then (L₁)ₐ₁ is a single bond,
Ar₁ to Ar₃ and R₁ to R₁₀ are each independently hydrogen, deuterium, —F, —Cl, -Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
b3 is an integer from 0 to 3,
b4 is an integer from 0 to 4,
b5 is an integer from 0 to 5,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, or a nitro group; or
a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof,
Condition 1
at least one selected from among R₆ to R₉ is a C₆-C₆₀ aryl group unsubstituted or substituted with at least one R₁₀ₐ or a C₁-C₆₀ heteroaryl group unsubstituted or substituted with at least one R₁₀ₐ
Condition 2
R₈ and R₉ are bonded to each other to form a C₃-C₆₀ carbocyclic group or a C₁-C₆₀ heterocyclic group.

11. The organic compound of claim 10, wherein the organic compound is represented by Formula 1-1: 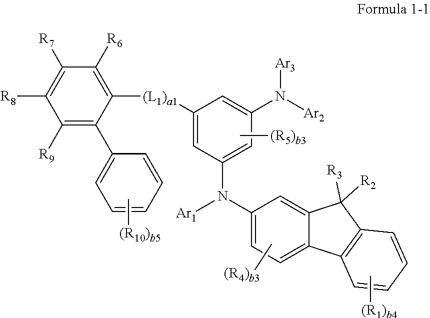
in Formula 1-1, L₁, a1, Ar₁ to Ar₃, R₁ to R₁₀, and b3 to b5 being each independently the same as defined in Formula 1.

12. The organic compound of claim 10, wherein
L₁ is a phenylene group, or
a1 is 0.

13. The organic compound of claim 10, wherein Ar₁ to Ar₃ are each independently a C₃-C₆₀ carbocyclic group unsubstituted or substituted with at least one R₁₀ₐ or a C₁-C₆₀ heterocyclic group unsubstituted or substituted with at least one R₁₀ₐ.

14. The organic compound of claim 10, wherein Ar₁ to Ar₃ are each independently a C₆-C₆₀ aryl group unsubstituted or substituted with at least one R₁₀ₐ, a C₁-C₆₀ heteroaryl group unsubstituted or substituted with at least one R₁₀ₐ, a monovalent non-aromatic condensed polycyclic group unsubstituted or substituted with at least one R₁₀ₐ, or a monovalent non-aromatic condensed heteropolycyclic group unsubstituted or substituted with at least one R₁₀ₐ.

15. The organic compound of claim 10, wherein R₁ to R₅ and R₁₀ are each independently hydrogen, deuterium, a C₁-C₆₀ alkyl group unsubstituted or substituted with at least one R₁₀ₐ, a C₆-C₆₀ aryl group unsubstituted or substituted with at least one R₁₀ₐ, or a C₁-C₆₀ heteroaryl group unsubstituted or substituted with at least one R₁₀ₐ.

16. The organic compound of claim 10, wherein the organic compound satisfies both Condition 1 and Condition 2.

17. The organic compound of claim 10, wherein, in Condition 1, at least one selected from among R₆ to R₉ is a phenyl group unsubstituted or substituted with at least one R₁₀ₐ or a naphthyl group unsubstituted or substituted with at least one R₁₀ₐ.

18. The organic compound of claim 10, wherein, in Condition 1, R₆ is a C₆-C₆₀ aryl group unsubstituted or substituted with at least one R₁₀ₐ or a C₁-C₆₀ heteroaryl group unsubstituted or substituted with at least one R₁₀ₐ, and
R₇ to R₉ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

19. The organic compound of claim 10, wherein, in Condition 2, R₈ and Rs are bonded to each other to form a benzene group or a naphthalene group.

20. The organic compound of claim 10, wherein the organic compound is any one selected from among Compounds 1 to 208: 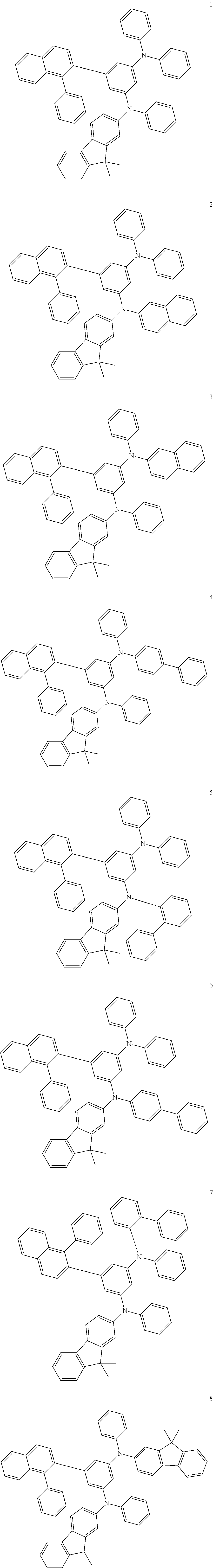 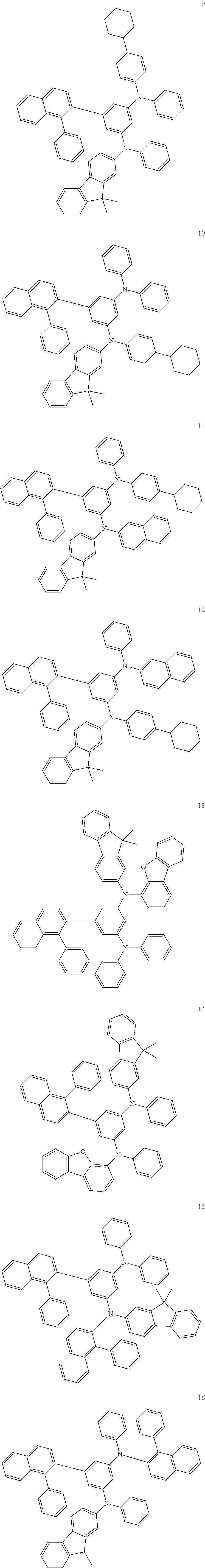 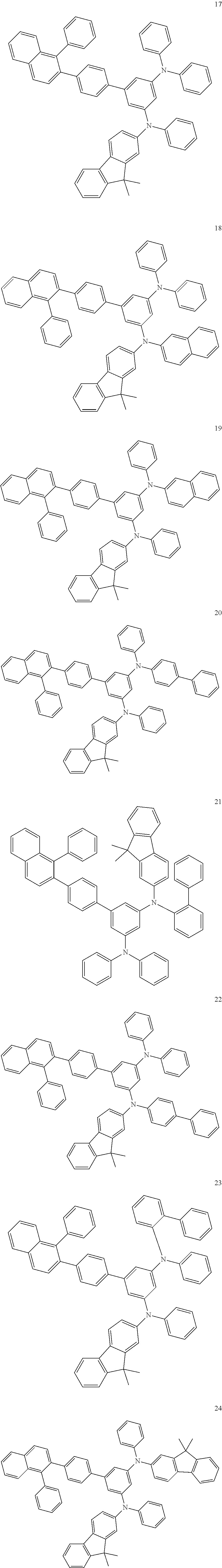 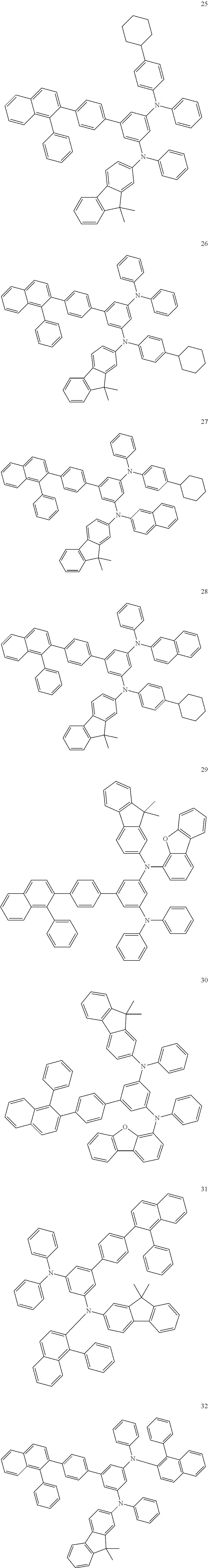 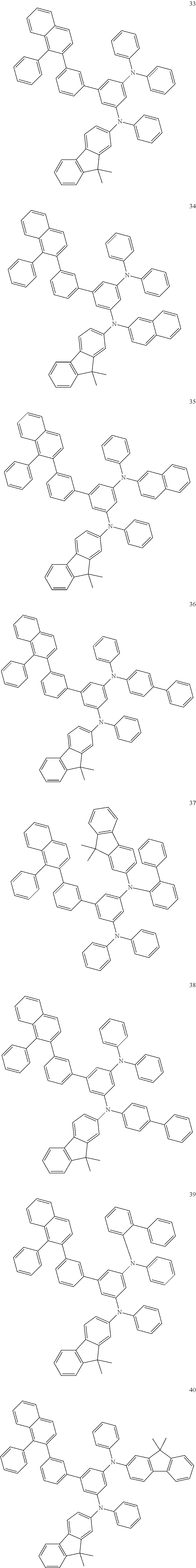 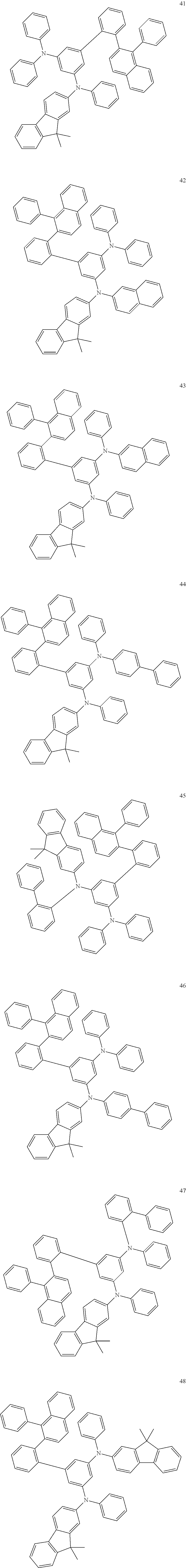 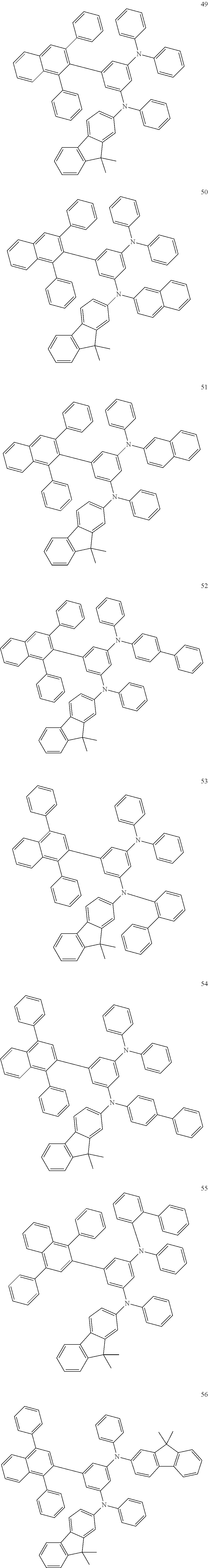 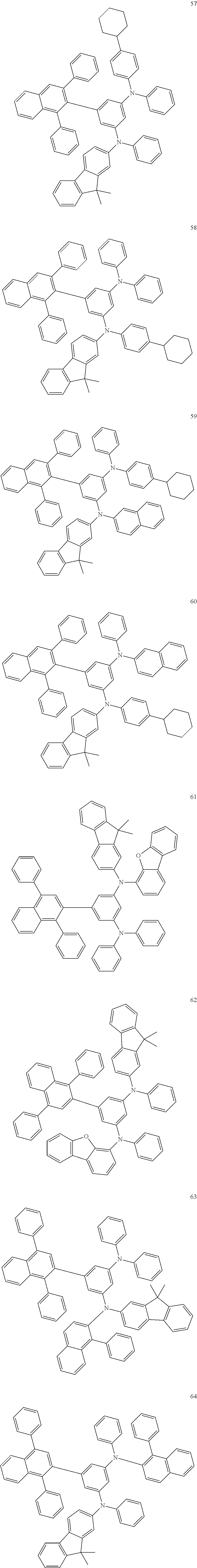 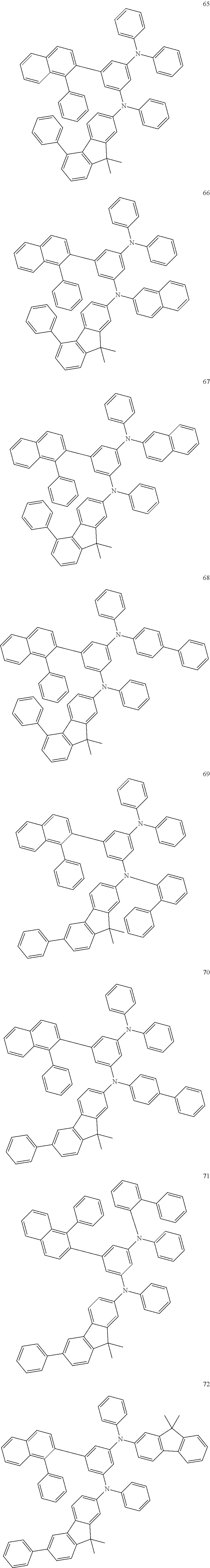 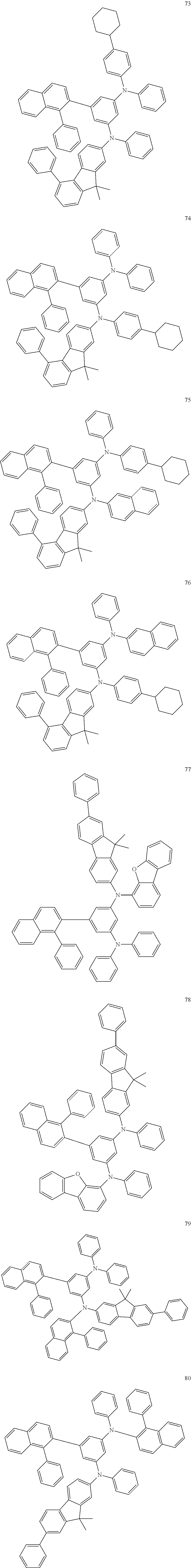 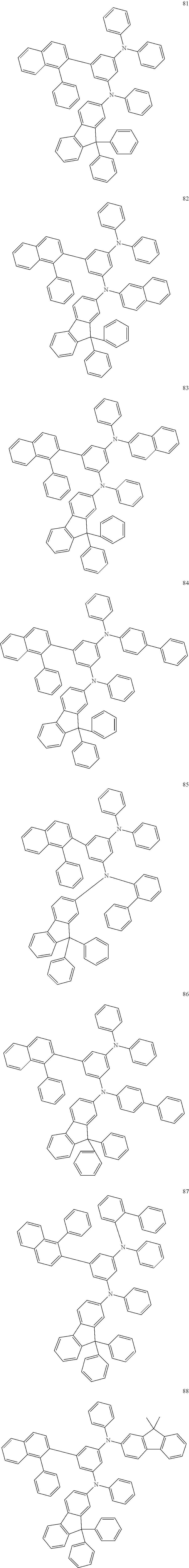 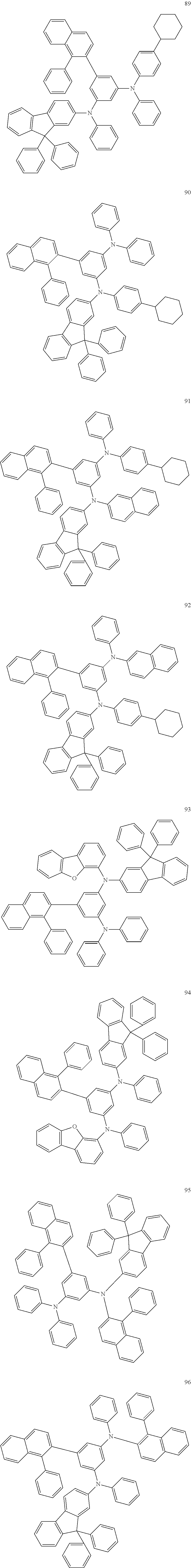 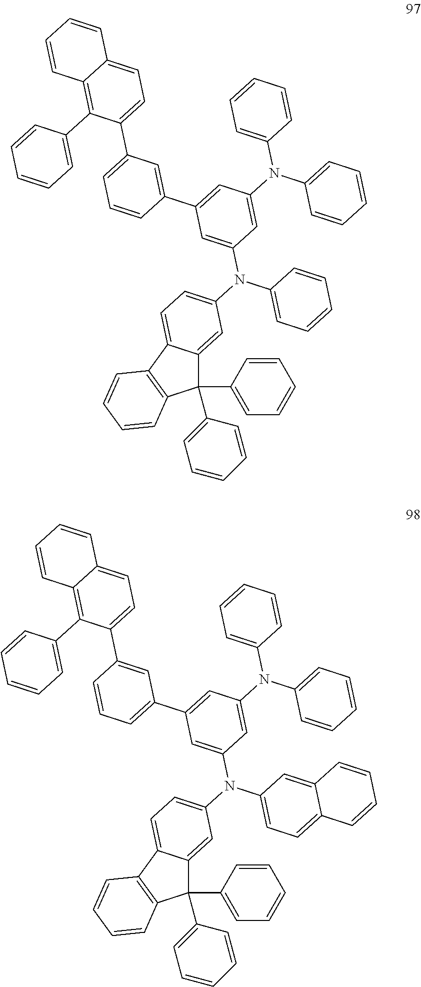 [SMILES: c1ccc(-c2c(-c3cccc(-c4cc(N(c5ccccc5)c5ccccc5)cc(N(c5ccccc5)c5ccc6c(c5)C(c5ccccc5)(c5ccccc5)c5ccccc5-6)c4)c3)ccc3ccccc23)cc1 ; c1ccc(-c2c(-c3cccc(-c4cc(N(c5ccccc5)c5ccccc5)cc(N(c5ccc6c(c5)C(c5ccccc5)(c5ccccc5)c5ccccc5-6)c5ccc6ccccc6c5)c4)c3)ccc3ccccc23)cc1] 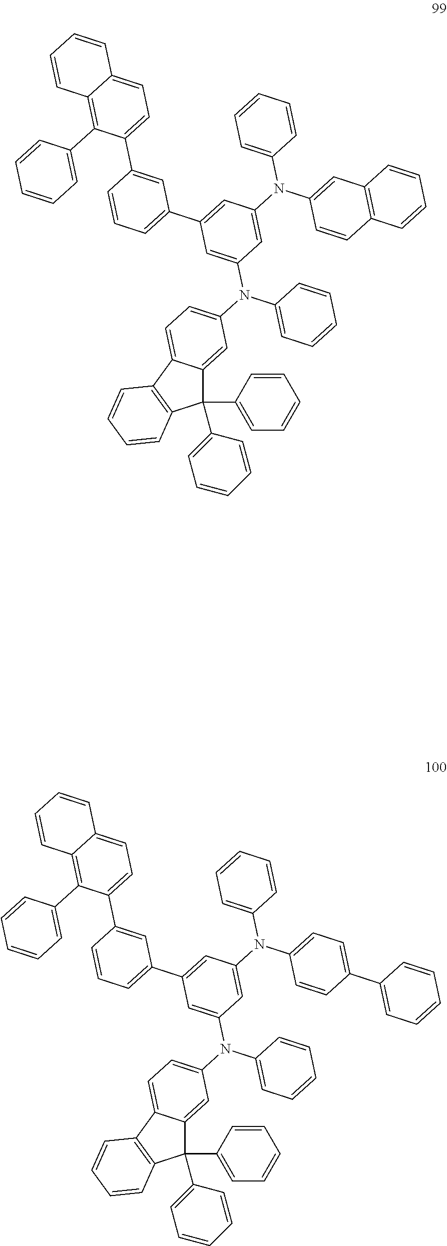 [SMILES: c1ccc(-c2c(-c3cccc(-c4cc(N(c5ccccc5)c5ccc6c(c5)C(c5ccccc5)(c5ccccc5)c5ccccc5-6)cc(N(c5ccccc5)c5ccc6ccccc6c5)c4)c3)ccc3ccccc23)cc1 ; c1ccc(-c2ccc(N(c3ccccc3)c3cc(-c4cccc(-c5ccc6ccccc6c5-c5ccccc5)c4)cc(N(c4ccccc4)c4ccc5c(c4)C(c4ccccc4)(c4ccccc4)c4ccccc4-5)c3)cc2)cc1] 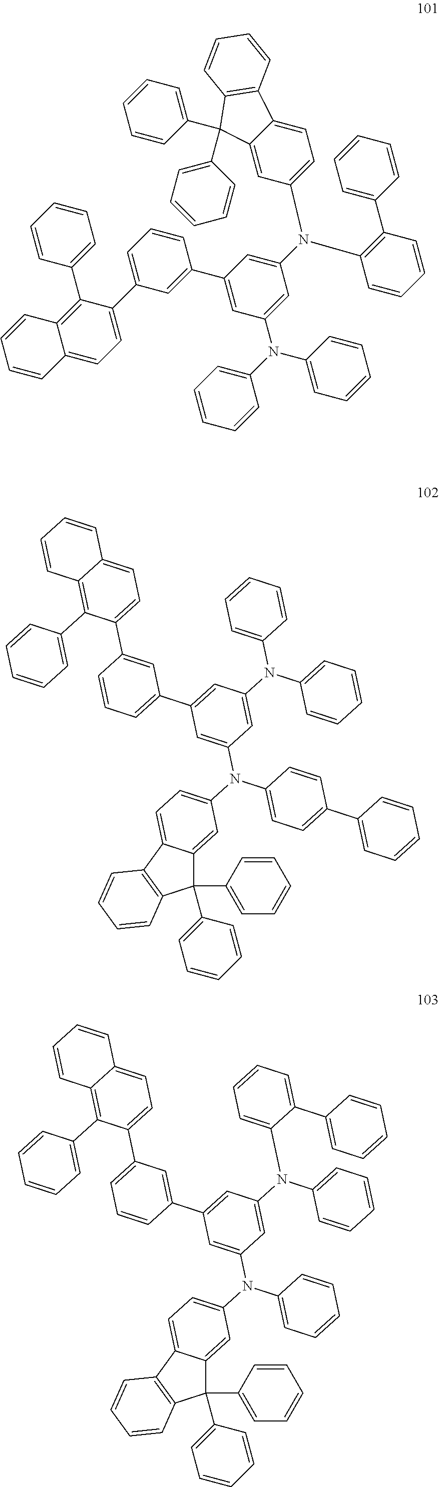 [SMILES: c1ccc(-c2ccccc2N(c2cc(-c3cccc(-c4ccc5ccccc5c4-c4ccccc4)c3)cc(N(c3ccccc3)c3ccccc3)c2)c2ccc3c(c2)C(c2ccccc2)(c2ccccc2)c2ccccc2-3)cc1 ; c1ccc(-c2ccc(N(c3cc(-c4cccc(-c5ccc6ccccc6c5-c5ccccc5)c4)cc(N(c4ccccc4)c4ccccc4)c3)c3ccc4c(c3)C(c3ccccc3)(c3ccccc3)c3ccccc3-4)cc2)cc1 ; c1ccc(-c2ccccc2N(c2ccccc2)c2cc(-c3cccc(-c4ccc5ccccc5c4-c4ccccc4)c3)cc(N(c3ccccc3)c3ccc4c(c3)C(c3ccccc3)(c3ccccc3)c3ccccc3-4)c2)cc1] 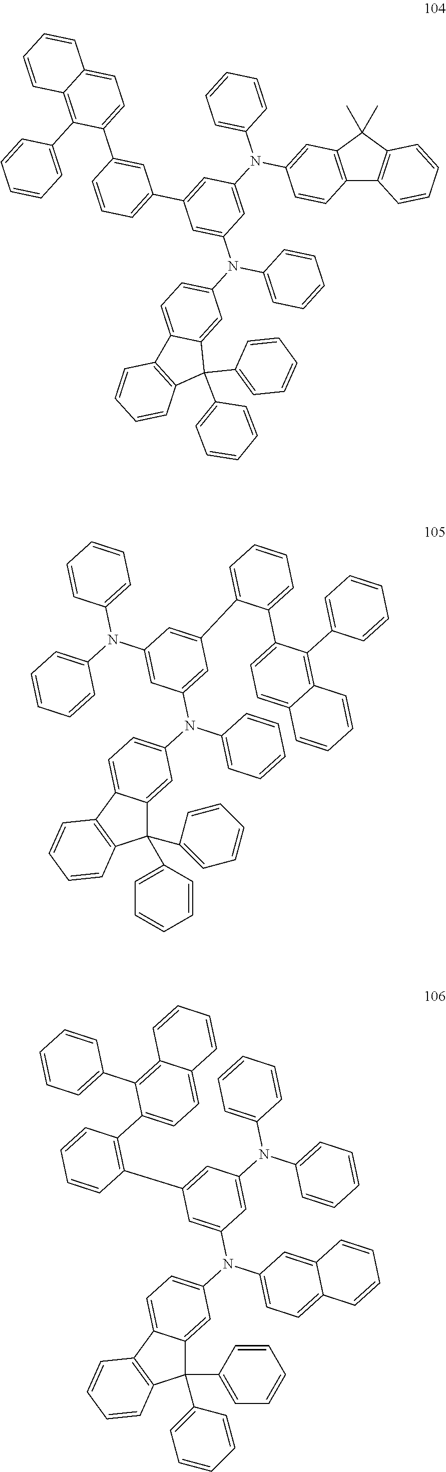 [SMILES: CC1(C)c2ccccc2-c2ccc(N(c3ccccc3)c3cc(-c4cccc(-c5ccc6ccccc6c5-c5ccccc5)c4)cc(N(c4ccccc4)c4ccc5c(c4)C(c4ccccc4)(c4ccccc4)c4ccccc4-5)c3)cc21 ; c1ccc(-c2c(-c3ccccc3-c3cc(N(c4ccccc4)c4ccccc4)cc(N(c4ccccc4)c4ccc5c(c4)C(c4ccccc4)(c4ccccc4)c4ccccc4-5)c3)ccc3ccccc23)cc1 ; c1ccc(-c2c(-c3ccccc3-c3cc(N(c4ccccc4)c4ccccc4)cc(N(c4ccc5c(c4)C(c4ccccc4)(c4ccccc4)c4ccccc4-5)c4ccc5ccccc5c4)c3)ccc3ccccc23)cc1] 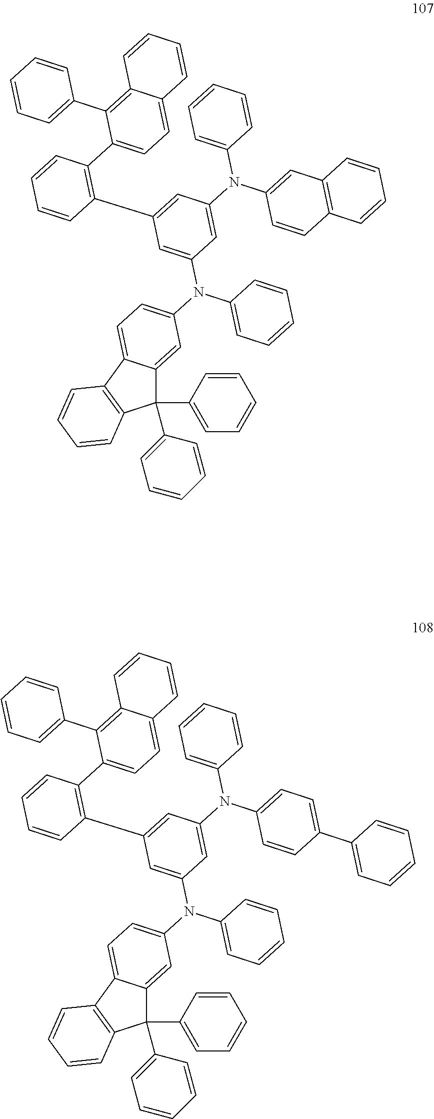 [SMILES: c1ccc(-c2c(-c3ccccc3-c3cc(N(c4ccccc4)c4ccc5c(c4)C(c4ccccc4)(c4ccccc4)c4ccccc4-5)cc(N(c4ccccc4)c4ccc5ccccc5c4)c3)ccc3ccccc23)cc1 ; c1ccc(-c2ccc(N(c3ccccc3)c3cc(-c4ccccc4-c4ccc5ccccc5c4-c4ccccc4)cc(N(c4ccccc4)c4ccc5c(c4)C(c4ccccc4)(c4ccccc4)c4ccccc4-5)c3)cc2)cc1] 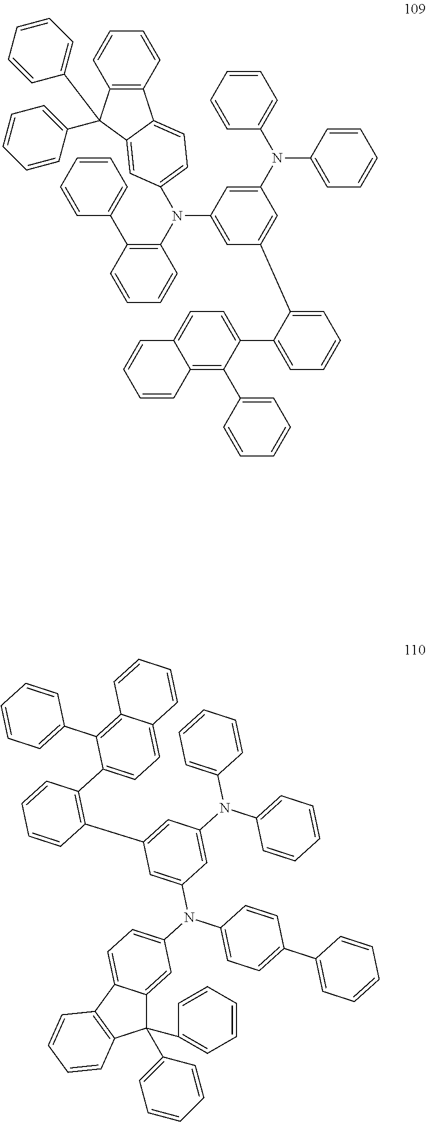 [SMILES: c1ccc(-c2ccccc2N(c2cc(-c3ccccc3-c3ccc4ccccc4c3-c3ccccc3)cc(N(c3ccccc3)c3ccccc3)c2)c2ccc3c(c2)C(c2ccccc2)(c2ccccc2)c2ccccc2-3)cc1 ; c1ccc(-c2ccc(N(c3cc(-c4ccccc4-c4ccc5ccccc5c4-c4ccccc4)cc(N(c4ccccc4)c4ccccc4)c3)c3ccc4c(c3)C(c3ccccc3)(c3ccccc3)c3ccccc3-4)cc2)cc1] 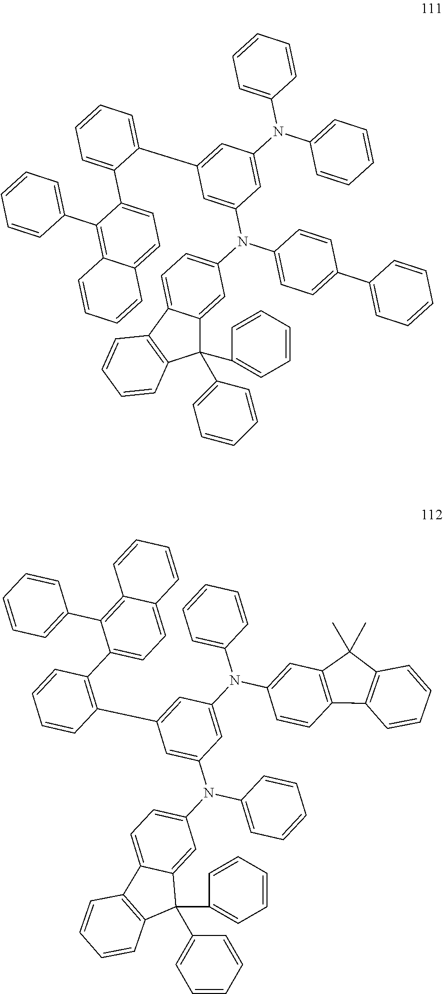 [SMILES: c1ccc(-c2ccc(N(c3cc(-c4ccccc4-c4ccc5ccccc5c4-c4ccccc4)cc(N(c4ccccc4)c4ccccc4)c3)c3ccc4c(c3)C(c3ccccc3)(c3ccccc3)c3ccccc3-4)cc2)cc1 ; CC1(C)c2ccccc2-c2ccc(N(c3ccccc3)c3cc(-c4ccccc4-c4ccc5ccccc5c4-c4ccccc4)cc(N(c4ccccc4)c4ccc5c(c4)C(c4ccccc4)(c4ccccc4)c4ccccc4-5)c3)cc21] 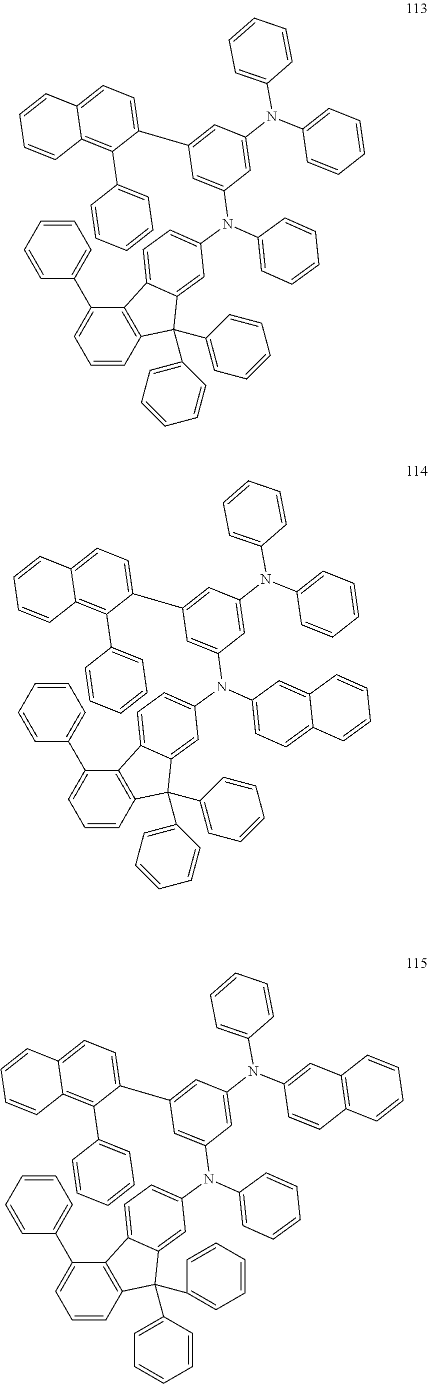 [SMILES: c1ccc(-c2cccc3c2-c2ccc(N(c4ccccc4)c4cc(-c5ccc6ccccc6c5-c5ccccc5)cc(N(c5ccccc5)c5ccccc5)c4)cc2C3(c2ccccc2)c2ccccc2)cc1 ; c1ccc(-c2cccc3c2-c2ccc(N(c4cc(-c5ccc6ccccc6c5-c5ccccc5)cc(N(c5ccccc5)c5ccccc5)c4)c4ccc5ccccc5c4)cc2C3(c2ccccc2)c2ccccc2)cc1 ; c1ccc(-c2cccc3c2-c2ccc(N(c4ccccc4)c4cc(-c5ccc6ccccc6c5-c5ccccc5)cc(N(c5ccccc5)c5ccc6ccccc6c5)c4)cc2C3(c2ccccc2)c2ccccc2)cc1] 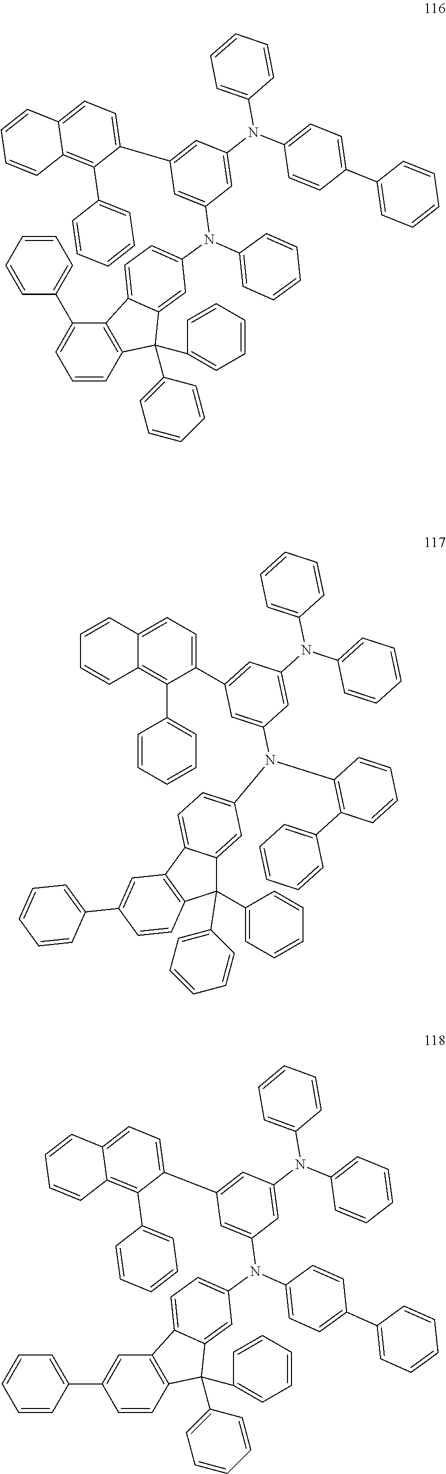 [SMILES: c1ccc(-c2ccc(N(c3ccccc3)c3cc(-c4ccc5ccccc5c4-c4ccccc4)cc(N(c4ccccc4)c4ccc5c(c4)C(c4ccccc4)(c4ccccc4)c4cccc(-c6ccccc6)c4-5)c3)cc2)cc1 ; c1ccc(-c2ccc3c(c2)-c2ccc(N(c4cc(-c5ccc6ccccc6c5-c5ccccc5)cc(N(c5ccccc5)c5ccccc5)c4)c4ccccc4-c4ccccc4)cc2C3(c2ccccc2)c2ccccc2)cc1 ; c1ccc(-c2ccc(N(c3cc(-c4ccc5ccccc5c4-c4ccccc4)cc(N(c4ccccc4)c4ccccc4)c3)c3ccc4c(c3)C(c3ccccc3)(c3ccccc3)c3ccc(-c5ccccc5)cc3-4)cc2)cc1] 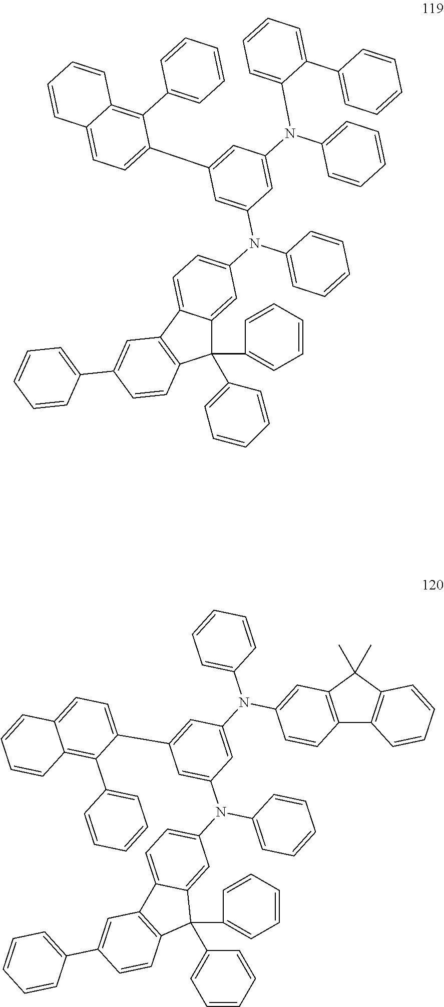 [SMILES: c1ccc(-c2ccc3c(c2)-c2ccc(N(c4ccccc4)c4cc(-c5ccc6ccccc6c5-c5ccccc5)cc(N(c5ccccc5)c5ccccc5-c5ccccc5)c4)cc2C3(c2ccccc2)c2ccccc2)cc1 ; CC1(C)c2ccccc2-c2ccc(N(c3ccccc3)c3cc(-c4ccc5ccccc5c4-c4ccccc4)cc(N(c4ccccc4)c4ccc5c(c4)C(c4ccccc4)(c4ccccc4)c4ccc(-c6ccccc6)cc4-5)c3)cc21] 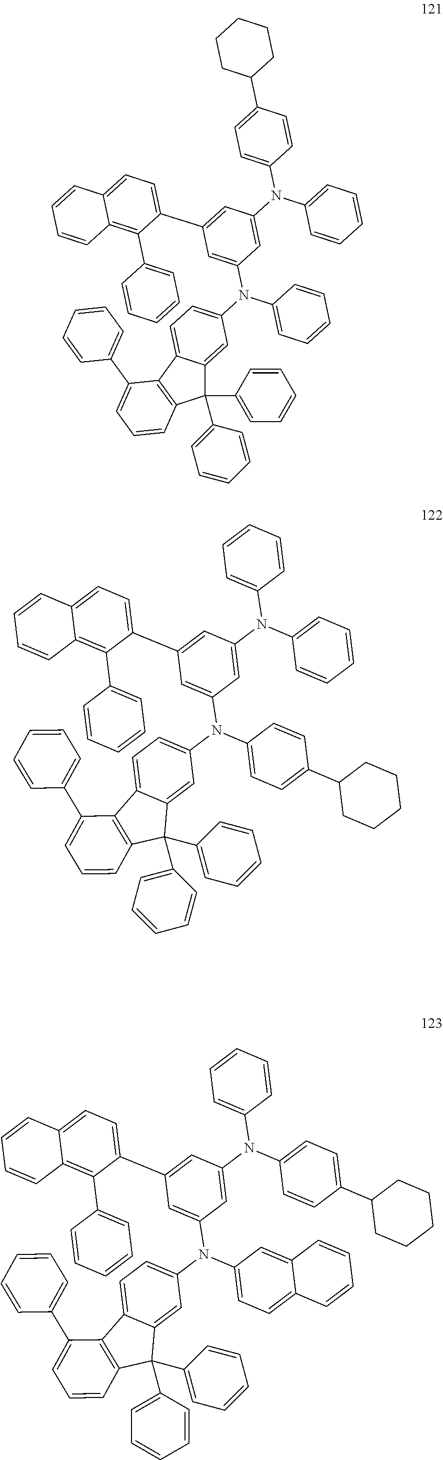 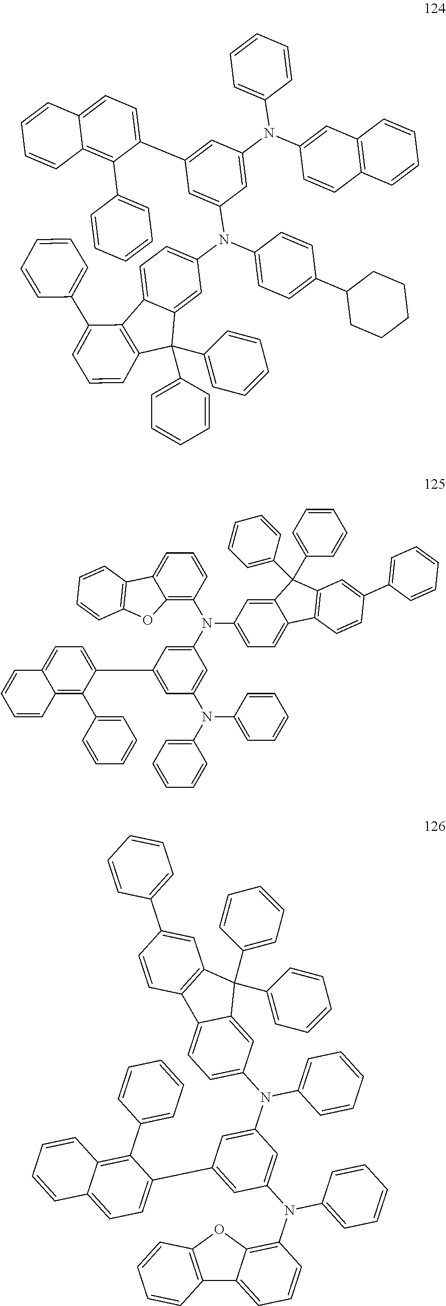 [SMILES: c1ccc(-c2cccc3c2-c2ccc(N(c4ccc(C5CCCCC5)cc4)c4cc(-c5ccc6ccccc6c5-c5ccccc5)cc(N(c5ccccc5)c5ccc6ccccc6c5)c4)cc2C3(c2ccccc2)c2ccccc2)cc1 ; c1ccc(-c2ccc3c(c2)C(c2ccccc2)(c2ccccc2)c2cc(N(c4cc(-c5ccc6ccccc6c5-c5ccccc5)cc(N(c5ccccc5)c5ccccc5)c4)c4cccc5c4oc4ccccc45)ccc2-3)cc1 ; c1ccc(-c2ccc3c(c2)C(c2ccccc2)(c2ccccc2)c2cc(N(c4ccccc4)c4cc(-c5ccc6ccccc6c5-c5ccccc5)cc(N(c5ccccc5)c5cccc6c5oc5ccccc56)c4)ccc2-3)cc1] 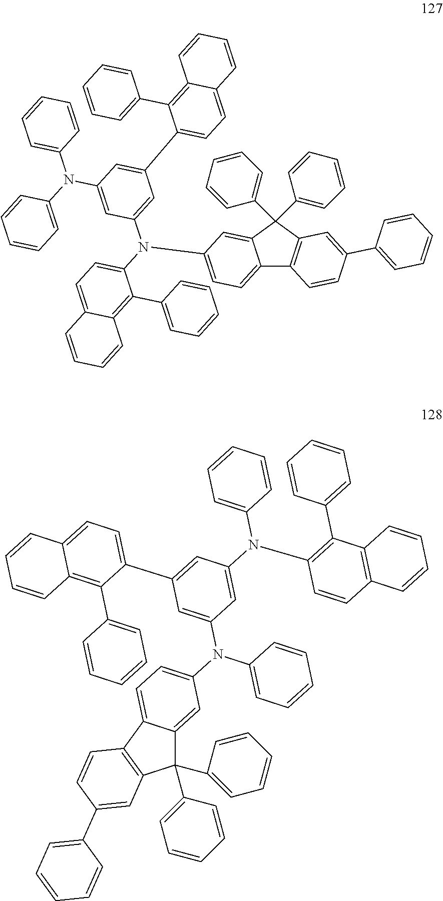 [SMILES: c1ccc(-c2ccc3c(c2)C(c2ccccc2)(c2ccccc2)c2cc(N(c4cc(-c5ccc6ccccc6c5-c5ccccc5)cc(N(c5ccccc5)c5ccccc5)c4)c4ccc5ccccc5c4-c4ccccc4)ccc2-3)cc1 ; c1ccc(-c2ccc3c(c2)C(c2ccccc2)(c2ccccc2)c2cc(N(c4ccccc4)c4cc(-c5ccc6ccccc6c5-c5ccccc5)cc(N(c5ccccc5)c5ccc6ccccc6c5-c5ccccc5)c4)ccc2-3)cc1] 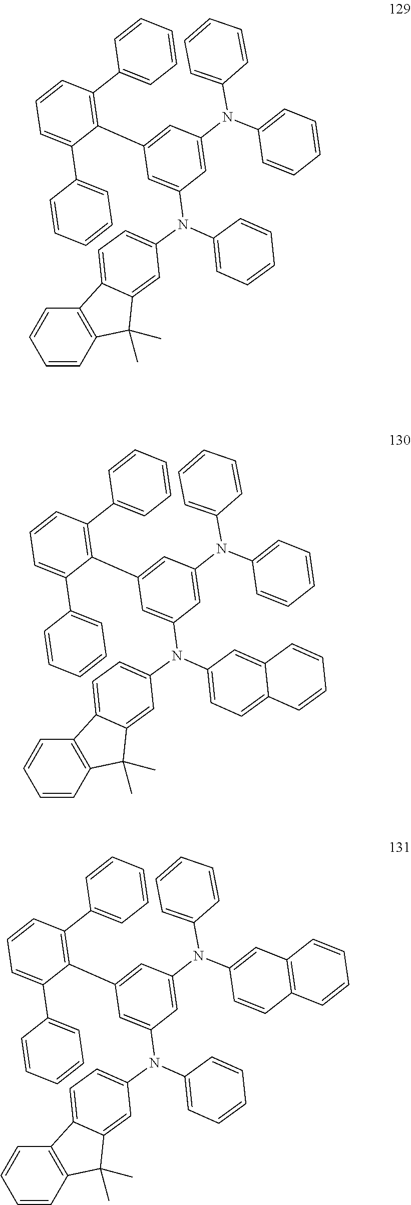 [SMILES: CC1(C)c2ccccc2-c2ccc(N(c3ccccc3)c3cc(-c4c(-c5ccccc5)cccc4-c4ccccc4)cc(N(c4ccccc4)c4ccccc4)c3)cc21 ; CC1(C)c2ccccc2-c2ccc(N(c3cc(-c4c(-c5ccccc5)cccc4-c4ccccc4)cc(N(c4ccccc4)c4ccccc4)c3)c3ccc4ccccc4c3)cc21 ; CC1(C)c2ccccc2-c2ccc(N(c3ccccc3)c3cc(-c4c(-c5ccccc5)cccc4-c4ccccc4)cc(N(c4ccccc4)c4ccc5ccccc5c4)c3)cc21] 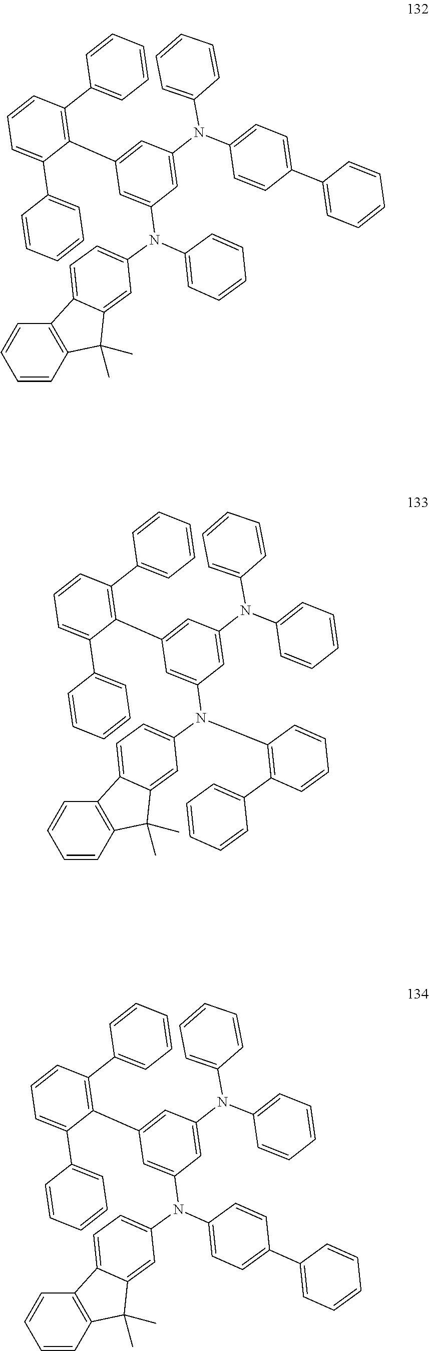 [SMILES: CC1(C)c2ccccc2-c2ccc(N(c3ccccc3)c3cc(-c4c(-c5ccccc5)cccc4-c4ccccc4)cc(N(c4ccccc4)c4ccc(-c5ccccc5)cc4)c3)cc21 ; CC1(C)c2ccccc2-c2ccc(N(c3cc(-c4c(-c5ccccc5)cccc4-c4ccccc4)cc(N(c4ccccc4)c4ccccc4)c3)c3ccccc3-c3ccccc3)cc21 ; CC1(C)c2ccccc2-c2ccc(N(c3ccc(-c4ccccc4)cc3)c3cc(-c4c(-c5ccccc5)cccc4-c4ccccc4)cc(N(c4ccccc4)c4ccccc4)c3)cc21] 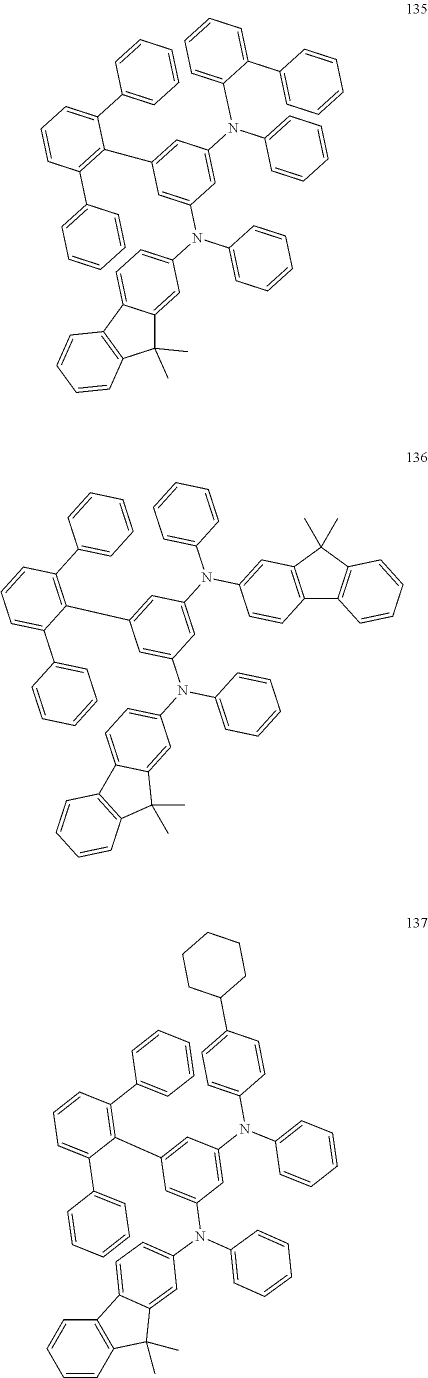 [SMILES: CC1(C)c2ccccc2-c2ccc(N(c3ccccc3)c3cc(-c4c(-c5ccccc5)cccc4-c4ccccc4)cc(N(c4ccccc4)c4ccccc4-c4ccccc4)c3)cc21 ; CC1(C)c2ccccc2-c2ccc(N(c3ccccc3)c3cc(-c4c(-c5ccccc5)cccc4-c4ccccc4)cc(N(c4ccccc4)c4ccc5c(c4)C(C)(C)c4ccccc4-5)c3)cc21 ; CC1(C)c2ccccc2-c2ccc(N(c3ccccc3)c3cc(-c4c(-c5ccccc5)cccc4-c4ccccc4)cc(N(c4ccccc4)c4ccc(C5CCCCC5)cc4)c3)cc21] 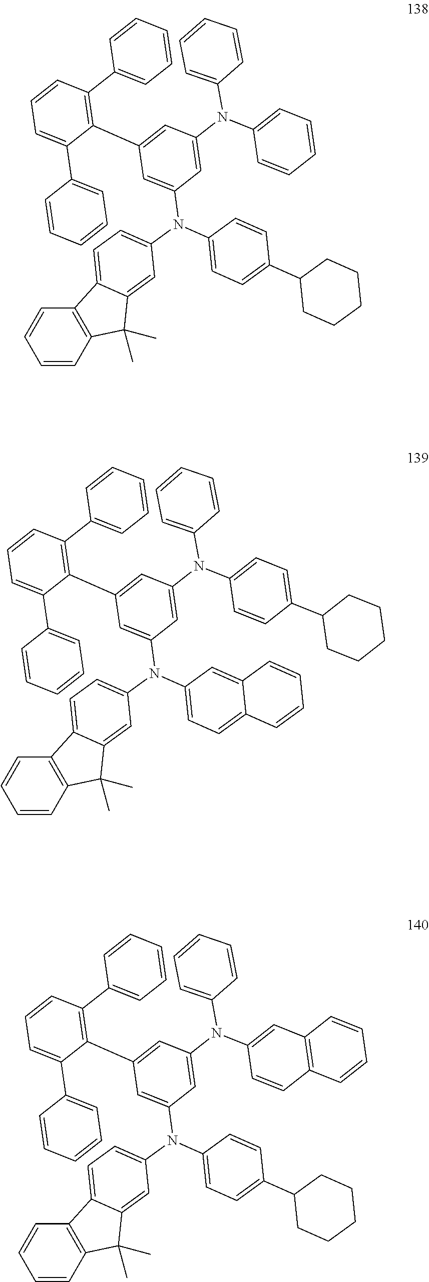 [SMILES: CC1(C)c2ccccc2-c2ccc(N(c3ccc(C4CCCCC4)cc3)c3cc(-c4c(-c5ccccc5)cccc4-c4ccccc4)cc(N(c4ccccc4)c4ccccc4)c3)cc21 ; CC1(C)c2ccccc2-c2ccc(N(c3cc(-c4c(-c5ccccc5)cccc4-c4ccccc4)cc(N(c4ccccc4)c4ccc(C5CCCCC5)cc4)c3)c3ccc4ccccc4c3)cc21 ; CC1(C)c2ccccc2-c2ccc(N(c3ccc(C4CCCCC4)cc3)c3cc(-c4c(-c5ccccc5)cccc4-c4ccccc4)cc(N(c4ccccc4)c4ccc5ccccc5c4)c3)cc21] 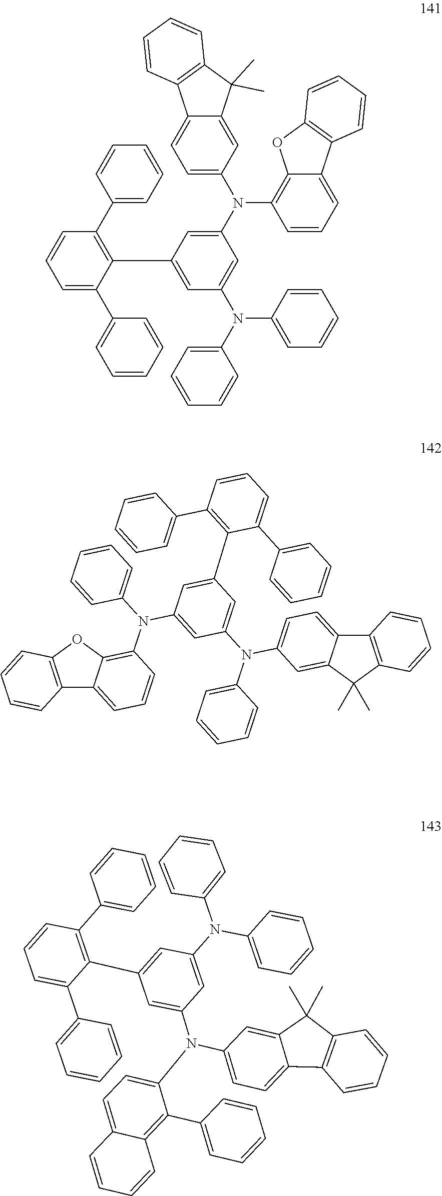 [SMILES: CC1(C)c2ccccc2-c2ccc(N(c3cc(-c4c(-c5ccccc5)cccc4-c4ccccc4)cc(N(c4ccccc4)c4ccccc4)c3)c3cccc4c3oc3ccccc34)cc21 ; CC1(C)c2ccccc2-c2ccc(N(c3ccccc3)c3cc(-c4c(-c5ccccc5)cccc4-c4ccccc4)cc(N(c4ccccc4)c4cccc5c4oc4ccccc45)c3)cc21 ; CC1(C)c2ccccc2-c2ccc(N(c3cc(-c4c(-c5ccccc5)cccc4-c4ccccc4)cc(N(c4ccccc4)c4ccccc4)c3)c3ccc4ccccc4c3-c3ccccc3)cc21] 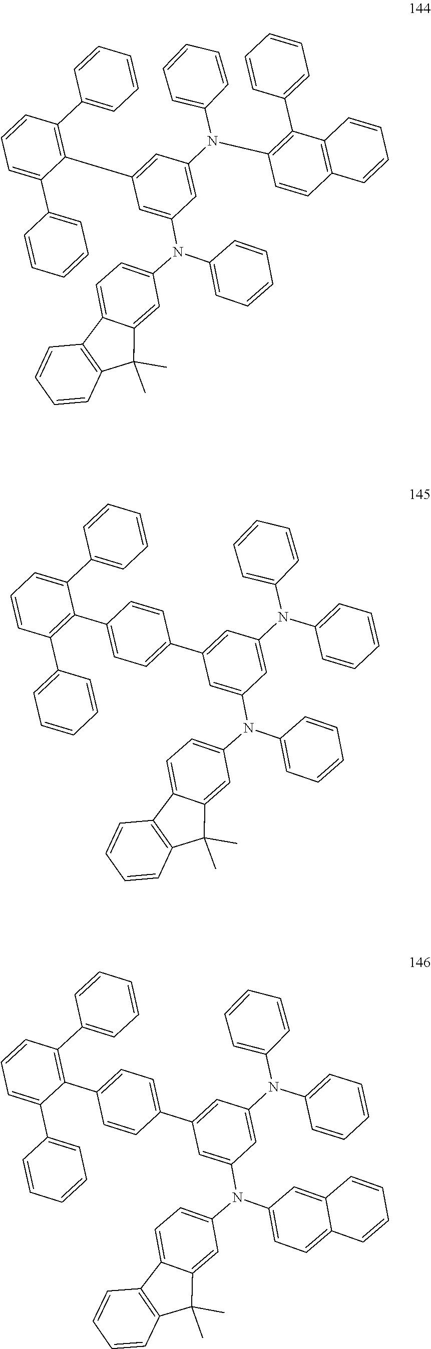 [SMILES: CC1(C)c2ccccc2-c2ccc(N(c3ccccc3)c3cc(-c4c(-c5ccccc5)cccc4-c4ccccc4)cc(N(c4ccccc4)c4ccc5ccccc5c4-c4ccccc4)c3)cc21 ; CC1(C)c2ccccc2-c2ccc(N(c3ccccc3)c3cc(-c4ccc(-c5c(-c6ccccc6)cccc5-c5ccccc5)cc4)cc(N(c4ccccc4)c4ccccc4)c3)cc21 ; CC1(C)c2ccccc2-c2ccc(N(c3cc(-c4ccc(-c5c(-c6ccccc6)cccc5-c5ccccc5)cc4)cc(N(c4ccccc4)c4ccccc4)c3)c3ccc4ccccc4c3)cc21] 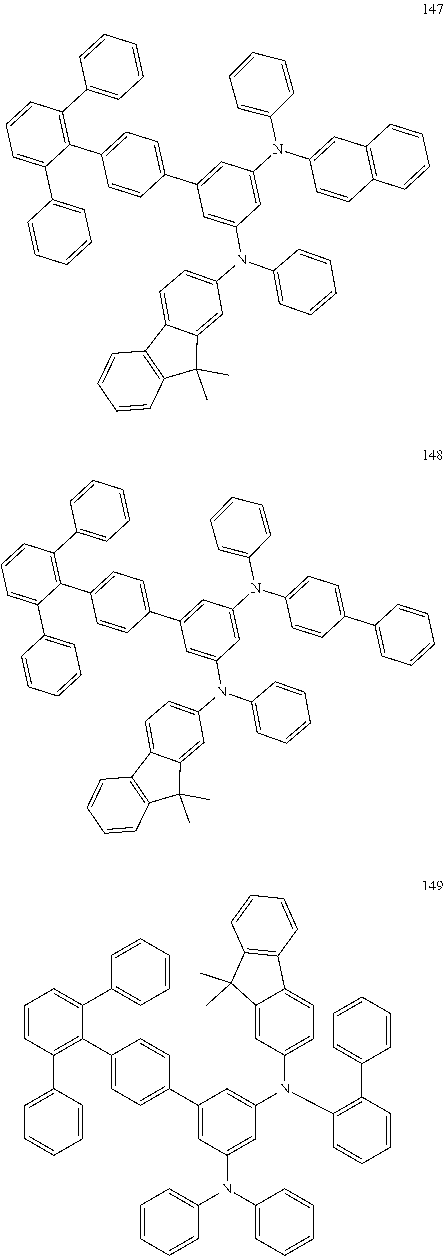 [SMILES: CC1(C)c2ccccc2-c2ccc(N(c3ccccc3)c3cc(-c4ccc(-c5c(-c6ccccc6)cccc5-c5ccccc5)cc4)cc(N(c4ccccc4)c4ccc5ccccc5c4)c3)cc21 ; CC1(C)c2ccccc2-c2ccc(N(c3ccccc3)c3cc(-c4ccc(-c5c(-c6ccccc6)cccc5-c5ccccc5)cc4)cc(N(c4ccccc4)c4ccc(-c5ccccc5)cc4)c3)cc21 ; CC1(C)c2ccccc2-c2ccc(N(c3cc(-c4ccc(-c5c(-c6ccccc6)cccc5-c5ccccc5)cc4)cc(N(c4ccccc4)c4ccccc4)c3)c3ccccc3-c3ccccc3)cc21] 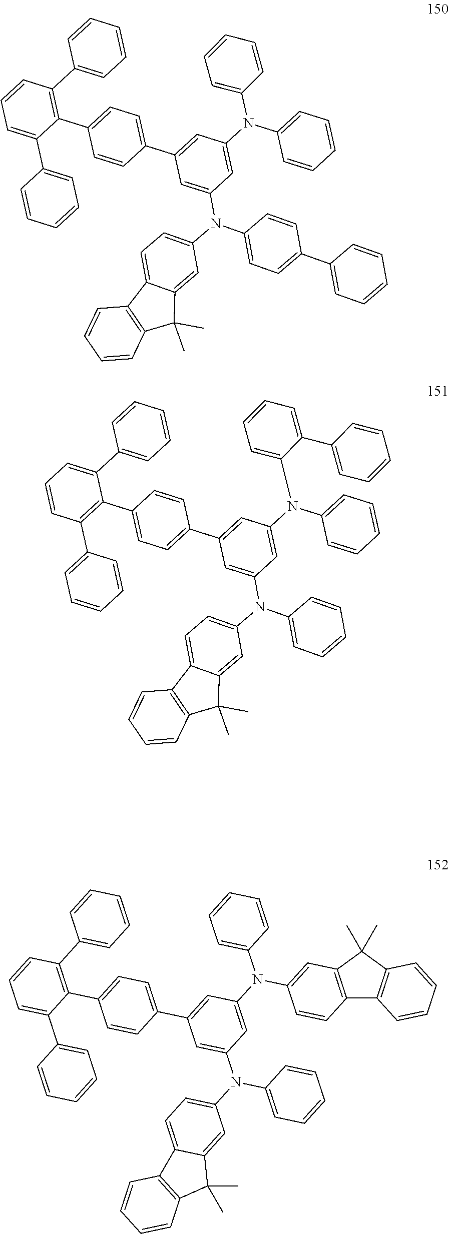 [SMILES: CC1(C)c2ccccc2-c2ccc(N(c3ccc(-c4ccccc4)cc3)c3cc(-c4ccc(-c5c(-c6ccccc6)cccc5-c5ccccc5)cc4)cc(N(c4ccccc4)c4ccccc4)c3)cc21 ; CC1(C)c2ccccc2-c2ccc(N(c3ccccc3)c3cc(-c4ccc(-c5c(-c6ccccc6)cccc5-c5ccccc5)cc4)cc(N(c4ccccc4)c4ccccc4-c4ccccc4)c3)cc21 ; CC1(C)c2ccccc2-c2ccc(N(c3ccccc3)c3cc(-c4ccc(-c5c(-c6ccccc6)cccc5-c5ccccc5)cc4)cc(N(c4ccccc4)c4ccc5c(c4)C(C)(C)c4ccccc4-5)c3)cc21] 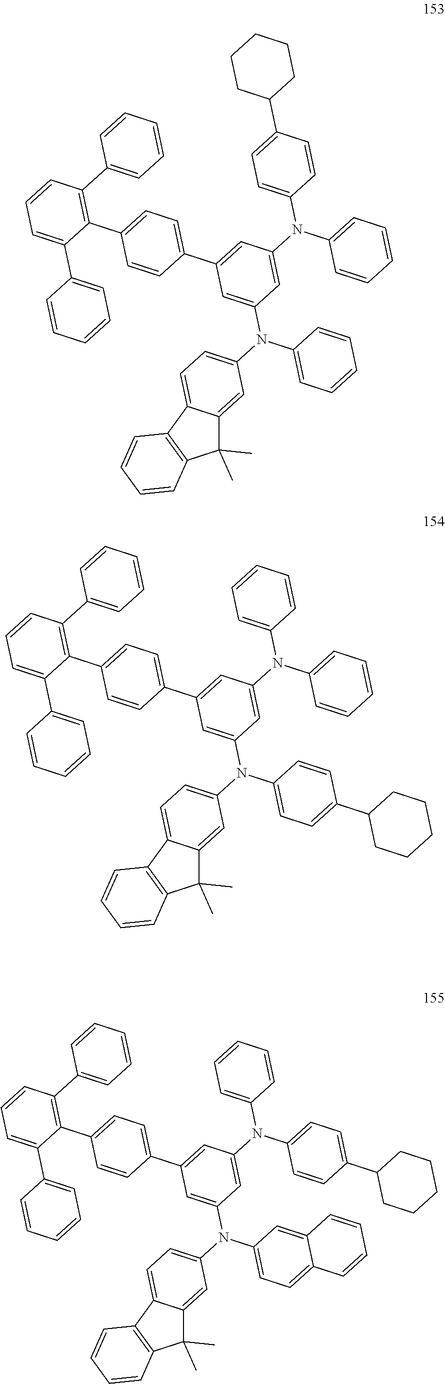 [SMILES: CC1(C)c2ccccc2-c2ccc(N(c3ccccc3)c3cc(-c4ccc(-c5c(-c6ccccc6)cccc5-c5ccccc5)cc4)cc(N(c4ccccc4)c4ccc(C5CCCCC5)cc4)c3)cc21 ; CC1(C)c2ccccc2-c2ccc(N(c3ccc(C4CCCCC4)cc3)c3cc(-c4ccc(-c5c(-c6ccccc6)cccc5-c5ccccc5)cc4)cc(N(c4ccccc4)c4ccccc4)c3)cc21 ; CC1(C)c2ccccc2-c2ccc(N(c3cc(-c4ccc(-c5c(-c6ccccc6)cccc5-c5ccccc5)cc4)cc(N(c4ccccc4)c4ccc(C5CCCCC5)cc4)c3)c3ccc4ccccc4c3)cc21] 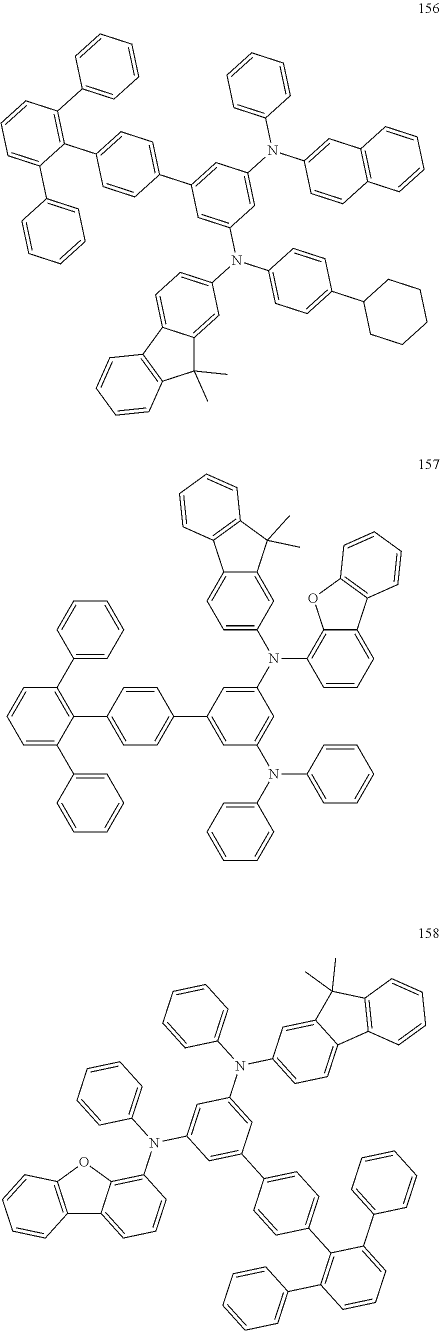 [SMILES: CC1(C)c2ccccc2-c2ccc(N(c3ccc(C4CCCCC4)cc3)c3cc(-c4ccc(-c5c(-c6ccccc6)cccc5-c5ccccc5)cc4)cc(N(c4ccccc4)c4ccc5ccccc5c4)c3)cc21 ; CC1(C)c2ccccc2-c2ccc(N(c3cc(-c4ccc(-c5c(-c6ccccc6)cccc5-c5ccccc5)cc4)cc(N(c4ccccc4)c4ccccc4)c3)c3cccc4c3oc3ccccc34)cc21 ; CC1(C)c2ccccc2-c2ccc(N(c3ccccc3)c3cc(-c4ccc(-c5c(-c6ccccc6)cccc5-c5ccccc5)cc4)cc(N(c4ccccc4)c4cccc5c4oc4ccccc45)c3)cc21] 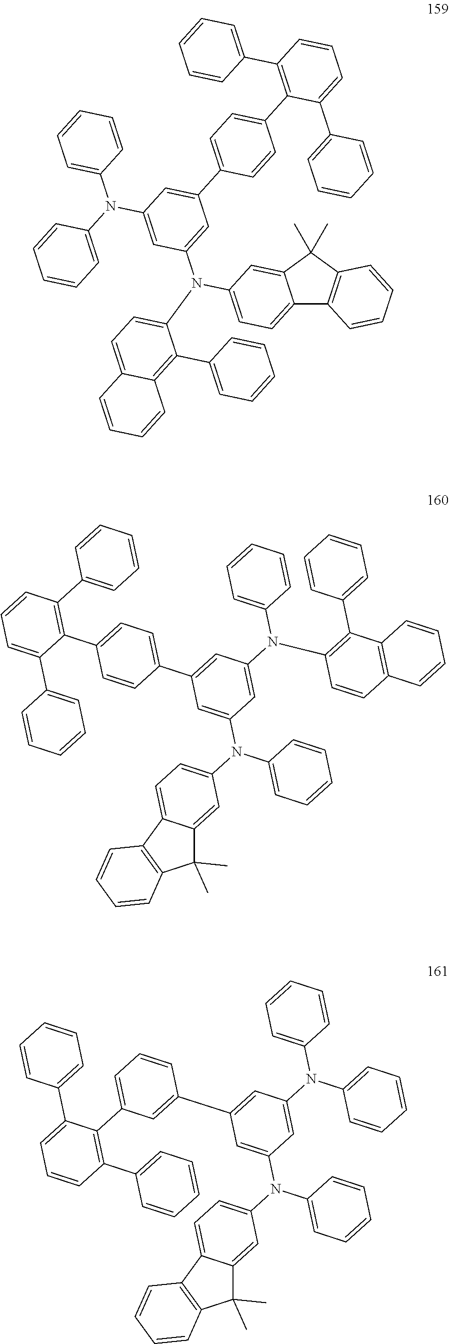 [SMILES: CC1(C)c2ccccc2-c2ccc(N(c3cc(-c4ccc(-c5c(-c6ccccc6)cccc5-c5ccccc5)cc4)cc(N(c4ccccc4)c4ccccc4)c3)c3ccc4ccccc4c3-c3ccccc3)cc21 ; CC1(C)c2ccccc2-c2ccc(N(c3ccccc3)c3cc(-c4ccc(-c5c(-c6ccccc6)cccc5-c5ccccc5)cc4)cc(N(c4ccccc4)c4ccc5ccccc5c4-c4ccccc4)c3)cc21 ; CC1(C)c2ccccc2-c2ccc(N(c3ccccc3)c3cc(-c4cccc(-c5c(-c6ccccc6)cccc5-c5ccccc5)c4)cc(N(c4ccccc4)c4ccccc4)c3)cc21] 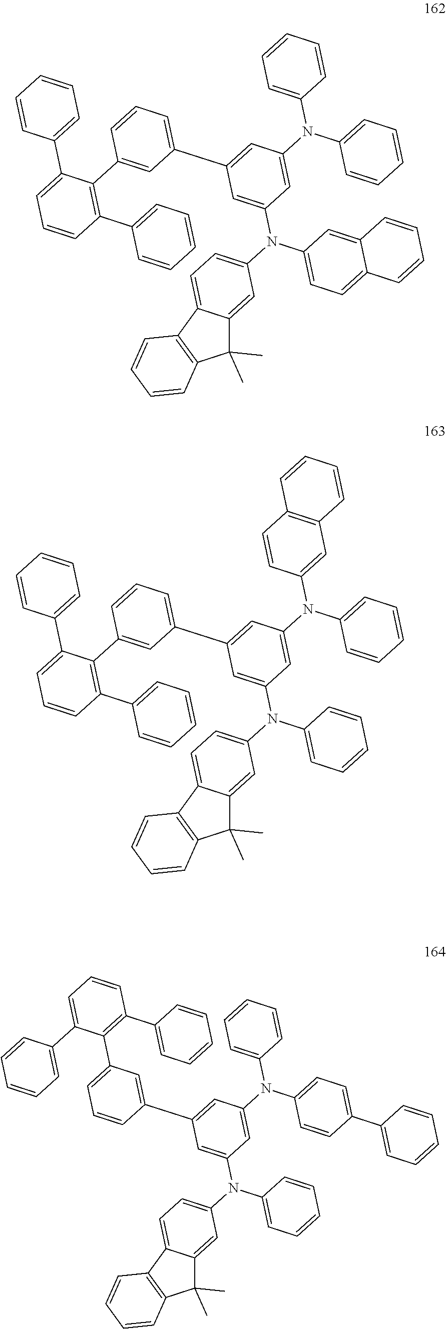 [SMILES: CC1(C)c2ccccc2-c2ccc(N(c3cc(-c4cccc(-c5c(-c6ccccc6)cccc5-c5ccccc5)c4)cc(N(c4ccccc4)c4ccccc4)c3)c3ccc4ccccc4c3)cc21 ; CC1(C)c2ccccc2-c2ccc(N(c3ccccc3)c3cc(-c4cccc(-c5c(-c6ccccc6)cccc5-c5ccccc5)c4)cc(N(c4ccccc4)c4ccc5ccccc5c4)c3)cc21 ; CC1(C)c2ccccc2-c2ccc(N(c3ccccc3)c3cc(-c4cccc(-c5c(-c6ccccc6)cccc5-c5ccccc5)c4)cc(N(c4ccccc4)c4ccc(-c5ccccc5)cc4)c3)cc21] 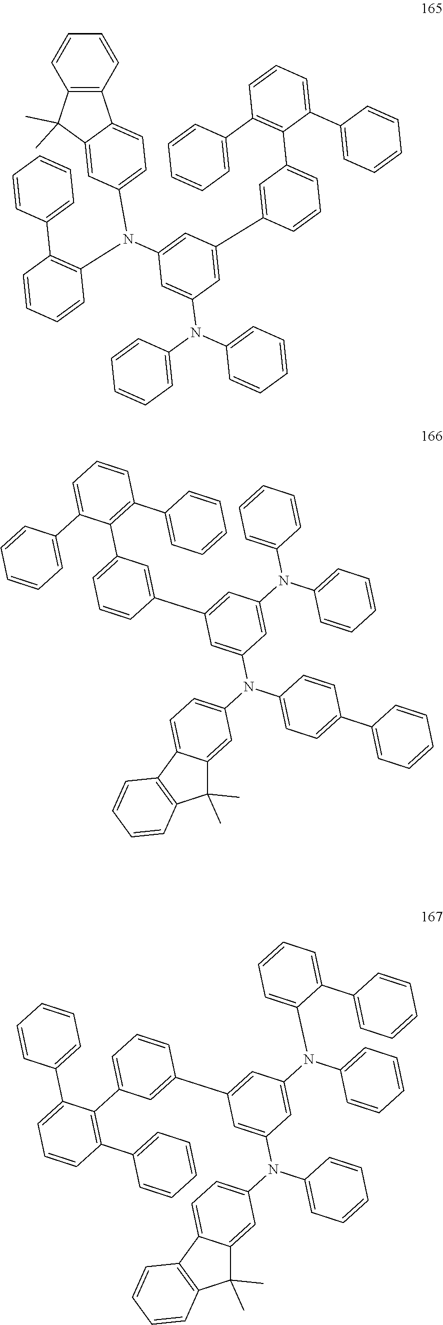 [SMILES: CC1(C)c2ccccc2-c2ccc(N(c3cc(-c4cccc(-c5c(-c6ccccc6)cccc5-c5ccccc5)c4)cc(N(c4ccccc4)c4ccccc4)c3)c3ccccc3-c3ccccc3)cc21 ; CC1(C)c2ccccc2-c2ccc(N(c3ccc(-c4ccccc4)cc3)c3cc(-c4cccc(-c5c(-c6ccccc6)cccc5-c5ccccc5)c4)cc(N(c4ccccc4)c4ccccc4)c3)cc21 ; CC1(C)c2ccccc2-c2ccc(N(c3ccccc3)c3cc(-c4cccc(-c5c(-c6ccccc6)cccc5-c5ccccc5)c4)cc(N(c4ccccc4)c4ccccc4-c4ccccc4)c3)cc21] 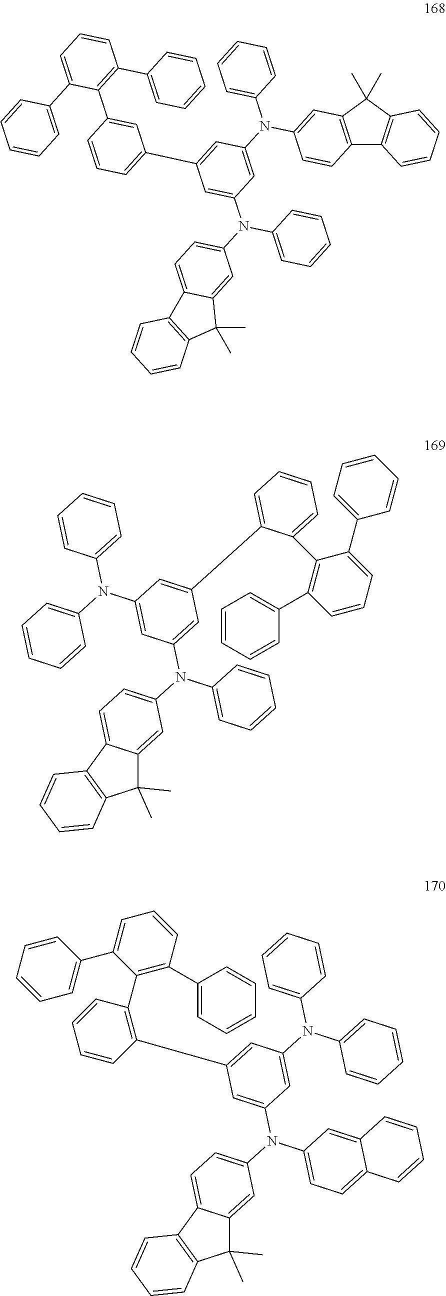 [SMILES: CC1(C)c2ccccc2-c2ccc(N(c3ccccc3)c3cc(-c4cccc(-c5c(-c6ccccc6)cccc5-c5ccccc5)c4)cc(N(c4ccccc4)c4ccc5c(c4)C(C)(C)c4ccccc4-5)c3)cc21 ; CC1(C)c2ccccc2-c2ccc(N(c3ccccc3)c3cc(-c4ccccc4-c4c(-c5ccccc5)cccc4-c4ccccc4)cc(N(c4ccccc4)c4ccccc4)c3)cc21 ; CC1(C)c2ccccc2-c2ccc(N(c3cc(-c4ccccc4-c4c(-c5ccccc5)cccc4-c4ccccc4)cc(N(c4ccccc4)c4ccccc4)c3)c3ccc4ccccc4c3)cc21] 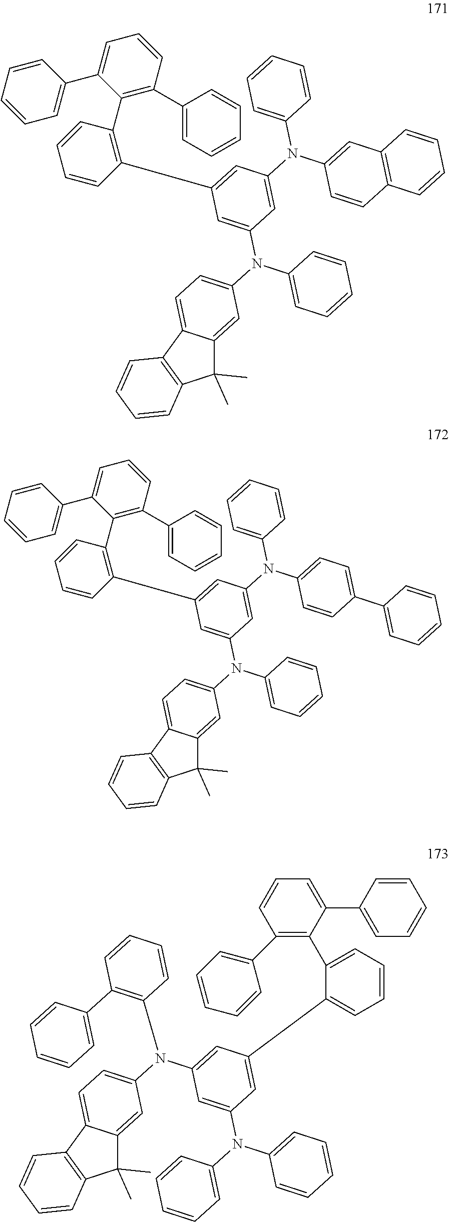 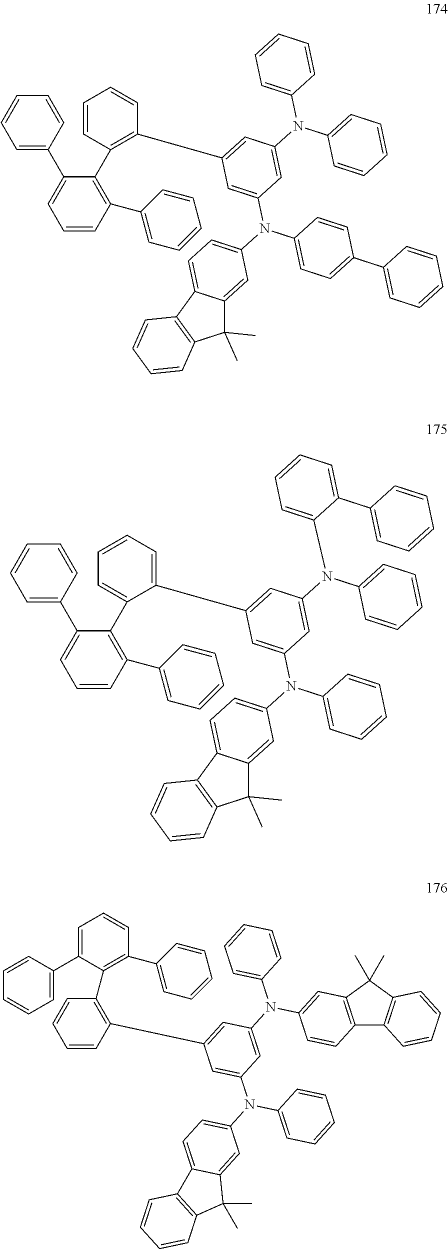 [SMILES: CC1(C)c2ccccc2-c2ccc(N(c3ccc(-c4ccccc4)cc3)c3cc(-c4ccccc4-c4c(-c5ccccc5)cccc4-c4ccccc4)cc(N(c4ccccc4)c4ccccc4)c3)cc21 ; CC1(C)c2ccccc2-c2ccc(N(c3ccccc3)c3cc(-c4ccccc4-c4c(-c5ccccc5)cccc4-c4ccccc4)cc(N(c4ccccc4)c4ccccc4-c4ccccc4)c3)cc21 ; CC1(C)c2ccccc2-c2ccc(N(c3ccccc3)c3cc(-c4ccccc4-c4c(-c5ccccc5)cccc4-c4ccccc4)cc(N(c4ccccc4)c4ccc5c(c4)C(C)(C)c4ccccc4-5)c3)cc21] 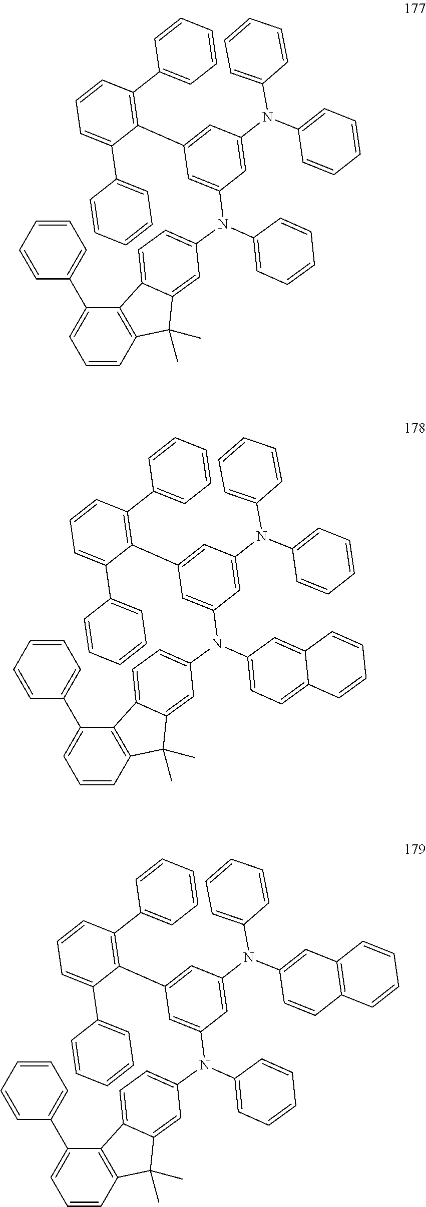 [SMILES: CC1(C)c2cc(N(c3ccccc3)c3cc(-c4c(-c5ccccc5)cccc4-c4ccccc4)cc(N(c4ccccc4)c4ccccc4)c3)ccc2-c2c(-c3ccccc3)cccc21 ; CC1(C)c2cc(N(c3cc(-c4c(-c5ccccc5)cccc4-c4ccccc4)cc(N(c4ccccc4)c4ccccc4)c3)c3ccc4ccccc4c3)ccc2-c2c(-c3ccccc3)cccc21 ; CC1(C)c2cc(N(c3ccccc3)c3cc(-c4c(-c5ccccc5)cccc4-c4ccccc4)cc(N(c4ccccc4)c4ccc5ccccc5c4)c3)ccc2-c2c(-c3ccccc3)cccc21] 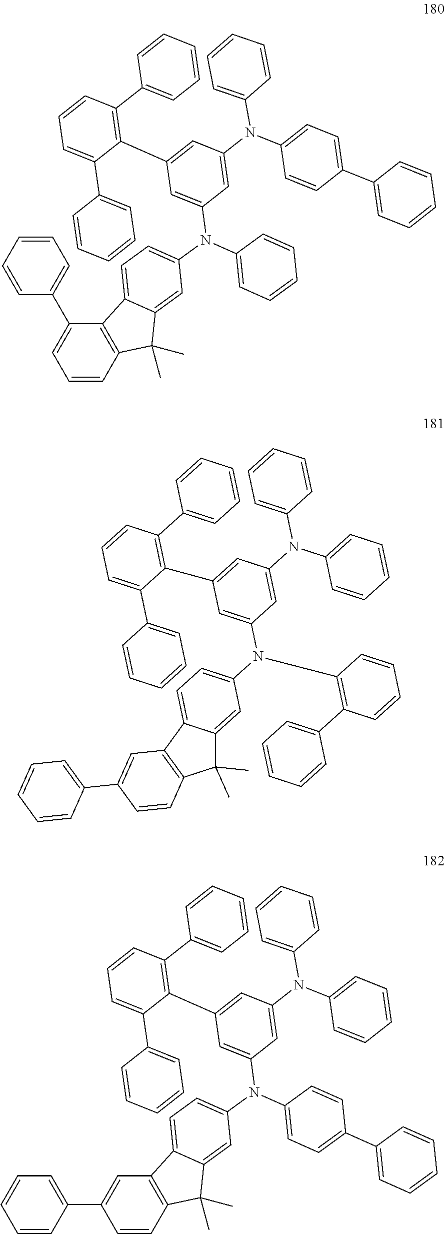 [SMILES: CC1(C)c2cc(N(c3ccccc3)c3cc(-c4c(-c5ccccc5)cccc4-c4ccccc4)cc(N(c4ccccc4)c4ccc(-c5ccccc5)cc4)c3)ccc2-c2c(-c3ccccc3)cccc21 ; CC1(C)c2ccc(-c3ccccc3)cc2-c2ccc(N(c3cc(-c4c(-c5ccccc5)cccc4-c4ccccc4)cc(N(c4ccccc4)c4ccccc4)c3)c3ccccc3-c3ccccc3)cc21 ; CC1(C)c2ccc(-c3ccccc3)cc2-c2ccc(N(c3ccc(-c4ccccc4)cc3)c3cc(-c4c(-c5ccccc5)cccc4-c4ccccc4)cc(N(c4ccccc4)c4ccccc4)c3)cc21] 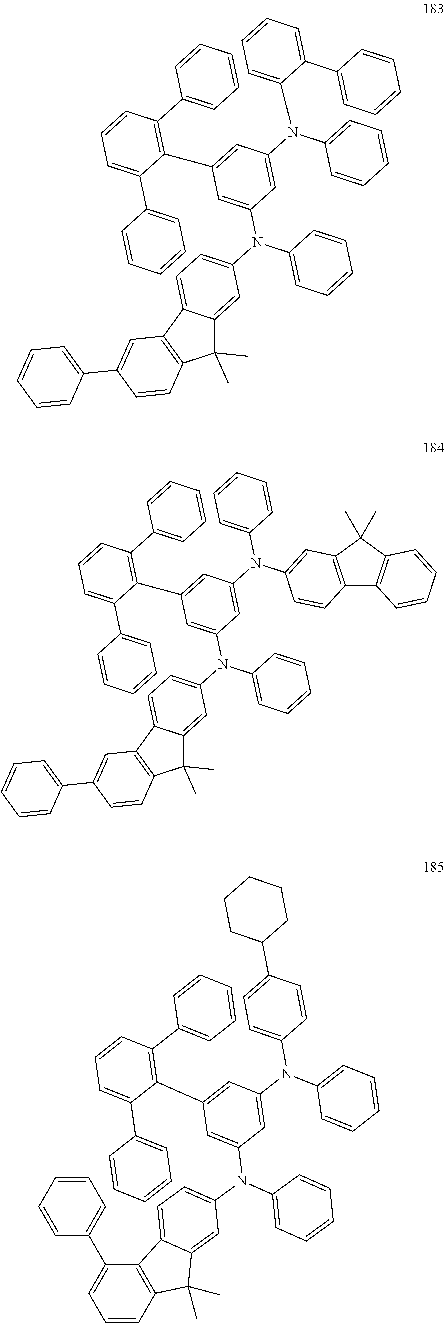 [SMILES: CC1(C)c2ccc(-c3ccccc3)cc2-c2ccc(N(c3ccccc3)c3cc(-c4c(-c5ccccc5)cccc4-c4ccccc4)cc(N(c4ccccc4)c4ccccc4-c4ccccc4)c3)cc21 ; CC1(C)c2ccccc2-c2ccc(N(c3ccccc3)c3cc(-c4c(-c5ccccc5)cccc4-c4ccccc4)cc(N(c4ccccc4)c4ccc5c(c4)C(C)(C)c4ccc(-c6ccccc6)cc4-5)c3)cc21 ; CC1(C)c2cc(N(c3ccccc3)c3cc(-c4c(-c5ccccc5)cccc4-c4ccccc4)cc(N(c4ccccc4)c4ccc(C5CCCCC5)cc4)c3)ccc2-c2c(-c3ccccc3)cccc21] 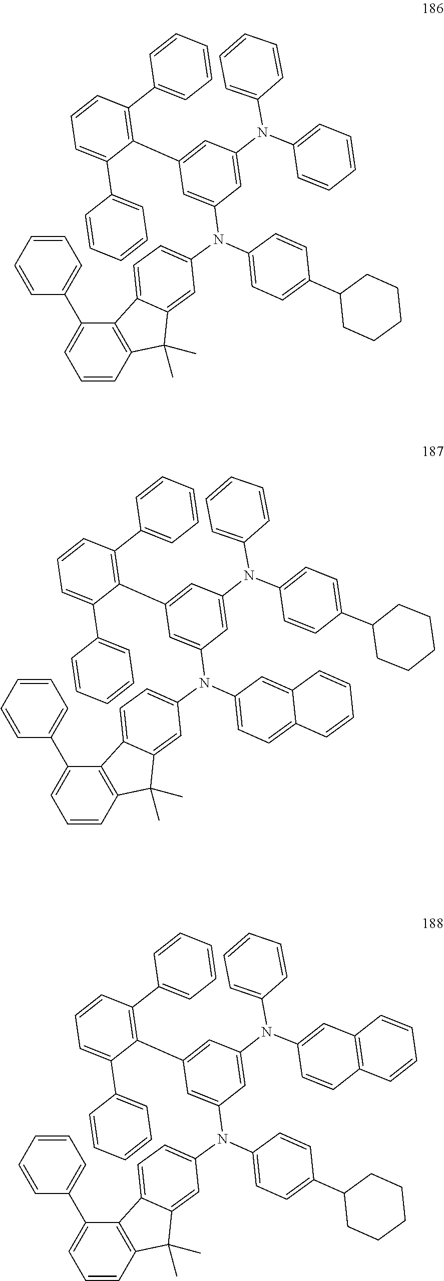 [SMILES: CC1(C)c2cc(N(c3ccc(C4CCCCC4)cc3)c3cc(-c4c(-c5ccccc5)cccc4-c4ccccc4)cc(N(c4ccccc4)c4ccccc4)c3)ccc2-c2c(-c3ccccc3)cccc21 ; CC1(C)c2cc(N(c3cc(-c4c(-c5ccccc5)cccc4-c4ccccc4)cc(N(c4ccccc4)c4ccc(C5CCCCC5)cc4)c3)c3ccc4ccccc4c3)ccc2-c2c(-c3ccccc3)cccc21 ; CC1(C)c2cc(N(c3ccc(C4CCCCC4)cc3)c3cc(-c4c(-c5ccccc5)cccc4-c4ccccc4)cc(N(c4ccccc4)c4ccc5ccccc5c4)c3)ccc2-c2c(-c3ccccc3)cccc21] 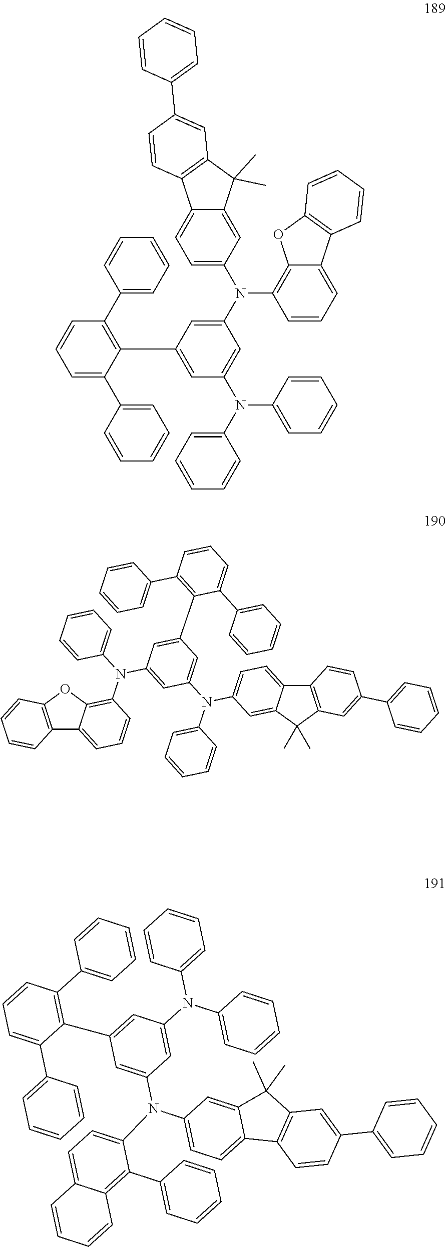 [SMILES: CC1(C)c2cc(-c3ccccc3)ccc2-c2ccc(N(c3cc(-c4c(-c5ccccc5)cccc4-c4ccccc4)cc(N(c4ccccc4)c4ccccc4)c3)c3cccc4c3oc3ccccc34)cc21 ; CC1(C)c2cc(-c3ccccc3)ccc2-c2ccc(N(c3ccccc3)c3cc(-c4c(-c5ccccc5)cccc4-c4ccccc4)cc(N(c4ccccc4)c4cccc5c4oc4ccccc45)c3)cc21 ; CC1(C)c2cc(-c3ccccc3)ccc2-c2ccc(N(c3cc(-c4c(-c5ccccc5)cccc4-c4ccccc4)cc(N(c4ccccc4)c4ccccc4)c3)c3ccc4ccccc4c3-c3ccccc3)cc21] 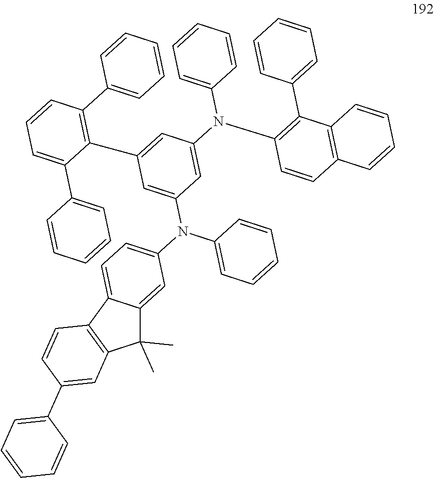 [SMILES: CC1(C)c2cc(-c3ccccc3)ccc2-c2ccc(N(c3ccccc3)c3cc(-c4c(-c5ccccc5)cccc4-c4ccccc4)cc(N(c4ccccc4)c4ccc5ccccc5c4-c4ccccc4)c3)cc21] 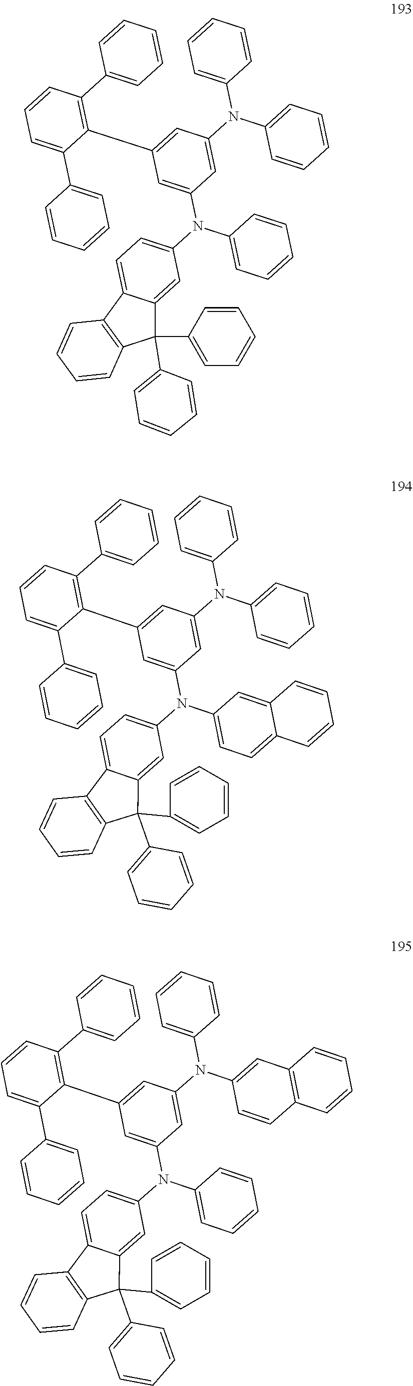 [SMILES: c1ccc(-c2cccc(-c3ccccc3)c2-c2cc(N(c3ccccc3)c3ccccc3)cc(N(c3ccccc3)c3ccc4c(c3)C(c3ccccc3)(c3ccccc3)c3ccccc3-4)c2)cc1 ; c1ccc(-c2cccc(-c3ccccc3)c2-c2cc(N(c3ccccc3)c3ccccc3)cc(N(c3ccc4c(c3)C(c3ccccc3)(c3ccccc3)c3ccccc3-4)c3ccc4ccccc4c3)c2)cc1 ; c1ccc(-c2cccc(-c3ccccc3)c2-c2cc(N(c3ccccc3)c3ccc4c(c3)C(c3ccccc3)(c3ccccc3)c3ccccc3-4)cc(N(c3ccccc3)c3ccc4ccccc4c3)c2)cc1] 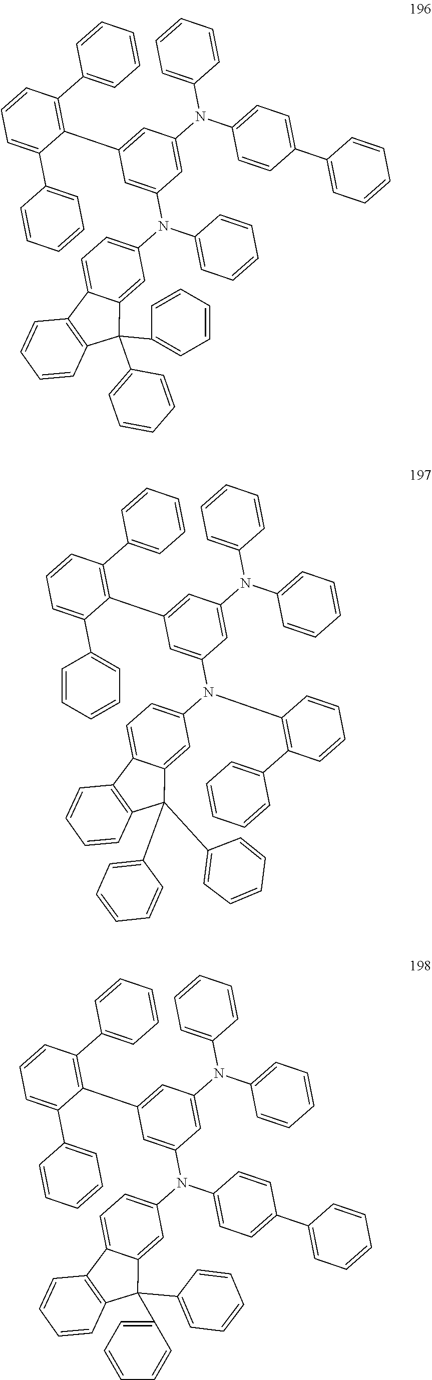 [SMILES: c1ccc(-c2ccc(N(c3ccccc3)c3cc(-c4c(-c5ccccc5)cccc4-c4ccccc4)cc(N(c4ccccc4)c4ccc5c(c4)C(c4ccccc4)(c4ccccc4)c4ccccc4-5)c3)cc2)cc1 ; c1ccc(-c2ccccc2N(c2cc(-c3c(-c4ccccc4)cccc3-c3ccccc3)cc(N(c3ccccc3)c3ccccc3)c2)c2ccc3c(c2)C(c2ccccc2)(c2ccccc2)c2ccccc2-3)cc1 ; c1ccc(-c2ccc(N(c3cc(-c4c(-c5ccccc5)cccc4-c4ccccc4)cc(N(c4ccccc4)c4ccccc4)c3)c3ccc4c(c3)C(c3ccccc3)(c3ccccc3)c3ccccc3-4)cc2)cc1] 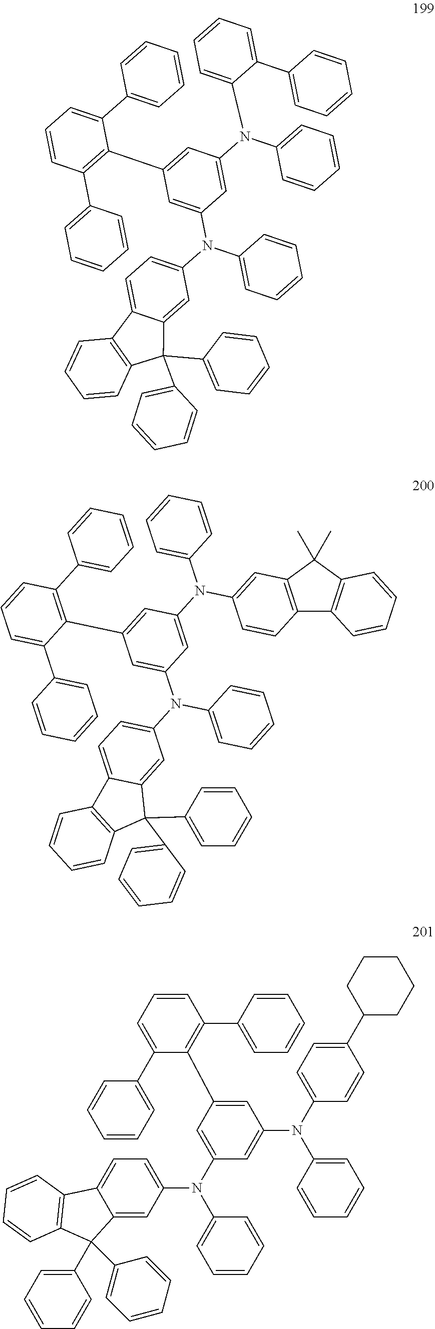 [SMILES: c1ccc(-c2ccccc2N(c2ccccc2)c2cc(-c3c(-c4ccccc4)cccc3-c3ccccc3)cc(N(c3ccccc3)c3ccc4c(c3)C(c3ccccc3)(c3ccccc3)c3ccccc3-4)c2)cc1 ; CC1(C)c2ccccc2-c2ccc(N(c3ccccc3)c3cc(-c4c(-c5ccccc5)cccc4-c4ccccc4)cc(N(c4ccccc4)c4ccc5c(c4)C(c4ccccc4)(c4ccccc4)c4ccccc4-5)c3)cc21 ; c1ccc(-c2cccc(-c3ccccc3)c2-c2cc(N(c3ccccc3)c3ccc(C4CCCCC4)cc3)cc(N(c3ccccc3)c3ccc4c(c3)C(c3ccccc3)(c3ccccc3)c3ccccc3-4)c2)cc1] 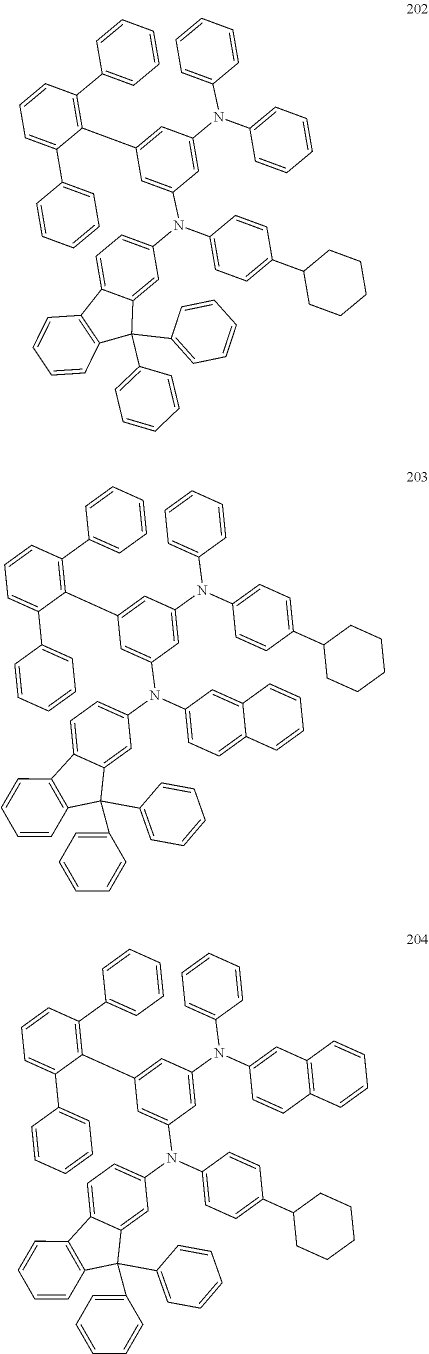 [SMILES: c1ccc(-c2cccc(-c3ccccc3)c2-c2cc(N(c3ccccc3)c3ccccc3)cc(N(c3ccc(C4CCCCC4)cc3)c3ccc4c(c3)C(c3ccccc3)(c3ccccc3)c3ccccc3-4)c2)cc1 ; c1ccc(-c2cccc(-c3ccccc3)c2-c2cc(N(c3ccccc3)c3ccc(C4CCCCC4)cc3)cc(N(c3ccc4c(c3)C(c3ccccc3)(c3ccccc3)c3ccccc3-4)c3ccc4ccccc4c3)c2)cc1 ; c1ccc(-c2cccc(-c3ccccc3)c2-c2cc(N(c3ccc(C4CCCCC4)cc3)c3ccc4c(c3)C(c3ccccc3)(c3ccccc3)c3ccccc3-4)cc(N(c3ccccc3)c3ccc4ccccc4c3)c2)cc1] 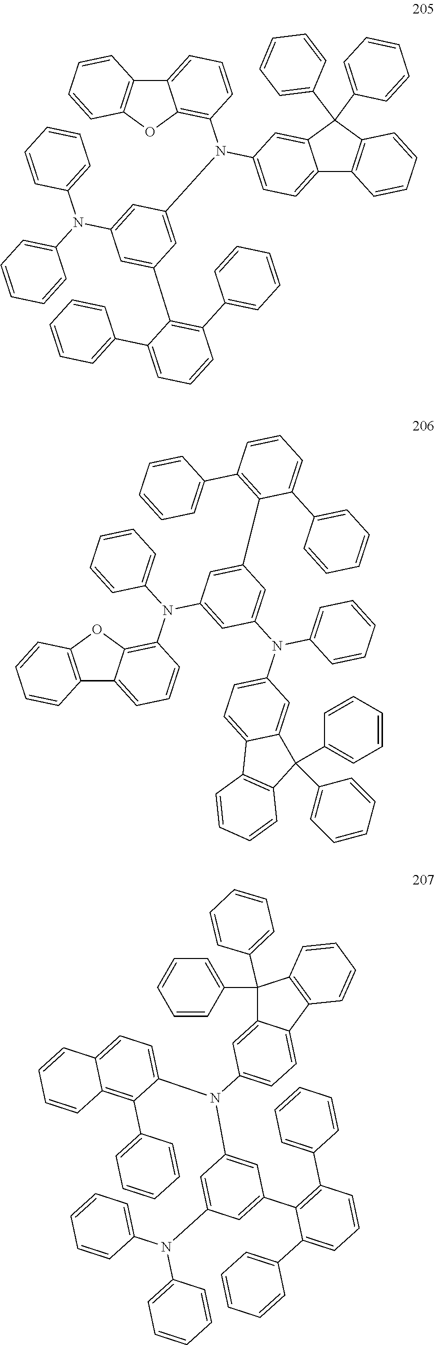 [SMILES: c1ccc(-c2cccc(-c3ccccc3)c2-c2cc(N(c3ccccc3)c3ccccc3)cc(N(c3ccc4c(c3)C(c3ccccc3)(c3ccccc3)c3ccccc3-4)c3cccc4c3oc3ccccc34)c2)cc1 ; c1ccc(-c2cccc(-c3ccccc3)c2-c2cc(N(c3ccccc3)c3ccc4c(c3)C(c3ccccc3)(c3ccccc3)c3ccccc3-4)cc(N(c3ccccc3)c3cccc4c3oc3ccccc34)c2)cc1 ; c1ccc(-c2cccc(-c3ccccc3)c2-c2cc(N(c3ccccc3)c3ccccc3)cc(N(c3ccc4c(c3)C(c3ccccc3)(c3ccccc3)c3ccccc3-4)c3ccc4ccccc4c3-c3ccccc3)c2)cc1] 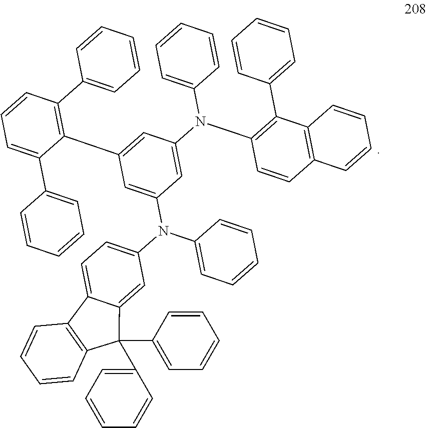 [SMILES: c1ccc(-c2cccc(-c3ccccc3)c2-c2cc(N(c3ccccc3)c3ccc4c(c3)C(c3ccccc3)(c3ccccc3)c3ccccc3-4)cc(N(c3ccccc3)c3ccc4ccccc4c3-c3ccccc3)c2)cc1]
